(19) United States
(12) Patent Application Publication
(10) Pub. No.: US 2022/0322647 A1
(43) Pub. Date: Oct. 13, 2022

(54) A METHOD OF GENERATING STERILE PROGENY

(71) Applicant: Center for Aquaculture Technologies, Inc., San Diego, CA (US)

(72) Inventors: Xavier Christophe Lauth, San Diego, CA (US); John Terrell Buchanan, San Diego, CA (US)

(73) Assignee: Center for Aquaculture Technologies, Inc., San Diego, CA (US)

(21) Appl. No.: 17/261,280

(22) PCT Filed: Jul. 19, 2019
(86) PCT No.: PCT/US2019/042543

§ 371 Date: Jan. 19, 2021

Related U.S. Application Data
(60) Provisional application No. 62/701,278, filed on Jul. 20, 2018.

(51) Int. Cl. A01K 67/027; A01K 67/033

(52) U.S. Cl. CPC A01K 67/0275; A01K 67/0334; A01K 67/0338; A01K 2227/40; A01K 2227/70; A01K 2217/058; A01K 2217/075; A01K 2217/15

(57) ABSTRACT

The disclosure provides a method of generating a sterile fish, crustacean, or mollusk. The method comprises breeding (i) a fertile hemizygous mutated female fish, crustacean, or mollusk with (ii) a fertile hemizygous mutated male fish, crustacean, or mollusk, selecting a female progenitor that is homozygous by genotypic selection, and breeding the homozygous female progenitor to produce the sterile fish, crustacean, or mollusk. The mutation disrupts the maternal-effect of a primordial germ cell (PGC) development gene and does not impair the viability, sex determination, fertility, or a combination thereof, of a homozygous progenitor. The disclosure also provides methods of making broodstock freshwater and seawater organisms for use in producing sterilized freshwater and seawater organisms, as well as the broodstock itself.

### STATEMENT OF GOVERNMENT RIGHTS

Aspects of the work described herein were supported by grant 2019-67030-29002 from the USDA-National Institute of Food and Agriculture. The United States Government may have certain rights in these inventions.

### FIELD

The present disclosure relates generally to methods of sterilizing freshwater and seawater organisms.

### BACKGROUND

The following paragraphs are not an admission that anything discussed in them is prior art or part of the knowledge of persons skilled in the art.

Facing decreasing yields from wild fisheries, global food supplies will have to rely more heavily on the food farming industry to fulfill an every-increasing public demand for seafood. In contrast to forms of animal agriculture, in aquaculture, many species sexually mature during production resulting in billions of dollars in lost productivity and downgraded product quality. Furthermore, farmed fish can escape and negatively impact aquatic ecosystems. As such, sterilization of farmed aquatic species is preferred for the aquaculture industry.

One approach for sterilizing fish is by induction of triploidy. The induction of triploidy is the most used and well-studied approach for producing sterile fish. Generally, triploid fish are produced by applying temperature or pressure shock to fertilized eggs, forcing the incorporation of the second polar body and producing cells with three chromosome sets (3N). Triploid fish do not develop normal gonads as the extra chromosome set disrupts meiosis. At the industrial scale, the logistics of reliably applying pressure or temperature shocks to batches of eggs is complicated and carries significant costs. An alternative to triploid induced by physical treatments is triploid induced by genetics, which results from crossing a tetraploid with a diploid fish. Tetraploid fish, however, are difficulty to generate due to poor embryonic survival and slow growth. In some examples, triploid males produce some normal haploid sperm cells thus allowing males to fertilize eggs, though at a reduced efficiency. Also, in some species, negative performance characteristics have been associated with triploid phenotype, including reduced growth and sensitivity to disease.

Another approach for sterilizing fish is by hormone treatment. However, in many cases, including intensive long-term treatments, such processes do not have a desirable efficacy of sterility, and/or has been associated with decreased fish growth performance. Furthermore, treatment involving a synthetic steroid may result in higher mortality rates.

Another approach for sterilizing fish is by transient silencing of genes governing germ line development, which includes a step of microinjecting antisense modified oligonucleotides into a single egg to ablate primordial germ cells. However, microinjecting eggs individually is not viable on a commercial scale.

Another approach for sterilizing fish is by using transgenic-based technologies, which include a step of integrating a transgene that induce germ cell death or disrupts their migration patterns resulting in their ablation in developing embryos. However, transgenes are subject to position effect as well as silencing. Consequently, such approaches are subject to extended regulatory review processes before being considered acceptable for commercial use.

Another approach for sterilizing fish is egg bathing treatment with a membrane permeable antisense oligonucleotide or small molecules inhibitor, which requires in vitro fertilization. However, handling eggs during the water-hardening process or early embryo development may impart mechanical, thermal, and/or chemical stresses, which may negatively affect the viability of the egg and/or embryo. Furthermore, hatcheries that are not equipped for egg bathing would incur an increase in production costs.

Improvements in generating sterile fish, crustaceans, or mollusks is desirable.

### INTRODUCTION

The following introduction is intended to introduce the reader to this specification but not to define any invention. One or more inventions may reside in a combination or sub-combination of the instrument elements or method steps described below or in other parts of this document. The inventors do not waive or disclaim their rights to any invention or inventions disclosed in this specification merely by not describing such other invention or inventions in the claims.

One or more of the previously proposed methods used for sterilizing freshwater and seawater organisms may result in: (1) an insufficient efficacy of sterilization, for example, by imparting mechanical, thermal, and/or chemical stresses on eggs and/or developing embryos; (2) an increase in operating costs by, for example, incorporating significant changes in husbandry practices, being untransferrable across multiple species, increasing production times, increasing the percentage of sterile organisms with reduced growth and increased sensitivity to disease, increasing mortality rates of sterile organisms, or a combination thereof; (3) gene flow to wild populations and colonization of new habitats by cultured, non-native species; (4) an insufficient efficiency of sterilization by, for example, inefficiently ablating primordial germ cells by microinjection; or (5) a combination thereof.

The present disclosure provides methods of producing sterilized freshwater and seawater organisms by disrupting their primordial germ cell development without impairing their ability to reach adult stage. One or more examples of the present disclosure may: (1) increase efficacy of sterilization by, for example, utilizing natural mating processes rather than in vitro fertilization; (2) decrease operating costs by, for example, decreasing the amount of costly equipment or treatments, being commercially scalable, being transferable across multiple species, decreasing feed, decreasing production times, increasing the percentage of organisms that achieve sexual maturity, increasing the physical size of sexually mature organisms, or a combination thereof; (3) decrease gene flow to wild populations and colonization of new habitats by cultured non-native species; (4) increase culture performance by, for example, decreasing loss of energy to gonad development; (5) increase efficiency of sterilization by, for example: a) decreasing or avoiding the incidence of position effect and silencing, and/or b) causing the creation of sterile progeny; or (6) a combination thereof, compared to one or more previously proposed methods used for sterilizing freshwater and seawater organisms.

The present disclosure also discusses methods of making broodstock freshwater and seawater organisms for use in producing sterilized freshwater and seawater organisms, as well as the broodstock itself.

The present disclosure provides a method of generating a sterile fish, crustacean, or mollusk. The method comprises the steps of: breeding (i) a fertile hemizygous mutated female fish, crustacean, or mollusk with (ii) a fertile hemizygous mutated male fish, crustacean, or mollusk, selecting a female progenitor that is homozygous by genotypic selection, and breeding the homozygous female progenitor to produce the sterile fish, crustacean, or mollusk. The mutation may disrupt the maternal-effect of a primordial germ cell (PGC) development gene and does not impair the viability, sex determination, fertility, or a combination thereof, of a homozygous progenitor.

The mutation may comprise: a mutation in a cis-acting 5′ or 3′ UTR regulatory sequence of the PGC development gene; a mutation in a gene encoding an RNA binding protein involved in the post-transcriptional regulation of the PGC development gene; a mutation in a gene involved in transport or formation of germ plasm; a mutation in a gene involved in germ cell specification, maintenance, or migration; or a combination thereof.

The gene encoding an RNA binding protein involved in the post-transcriptional regulation of the PGC development gene may be: Hnrnpab, Elavl1, Ptbp1a, Igf2bp3, Tia1, TIAR, Rbpms42, Rbpms24, KHSRP, or DHX9. The gene involved in transport or formation of germ plasm may encode a multi-tudor domain-containing protein, a kinesin-like protein, or an adaptor protein. The multi-tudor domain-containing protein may be Tdrd6. The adaptor protein may be hook2. The gene involved in germ cell specification, maintenance, or migration may be a gene expressing non-coding RNA. The non-coding RNA may be miR202-5p.

The mutation in a cis-acting 5′ or 3′ UTR regulatory sequence may disrupt the maternal activity of the PGC development gene, and does not disrupt the function of the PGC development gene during later stages of development. The PGC development gene may be nanos3, dnd1, or a piwi-like gene.

The present disclosure also provides a fertile homozygous mutated female fish, crustacean, or mollusk for producing a sterile fish, crustacean, or mollusk. The mutation disrupts the post-transcriptional regulation of a primordial germ cell (PGC) development gene to reduce the maternal-effect of the PGC development gene and does not impair somatic function of the gene.

The mutation may comprise: a mutation in a cis-acting 5′ or 3′ UTR regulatory sequence of the PGC development gene; a mutation in a gene encoding an RNA binding protein involved in the post-transcriptional regulation of the PGC development gene; a mutation in a gene involved in transport or formation of germ plasm; a mutation in a gene involved in germ cell specification, maintenance, or migration; or a combination thereof. The gene encoding an RNA binding protein involved in the post-transcriptional regulation of the PGC development gene may be: Hnrnpab, Elavl1, Ptbp1a, Igf2bp3, Tia1, TIAR, Rbpms42, Rbpms24, KHSRP, or DHX9. The gene involved in transport or formation of germ plasm may encode a multi-tudor domain-containing protein, a kinesin-like protein, or an adaptor protein. The multi-tudor domain-containing protein may be Tdrd6. The adaptor protein may be hook2. The gene involved in germ cell specification, maintenance, or migration may be a gene expressing non-coding RNA. The non-coding RNA may be miR202-5p. The mutation in a cis-acting 5′ or 3′ UTR regulatory sequence may disrupt the maternal activity of the PGC development gene, and does not disrupt the function of the PGC development gene during later stages of development. The PGC development gene may be nanos3, dnd1, or a piwi-like gene.

The present disclosure also provides a method of breeding a fertile homozygous mutated female fish, crustacean, or mollusk to generate a sterile fish, crustacean, or mollusk. The method comprises the steps of: breeding a fertile homozygous mutated female fish, crustacean, or mollusk with a wild-type male fish, crustacean, or mollusk, a hemizygous mutated male fish, crustacean, or mollusk, or a homozygous mutated male fish, crustacean, or mollusk to produce the sterile fish, crustacean, or mollusk. The mutation may disrupt the maternal-effect of a primordial germ cell (PGC) development gene and does not impair the viability, sex determination, fertility, or a combination thereof, of a homozygous progenitor.

The mutation may comprise: a mutation in a cis-acting 5′ or 3′ UTR regulatory sequence of the PGC development gene; a mutation in a gene encoding an RNA binding protein involved in the post-transcriptional regulation of the PGC development gene; a mutation in a gene involved in transport or formation of germ plasm; a mutation in a gene involved in germ cell specification, maintenance, or migration; or a combination thereof. The gene encoding an RNA binding protein involved in the post-transcriptional regulation of the PGC development gene may be: Hnrnpab, Elavl1, Ptbp1a, Igf2bp3, Tia1, TIAR, Rbpms42, Rbpms24, KHSRP, or DHX9. The gene involved in transport or formation of germ plasm may encode a multi-tudor domain-containing protein, a kinesin-like protein, or an adaptor protein. The multi-tudor domain-containing protein may be Tdrd6. The adaptor protein may be hook2. The gene involved in germ cell specification, maintenance, or migration may be a gene expressing non-coding RNA. The non-coding RNA may be miR202-5p.

The mutation in a cis-acting 5′ or 3′ UTR regulatory sequence may disrupt the maternal activity of the PGC development gene, and does not disrupt the function of the PGC development gene during later stages of development. The PGC development gene may be nanos3, dnd1, or a piwi-like gene.

The present disclosure also provides a method of making a fertile homozygous mutated female fish, crustacean, or mollusk that generates a sterile fish, crustacean, or mollusk. The method steps comprising: breeding (i) a fertile hemizygous mutated female fish, crustacean, or mollusk with (ii) a fertile hemizygous mutated male fish, crustacean, or mollusk or a homozygous mutated male fish male fish, crustacean, or mollusk, and selecting a female progenitor that is homozygous by genotypic selection. The mutation may disrupt the maternal-effect of a primordial germ cell (PGC) development gene and does not impair the viability, sex determination, fertility, or a combination thereof, of a homozygous progenitor.

The mutation may comprise: a mutation in a cis-acting 5′ or 3′ UTR regulatory sequence of the PGC development gene; a mutation in a gene encoding an RNA binding protein involved in the post-transcriptional regulation of the PGC development gene; a mutation in a gene involved in transport or formation of germ plasm; a mutation in a gene involved in germ cell specification, maintenance, or migration; or a combination thereof. The gene encoding an RNA binding protein involved in the post-transcriptional regulation of the PGC development gene may be: Hnrnpab, Elavl1, Ptbp1a, Igf2bp3, Tia1, TIAR, Rbpms42, Rbpms24, KHSRP, or DHX9. The gene involved in transport or formation of germ plasm may encode a multi-tudor domain-containing protein, a kinesin-like protein, or an adaptor protein. The multi-tudor domain-containing protein may be Tdrd6. The adaptor protein may be hook2. The gene involved in germ cell specification, maintenance, or migration may be a gene expressing non-coding RNA. The non-coding RNA may be miR202-5p.

The mutation in a cis-acting 5′ or 3′ UTR regulatory sequence may disrupt the maternal activity of the PGC development gene, and does not disrupt the function of the PGC development gene during later stages of development. The PGC development gene may be nanos3, dnd1, or a piwi-like gene.

Other aspects and features of the present disclosure will become apparent to those ordinarily skilled in the art upon review of the following description of specific examples in conjunction with the accompanying figures.

### BRIEF DESCRIPTION OF THE DRAWINGS

Examples of the presently disclosed methods and organisms will now be described, by way of example only, with reference to the attached Figures.

FIG. 1 is a flowchart illustrating an example of a method of generating a sterile fish, crustacean, or mollusk and propagating a mutated line.

FIGS. 2A and B are flowcharts illustrating an overview of the herein described mutagenesis strategy to identify maternal effect mutants affecting PGCs development and further propagation of the selected mutant alleles.

FIG. 3 panels A to D are photographs of different stages of growth of a Tilapia F0 generation comprising a double-allelic knockout.

FIG. 4 panels A and B are photographs of Tilapia after multi-gene targeting.

FIG. 5 panels A to C are representations and photographs of a stable transgenic line of tilapia expressing Green Fluorescent Protein (GFP) in primordial germ cells. Zpc5:eGFP:tnos 3′UTR construct: The tilapia Zpc5 promoter is an oocyte-specific promoter, active during oogenesis prior to the first meiotic division. As such, all embryos from a heterozygous transgenic female (FIG. 5 panel B) inherit the eGFP:tnos 3′UTR mRNA, which localizes and becomes expressed exclusively in PGCs through the action of cis-acting RNA elements in their 3′UTR (tilapia nanos 3′UTR) (FIG. 5 panel C).

FIG. 6 is an illustration of a process to introduce custom nucleotide changes to the DNA sequence. mHDR=microHomology-directed repair; HA=Homology arm. Scissor symbols represent target sites expected to be cleaved. This approach was used to edit the conserved motif in dnd1 3′UTR illustrated in FIG. 35.

FIG. 7 is illustrations and graphs illustrating F0 mosaic founder mutant identification and selection strategy. Mutant alleles were identified by fluorescence PCR with genes specific primers designed to amplify the regions around the targeted loci (120-300 bp). For fluorescent PCR, both combination of gene specific primers and two forward oligos with the fluorophore 6-FAM or NED attached were added to the reaction. A control reaction using wild type DNA is used to confirm the presence of single Peak amplification at each loci. The resulting amplicon were resolved via capillary electrophoresis (CE) with an added LIZ labeled size standard to determine the amplicon sizes accurate to base-pair resolution (Retrogen). The raw trace files were analyzed on Peak Scanner software (ThermoFisher). The size of the peak relative to the wild-type peak control determines the nature (insertion or deletion) and length of the mutation. The number of peaks indicate the level of mosaicism. We selected F0 mosaic founder carrying the fewest number of mutant alleles (2-4 peak preferentially).

FIG. 8 is a graph illustrating Melt Curve plot allows visualizing the genotypes of heterozygous, homozygous mutant and wild type samples. The negative change in fluorescence is plotted versus temperature (−dF/dT). Each trace represents a sample. The melting temperature of the wild-type allele in this example is ˜81° C. (wild type peak), the melting temperature of the homozygous mutant product (homozygous deletion peak) is ˜79° C. The remaining trace represents a heterozygote.

FIG. 9 panels A and B are illustrations of mutations at the nanos3 3′UTR loci. FIG. 9 panel A is a schematic of the nanos3 gene. Exon 1 is shown as the shaded box; translational start and stop sites as ATG and TAA, respectively. FIG. 9 panel B is the wild-type reference sequence and sequences of the seven germ-line mutant alleles from different offspring of nanos33′UTR mutated tilapia. Deletions and insertions are indicated by dashes and highlighted uppercase letters, respectively.

FIG. 10 is photographs of cranio-facial and tail deformities in the F3 homozygous KIF5B^{Δ1/Δ1} mutant. The arrows indicate skeletal deformities.

FIG. 11 panels A to D are graphs and photographs illustrating maternal effect sterility phenotype from TIAR, KSHRP, TIA1, DHX9, Igf2bp3, Elavl1, Elavl2, Cxcr4a, Ptbp1a, Hnrnpab, Rbm24, Rbm42, TDRD6, Hook2, miR-202-5p mutated F0 females. FIG. 11 panels A and B illustrate the average number of PGCs in 4-day old embryos (12 embryos) from F0 mutated females. There is a significant difference (p 0.01) comparing the embryos progeny from wild type control female. Vertical bars show standard deviation. FIG. 11 panel C represents 4 dpf tilapia embryo progeny of female transgenic line Tg (Zpc5: EGFP: nos 3′UTR) showing a normal PGC count. The GFP (+) germ cells (n=40) cluster longitudinally around anterior part of the gut. FIG. 11 panel D represents trunk regions of progenies from F0 Tg(Zpc5: EGFP: nos3 3′UTR) female lines carrying targeted gene mutations and showing different PGC count at 4 dpf (from n=1 to 15). The arrows are showing GFP (+) cells (green).

FIG. 12 panels A to H are photographs and graphs illustrating the maternal effect sterility phenotype in the progeny from F0 mutant females. FIG. 12 panel A shows the peritoneal cavity and atrophic testis (shown arrows) of 4 months old tilapia males' progeny (4 months old) from F0 female carrying mutation in nos3 3′UTR (right side) compared to aged match control testis. FIG. 12 panels B and C represent the average gonadosomatic index in F1 male progeny from F0 nos3 3′UTR mutated females (n=15/group). The mean±SD is shown. FIG. 12 panel D shows a dissected translucent testis from 6 months old F1 progeny of F0 nos3 3′UTR-mutated females. FIG. 12 panel E shows dissected gonads of F1 progenies derived from F0 female carrying mutations in TIA1. Progeny with low PGC count (<SPGC/embryo) developed translucid testes and atrophic ovaries at 6 months of age while F1 progeny with higher PGC count (>15 PGC/embryos) show ripe gonads. FIG. 12 panel F represents the average gonadosomatic index in F1 progeny with high or low PGC count. FIG. 12 panel G shows the peritoneal cavity of F1 females' progeny derived from F0 female (right side) or male (left side) carrying mutations in RBMS42. Arrows point to ovaries and white arrow point to an atrophic string like ovary. FIG. 12 panel H shows the peritoneal cavity of tilapia females' progeny from F0 female (lower side) or F0 male (upper side) carrying mutations in Ptbp1a.

FIG. 13 panels A to C are illustrations of selected nuclease-induced deletions at the KIF5Ba loci. FIG. 13 panel A is a schematic of the KIF5B gene. Exons (E1-25) are shown as shaded boxes; 5′ and 3′ untranslated regions are shown as open boxes. FIG. 13 panel B is the wild-type reference sequence (SEQ ID NO: 88) with the sequence of the selected germ-line mutant allele (SEQ ID NO: 89) from an offspring of KIF5B F0 mutated tilapia showing a 1 nt deletion (one dash in the sequence). This frameshift is predicted to create a truncated protein that terminates at amino acid 110 rather than position 962. FIG. 13 panel C is the predicted protein sequences of WT (SEQ ID NO: 90) and mutant KIF5B allele (SEQ ID NO: 91) in which the first 110 amino acids are identical to those of the wild-type TIAR protein.

FIG. 14 panels A to C are illustrations of selected mutant alleles at the TIAR loci. FIG. 14 panel A is a schematic of the TIAR gene. Exons (E1-12) are shown as shaded boxes, 5′ and 3′ untranslated regions are shown as open boxes; translational start and stop sites as ATG and TAA, respectively. FIG. 14 panel B is the wild-type reference sequence (SEQ ID NO: 92) with the selected germ-line mutant allele (SEQ ID NO: 93) from an offspring of TIAR F0 mutated tilapia. This 11 nt insertion is predicted to create a truncated protein that terminates at amino acid 119 rather than position 382. FIG. 14 panel C is the predicted protein sequences of WT (SEQ ID NO: 94) and mutant TIAR allele (SEQ ID NO: 95) in which the first 118 amino acids are identical to those of the wild-type TIAR protein with one following miscoded amino acid. Altered amino acids are highlighted.

FIG. 15 panels A to C are illustrations of selected mutant alleles at the KHSRP loci. FIG. 15 panel A is a schematic of the tilapia KHSRP gene. Exons (E1-22) are shown as shaded boxes, translational start and stop sites as ATG and TGA, respectively. Arrows point to targeted exons. FIG. 15 panel B shows the wild-type reference (SEQ ID NO: 96) and the selected mutant allele (SEQ ID NO: 97) from an offspring of KHSRP F0 mutant tilapia. Deletions are indicated by dashes. These consecutive deletions are predicted to create a truncated protein that terminates at amino acid 410 rather than position 695. FIG. 15 panel C is the predicted protein sequences of WT (SEQ ID NO: 98) and truncated mutant KHSRP protein (SEQ ID NO: 99) in which the first 387 amino acids are identical to those of the wild-type KSHRP protein and the following 23 amino acids are miscoded. Altered amino acids are highlighted.

FIG. 16 panels A to C are illustrations of selected mutations at the DHX9 loci. FIG. 16 panel A is a schematic of the tilapia DHX9 gene. Exons (E1-26) are shown as shaded boxes; 5′ and 3′ untranslated regions are shown as shaded boxes. Arrows point to targeted exons. FIG. 16 panel B is the wild-type reference sequence (SEQ ID NO: 100) with the sequence of the selected germ-line mutant allele (SEQ ID NO: 101) from an offspring of DHX9 F0 mutated tilapia. Location of the 7 nucleotide deletion is shown by dashes. This frameshift mutation is predicted to create a truncated protein that terminates at amino acid 82 rather than position 1286. FIG. 16 panel C shows the predicted protein sequences of WT (SEQ ID NO: 102) and truncated mutant DHX9 protein (SEQ ID NO: 103) in which the first 81 amino acids are identical to those of the wild-type DHX9 protein and the following amino acid is miscoded. Altered amino acids are highlighted.

FIG. 17 panels A to C are illustrations of selected mutation at the TIA1 loci. FIG. 17 panel A is a schematic of the tilapia Tia1 gene. Exons (E1-12) are shown as shaded boxes, 5′ and 3′ untranslated regions are shown as open boxes; translational start and stop sites as ATG and TAA, respectively. FIG. 17 panel B shows the wild-type reference sequence (SEQ ID NO: 104) and sequence of the selected germ-line mutant allele (SEQ ID NO: 105) from an offspring of Tia1 F0 mutated tilapia. The 10 nucleotide deletion is indicated by dashes in the sequence. This frameshift in the sequence is predicted to create a truncated protein that terminates at amino acid 27 rather than position 387. FIG. 17 panel C is the predicted protein sequences of WT (SEQ ID NO: 106) and truncated mutant TIA1 protein in which the first 15 amino acids are identical to those of the wild-type TIA1 protein (SEQ ID NO: 107) and the following 12 amino acids are miscoded. Altered amino acids are highlighted.

FIG. 18 panels A to C are illustrations of selected mutation at the Igf2pb3 loci. FIG. 18 panel A is a schematic of the tilapia Igf2pb3 gene. Exons (E1-15) are shown as shaded boxes. Arrows point to targeted exons. FIG. 18 panel B is the wild-type reference sequence (SEQ ID NO: 108) with the sequence of the selected germ-line mutant allele (SEQ ID NO: 109) from an offspring of Igf2bp3 F0 mutated tilapia. Inserted nucleotides are indicated in bold font and underlined. This frameshift is predicted to create a truncated protein that terminates at amino acid 206 rather than position 589. FIG. 18 panel C is the predicted protein sequences of WT (SEQ ID NO: 110) and truncated mutant protein (SEQ ID NO: 111) in which the first 173 amino acids are identical to those of the wild-type Igfpbp3 protein and the following 33 amino acids are miscoded. Altered amino acids are highlighted.

FIG. 19 panels A to C are illustrations of selected mutation at the Elavl1 loci. FIG. 19 panel A is a schematic of the tilapia Elavl1 gene. Exons (E1-7) are shown as shaded boxes; 5′ and 3′ untranslated regions are shown as open boxes. Arrows point to targeted exons. FIG. 19 panel B is the wild-type reference sequence (SEQ ID NO: 112) with the sequence of the selected germ-line mutant allele (SEQ ID NO: 113) from an offspring of Elavl1 F0 mutated tilapia. The 3 kb deletion is indicated by dashes. This frameshift is predicted to create a truncated protein that terminates at amino acid 105 rather than position 359. FIG. 19 panel C is the predicted protein sequences of WT (SEQ ID NO: 114) and truncated mutant protein (SEQ ID NO: 115) in which the first 45 amino acids are identical to those of the wild-type Elavl1 protein and the following 60 amino acids are miscoded. Altered amino acids are highlighted.

FIG. 20 panels A to C are illustrations of selected mutation at the Elavl2 loci. FIG. 20 panel A is a schematic of the tilapia Elavl2 gene. Exons (E1-7) are shown as shaded boxes. Arrows point to targeted exons. FIG. 20 panel B is the wild-type reference sequence (SEQ ID NO: 116) with the sequence of the selected germ-line mutant allele (SEQ ID NO: 117) from an offspring of Elavl2 F0 mutated tilapia. The 8 nucleotides deletion is indicated by dashes. This frameshift is predicted to create a truncated protein that terminates at amino acid 40 rather than position 372. FIG. 20 panel C is the predicted protein sequences of WT (SEQ ID NO: 118) and truncated mutant protein (SEQ ID NO: 119) in which the first 12 amino acids are identical to those of the wild-type Elavl2 protein and the following 28 amino acids are miscoded. Altered amino acids are highlighted.

FIG. 21 panels A to C are illustrations of the selected mutation at the Cxcr4a loci. FIG. 21 panel A is a schematic of the tilapia Cxcr4a gene. Exons (E1-2) are shown as shaded boxes; 5′ and 3′ untranslated regions are shown as open boxes. Arrows point to targeted exons. FIG. 21 panel B is the wild-type reference sequence (SEQ ID NO: 120) with the sequence of the selected germ-line mutant allele from an offspring of Cxcr4a F0 mutated tilapia (SEQ ID NO: 121). The 8 nucleotides deletion is indicated by dashes. This frameshift is predicted to create a truncated protein that terminates at amino acid 26 rather than position 372. FIG. 21 panel C is the predicted protein sequences of WT (SEQ ID NO: 122) and truncated mutant protein (SEQ ID NO: 123) in which the first 169 amino acids are identical to those of the wild-type CXCR4a protein and the following 8 amino acids are miscoded. Altered amino acids are highlighted.

FIG. 22 panels A to C are illustrations of the selected mutation at the Ptbp1a loci. FIG. 22 panel A is a schematic of the tilapia Ptbp1a gene. Exons (E1-16) are shown as shaded boxes. 5′ and 3′ untranslated regions are shown as open boxes. Arrows point to targeted exons. FIG. 22 panel B is the wild-type reference sequence (SEQ ID NO: 124) with the sequences of the selected germ-line mutant alleles from Ptbp1a F0 mutated tilapia (SEQ ID NOs: 125 and 126). The 13 nucleotides and 1.5 kb deletions are indicated by dashes. These frameshift mutations are predicted to create truncated proteins that terminate at amino acid 80 and 346 rather than position 538. FIG. 22 panel C is the predicted protein sequences of WT (SEQ ID NO: 127) and truncated mutant proteins (SEQ ID NOs: 128 and 129), in which the first 71 and 72 amino acids are identical to those of the wild-type Ptbp1a protein and the following 9 and 274 amino acids are miscoded. Altered amino acids are highlighted.

FIG. 23 panels A to C are illustrations of selected mutation at the nos3 loci. FIG. 23 panel A is a schematic of the tilapia nos3 gene. Exon (E1) is shown as a shaded box. Arrows point to targeted loci in exon1. FIG. 23 panel B is the wild-type reference sequence (SEQ ID NO: 130) with the sequence of the selected germ-line mutant allele from an offspring of nos3 F0 mutated tilapia (SEQ ID NO: 131). The 5 nucleotides deletion indicated by dashes is predicted to create a truncated protein that terminates at amino acid 145 rather than position 219. FIG. 23 panel C is the predicted protein sequences of WT (SEQ ID NO: 132) and truncated mutant protein (SEQ ID NO: 133) in which the first 140 amino acids are identical to those of the wild-type NANOS3 protein and the following 5 amino acids are miscoded. Altered amino acids are highlighted.

FIG. 24 panels A to C are illustrations of selected mutation at the dnd1 loci. FIG. 24 panel A is a schematic of the tilapia dnd1 gene. Exons (E1-E6) are shown as shaded boxes. 5′ and 3′ untranslated regions are shown as open boxes. Arrow point to targeted loci in exon6. FIG. 24 panel B is the wild-type reference sequence (SEQ ID NO: 134) with the sequence of the selected germ-line mutant allele from an offspring of dnd1 F0 mutated tilapia (SEQ ID NO: 135). The 5 nucleotides deletion indicated by dashes is predicted to create an elongated protein that terminates at amino acid 324 rather than position 320. FIG. 24 panel C is the predicted protein sequences of WT (SEQ ID NO: 136) and truncated mutant protein (SEQ ID NO: 137) in which the first 316 amino acids are identical to those of the wild-type DND1 protein and the following 8 amino acids are miscoded. Altered amino acids are highlighted.

FIG. 25 panels A to C are illustrations of selected mutation in the coding region of Hnrnpab. FIG. 25 panel A is a schematic of the tilapia Hnrnpab gene. Exon (E1-E7) are shown as shaded boxes. Arrows point to targeted loci. FIG. 25 panel B is the wild-type reference sequence (SEQ ID NO: 138) with the sequence of the selected germ-line mutant allele from an offspring of Hnrnpab F0 mutated tilapia (SEQ ID NO: 139). The 8 nucleotides deletion indicated by dashes is predicted to create a truncated protein that terminates at amino acid 29 rather than position 332. FIG. 25 panel C is the predicted protein sequences of WT (SEQ ID NO: 140) and truncated mutant protein (SEQ ID NO: 141) in which the first 27 amino acids are identical to those of the wild-type Hnrnpab protein and the following 2 amino acids are miscoded. Altered amino acids are highlighted.

FIG. 26 panels A to C are illustrations of selected mutation at the Hermes (Rbms) loci. FIG. 26 panel A is a schematic of the tilapia Hermes gene. Exon (E1-E6) are shown as shaded boxes. Arrows point to targeted loci. FIG. 26 panel B is the wild-type reference sequence (SEQ ID NO: 142) with the sequence of the selected germ-line mutant allele from an offspring of Hermes F0 mutated tilapia (SEQ ID NO: 143). The 16 nucleotides insertion indicated in bold font and underlined is predicted to create a truncated protein that terminates at amino acid 61 rather than position 174. FIG. 26 panel C is the predicted protein sequences of WT (SEQ ID NO: 144) and truncated mutant protein (SEQ ID NO: 145) in which the first 52 amino acids are identical to those of the wild-type Hermes protein and the following 9 amino acids are miscoded. Altered amino acids are highlighted.

FIG. 27 panels A to C are illustrations of selected mutation at the RBM24 loci. FIG. 27 panel A is a schematic of the tilapia RBM24 gene. Exon (E1-E4) are shown as shaded boxes. Arrows point to targeted loci. FIG. 27 panel B is the wild-type reference sequence (SEQ ID NO: 146) with the sequence of the selected germ-line mutant allele from an offspring of RBM42 F0 mutated tilapia (SEQ ID NO: 147). The 7 nucleotides deletion indicated by dashes is predicted to create a truncated protein that terminates at amino acid 54 rather than position 235. FIG. 27 panel C is the predicted protein sequences of WT (SEQ ID NO: 148) and truncated mutant protein (SEQ ID NO: 149) in which the first 42 amino acids are identical to those of the wild-type RBM24 protein and the following 12 amino acids are miscoded. Altered amino acids are highlighted.

FIG. 28 panels A to C are illustrations of selected mutation at the RBM42 loci. FIG. 28 panel A is a schematic of the tilapia RBM42 gene. Exon (E1-E11) are shown as shaded boxes. Arrows point to the targeted loci. FIG. 28 panel B is the wild-type reference sequence (SEQ ID NO: 150) with the sequence of the selected germ-line mutant allele from an offspring of RBM42 F0 mutated tilapia (SEQ ID NO: 151). The 7 nucleotides deletion indicated by dashes is predicted to create a truncated protein that terminates at amino acid 178 rather than position 408. FIG. 28 panel C is the predicted protein sequences of WT (SEQ ID NO: 152) and truncated mutant protein (SEQ ID NO: 153) in which the first 158 amino acids are identical to those of the wild-type RBM42 protein and the following 20 amino acids are miscoded. Altered amino acids are highlighted.

FIG. 29 panels A to C are illustrations of selected mutation at the TDRD6 loci. FIG. 29 panel A is a schematic of the tilapia TDRD6 gene. Exon (E1-E2) are shown as shaded boxes. Arrows point to targeted loci. FIG. 29 panel B is the wild-type reference sequence (SEQ ID NO: 154) with the sequence of the selected germ-line mutant allele from an offspring of TDRD6 F0 mutated tilapia (SEQ ID NO: 155). The 10 nucleotides deletion indicated by dashes is predicted to create a truncated protein that terminates at amino acid 43 rather than position 1630. FIG. 29 panel C is the predicted protein sequence of WT (SEQ ID NO: 156) and truncated mutant protein (SEQ ID NO: 157) in which the first 31 amino acids are identical to those of the wild-type TDRD6 protein and the following 12 amino acids are miscoded. Altered amino acids are highlighted.

FIG. 30 panels A to C are illustrations of selected mutation at the Hook2 loci. FIG. 30 panel A is a schematic of the tilapia Hook2 gene. Exons (E1-E22) are shown as shaded boxes. 5′ and 3′ untranslated regions are shown as open boxes. Arrows point to targeted loci. FIG. 30 panel B is the wild-type reference sequence (SEQ ID NO: 158) with the sequence of the selected germ-line mutant allele (SEQ ID NO: 159) from an offspring of Hook2 F0 mutated tilapia. The 2 nucleotides deletion indicated by dashes is predicted to create a truncated protein that terminates at amino acid 158 rather than position 708. FIG. 30 panel C is the predicted protein sequences of WT (SEQ ID NO: 160) and truncated mutant protein (SEQ ID NO: 161) in which the first 102 amino acids are identical to those of the wild-type Hook2 protein and the following 56 amino acids are miscoded. Altered amino acids are highlighted.

FIG. 31 panels A to C are illustrations of selected mutation at the miR-202 loci. FIG. 31 panel A shows the secondary structure tilapia (Oreochromis niloticus) pre miR-202 as projected from forna (force-directed RNA) RNA visualization tool (Kerpedjiev, Hammer et al. 2015). Arrows point to the position of the first and last nucleotides of two mature miR-202. FIG. 31 panel B shows the nucleotide sequence alignment of wild-type (SEQ ID NO: 162) and selected mutants (SEQ ID NOs: 163 to 165) with deletions indicated by dashes covering the miR-202-5p region. The miR-202-5p sequence is underlined once and the miR-202-3p sequence is underlined twice. The seed sequence of miR-202-5p is shown in doted box. FIG. 31 panel C shows secondary structure of pre miR-202 mutant alleles (miR-202^{Δ7/+}, miR-202^{Δ8/+}) from forna RNA visualization tool. Arrows indicate the first and last nucleotide of two mature miR-202.

FIG. 32 panels A to C are illustrations that show results of MEME analysis of varied teleost nos3 3′UTR. FIG. 32 panel A shows MEME block diagram with the distribution of conserved motifs in the 3′UTR of nos3 genes from varied teleost species (Olive Flounder (Paralichthys olivaceus) (SEQ ID NO: 166), channel catfish (Ictalurus punctatus) (SEQ ID NO: 170), rainbow trout (Oncorhynchus mykiss) (SEQ ID NO: 171), zebrafish (Danio rerio) (SEQ ID NO: 168), Nile tilapia (Oreochromis niloticus) (SEQ ID NO: 169), medaka (Oryzias latipes) (SEQ ID NO: 172), common carp (Cyprinus carpio) (SEQ ID NO: 167), fugu (tetraodon) (SEQ ID NO: 173). The 3′UTR were drawn in scale. Conserved motifs 1 (17-nt long) and 2 (40-nt long) are indicated in black and gray boxes, respectively. FIG. 32 panel B shows a sequence of the 17-nt long logos showing the top conserved motifs identified by the MEME tool. Height of the letters specifies the probability of appearing at the position in the motif. Primary sequence Alignment in block format showing sequence name, strand (+), SEQ ID #, starting nucleotide position and P-value Site (sites sorted by position p-value). FIG. 32 panel C shows a sequence of the 40-nt long logos showing the top conserved motifs identified by the MEME tool. Height of the letters specifies the probability of appearing at the position in the motif. Primary sequence Alignment in block format showing sequence name, strand (+) starting nucleotide position and P-value Site (sites sorted by position p-value).

FIG. 33 panels A and B are illustrations of selected nuclease-induced deletions in the conserved 19-nt motif1 of the tilapia nos33′UTR. FIG. 33 panel A is the wild-type reference sequence (SEQ ID NO: 169) with the sequences of two selected germ-line mutant alleles (8 nt and 32 nt-long deletions, SEQ ID NOs: 188 and 189, respectively) from an offspring of nos3 3′UTR F0 mutated tilapia. The deletions indicated by dashes are predicted to partially or completely remove the 17-nt long conserved motif1 identified by MEME (as shown in FIG. 32). The miR-430 putative target sequence GCACUU (Giraldez, Mishima et al. 2006) is shown in the doted box. FIG. 33 panel B shows the predicted secondary structure of the conserved motif1 from forna RNA visualization tool (Kerpedjiev, Hammer et al. 2015). Arrows point to the first and last nucleotide of motif1.

FIG. 34 panels A to C are illustrations that show results of MEME analysis of varied teleost dnd1 3′UTR. FIG. 34 panel A shows MEME block diagram showing the distribution of conserved motifs in the 3′UTR of dnd1 gene from varied species from fish to frog (Atlantic salmon (Salmo salar) (SEQ ID NO: 174), Atlantic cod (Gadus morhua) (SEQ ID NO: 175), rainbow trout (Oncorhynchus mykiss) (SEQ ID NO: 176), Nile tilapia (Oreochromis niloticus) (SEQ ID NO: 177), fugu (Takifugu rubripes) (SEQ ID NO: 178), zebrafish (Danio rerio) (SEQ ID NO: 179), Channel catfish (Ictalurus punctatus) (SEQ ID NO: 180), Xenope (Xenopus tropicalis) (SEQ ID NO: 181)). The 3′UTR were drawn in scale. Conserved motifs 1 and 2 are indicated in black and gray boxes, respectively. FIG. 34 panels B and C show the sequences of the 19-nt and 46-nt long logos corresponding to the two top conserved motifs identified by the MEME tool. Height of the letters specifies the probability of appearing at the position in the motif. Primary sequences alignment in block format showing sequence name, strand (+) Starting nucleotide position and P-value Site (sites sorted by position p-value).

FIG. 35 panels A and B are illustrations of the selected nuclease-induced nucleotide substitutions in the conserved 19-nt motif1 of the tilapia dnd1 3′UTR. FIG. 35 panel A is the wild-type reference sequence (SEQ ID NO: 177) with the sequences of the conserved dnd1 19 nt-motif1 sequence highlighted in a black box and its predicted minimum free energy (MFE) secondary structure from forna RNA visualization tool (Kerpedjiev, Hammer et al. 2015). The miR-23d putative target sequence AGTGATT (MIMAT0043480) (Eshel, Shirak et al. 2014) is shown in the doted box. FIG. 35 panel B is the edited sequence after allelic replacement (method described in FIG. 6) with substitution of the most conserved motif1-nucleotides (SEQ ID NO: 190). The RNAfold web server does not predict a secondary structure in the edited dnd1 motif1 (forna RNA visualization tool (Kerpedjiev, Hammer et al. 2015)).

FIG. 36 panels A to C are illustrations that show results of MEME analysis of varied teleost Elavl23′UTR. FIG. 36 panel A shows MEME block diagram showing the distribution of conserved motifs in the 3′UTR of Elavl2 genes from varied species from fish to frog (zebrafish (Danio rerio) (SEQ ID NO: 184), Catfish (Ictalurus punctatus) (SEQ ID NO: 185), Nile tilapia (Oreochromis niloticus) (SEQ ID NO: 183), medaka (Oryzias latipes) (SEQ ID NO: 186), Atlantic salmon (Salmo salar) (SEQ ID NO: 182), Xenope (Xenopus tropicalis) (SEQ ID NO: 187)). The 3′UTR were drawn with accurate proportions. Conserved motifs 1 and 2 are indicated in black and gray boxes, respectively. FIG. 36 panels B and C show sequences of the 30-nt long logos of conserved motifs 1 and 2 identified by the MEME tool. Height of the letters specifies the probability of appearing at the position in the motif. Primary sequence Alignment in block format showing: Sequence name, Strand (+), SEQ ID #, Starting nucleotide position and P-value Site (sites sorted by position p-value).

FIG. 37 panels A and B are graphs illustrating statistical analysis of PGC numbers in the progeny from TIAR, KSHRP, TIA1, DHX9, Igf2bp3, Elavl1, Elavl2, Cxcr4a, Ptbp1a, Hnrnpab, Rbm24, Rbm42, TDRD6, Hook2, miR-202-5p mutant F1 females. Columns show the average number of PGCs in 4 days old embryos (12 embryos) from individual F0 mutated females. There is a very significant difference (p 0.01) in comparison to the wild type control female progeny for all groups tested except for KHSRP and Elavl1. Vertical bars show standard deviation.

FIG. 38 panels A and B are illustrations and a photograph showing the generation, genotypes and associated phenotypes of the selected tilapia dnd1 mutant. FIG. 38 panel A: Dnd mutants were produced by microinjecting of engineered nucleases targeting dnd1 coding sequence into the blastodisc of tilapia embryos before the cell-cleavage stage. One of the resulting founder males was mated with a wild-type female, and produced heterozygous mutants in the F1 generation. Mating of these F1 mutants Dnd^{Δ4/+} produced an F2 generation with approximately 25% of the clutch being homozygous mutant (dnd-knockout Dnd^{Δ5/Δ5}) male, and lacking germ cells (as confirmed by analyses of dissected gonads). FIG. 38 panel B: Morphology of the male gonad in 1 yo (411 gr) dnd-knockout Dnd^{Δ5/Δ5} showing translucid testicular anatomy with normal size testis.

FIG. 39 panels A and B are illustrations and a photograph showing the generation, genotypes and associated phenotypes of the selected tilapia nos3 mutant. FIG. 39 panel A: Nos3 mutants were produced by microinjecting of engineered nucleases targeting nos3 coding sequence into the blastodisc of tilapia embryos before the cell-cleavage stage. One of the resulting founder males was mated with a wild-type female, and produced heterozygous mutants in the F1 generation. Mating of these F1 mutants nos3^{Δ5/+} produced an F2 generation with approximately 25% of the clutch being homozygous mutant (nos3-knockout nos3^{Δ5/Δ5}) of both sexes, with females lacking germ cells (as confirmed by analyses of dissected gonads). FIG. 39 panel B: Morphology of the male gonad in nos3-knockout nos3^{Δ5/Δ5} showing string like ovaries when compare to hemizygous sibling nos3^{Δ5/+}.

FIG. 40 panels A and B are illustrations and a photograph showing the generation, genotypes and associated phenotypes of selected tilapia Elavl2 mutation. FIG. 40 panel A: Elavl2 mutants were produced by microinjecting of engineered nucleases targeting Elavl2 coding sequence into the blastodisc of tilapia embryos before the cell-cleavage stage. One of the resulting founder males was mated with a wild-type female, and produced heterozygous mutants in the F1 generation. Mating of these F1 mutants Elavl2^{Δ8/+} produced an F2 generation with approximately 25% of the clutch being homozygous mutant (Elavl2-knockout Elavl2^{Δ8/Δ8}) of both sexes, with females lacking germ cells (as confirmed by analyses of dissected gonads). FIG. 40 panel B: Morphology of the male gonad in Elavl2-knockout Elavl2^{Δ8/Δ8} showing string like ovaries when compare to hemizygous sibling Elavl2^{Δ8/+}.

FIG. 41 panels A to D are illustrations and a photograph showing the dnd1 to β-globin 3′UTR swapping experiment. FIG. 41 panel A is a schematic of the tilapia dnd1 gene after targeted integration of β-globin 3′UTR. The primers (arrows) were used to confirm the integration of the 6-globin 3′UTR cassette into the tilapia genome. FIG. 41 panel B is a gel electrophoresis of gDNA PCR products from different treated fish. The 497 bp specific PCR amplicon in lanes 1, 3-5, 7 and 9-14 indicate successful integration of 3-globin 3′UTR downstream of the dnd1 (dead end1) open reading frame. FIG. 41 panel C shows translucid testes in the peritoneal cavity of a tilapia homozygous for this integration (DND1^{bglo 3′UTR/bglo3′UTR}) FIG. 41 panel D is a gel that indicates that vasa specific RT PCR amplicon are absent in the testes from DND1^{bglo 3′UTR/bglo3′UTR} tilapia.

FIG. 42 panels A and B are photographs and graphs showing the maternal effect sterility phenotypes in the progeny from nos3 3′UTR homozygous female (nos3 3′UTR^{Δ32/Δ32}). FIG. 42 panel A shows the dissected gonads in 6-month-old progeny with complete (transparent testis and string like ovaries) to partial sterility phenotypes in males and females. FIG. 42 panel B: Statistic analysis of PGC numbers in 4-day old embryos progeny of nos3 3′UTR^{Δ32/Δ32} females. The average PGC number (12 embryos/column) was reduced by 93% compare to control.

FIG. 43 panels A and B are photographs and graphs showing the maternal effect sterility phenotypes in the progeny from TIAR homozygous mutant female (TIAR^{-/-}). FIG. 43 panel A shows the dissected gonads in 6-month-old progeny with severe sterility phenotypes in male (left image showing peritoneal cavity) and female (right image showing peritoneal cavity). FIG. 43 panel B: Statistic analysis of gonadosomatic indexes in six-month-old progeny yielded by the TIAR mutant females. The average PGC number (12 embryos/column) was reduced by 93% compare to control.

FIG. 44 panels A to C are graphs showing the nature of the interactions of maternal effect mutations in two components system (epistasis). Using the additive assumption epistasis, the absence of epistasis in the double KO line is expected to be the sum of the effects of single KO. We measured the sum expectation of single KO with the formula: TPA=LA1+(1−LA1)×LA2 where LA1 is the level of PGC ablation from KO #1 and LA2 is the level of PGC ablation caused by KO #2. We calculated the Total PGCs Ablation level to be within a few percentage points away from the measured level of ablation, suggesting no epistasis. Calculated LA versus measured LA are shown in parenthesis below each graph.

FIG. 45 is a graph illustrating statistical analysis of PGC numbers in the progeny from TIAR, KSHRP, TIA1, DHX9, Elavl1, Cxcr4a, and nos3 3′UTR homozygous mutant F2 females. Columns show the average number of PGCs in 4-day old embryos (12 embryos) from individual F0 mutated females. There is a very significant difference (p 0.01) compared to the wild type control female progeny for all groups tested. Vertical bars show standard deviation.

### DETAILED DESCRIPTION

Generally, the present disclosure provides a method of generating a sterile fish, crustacean, or mollusk. The method comprises breeding (i) a fertile hemizygous mutated female fish, crustacean, or mollusk with (ii) a fertile hemizygous mutated male fish, crustacean, or mollusk, selecting a female progenitor that is homozygous by genotypic selection, and breeding the homozygous female progenitor to produce the sterile fish, crustacean, or mollusk. The mutation disrupts the maternal-effect of a primordial germ cell (PGC) development gene and does not impair the viability, sex determination, fertility, or a combination thereof, of a homozygous progenitor.

The present disclosure also provides a method of breeding a fertile homozygous mutated female fish, crustacean, or mollusk to generate a sterile fish, crustacean, or mollusk. The method comprises breeding a fertile homozygous mutated female fish, crustacean, or mollusk with a wild-type male fish, crustacean, or mollusk, a hemizygous mutated male fish, crustacean, or mollusk, or a homozygous mutated male fish, crustacean, or mollusk to produce the sterile fish, crustacean, or mollusk. The mutation disrupts the maternal-effect of a primordial germ cell (PGC) development gene and does not impair the viability, sex determination, fertility, or a combination thereof, of a homozygous progenitor.

The present disclosure further provides a method of making a fertile homozygous mutated female fish, crustacean, or mollusk that generates a sterile fish, crustacean, or mollusk. The method comprises breeding (i) a fertile hemizygous mutated female fish, crustacean, or mollusk with (ii) a fertile hemizygous mutated male fish, crustacean, or mollusk, or a homozygous mutated male fish male fish, crustacean, or mollusk, and selecting a female progenitor that is homozygous by genotypic selection. The mutation disrupts the maternal-effect of a primordial germ cell (PGC) development gene and does not impair the viability, sex determination, fertility, or a combination thereof, of a homozygous progenitor.

In the context of the present disclosure, a fish refers to any gill-bearing craniate animal that lacks limbs with digits. Examples of fish are carp, tilapia, salmon, trout, and catfish. In the context of the present disclosure, a crustacean refers to any arthropod taxon. Examples of crustaceans are crabs, lobsters, crayfish, and shrimp. In the context of the present disclosure, a mollusk refers to any invertebrate animal with a soft unsegmented body usually enclosed in a calcareous shell. Examples of mollusks are clams, scallops, oysters, octopus, squid and chitons. A hemizygous fish, crustacean, or mollusk refers to any diploid fish, crustacean, or mollusk that carries one copy of the chromosome containing the mutation but the matching chromosome does not have the mutation. A homozygous fish, crustacean, or mollusk refers to any diploid fish, crustacean, or mollusk that carries two copies of the chromosome containing the mutation.

A sterile fish, crustacean, or mollusk refers to any fish, crustacean, or mollusk with a diminished ability to generate progeny through breeding or crossing as compared to its wild-type counterpart; for example, a sterile fish, crustacean, or mollusk may have an about 50%, about 75%, about 90%, about 95%, or 100% reduced likelihood of producing progeny. In contrast, a fertile fish, crustacean, or mollusk refers to any fish, crustacean, or mollusk that possesses the ability to produce progeny through breeding or crossing. Breeding and crossing refer to any process in which a male species and a female species mate to produce progeny or offspring.

Maternal-effect refers to a situation where the phenotype of an organism is expected from the genotype of its mother due to the mother supplying RNA, proteins, or a combination thereof to the oocyte. Disrupting the maternal-effect of a PGC development gene refers to impairing or abolishing the function of one or a combination of genes that are maternally expressed in the oocyte and function in PGC development, maintenance, migration, or a combination thereof. The disruption of the one or combination of genes that are maternally expressed in the oocyte and function in PGC development, maintenance, migration, or a combination thereof does not impair or abolish a zygotic function of the one or combination of genes involved in the viability, sex determination, fertility, or a combination thereof of a homozygous progenitor carrying said impairment or abolishment gene function. Of note, disruption of the one or combination of genes that are maternally expressed in the oocyte and function in PGC development, maintenance, migration, or a combination thereof may impair or abolish the zygotic function of the one or combination of genes that are not involved in the viability, sex determination, fertility, or a combination thereof of a homozygous progenitor, for example, those involved in immunity, metabolism, stress or disease response. Disrupting the one or combination of genes that are maternally expressed in the oocyte and function in PGC development, maintenance, migration, or a combination thereof disrupts the formation of gametes and may result in sterile and sexually immature organisms.

Germ plasm genes have been subjected to knockout experiments resulting in their inactivation. However, after some germ plasm genes were knocked out, the expected phenotype was not observed and/or pleiotropic phenotypes were detected resulting in: 1) the development into defective fish that cannot breed to produce sterile progeny; or 2) a developed fish that produces non-viable progeny. Yet other germ plasm genes were knocked out resulting in a homozygous mutant having impaired development of the ovary, testis, or both and therefore cannot breed to produce a sterile progeny. The inventors have discovered that by introducing one or more specific mutations that affect PGC function without impairing or abolishing the ability of the mutated organism to develop into a sexually mature adult, i.e., does not impair their viability, sex determination, fertility, or a combination thereof, allows for the generation of a broodstock that can be used to produce sterile progeny. Importantly, the one or more specific mutations disrupt the maternal function of PGC formation such that the progeny of the homozygous mutant female is normal but depleted in their germ cells.

A mutation that disrupts the maternal-effect function of a PGC development gene refers to any genetic mutation that directly or indirectly impairs or abolishes a PGC development gene's maternal-effect function. Directly or indirectly affecting gene function refers to: (1) mutating the coding sequence of one or more PGC development genes; (2) mutating a non-coding sequence that has at least some control over the transcription or post transcriptional regulation of one or more PGC development genes; (3) mutating the coding sequence of another gene that is involved in post-transcriptional regulation of one or more PGC development genes; (4) mutating the coding sequence of another gene that is involved in the transport, formation, or combination thereof of germ plasm, for example, a gene product of one or more PGC development genes; (5) mutating the coding sequence of another gene that is involved in germ cell specification, maintenance, migration, or a combination thereof; (6) mutating the coding sequence of another gene that is involved in the epigenetic regulation of one or more PGC development genes; or (7) a combination thereof, to impair or ablate the PGC development gene's function. Gene function refers to the direct function of the gene itself and to the function of molecules produced during expression of the gene, for example, the function of RNA and proteins. Impairing gene function refers to decreasing the amount of gene function compared to the function of the gene's wild-type counterpart by, for example, about 10%, about 25%, about 50%, about 75%, about 90%, or about 95%. Abolishing gene function, or loss of function, refers to decreasing the amount of gene function compared to the function of the gene's wild-type counterpart by 100%. As used herein, “wild-type” refers generally to an organism where the maternal-effect function is undisrupted. “Wild-type counterpart” refers generally to normal organisms of the same age, species, etc.

A mutation may be any type of alteration of a nucleotide sequence of interest, for example, nucleotide insertions, nucleotide deletions, nucleotide substitutions. Preferred mutations in the coding sequence of one or more PGC development genes are nucleotide insertions or nucleotide deletions that cause a frameshift mutation, which may result in the production of a non-functional protein.

Mutating the coding sequence of one or more PGC development genes refers to any type of mutation to the coding sequence that: (1) impairs or abolishes the maternal-effect function of the PGC development genes involved in PGC development, maintenance, migration, or a combination thereof; and (2) does not impair or abolish the viability, sex determination, fertility, or a combination thereof of a homozygous progenitor carrying said mutation. Examples of mutations to the coding sequence of the primordial germ cell development gene are mutations in the coding sequence of Tia1, TIAR, KHSRP, DHX9, Elavl1, Igf2bp3, Ptbp1a, TDRD6, Hook2 and Hnrnpab. The inventors discovered that mutating the coding sequence of certain PGC genes that impaired or abolished the maternal-effect function of the PGC development genes involved in PGC development, maintenance, migration, or a combination thereof also impaired or abolished the viability, sex determination, fertility, or a combination thereof of a homozygous progenitor carrying said mutation, for example, Hnrnph1, Hermes, Elavl2, KIF5B.

Surprisingly, the inventors discovered that mutating: (1) a non-coding sequence that has at least some control in the transcription or post transcriptional regulation of one or more PGC development genes; (2) mutating the coding sequence of another gene that is involved in post-transcriptional regulation of the PGC development gene; (3) mutating the coding sequence of another gene that is involved in the transport, formation, or combination thereof of germ plasm; (4) mutating the coding sequence of another gene that is involved in germ cell specification, maintenance, migration, or a combination thereof; or (5) a combination thereof, may avoid impairing or abolishing the viability, sex determination, fertility, or a combination thereof of a homozygous progenitor carrying said mutation. See Examples 10-13 and 16-18.

Mutating a non-coding sequence that has at least some control over the transcription or post transcriptional regulation of one or more PGC development genes refers to any type of mutation of a non-coding region that: (1) impairs or abolishes the maternal-effect function of the PGC development genes involved in PGC development, maintenance, migration, or a combination thereof; and (2) does not impair or abolish the viability, sex determination, fertility, or a combination thereof of a homozygous progenitor carrying said mutation. Examples of mutating the non-coding sequence of one or more PGC development gene are mutations in: (1) one or more cis-acting 5′ UTR regulatory sequences of the one or more PGC development genes; (2) one or more cis-acting 3′ UTR regulatory sequences of the one or more PGC development genes; (4) promoters of the one or more PGC development genes; or (4) a combination thereof. Examples of cis-acting 5′ UTR regulatory sequences are the 5′ UTR regulatory sequence of nanos3, dnd1, and piwi-like genes, for example, ziwi. Examples of cis-acting 3′ UTR regulatory sequences are the 3′ UTR regulatory sequence of nanos3, dnd1, and piwi-like genes.

Mutating the coding sequence of another gene that is involved in post-transcriptional regulation of one or more PGC development genes refers to any type of mutation of a gene other than the one or more PGC development genes that: (1) impairs or abolishes the maternal-effect function of the PGC development genes involved in PGC development, maintenance, migration, or a combination thereof; and (2) does not impair or abolish the viability, sex determination, fertility, or a combination thereof of a homozygous progenitor carrying said mutation. Examples of mutating the coding sequence of another gene that is involved in post-transcriptional regulation of one or more PGC development genes are mutating a gene encoding an RNA binding protein involved in the post-transcriptional regulation of the one or more PGC development genes and mutating a gene encoding an microRNA involved in the post-transcriptional regulation of the one or more PGC development genes. Examples of RNA binding proteins that are involved in the post-transcriptional regulation of one or more PGC development genes are Hnrnpab, Elavl1, Ptbp1a, Igf2bp3, Tia1, TIAR, Rbpm42, Rbpm24, KHSRP, and DHX9.

Mutating the coding sequence of another gene that is involved in the transport, formation, or combination thereof of germ plasm refers to any type of mutation of a gene other than the one or more PGC development genes that: (1) impairs or abolishes the maternal-effect function of the PGC development genes involved in PGC development, maintenance, migration, or a combination thereof; and (2) does not impair or abolish the viability, sex determination, fertility, or a combination thereof of a homozygous progenitor carrying said mutation. Examples of mutating a coding sequence of another gene that is involved in the transport, formation, or combination thereof of germ plasm are one or more genes that encode a multi-tudor domain-containing protein, a kinesin-like protein, or an adaptor protein. An example of a multi-tudor domain-containing protein is Tdrd6. An example of an adaptor protein is hook2.

Mutating a coding sequence of another gene that is involved in germ cell specification, maintenance, migration, or a combination thereof refers to any type of mutation of a gene other than the one or more PGC development genes that: (1) impairs or abolishes the maternal-effect function of the PGC development genes involved in PGC development, maintenance, migration, or a combination thereof; and (2) does not impair or abolish the viability, sex determination, fertility, or a combination thereof of a homozygous progenitor carrying said mutation. An example of mutating a coding sequence of another gene that is involved in germ cell specification, maintenance, migration, or a combination is mutating a gene expressing a non-coding RNA. An example of a non-coding RNA is miR202-5p.

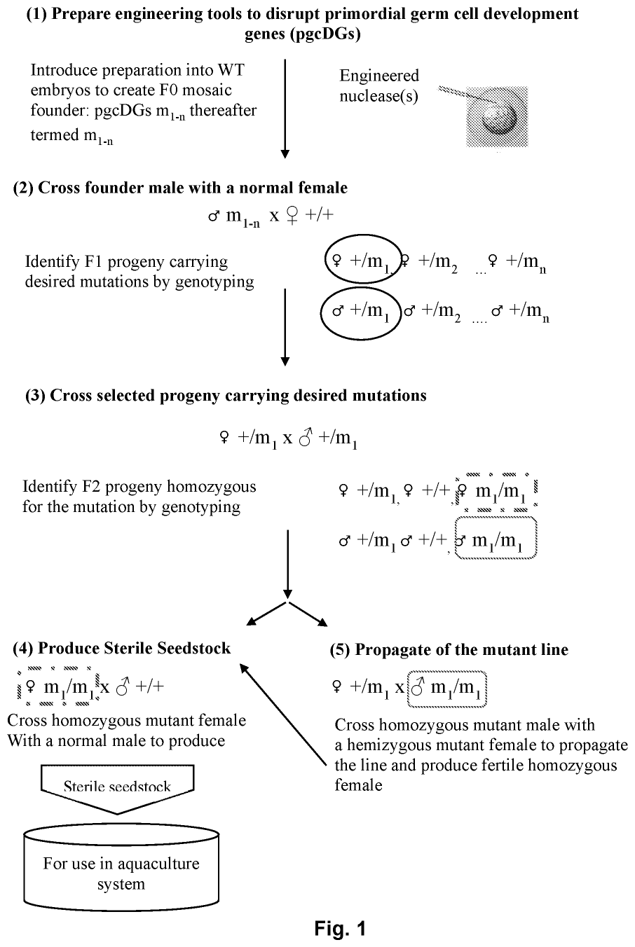

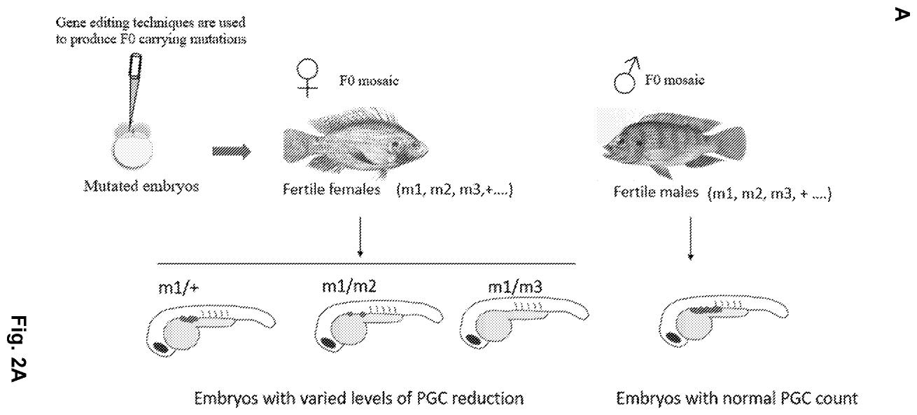

FIG. 1 illustrates an example according to the present disclosure of how a broodstock can either be maintained or used to produce a sterile fish, crustacean, or mollusk. In step 1, one or more gene mutations that disrupt the maternal-function of one or more PGC development genes is introduced into a wild-type embryo of a fish, crustacean, or mollusk to create an F0 mosaic founder, represented by “pgcDGsm₁₋ₙ” in FIG. 1. Any biotechnology technique known to the skilled person that directly manipulates one or more genes in an organism may be used to produce the F0 mosaic founder. The F0 mosaic founder may be fertile given that the biological material necessary to make PGCs was provided by a mother whose genome did not carry the one or more mutations.

In step 2, a male F0 mosaic founder is crossed with a wild-type female to produce F1 progeny. The progeny may be fertile given that the one or more PGC development genes are provided by the wild-type mother. Given that the male F0 mosaic founder carries different types of mutant alleles in different cells, the progeny are screened to locate progeny carrying the desired mutation(s), which is designated by “m₁” in FIG. 1. Any biotechnology technique known to the skilled person that identifies one or more gene mutations in an organism may be used to screen the progeny, for example, genotypic selection. The F0 mosaic founder male may also be crossed with a female carrying no more than one mutant allele for any maternal effect gene or combination of maternal effect genes. Such crosses may be used, for example, to speed up the generation of double knockout lines.

In step 3, a hemizygous mutated male F1 and a hemizygous mutated female F1 from step 2 are identified as carrying the same mutation(s) of interest and are crossed to produce F2 progeny. The progeny may be fertile given that the hemizygous mutated female F1 carries one wild-type copy of the mutated gene(s). An F2 homozygous mutated female is identified and may be used as a homozygous broodstock, which is designated by the checkered outline in FIG. 1.

In step 4, the F2 homozygous mutated female broodstock is crossed with a wild-type male fish, crustacean, or mollusk to produce F3 progeny that are sterile, which may be referred to as sterile seedstock. Alternatively, the F2 homozygous mutated female broodstock is crossed with a hemizygous mutated male fish, crustacean, or mollusk or a homozygous mutated male fish, crustacean, or mollusk wild-type male fish, crustacean, or mollusk to produce F3 progeny that are sterile. The sterility of the progeny stems from the homozygous mutation in the F2 mother, which does not carry a wild-type copy of the mutated gene(s). Preferably, the F2 homozygous mutated female broodstock is crossed with a wild-type male fish, crustacean, or mollusk to produce F3 progeny that are sterile because crossing the F2 homozygous mutated female broodstock with a hemizygous mutated male fish, crustacean, or mollusk or a homozygous mutated male fish, crustacean, or mollusk wild-type male fish, crustacean, or mollusk may generate 50% or 100% of F3 progeny that is homozygous for the mutation. If the mutated gene has pleiotropic function beyond its role in PGC development, the F3 progeny may be impaired for the alternative function, for example, metabolism and immunity.

In step 5, an F2 homozygous mutated male, which is designated by the solid outline in FIG. 1, is identified and crossed with an identified F2 hemizygous mutated female to produce F3 progeny. The F3 progeny are fertile given that the hemizygous mutated female F2 carries one wild-type copy of the mutated gene(s). A homozygous mutated female may be identified and used as broodstock in step 4. A F3 hemizygous male and a F3 hemizygous female may be identified as hemizygous broodstock that may be crossed as in step 3.

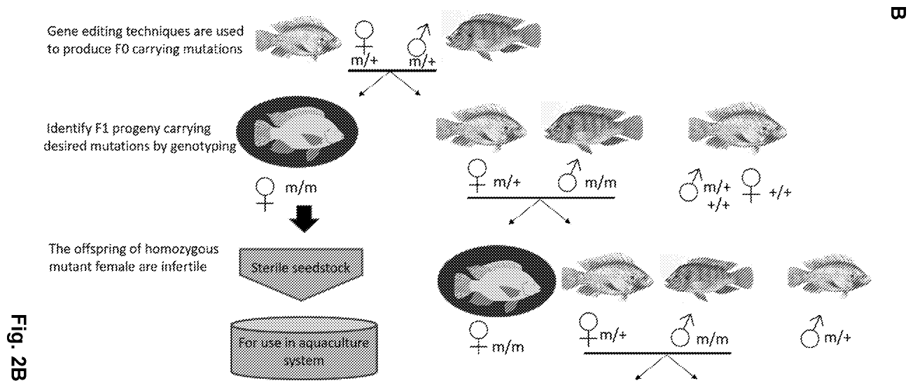

FIGS. 2A and B are flowcharts illustrating an overview of the herein described mutagenesis strategy to identify maternal effect mutants affecting PGCs development and further propagation of the selected mutant alleles. FIG. 2A is a flowchart illustrating gene editing techniques used herein to induce indels at desired locations in selected genes. Treated embryos were derived from a transgenic line expressing GFP:nos3 3′UTR from an oocyte specific promoter. “m” refers to any germ-line mutation and numbers indicate the possibility of varied indels in mosaic F0. Progeny from F0 females crossed with WT males and F0 males crossed with transgenic GFP female were analyzed under fluorescent microscopy at 4 dpf and GFP-PGCs scored and recorded. The average PGC count from at least twelve progenies from each F0 crosses were compared. Mutations causing reduced PGC count in the progeny from F0 females and normal PGC count in the progeny from F0 males were selected and propagated. FIG. 2B is a flowchart illustrating propagation of mutations haplosufficient for both somatic and germline development. F1 fish carrying the same gene mutant allele were intercrossed to produce F2 fish. One quarter of F2 are expected to be homozygous for the gene mutant allele. Mutations did not affect development, sex determination and fertility, and produced homozygous mutant fertile females. If the mutation only disrupts the maternal function of PGC formation, the progeny of these F2 homozygous female crossed with male of any genetic background should all display a PGC ablation phenotype.

### EXAMPLES

### Example 1—Use of a Gene Editing Tool to Induce Double-Allelic Knockout in Tilapia F0 Generation

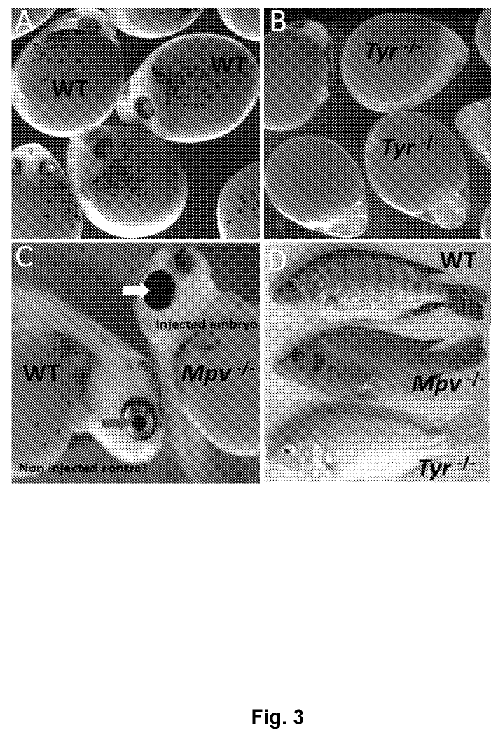

We have independently targeted two genes involved in pigmentation, namely the genes encoding tyrosinase (tyr) [1] and the mitochondrial inner membrane protein MpV17 (mpv17) [2]. We found that 50% and 46% of all injected embryos showed a high degree of mutation at the tyr and mpv17 loci respectively (FIG. 3). Loss-of-function alleles cell-autonomously lead to unpigmented melanophores in the embryo body (FIG. 3 panel B) and in the retinal pigment epithelium (FIG. 3 panel C), producing embryonic phenotypes ranging from complete to partial loss of melanine and iridophore pigmentation that are easy to identify against wild-type phenotype (FIG. 3 panel A). Embryos showing a complete lack of pigmentation (10-30% of treated fish) were raised to 3 months of age and all lacked wild type tyr and mpv17 sequences. These fish display transparent and albino phenotypes (FIG. 3 panel D), indicating that functional studies can be performed in F0 tilapia.

### Example 2—Multi-Gene Targeting in Tilapia

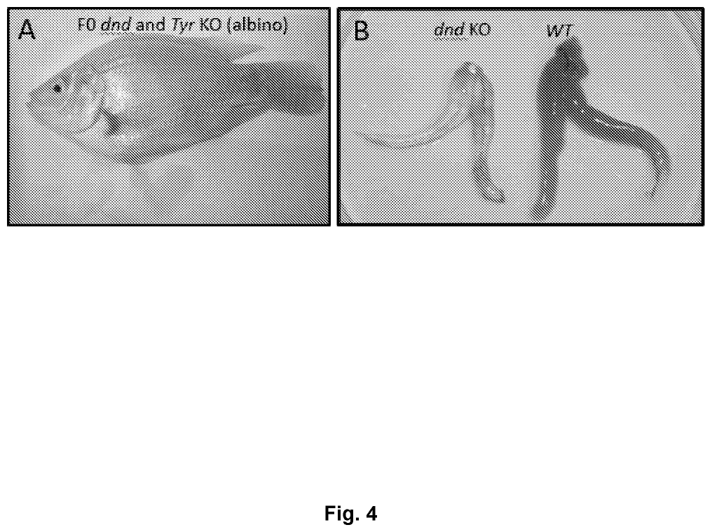

We tested whether multiple genomic loci can be targeted simultaneously and whether mutagenic efficiency measured at one loci is predictable of mutation at other loci in the tilapia genome. To test our hypothesis, we co-targeted tyr and Dead-end1 (dnd1). Dnd1 is a PGC-specific RNA binding protein (RBP) that maintains germ cell fate and migration ability [3]. Following injection of programmed nucleases, we found that mutations in both gene targets tyr and dnd1 were highly correlated. Approximately 95% of abino (tyr; see adult phenotype in FIG. 4 panel A) mutants also carried mutations at the dnd1 loci, demonstrating the suitability of the pigmentation defect as a selection marker (FIG. 4). Upon further analysis of the gonads from 10 albino fish, 6 were translucid germ cell-free testes (FIG. 4 panel B). Expression of vasa, a germ cell specific marker strongly expressed in wild type testes, was strikingly not detected in dnd1 mutant testes. This result indicates that zygotic dnd1 expression is necessary for the maintenance of germ cells and that maternally contributed dnd1 mRNA and/or protein cannot rescue the zygotic loss of this gene.

### Example 3—Generation of F0 Mutants

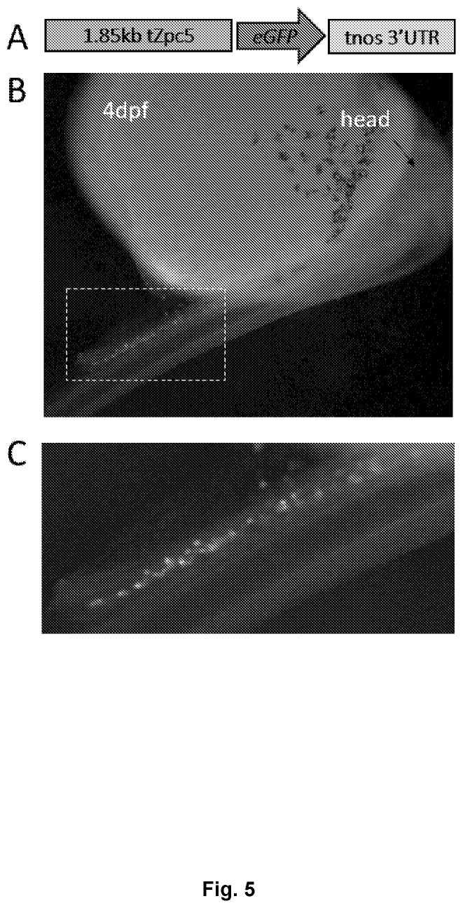

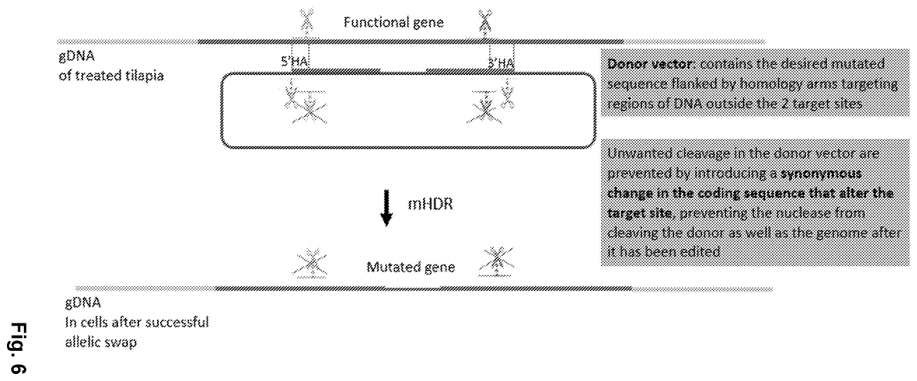

Tilapia orthologues of the selected genes and cis-acting elements in nos-3 and dnd1 3′UTR have been identified in silico from genomic databases and from software motif discovery algorithm searches [4-7]. To enhance the frequency of generating null mutations in the gene of interest, we targeted 2 separate exons simultaneously. Alongside the gene of interest, we co-targeted a pigmentation gene to serve as a mutagenesis selection marker. All mutants were created in tilapia lines containing the ZPC5:eGFP:nos 3′UTR construct (FIG. 5), with the exception of those targeting nos3 3′UTR. Based on our previous work, we expected that injection of 200 embryos will produce 20-60 embryos with complete pigmentation defect. Five of these embryos were quantitatively assayed for genome modifications by PCR fragment analysis [8]. Furthermore, we only raised batches of embryos in which mutations were produced at the one or two cell stage, i.e. detection of 2 or 4 mutant alleles per targeted loci by fragment analysis assay.

### Example 4—Phenotypic Analysis of Each Group of Mutants from Example 3

Selected F0 mutants were screened for morphological malformations, developmental delays and sex differentiation. If the mutated fish develop normally, fertility of 3 males and 3 females were assessed at 4 and 6 months respectively by crossing them with ZPC5:eGFP:tnos 3′UTR tilapia. For each cross, 30 F1 progeny were genotyped and an additional 20 were analyzed by fluorescent microscopy. Since these lines express GFP selectively in PGCs, labelled-PGCs can be counted at 4 dpf when all PGCs have completed their migration to the genital ridges. The mean total PGC numbers were statistically compared across F1 progenies using an unpaired t test. If the engineered mutations function as hypothesized, we expected F1 embryos produced from F0 females to have reduced or absent GFP-PGC counts. Likewise, if the mutations are indeed maternal-effect specific, we expected F0 males to produce F1 progeny with a normal PGC counts (˜35+/−5 PGCs/embryo) (see FIG. 2A).

### Example 5—Generation of F1 and F2 Lines from Example 4

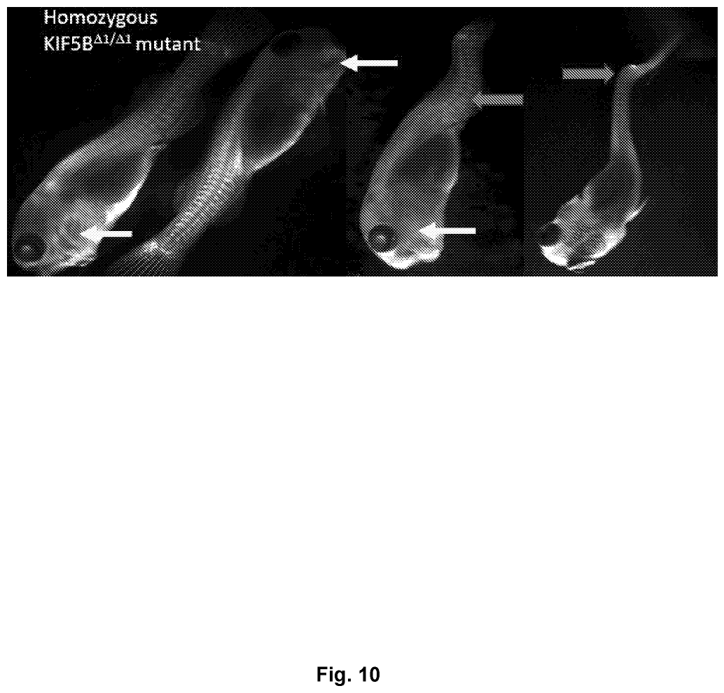

To select F1 hemizygous (outcrossed with WT fish of different genetic backgrounds) and F2 homozygous lines, we used QPCR melt analysis (MA) on amplicons spanning the target regions (FIG. 8). Because each heterozygous lesion produces a characteristic melt curve, it is possible to regroup and breed F1 progeny carrying the same indels. To fully characterize the indels, we sequenced the PCR products from F1 individuals. The mutant read can be extracted from heterozygous sequencing by subtracting the WT sequence.

### Example 6—Confirmation of Sterility at the Molecular, Cellular, and Morphological Level from Example 5

For each RBP and 3′UTR target, F3 embryos from F2 homozygous mutant males and females crossed with WT broodstock (n=30/group), were produced and raised to 2-3 months of age. Gonads from 10 juveniles were dissected and RNA/cDNA were screened by QPCR using vasa, a germ cell specific gene [9]. Q-PCRs for each sample was performed in triplicate and the level of vasa expression was normalized to a set of host house-keeping genes [57] (β-actin and ef1α). We expected no expression of vasa in sterile fish. At 5 months of age, we expect sterile males to have translucid testes and sterile females to yield a string-like ovary. An additional subset of dissected gonads was fixed (n=10/group) in Bouin's solution, dehydrated and infiltrated with paraffin for sectioning. Sterility was apparent from a complete absence of germ cells.

### Example 7—Quantify Production Traits and Growth Rate of Sterile Populations

To generate 3 half sibling groups for these trials, embryos from 3 WT males crossed with 3 F2 homozygous mutant females (sterile groups) and 3 WT females (fertile groups) will be reared separately using established hatchery procedures. At ˜1 month of age, tilapia progeny (n=100/group) will be weighed, pit-tagged and held together in 3×300-Liters tanks in a recirculating culture system maintained at 27° C. All fish will be fed twice daily, to satiation, using a commercially prepared grow-out diet. Each fish will be individually weighed and measured at 4-week intervals over a 24-week period. At the end of the experiment, fish will be sacrificed, sexed, the mean total fish length, weight, filet yield and growth curves will be statistically compared using an unpaired t test.

### Example 8—Materials and Methods

Generation of nucleases and strategies: To create DNA double strand breaks (DSBs) at specific genomic site, we used engineered nucleases. In most applications a single DSB is produced in the absence of a repair template, leading to the activation of the non-homologous end joining (NHEJ) repair pathway. In a percentage of cases NHEJ can be an imperfect repair process, generating insertions or deletions (indels) at the target site. Introduction of an indel can create a frameshift within the coding region of the gene or a change in its regulatory region, disrupting the gene translation or its spatio-temporal regulation, respectively. To enhance the frequency of generating null mutations in the gene of interest, we targeted 2 separate exons simultaneously with the exception of those targeting nos 3′UTR and miR202. Alongside the gene of interest, we co-targeted a pigmentation gene to serve as a mutagenesis selection marker.

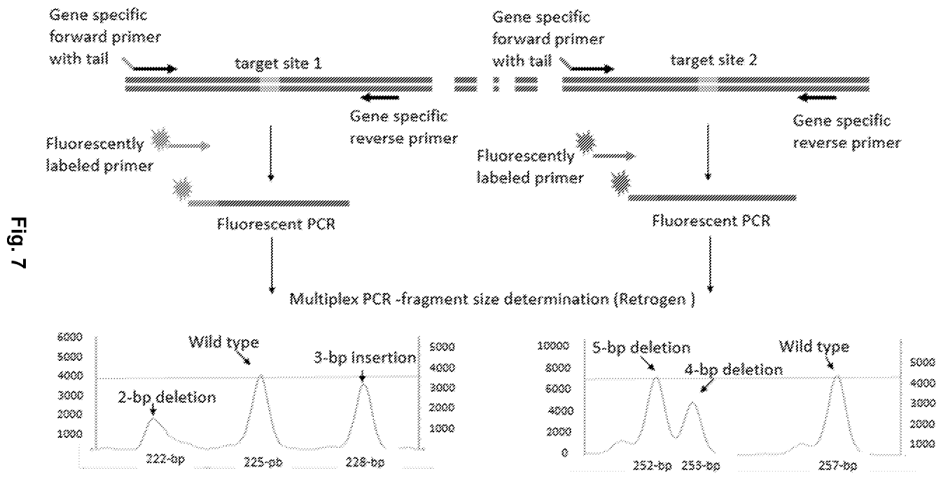

In some embodiments, to introduce custom nucleotide changes to the DNA sequence, two target sites were used to cut out the region to be modified. This strategy requires a donor vector which contain, the dsDNA with the desired mutations flanked by homology arms targeting regions of DNA outside the 2 target sites. This strategy activates the microhomology-directed repair (mHDR). The end result is that the DNA sequence included in the donor vector is incorporated into the native locus (FIG. 6).

The template DNA coding for the engineered nuclease were linearized and purified using a DNA Clean & concentrator-5 column (Zymo Resarch). One microgram of linearized template was used to synthesize capped RNA using the mMESSAGE mMACHINE T3 kit (Invitrogen), purified using Qiaquick (Qiagen) columns and stored at −80° in RNase-free water at a final concentration of 800 ng/μl.

Embryo injections: All animal husbandry procedures were performed according to IACUC-approved CAT animal protocol CAT-003. All injections were performed in tilapia lines containing the ZPC5:eGFP:tnos 3′UTR construct or a wild-type strain. Approximately 10 nL total volume of solution containing the programmed nucleases were co-injected into the cytoplasm of one-cell stage embryos. Injection of 200 embryos typically produce 10-60 embryos with complete pigmentation defect (albino phenotype). Embryo/larvae survival was monitored for the first 10-12 days post injection.

Selection of founders: Selected albino F0 mutants were screened for morphological malformations, developmental delays and sex differentiation. If the mutated fish developed normally, fertility of 3 males and 3 females were assessed at 4 and 6 months respectively by crossing them with ZPC5:eGFP:tnos 3′UTR tilapia. For each cross 20 F1 progeny were analyzed by fluorescent microscopy. Since these lines express GFP selectively in PGCs, labelled-PGCs were counted at 4 dpf when all PGCs have completed their migration to the genital ridges (see Example 9). The mean total PGC numbers was then statistically compared across F1 progenies using an unpaired t test. If the engineered mutations function as hypothesized, we expect F1 embryos produced from F0 females to have reduced or absent GFP-PGC counts. Likewise, if the mutations are indeed maternal-effect specific, we expect F0 males to produce F1 progeny with a normal PGC counts (˜35+/−5 PGCs/embryo).

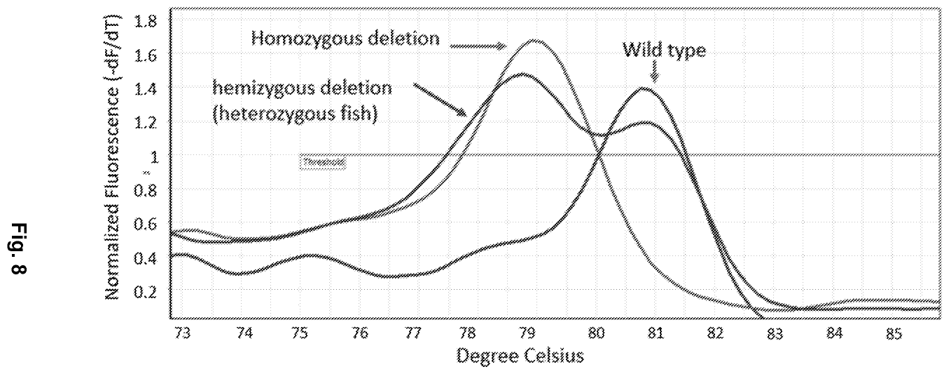

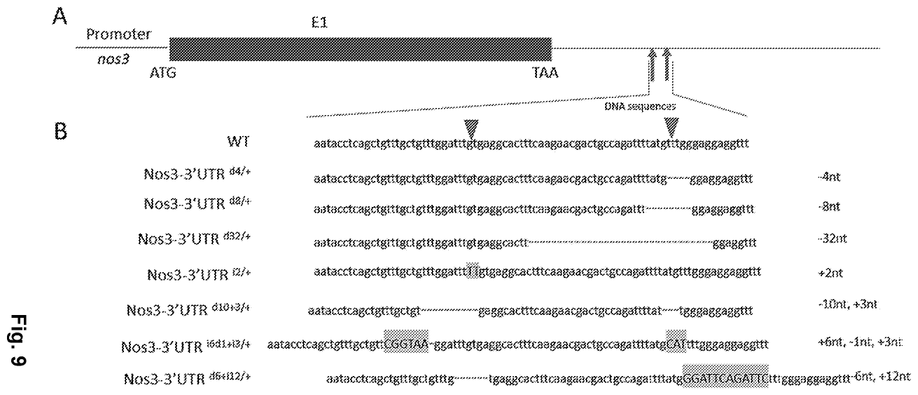

For mutant lines that confer a maternal effect specific PGC reduction, 3-5 F0 males were quantitatively assayed for genome modifications by fluorescence PCR fragment analysis (see Tables 1 and 2 for gene specific genotyping primers). We selected males in which mutations were produced at the one or two cell stage (detection of 2 or 4 mutant alleles per target loci by fragment analysis (FIG. 7).

| | | | | | Genotyping primers3 | | | | | | | |
|---|---|---|---|---|---|---|---|---|---|---|---|---|
| | Tilapia | NCBI& | | | | | | | | | | Ampli- |
| full | homolog | Ensembl | Tar- | | Forward | SEQ | | For- | Reverse | SEQ | | con |
| gene | gene | Accession | geted | site | primer | ID | Mark- | ward | primer | ID | Reverse | size |
| name | (alias) | # | exon | ref# | exon | NO | er | primer | exon | NO | primer | (bp) |
| kinesin | KIF5B | Acc: | 1 | 61 | 1 | SEQ | NED | GTGAA | 2 | SEQ | gaaga | 352 |
| family | | 100700741 | | | | 1 | | TTTCC | | 2 | caTAG | |
| member 5 | | | | | | | | ATTCG | | | CGCGT | |
| | | | | | | | | TGAAC | | | TATAT | |
| | | | | | | | | CG | | | G | |
| | | ENSONIG00 | 4 | 72 | 3 | SEQ | FAM | TTTGC | 5 | SEQ | agtct | 365 |
| | | 000015032 | | | | 3 | | ATATG | | 4 | cagat | |
| | | | | | | | | GGCAG | | | cttaa | |
| | | | | | | | | ACATC | | | ccata | |
| | | | | | | | | | | | ta | |
| TIA 1 | TIAR | Acc: | 2 | 71 | 2 | SEQ | FAM | TGATT | Intron | SEQ | tggtt | 163 |
| cytotoxic | (TIAL 1) | 100701620 | | | | 11 | | TGAAT | 2-3 | 12 | ggact | |
| granule- | | | | | | | | CCAGA | | | gaaac | |
| protein- | | | | | | | | GATTA | | | atatt | |
| like 1 | | | | | | | | CT | | | gt | |
| associated | | ENSONIG00 | 11 | 65 | 11 | SEQ | NED | tgtcc | 11 | SEQ | gtcaa | 188 |
| RNA | | 000010290 | | | | 13 | | ttcag | | 14 | actca | |
| binding | | | | | | | | GTTGA | | | cTCTA | |
| | | | | | | | | TTACA | | | CTCCA | |
| | | | | | | | | G | | | A | |
| KH-type | KHSRP | Acc: | 13 | 67 | 13 | SEQ | NED | tcttt | 13 | SEQ | TGACG | 207 |
| splicing | | 100698089 | | | | 9 | | cacag | | 10 | AGATA | |
| regulatory | | | | | | | | GGTCC | | | TCTCC | |
| protein | | | | | | | | ACCG | | | ACAAA | |
| | | | | | | | | | | | TGC | |
| | | ENSONIT000 | 13 | 71 | 13 | SEQ | NED | tcttt | 13 | SEQ | TGACG | 207 |
| | | 00022355.1 | | | | 9 | | cacag | | 10 | AGATA | |
| | | | | | | | | GGTCC | | | TCTCC | |
| | | | | | | | | ACCG | | | ACAAA | |
| | | | | | | | | | | | TGC | |
| TIA 1 | TIA 1 | Acc: | 5 | 75 | Intron | SEQ | NED | tataa | Intron | SEQ | tgaca | 317 |
| cytotoxic | | 100709521 | | | 4-5 | 5 | | ttcat | 5-6 | 6 | cattg | |
| granule- | | | | | | | | tgttg | | | gctga | |
| protein | | | | | | | | tgggt | | | gactt | |
| associated | | | | | | | | tgta | | | tc | |
| RNA | | | | | | | | | | | | |
| binding | | ENSONIG000 | 11 | 87 | 9 | SEQ | FAM | CCATT | 11 | SEQ | TCATA | 297 |
| | | 00015897 | | | | 7 | | CTGAA | | 8 | GCTCC | |
| | | | | | | | | GTTAT | | | CTCCT | |
| | | | | | | | | CCTTT | | | GTGGC | |
| | | | | | | | | T | | | | |
| DEAH | DHX9 | Acc: | 2 | 75 | 2 | SEQ | NED | GGCTT | 3 | SEQ | GGGAG | 299 |
| (Asp-Glu- | | 100697569 | | | | 15 | | CAACT | | 16 | GTTTC | |
| Ala-His) | | | | | | | | ACATT | | | CAAAG | |
| box | | | | | | | | GGGAT | | | CCAGC | |
| helicase 9 | | | | | | | | GGG | | | AT | |
| | | ENSONIG00 | 4 | 72 | 4 | SEQ | FAM | ttctc | 4 | SEQ | GTCTC | 156 |
| | | 000000293 | | | | 17 | | agGGG | | 18 | TCTTG | |
| | | | | | | | | ACGG | | | GCGTA | |
| | | | | | | | | CAGCG | | | ATACT | |
| | | | | | | | | | | | CC | |
| insulin- | Igf2bp3 | Acc: | 6 | 67 | 6 | SEQ | NED | GGCAA | 6 | SEQ | GAACC | 203 |
| like | | 100702462 | | | | 19 | | TGGAG | | 20 | AGCAT | |
| growth | | | | | | | | AAGCT | | | GCGTA | |
| mRNA factor 2 | | | | | | | | GAATG | | | GCGGG | |
| binding | | | | | | | | GAT | | | AT | |
| protein 3 | | ENSONIG00 | 9 | 69 | Intron | SEQ | FAM | agaag | 9 | SEQ | GTTCA | 177 |
| | | 000004506 | | | 8-9 | 21 | | ttcta | | 22 | TAGCA | |
| | | | | | | | | atgca | | | GCCAT | |
| | | | | | | | | cctcc | | | GTCAC | |
| | | | | | | | | aa | | | TCT | |
| ELAV | Elavl 1 | Acc: | 3 | 63 | 3 | SEQ | NED | CGCTA | 3 | SEQ | ACTGC | 247 |
| like | | 100695900 | | | | 23 | | AAGGA | | 24 | TGAAG | |
| protein 1 | | | | | | | | GCTGC | | | AGGCT | |
| | | | | | | | | TGGAA | | | GCGTA | |
| | | | | | | | | ATg | | | G | |
| | | ENSONIT00 | 7 | 77 | 7 | SEQ | FAM | GGGAG | 7 | SEQ | TGCCC | 263 |
| | | 000004011 | | | | 25 | | CTCAT | | 26 | CTTGC | |
| | | | | | | | | CCTCT | | | TGGTC | |
| | | | | | | | | GGTTG | | | TTGAA | |
| | | | | | | | | GTG | | | T | |
| ELAV like | Elavl 2 | Acc:HGNC: | 1 | 65 | Intron | SEQ | FAM | ttttc | 1 | SEQ | GTTTT | 149 |
| neuron- | | 3313 | | | 0-1 | 27 | | tttgt | | 28 | ACTGT | |
| specific | | | | | | | | ctctt | | | CCTCT | |
| RNA | | | | | | | | tagCA | | | ACGC | |
| binding | | | | | | | | GGT | | | | |
| protein 2 | | | | | | | | | | | | |
| | | ENSONIG00 | 7 | 73 | 7 | SEQ | NED | ttgtg | 7 | SEQ | CACTT | 212 |
| | | 000007552 | | | | 29 | | tttta | | 30 | GTTTG | |
| | | | | | | | | acagC | | | TGTTA | |
| | | | | | | | | AGGT | | | AAGTC | |
| | | | | | | | | TCTC | | | GC | |
| Chemokine | Cxcr4a | Acc: | 2 | 69 | 2 | SEQ | FAM | TGGGA | 2 | SEQ | TTGAT | 204 |
| (C-X-C | | 100703262 | | | | 31 | | TAGTT | | 32 | GGTGT | |
| motif) | | | | | | | | GGTAA | | | AGATC | |
| receptor | | | | | | | | TGGAT | | | ATGTG | |
| 4a | | | | | | | | T | | | CA | |
| | | ENSONIG00 | 2 | 67 | 2 | SEQ | NED | GTCTG | 2 | SEQ | ACTGT | 315 |
| | | 000004410 | | | | 33 | | TGCAC | | 34 | CCATA | |
| | | | | | | | | ATGAT | | | TGACG | |
| | | | | | | | | CTACA | | | TTACT | |
| | | | | | | | | CCA | | | TTC | |
| Poly- | Ptbp1a | Acc: | 4 | 75 | 4 | SEQ | NED | CCAAT | 4 | SEQ | tgtgt | 191 |
| pyrimidine | | 100710677 | | | | 35 | | GGCAA | | 36 | acagt | |
| tract | | | | | | | | CGACA | | | gtgtg | |
| binding | | | | | | | | GCAAA | | | tacCT | |
| protein 1a | | | | | | | | AAG | | | GGT | |
| | | ENSONIG00 | 8 | 76 | 8 | SEQ | FAM | TCTCT | Intron | SEQ | ctgca | 238 |
| | | 000007784 | | | | 37 | | GGACG | 8-9 | 38 | tcaca | |
| | | | | | | | | GTCAG | | | gtttt | |
| | | | | | | | | AACAT | | | tgagc | |
| | | | | | | | | CTA | | | aca | |
| nanos | Nos3 | Acc: | 1 | 66 | 1 | SEQ | NED | ATGAA | 1 | SEQ | TAGCT | 196 |
| homolog 3 | | 100698891 | | | | 39 | | CGGAA | | 40 | CGGCT | |
| | | | | | | | | TGGTT | | | CATGT | |
| | | | | | | | | TGG | | | CACAC | |
| | | ENSONIG00 | 1 | 83 | 1 | SEQ | FAM | CCCGC | 1 | SEQ | TCTTG | 256 |
| | | 000020588 | | | | 41 | | GAATG | | 42 | GTTCT | |
| | | | | | | | | TGCAC | | | TCAGC | |
| | | | | | | | | TAACG | | | CAGTG | |
| | | | | | | | | AG | | | GGA | |
| DND | dnd1 | ENSONG000 | 6 | 65 | 5 | SEQ | NED | TTTCC | 3′UTR | SEQ | GTGAA | 335 |
| microRNA- | | 00011554/ | | | | 43 | | CAATT | | 44 | ACAGA | |
| mediated | | ZFIN:Acc: | | | | | | CCTCC | | | ACTGC | |
| repression | | ZDB-GENE- | | | | | | ACCCA | | | AGGAC | |
| inhibitor 1 | | 030828- | | | | | | AG | | | G | |
| Hetero- | Hnmpab | Acc: | 1 | 68 | 1 | SEQ | NED | ATGTC | 1 | SEQ | TCCCT | 183 |
| geneous | | 100707314 | | | | 45 | | TGAGT | | 46 | CCGTG | |
| nuclear | | | | | | | | CAGAG | | | CCGCC | |
| ribonucleo- | | | | | | | | CAACA | | | GTTTT | |
| protein A/B | | | | | | | | GTA | | | | |
| | | ENSONIG00 | 3 | 63 | 3 | SEQ | FAM | GAAGA | 4 | SEQ | TGGAA | 204 |
| | | 000012982 | | | | 47 | | AGGAT | | 48 | GCTCT | |
| | | | | | | | | CCAGT | | | ATGGT | |
| | | | | | | | | AAAGA | | | CTCAA | |
| | | | | | | | | AAA | | | Tct | |
| hetero- | Hnmph1 | 100691632 | | | | | | | | | | |
| geneous | | ENSONIG00 | | | | | | | | | | |
| nuclear | | 000017754 | | | | | | | | | | |
| ribonucleo- | | | | | | | | | | | | |
| protein H1 | | | | | | | | | | | | |
| RNA | Rbms | Acc:HGNC: | 3 | 73 | Intron | SEQ | NED | tttgt | Intron | SEQ | acaac | 111 |
| binding | (Hermes) | 19097 | | | 2-3 | 49 | | tctgt | 3-4 | 50 | gaggg | |
| protein, | | | | | | | | ctcct | | | catga | |
| mRNA | | | | | | | | tgtct | | | cactt | |
| processing | | | | | | | | ccc | | | acG | |
| factor | | ENSONIG00 | 4 | 75 | 4 | SEQ | FAM | CCAAG | Intron | SEQ | ggaag | 169 |
| | | 000007546 | | | | 51 | | ATGGC | 4-5 | 52 | tagca | |
| | | | | | | | | CAAAA | | | atgca | |
| | | | | | | | | ACAAG | | | gacgg | |
| | | | | | | | | C | | | aca | |
| RNA | Rbm24 | Acc: | 1 | 65 | 1 | SEQ | NED | CACAC | Intron | SEQ | tttac | 159 |
| binding | | 100695600 | | | | 53 | | AACCG | 1-2 | 54 | tcgtc | |
| motif | | | | | | | | ACTCA | | | cagct | |
| protein 24 | | | | | | | | AGT | | | gaccg | |
| | | | | | | | | | | | g | |
| | | ENSONIG00 | 4 | 77 | 4 | SEQ | FAM | TTCCC | 4 | SEQ | GCGGT | 177 |
| | | 000003010 | | | | 55 | | CATAC | | 56 | GGCGA | |
| | | | | | | | | CTTGA | | | GCGGC | |
| | | | | | | | | CTATA | | | TG | |
| | | | | | | | | CTG | | | | |
| RNA | Rbm42 | Acc:ZDB- | 5 | 72 | 5 | SEQ | NED | ACCTC | 6 | SEQ | CATTG | 221 |
| binding | | GENE- | | | | 57 | | CACCC | | 58 | AAACC | |
| motif | | 040809-2/ | | | | | | ATGAT | | | ATATC | |
| protein 42 | | ENSONIG00 | | | | | | GCTCC | | | ACCAA | |
| | | 000008029 | | | | | | C | | | CCt | |
| Tudor | TDRD6 | AccZDB- | 1 | Top | 1 | SEQ | FAM | tgcca | 1 | SEQ | CCCAG | 172 |
| domain | | GENE- | | (64) | | 59 | | aaATG | | 60 | GGGAC | |
| containing | | 041001- | | | | | | TC | | | TGAAT | |
| 6 | | 210 | | | | | | ATCAA | | | GTCTT | |
| | | | | | | | | TCTTA | | | TAG | |
| | | | | | | | | G | | | | |
| | | ENSONIG00 | 2 | Bottom | 2 | SEQ | NED | AATTG | 2 | SEQ | tccgt | 148 |
| | | 000001218 | | (85) | | 61 | | TCTGC | | 62 | tatga | |
| | | | | | | | | ACTTA | | | aGCTC | |
| | | | | | | | | TAGAT | | | TTCCA | |
| | | | | | | | | GTC | | | CC | |
| Hook | Hook2 | Acc: | 5 | Bottom | 5 | SEQ | FAM | CTCGG | 5 | SEQ | TCTTC | 124 |
| microtubule | | 100710484 | | (64) | | 63 | | TCACC | | 64 | TCGCA | |
| tethering | | | | | | | | AGGTG | | | GCTGA | |
| protein 2 | | | | | | | | TCTGA | | | CTGCA | |
| | | | | | | | | T | | | C | |
| | | ENSONIG00 | 9 | Top | 9 | SEQ | NED | AAGCT | Intron | SEQ | ttttc | 137 |
| | | 000008175 | | | | 65 | | CAGCC | 9-10 | 66 | ctaag | |
| | | | | | | | | TCAGC | | | tactt | |
| | | | | | | | | GAATC | | | atgta | |
| | | | | | | | | TCT | | | cca | |
| microRNA- | miR202- | | NA | 49 | NA | SEQ | FAM | gttcc | NA | SEQ | ctGGT | 136 |
| 202 | 5p | | | | | 67 | | agtgt | | 68 | GGAAT | |
| | | | | | | | | ccaga | | | ACCTC | |
| | | | | | | | | atcggg | | | TGC | |
| nanos3 | nos3- | ENSONIT000 | NA | 42 | NA | SEQ | FAM | CTCCG | NA | SEQ | gacag | 436 |
| transcript | 3′UTR | 00025914.1 | | | | 69 | | TGTAC | | 70 | tgtta | |
| 3′UTR | (motif1) | | | | | | | GCCAA | | | taatc | |
| | | | | | | | | GTCCA | | | cttca | |
| | | | | | | | | GA | | | atg | |
| | | | NA | 41 | NA | SEQ | FAM | CTCCG | NA | SEQ | gacag | 436 |
| | | | | | | 69 | | TGTAC | | 70 | tgtta | |
| | | | | | | | | GCCAA | | | taatc | |
| | | | | | | | | GTCCA | | | cttca | |
| | | | | | | | | GA | | | atg | |

TABLE 2
Primers

[symbol]

[symbol]

indicates data missing or illegible when filed

### Example 9—Quantitation of PGC Number in Early Embryos

In the transgenic line, Tg(Zpc5:eGFP:tnos 3′UTR) the tilapia Zpc5 promoter is an oocyte-specific promoter, active during oogenesis prior to the first meiotic division. As such, all embryos from a heterozygous or homozygous transgenic female inherit the eGFP:tnos 3′UTR mRNA, which localizes and becomes expressed exclusively in PGCs through the action of cis-acting RNA elements in their 3′UTR (tilapia nos3 3′UTR). Embryos (4 days post fertilization) were euthanized by an overdose of tricaine methanesulfonate (MS-222, 200-300 mg/I) by prolonged immersion for at least 10 minutes. Stock preparation is 4 g/L buffered to pH 7 in sodium bicarbonate (at 2:1 bicarb to MS-222). The embryo were transferred onto a glass surface in PBS and their yolk removed. Deyolked embryos were squashed between a microscope slide and a cover slip and analyzed under fluorescent microscopy equipped with camera for imaging.

F1 genotyping: The selected male founders were crossed with tilapia female carrying the ZPC5:eGFP:tnos 3′UTR construct. Their F1 progeny were raised to 2 months of age, anesthetized by immersion in 200 mg/L MS-222 (tricaine) and transferred onto a clean surface using a plastic spoon. Their fin was clipped with a razor blade, and place onto a well (96 well plate with caps). Fin clipped fish were then placed in individual jars while their fin DNA was analyzed by fluorescence PCR. In brief, 60 μl of a solution containing 9.4% Chelex and 0.625 mg/ml proteinase K is added to each well for overnight tissue digestion and gDNA extraction in a 55° C. incubator. The plate is then vortexed and centrifuged. gDNA extraction solution was then diluted 10× with ultra-clean water to remove any PCR inhibitors in the mixture. Typically, we analyzed 80 juveniles/founder to select and raised batches of approximately 20 juveniles carrying identical size mutations.

Fluorescence PCR (see FIG. 7): PCR reactions used 3.8 μL of water, 0.2 μL of fin-DNA and 5 μL of PCR master mix (Quiagen Multiplex PCR) with 1 ul of primer mix consisting of the following three primers: the Labeled tail primer with fluorescent tag (6-FAM, NED), amplicon-specific forward primer with forward tail (5′-TGTAAAACGACGGCCAGT-3′ and 5′-TAGGAGTGCAGCAAGCAT-3′) amplicon-specific reverse primer (gene-specific primers are listed in Tables 1 and 2). PCR conditions were as follows: denaturation at 95° C. for 15 min, followed by 30 cycles of amplification (94° C. for 30 sec, 57° C. for 45 sec, and 72° C. for 45 sec), followed by 8 cycles of amplification (94° C. for 30 sec, 53° C. for 45 sec, and 72° C. for 45 sec) and final extension at 72° C. for 10 min, and an indefinite hold at 4° C.

One-two microliters of 1:10 dilution of the resulting amplicon were resolved via capillary electrophoresis (CE) with an added LIZ labeled size standard to determine the amplicon sizes accurate to base-pair resolution (Retrogen Inc., San Diego). The raw trace files were analyzed on Peak Scanner software (ThermoFisher). The size of the peak relative to the wild-type peak control determines the nature (insertion or deletion) and length of the mutation. The number of peak(s) indicate the level of mosaicism. We selected F0 mosaic founder carrying the fewest number of mutant alleles (2-4 peak preferentially).

The allele sizes were used to calculate the observed indel mutations. Mutations that are not in multiples of 3 bp and thus predicted to be frameshift mutations were selected for further confirmation by sequencing except for mutation in the non-coding sequence of genes targeted. Mutations of size greater than 8 bp but smaller than 30 bp were preferentially selected to ease genotyping by QPCR melt analysis for subsequent generations. For sequence confirmation, the PCR product of the selected indel is further submitted to sequencing. Sequencing chromatography of PCR showing two simultaneous reads are indicative of the presence of indels. The start of the deletion or insertion typically begins when the sequence read become divergent. The dual sequences are than carefully analyze to detect unique nucleotide reads. The pattern of unique nucleotide read is then analyzed against series of artificial single read patterns generated from shifting the wild type sequence over itself incrementally.

### Example 10—Analysis of Mutant Fish for Embryos Viability, Developmental Deformities and Presence of Both Sexes in Adults

The embryos generated from pairwise breeding of single gene heterozygote mutant fish were analyzed under stereomicroscopy (both bright and fluorescent lights) for gross visible deformities. Clutches of progeny were grown to adulthood (3-6 months). Fin clips from adult fish were processed for DNA extraction with Chelex Resin and used for genotyping by melt analysis: Example 10—F2 and subsequent generation Genotyping by melt analysis (see below)

Real-time qPCR was performed ROTOR-GENE RG-3000 REAL TIME PCR SYSTEM (Corbett Research). 1-μL genomic DNA (gDNA) template (diluted at 5-20 ng/μl) was used in a total volume of 10 μL containing 0.15 μM concentrations each of the forward and reverse primers and 5 μL of QPCR 2× Master Mix (Apex Bio-research products). qPCR primers used are presented in Tables 1 and 2 (Genotyping RT-PCR primers in Table 2). The qPCR was performed using 40 cycles of 15 seconds at 95° C., 60 seconds at 60° C., followed by melting curve analysis to confirm the specificity of the assay (67° C. to 97° C.). In this approach, short PCR amplicons (approx 120-200 bp) that include the region of interest are generated from a gDNA sample, subjected to temperature-dependent dissociation (melting curve). When induced indels are present in hemizygous gDNA, heteroduplex as well as different homoduplex molecules are formed. The presence of multiple forms of duplex molecules is detected by Melt profile, showing whether duplex melting acts as a single species or more than one species. Generally, the symmetry of the melting curve and melting temperature infers on the homogeneity of the dsDNA sequence and its length. Thus, homozygous and wild type (WT) show symmetric melt curved that are distinguishable by varied melting temperature. The Melt analysis is performed by comparison with reference DNA sample (from control wild type DNA) amplified in parallel with the same master mix reaction. In short, variation in melt profile distinguishes amplicons generated from homozygous, hemizygous and WT gDNA (see FIG. 8).

The genotyping data were used to analyze for Mendelian ratios of surviving homozygous knockout fish compared to the homozygote WT and heterozygous fish. Under the null hypothesis of no viability selection, progeny genotypes should conform to an expected Mendelian ratio of 1:2:1. Deviations from expected number of homozygous knockouts (25%) were tested with goodness-of-fit Chi-square statistical analysis.

Sex Ratio Determinations: At 3-4 months of age, progeny (n=40/group) were sexed. Males and females were identified, visually, based on their sex-specific uro-genital papillae.

Morphological and cellular analysis of the gonads: Sterility was evaluated by comparing the overall morphology of the gonads. Gonadal structure in the homozygous maternal progeny (n=20 per cross) was compared to age-matched (3 months old) paternal progeny (fertile control). To analyze the cellular structure of the gonads we fixed gonads in Bouin's solution for 48 h. After dehydration in ethanol and clearing in toluene, the specimens were infiltrated with paraffin, embedded, and sectioned. Each section was read blind by two reviewers. Sterility in male is apparent from a complete absence of spermatozoa in the tubule lumen. Sterility in female is apparent by a gonad reduced to a string like structure and histology sections revealing no oocytes.

Confirmation of sterility at the molecular level: Total RNA was extracted from dissected gonads (from each paternal and maternal group/line) and the corresponding cDNA were screened to quantify expression of germ cell specific genes (tilapia vasa accession #AB03246766) and gonad specific supporting somatic cells (tilapia Sox 9a and tilapia cyp19a1a for male and female gonad respectively). Q-PCRs were performed in triplicate and level of expression was normalized against host house-keeping gene (tilapia b-actin). Relative copy number estimates were generated using established procedures. We expected no expression of vasa in sterile fish but normal expression of sox9a relative to wild type testis.

### Example 11—F0 Phenotypes Associated with Mutations in Selected Genes and Regulatory Sequences

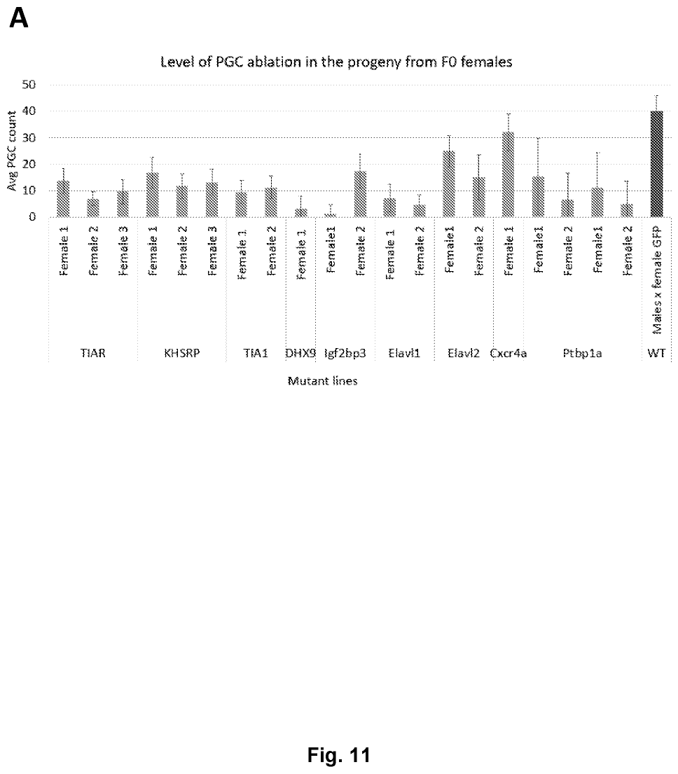

To test if the coding sequence or regulatory sequences of selected genes are strictly essential for PGC development, we generated tilapia mutants using programmable nucleases with or without donor DNA. To enhance the frequency of generating null mutations in Nanos3 (nos3), Dead end-1 (dnd1), TIAR, Tia1, KHSRP, DHX9, Elavl1, Elavl2, Igf2bp3, Rbm42, Rbms (Hermes), Rbm24, Hnmph1, Hnrmpab, Tdrd6, Hook2, Ptbp1a, KIF5B, Cxcr4a genes, we targeted two separate exons of each gene simultaneously. To maximize the chances of generating loss-of-function mutations, we preferentially selected target sites in the first half of the coding region. Alongside the gene of interest, we co-targeted a pigmentation gene to serve as a mutagenesis selection marker (FIG. 3). In addition, to test if the 3′UTR of dnd1 is necessary for its zygotic function we performed a targeted integration of the 3-globin 3′UTR downstream of dnd1 coding sequence. Furthermore, we targeted evolutionary conserved motifs in nos3 3′UTR and dnd1 3′UTR that we hypothesized are involved in the spatio-temporal regulation of the corresponding mRNA in oocyte and early embryos (FIG. 9). These motifs were identified and preferentially selected based on i) their joint presence on the 3′ UTR of orthologue mRNAs ii) their juxtaposition to putative miRNA binding sequence and iii) secondary structures analysis (see details in Example 17). We also targeted a miRNA which localized to PGCs in developing embryos (miRNA202-5p) (Zhang, Liu et al. 2017).

Survival and deformities of F0 treated embryos were analyzed and compared to non-injected controls. We found that Rbms and Hnmph1 F0 treated embryos had low survival rates and no albino fish were recovered, suggesting that these genes play an essential role in embryo morphogenesis. Similarly, KIF5B treated embryos had poor viability. Nonetheless, we successfully recovered and propagated one viable F0 KIF5B mutant displaying severe morphological deformities.

We did not observe a significant difference in viability or visible gross developmental abnormalities between the treatment groups and controls in any other gene mutant fish for the remaining 17 genes targeted. For each treatment group, a minimum of 20 albinos were selected and propagated. All F0 mutant treated groups developed with a normal sex ratio at 5 months of age with the exception of nos3 (88% males, n=42), dnd1 (83% males, n=41), Tia1 (80% males, n=20) and Elavl1 (90% males, n=20) (see Table 3). Furthermore, we found that disruption of the coding sequences of nos3 and dnd1 caused 30% (n=3/10) of nos3 F0 females and 60% (n=4/10) of dnd1 F0 males to develop into agametic adult. In those fish, stripping procedures at maturity yielded no gametes. Upon further analysis of their gonads, we found string-like oocytes-free ovaries in F0 nos3 mutant females and translucid sperm-free testes in F0 dnd mutant males (FIG. 4). Expression of vasa, a germ cell specific marker strongly expressed in wild-type testes and ovaries, was strikingly not detected in the gonads from these fish (nos3 and dnd1 F0 gonads). Interestingly, successful bi-allelic integration of 3-globin 3′UTR downstream of dnd1 coding sequence caused male sterility.

TABLE 3
Targeted genes and associated phenotypes.

| | Protein family and | F0 Embryo | F0 Adult sex | F0 Maternal | F1 hemizygous |
|---|---|---|---|---|---|
| Gene | expected cellular | mutant | differentiation | effect sterility | selected |
| name | function | phenotypes | and fertility | (% PGC ablation) * | mutations |
| Hermes | RNA | lethal | NA | NA | +/I16 |
| (Rbms) | binding/localization | | | | |
| KIF5B | Motor | Lethal | NA | NA | +/Δ1 |
| | protein/microtubule | (Ventralization) | | | |
| | transport | | | | |
| Hnrnph1 | RNA | lethal | NA | NA | NA |
| | binding/localization | | | | |
| TIAR | RNA | ND | N | Yes (>65%) | +/I11 |
| | binding/localization | | | | |
| KHSRP | KH domain nucleic | ND | N | Yes (>70%) | +/Δ17 |
| | acid binding protein | | | | |
| DHX9 | ATP dependent nucleic | ND | N | Yes (>70%) | +/Δ7 |
| | acid unwinding | | | | |
| Elavl1 | RNA | ND | 90% males | Yes (>81%) | +/Δ3 kb |
| | binding/localization | | | | |
| Elavl2 | RNA | ND | N | Yes (>60%) | +/Δ8 |
| (HuB) | binding/localization | | | | |
| TIA1 | RNA | ND | 80% males | Yes (>70%) | +/Δ10 |
| | binding/localization | | | | |
| Igf2bp3 | RNA | ND | N | Yes (>55%) | +/I2 |
| | binding/localization | | | | |
| Ptbp1a | RNA | ND | N | Yes (>60%) | +/Δ13, |
| | binding/localization | | | | +/Δ1.5 kb |
| Tdrd6 | Interact with GP | ND | N | Yes (>55%) | +/Δ10 |
| | organizer protein | | | | |
| | BB | | | | |
| Hook2 | Microtubule binding | ND | N | Yes (>85%) | +/Δ2 |
| Rbm24 | RNA | ND | N | Yes (>80%) | +/Δ7 |
| | binding/localization | | | | |
| Rbm42 | RNA | ND | N | Yes (>80%) | +/Δ7 |
| | binding/localization | | | | |
| Hnrnpab | RNA | ND | N | Yes >65% | +/Δ8 |
| | binding/localization | | | | |
| Nanos3 | RNA- | ND | 90% males | NA (female | +/Δ5 |
| | binding/translational | | Sterility | sterility) | |
| | regulator | | in females | | |
| Dnd1 | RNA | ND | Sterile | NA (no | +/Δ5 |
| | binding/localization | | | female) | |
| Cxcr4 | Endodermal cell | ND | N | Yes (>20%) | +/Δ8 |
| | abnormalities | | | | |
| miR202-5p | | ND | N | Yes (>65%) | +/Δ7, +/Δ8, |
| | | | | | +/Δ19 |
| Nos3 | Likely motif | ND | 90% male | NA (no GFP | +/Δ32, +/Δ8 |
| 3′UTR | protecting against | | | marker | |
| Del32/Del8 | miR-430 degradation | | | present | |
| Dnd1 | Likely motif | ND | N | | |
| 3′UTR AR | protecting against | | | | |
| | miR-23 degradation | | | | |

The mutant alleles selected for each gene are presented.
ND: Not Detected,
NA, Not Applicable,
N: Normal,
* minimum level of PGC ablation measured

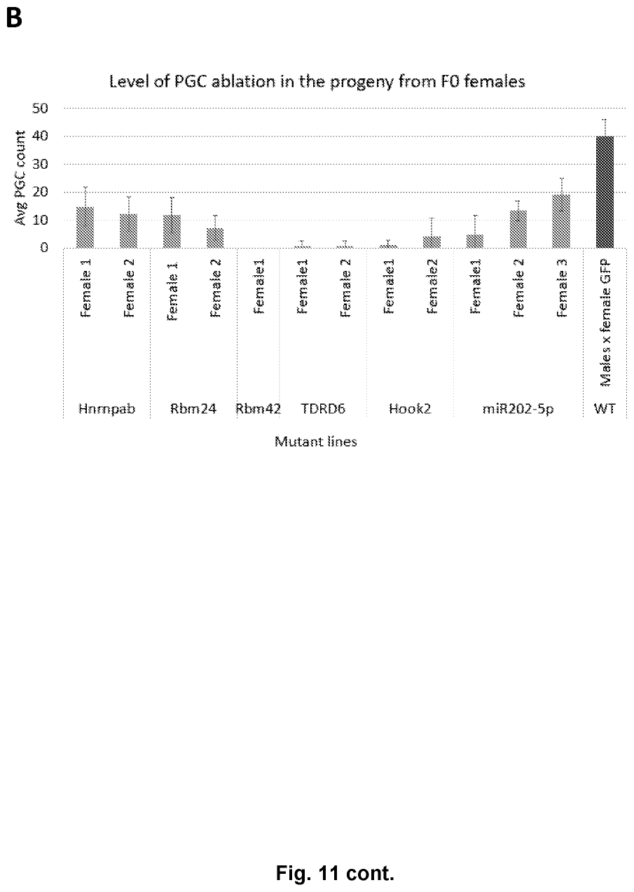

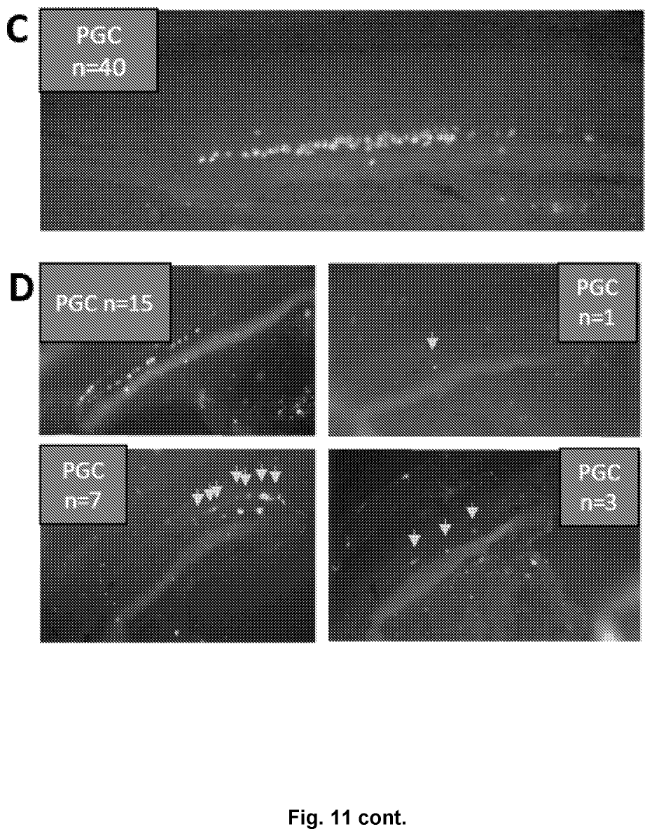

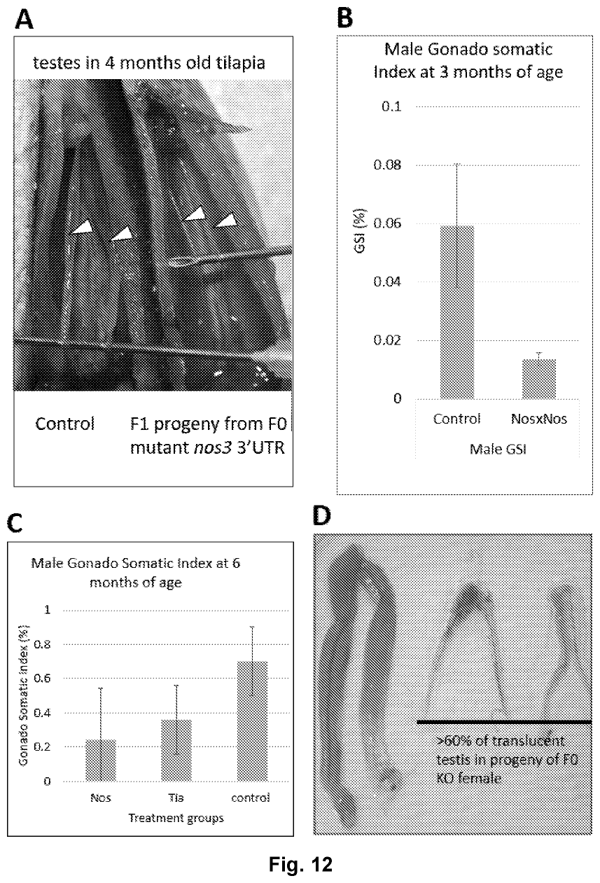

Next, we investigated the maternal effect of the mutations to determine if they altered the PGC development pathways. For this, 2-4 sibling F0 female tilapia in each treatment group were bred with wild type male and their embryo progeny was analyzed under fluorescent microscopy to score their PGC count. The average PGC ablation level ranged from 20% to 85%, depending on the gene targeted (FIG. 11 and Table 3). Different F0 females in each treatment group produced embryos with varied PGC ablation levels likely due to the mosaicism of sequence outcomes at the target sites. In contrast, all F0 mutant males crossed with females Tg(Zpc5:eGFP:nanos 3′UTR) produced embryos with normal PGC count (averaging 35-42 PGCs/embryo).

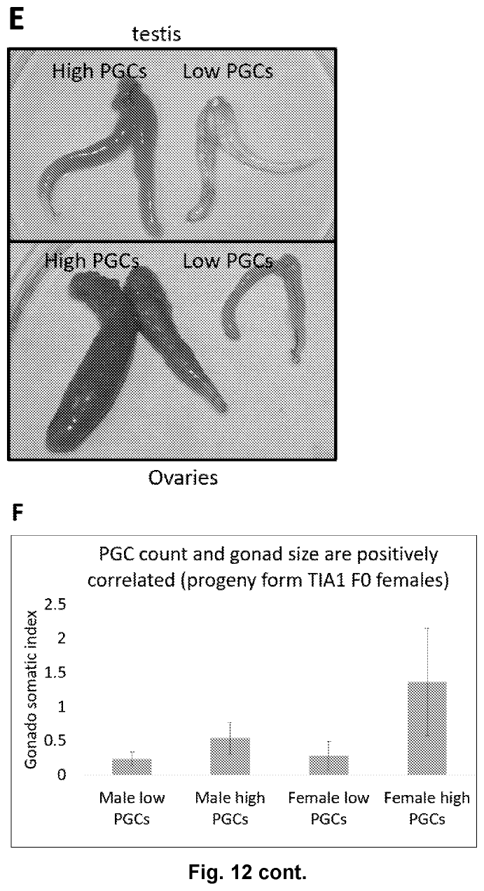

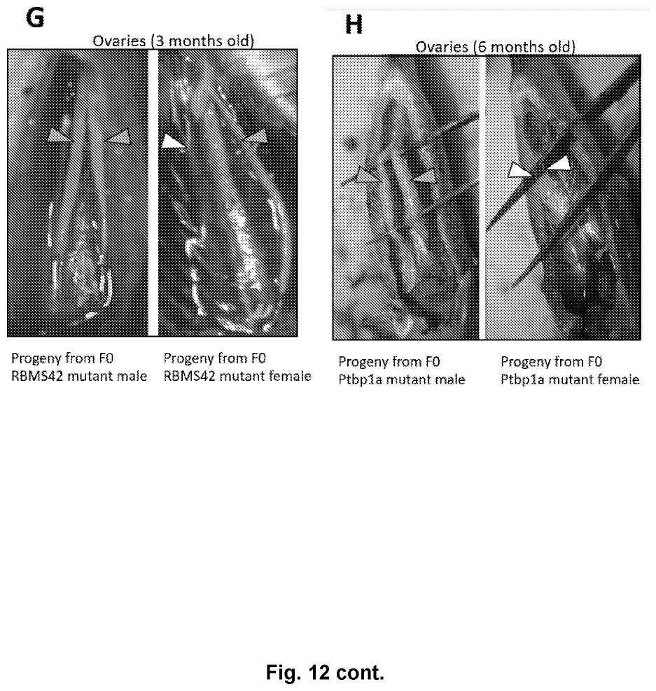

To determine if F0 females carrying mutation in nos33′UTR produced embryos with reduced PGC count, we analyzed the gonads of these progeny at 4 months and 6 months of age (since F0 female in this treatment group do not carry the GFP transgene, PGC count is not possible). Surprisingly, we observed a strong maternal effect sterility characterized by reduced gonadosomatic index with translucent testis and string-like ovaries (FIG. 12 panels A to D). Thus, while mutations in nos3 coding sequence resulted in female sterility, discrete mutations in a nos33′UTR conserved motif1 did not impair oocytes development. Instead, such mutations only appear to disrupt the post-translational regulation of nos3 mRNA in embryos progeny of mutant females. The maternal effect of the mutation on PGCs development was confirmed in subsequent generation and further discussed in the Example 16 below.

We further analyzed the development of PGC depleted gonads in the progeny from F0 females carrying mutations in TIA1, Rbms42 and Ptbp1a. We compared individuals with low PGC and high PGC counts and found a positive correlation between PGC reduction level and gonad size reduction (FIG. 12 panels E to F). We observed maternal effect sterility phenotypes including translucid testes and atrophic string-like ovaries in individuals with severely depleted PGCs (FIG. 12 panels E to H).

Altogether, our results identified several genes whose loss of function or misexpression confer a maternal-effect PGC depletion and associated sterile phenotype.

### Example 12—Validation of the Phenotypes in F1 and F2 Generations

Since F0 mutant tilapia have unpredictable plurality of sequence outcomes at the site of targeted DNA double stranded breaks, and the extent to which remaining wildtype or in-frame indel sequences are capable of obscuring the phenotype is unknown, we performed additional phenotypic characterization. Furthermore, off-target nuclease activity could have contributed to the phenotype. Thus, we propagated the intended mutation selectively, to ensure that putative off-target mutations are segregated and eliminated from subsequent generations of offspring. Eventually, the full phenotype can be measured when identical mutations are found in every cell of the animal in the F2 homozygous generations. Accordingly, for each treated group, we outbred the selected founder males with germline transmitting mutations with females Tg(Zpc5:eGFP:nanos 3′UTR) to generate F1 fish heterozygous for either frameshift mutations, insertion or precise edits in targeted gene.

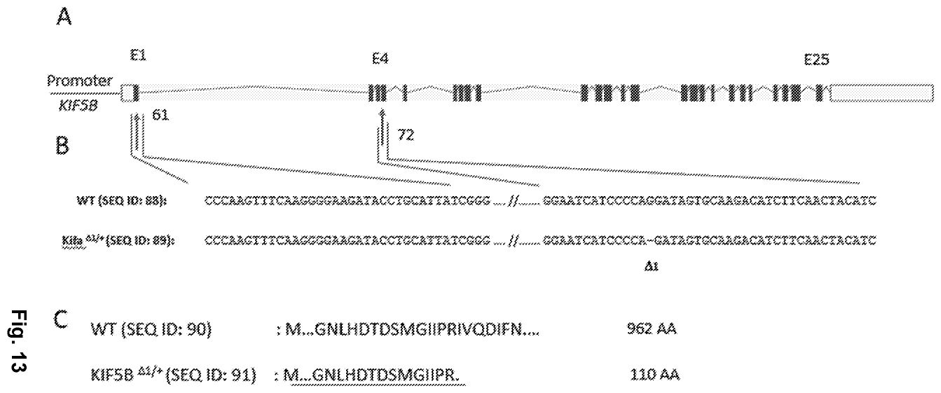

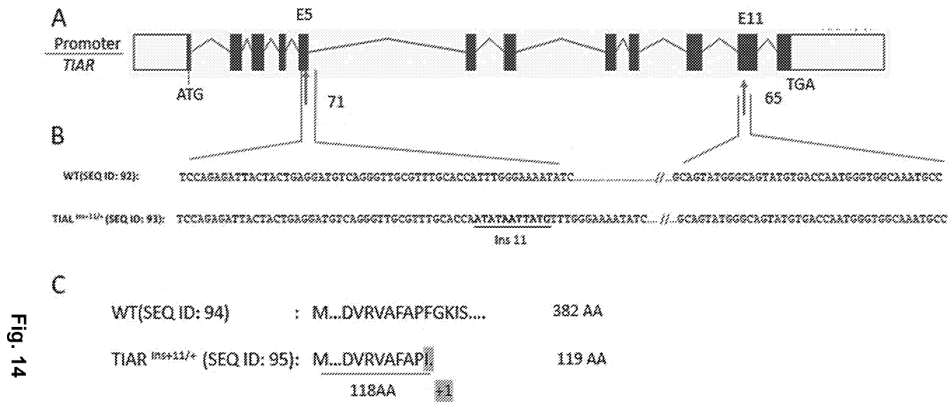

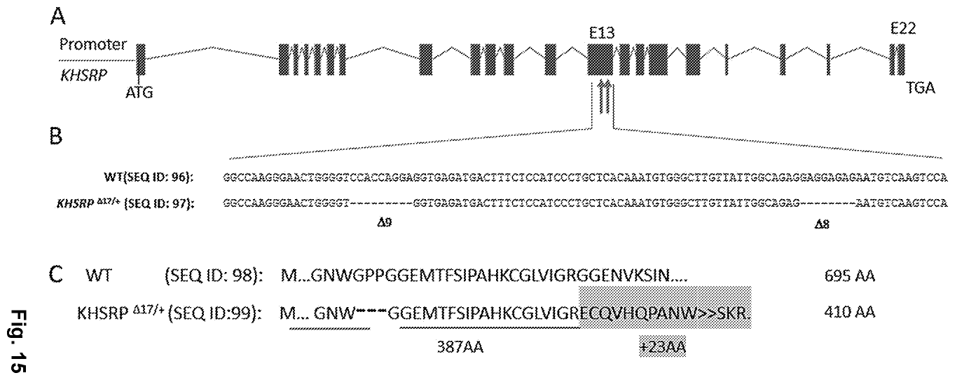

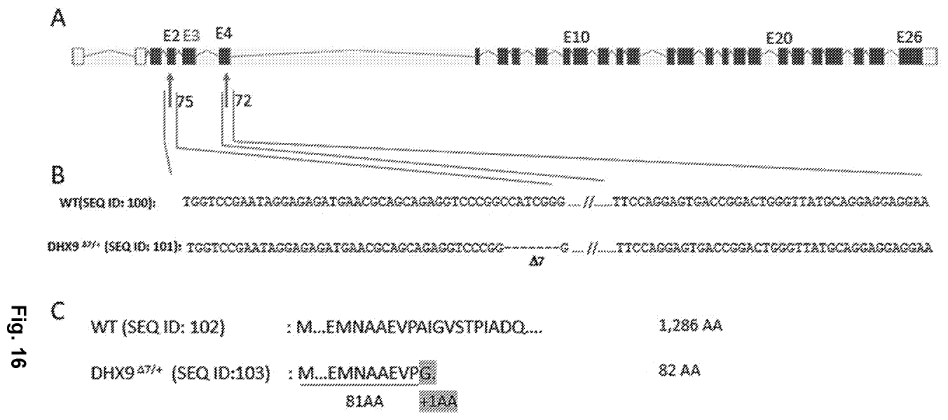

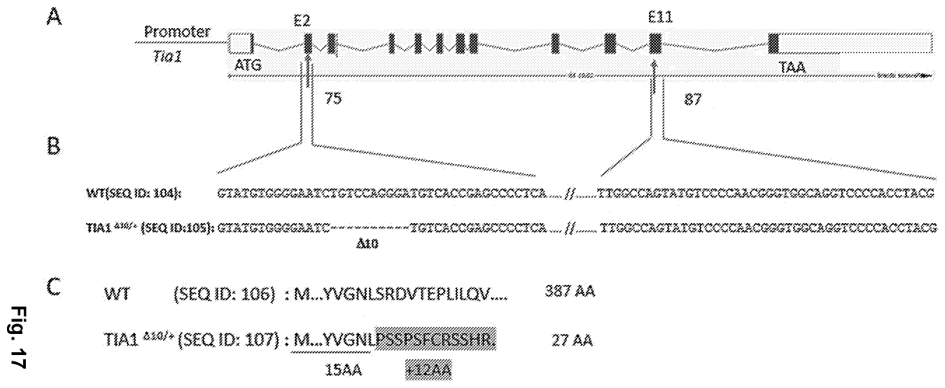

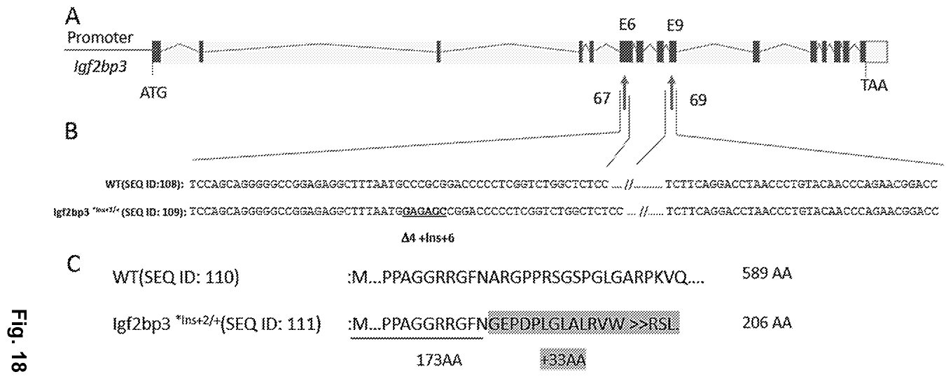

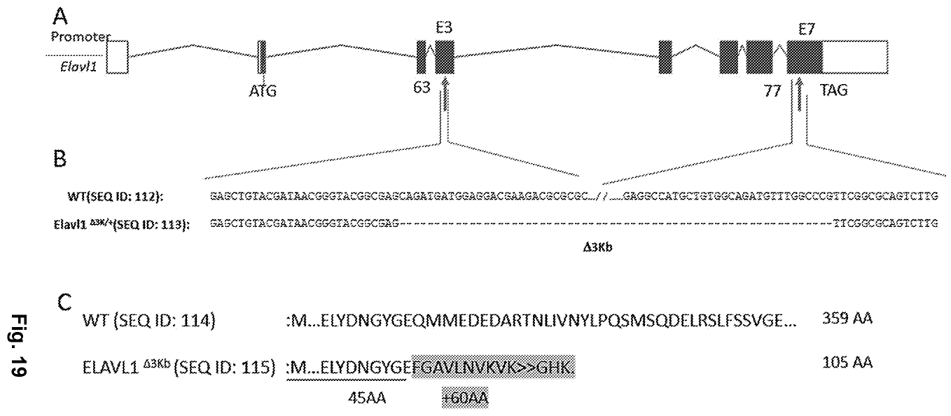

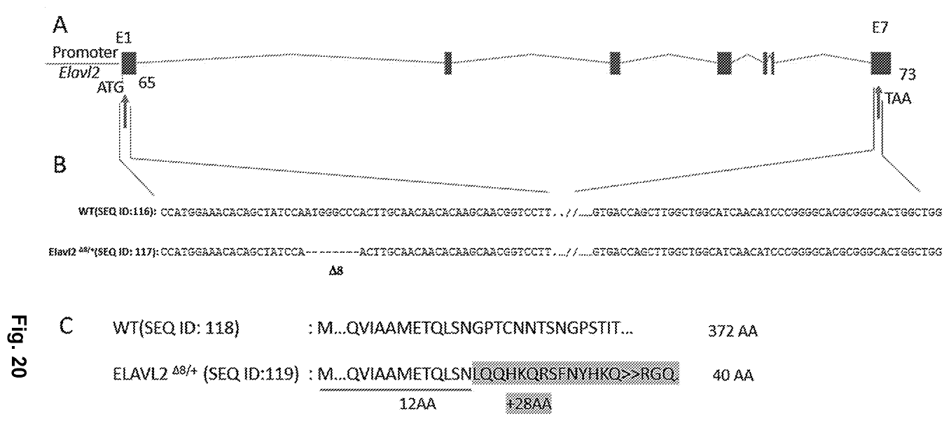

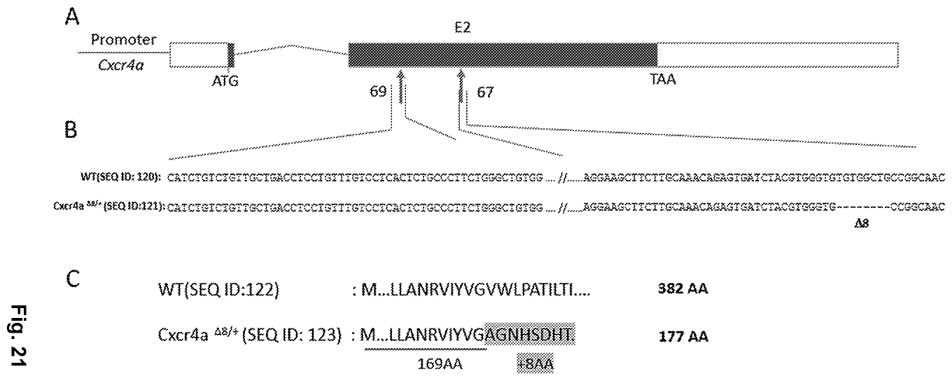

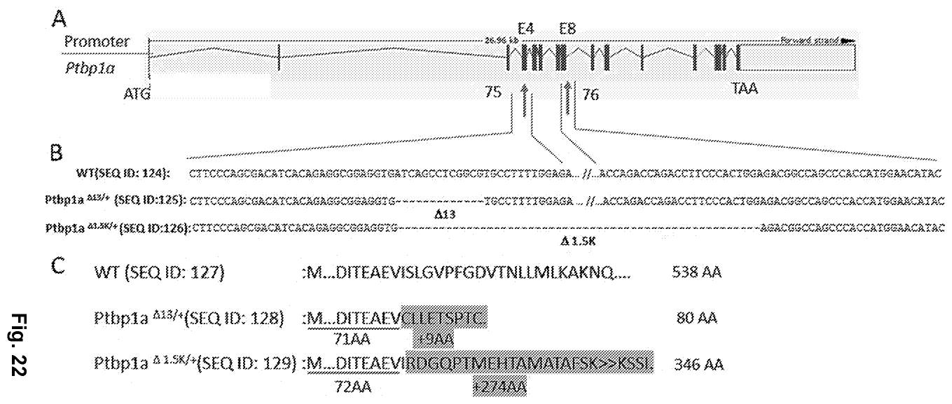

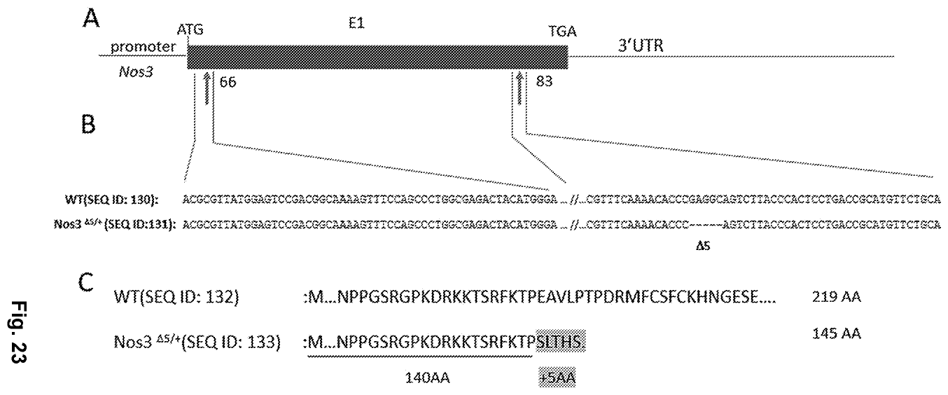

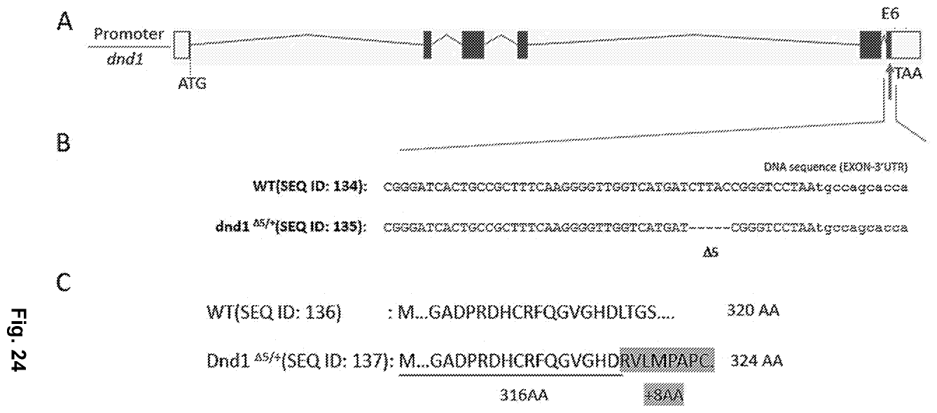

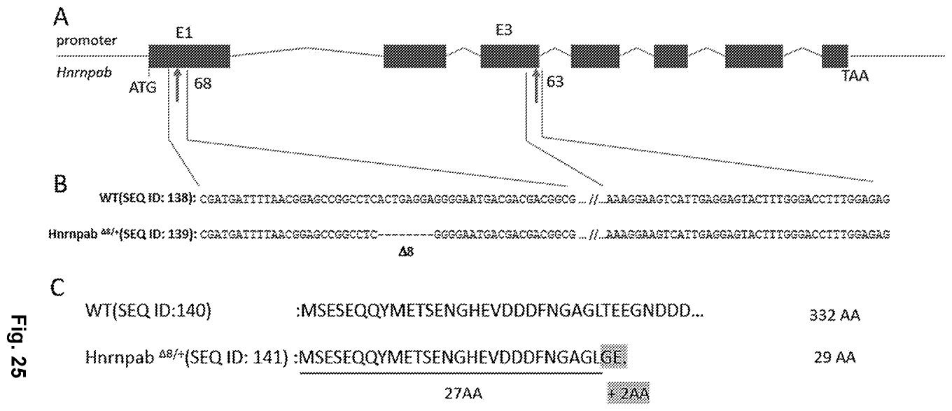

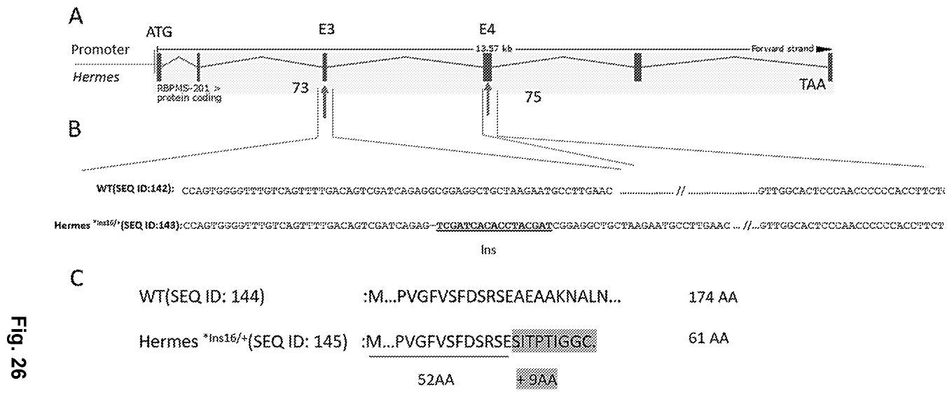

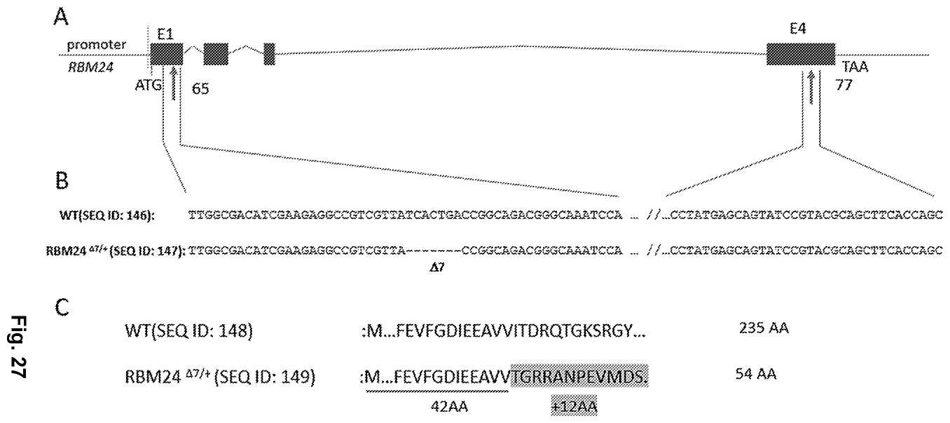

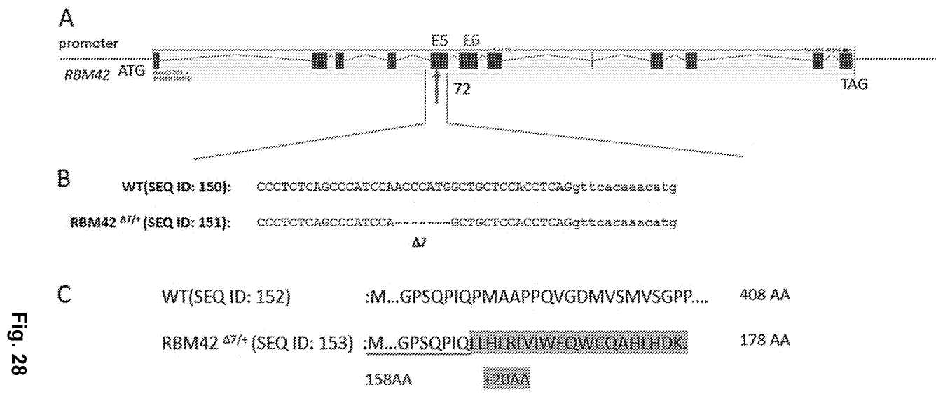

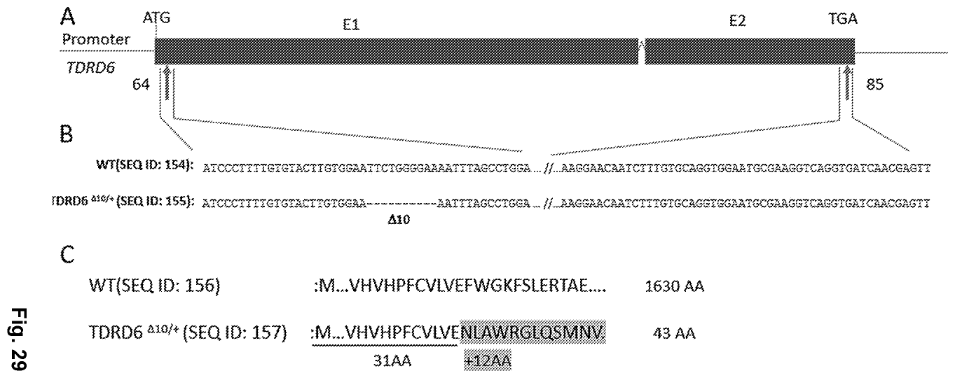

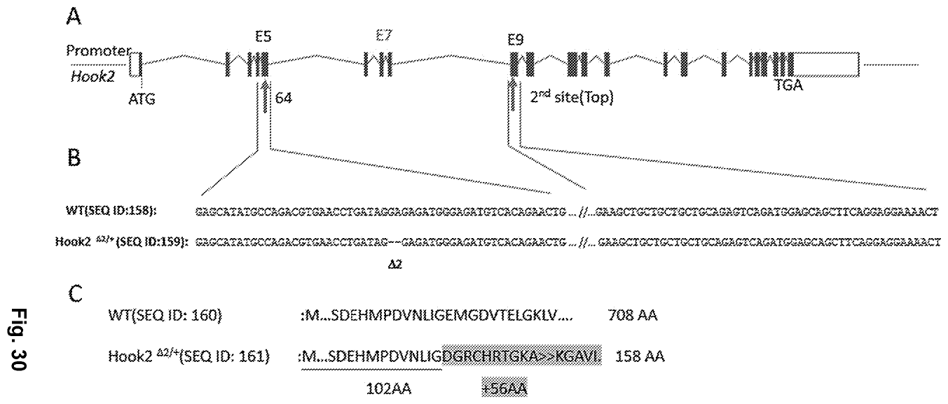

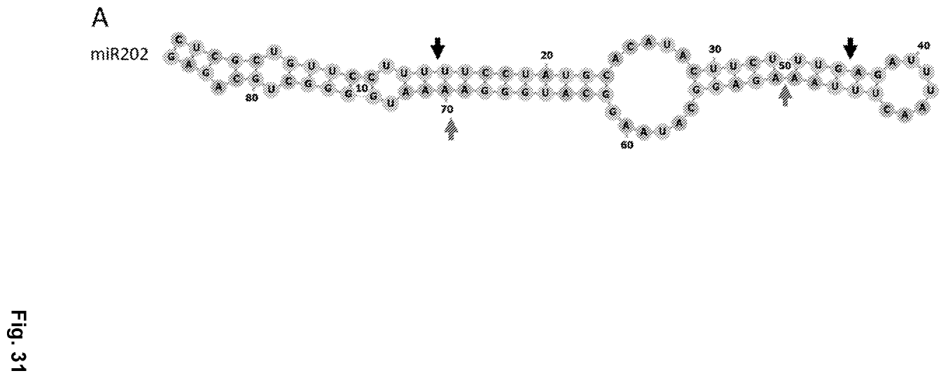

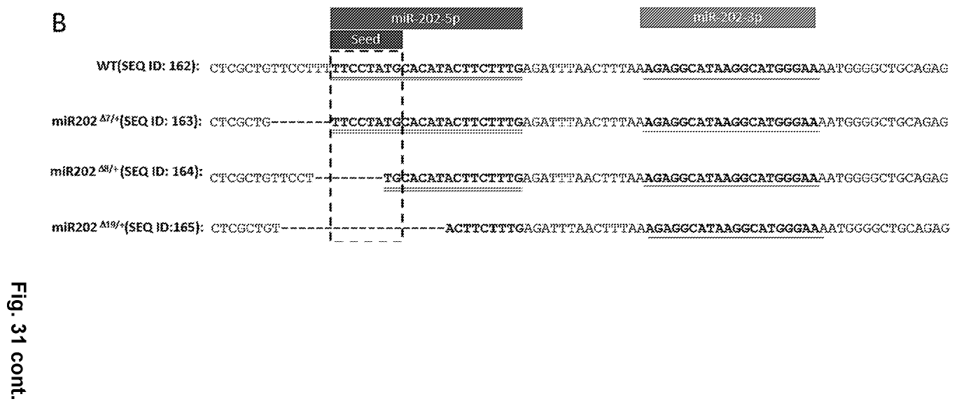

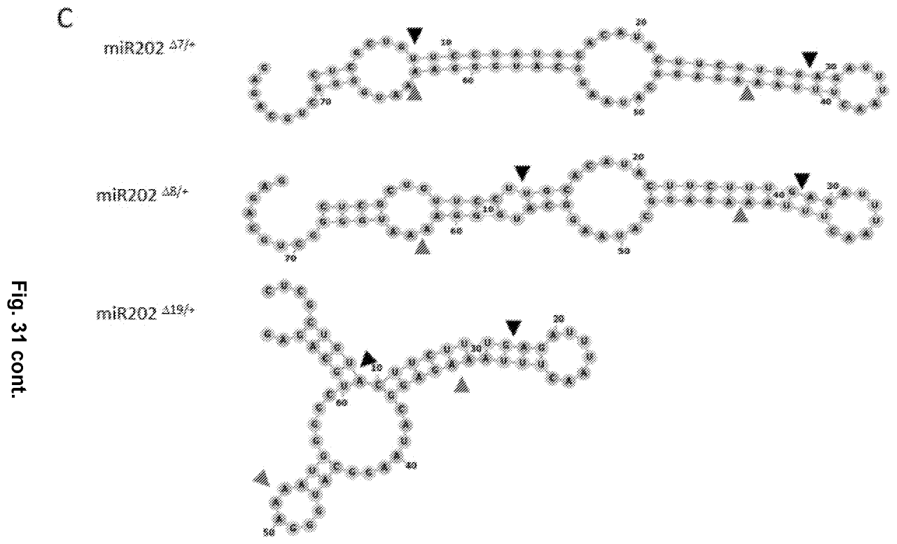

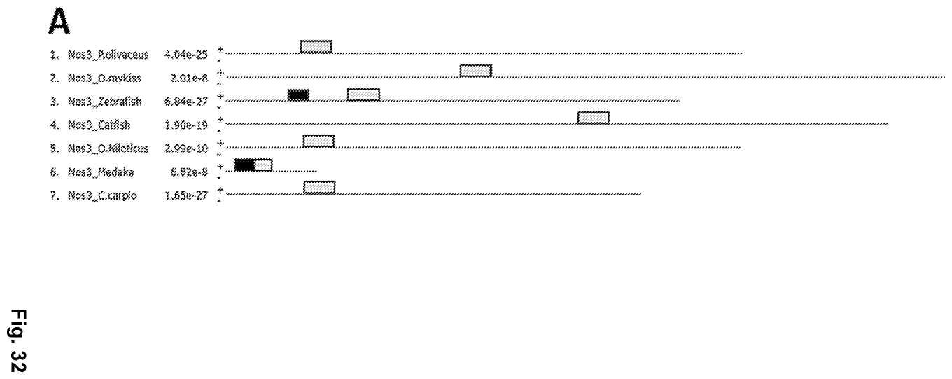

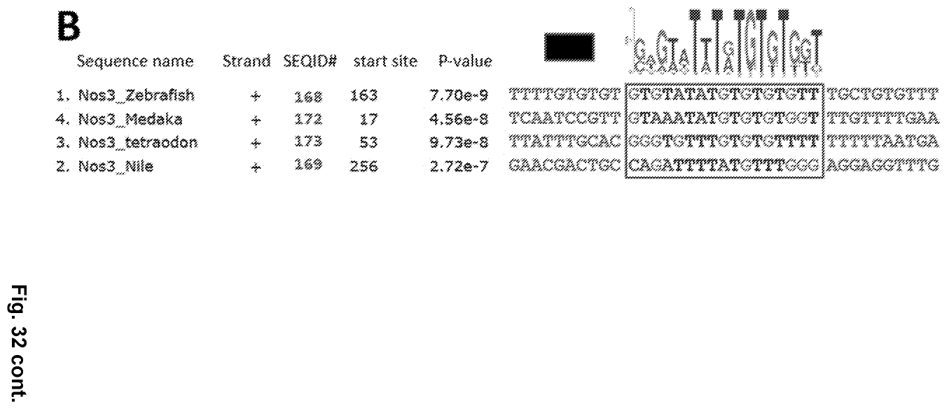

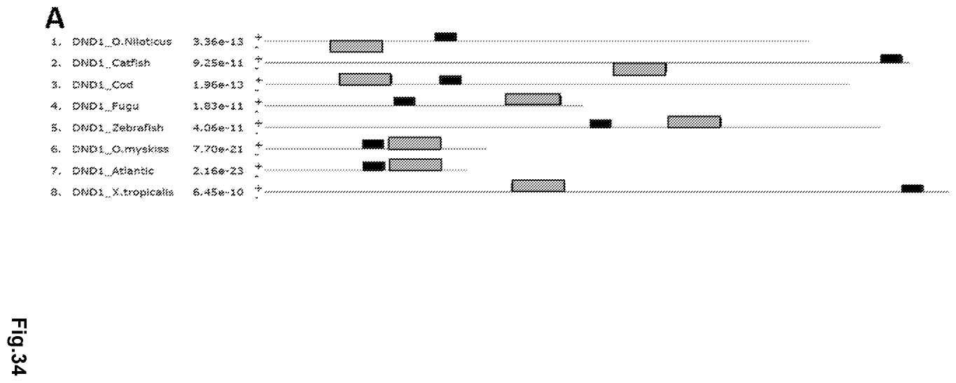

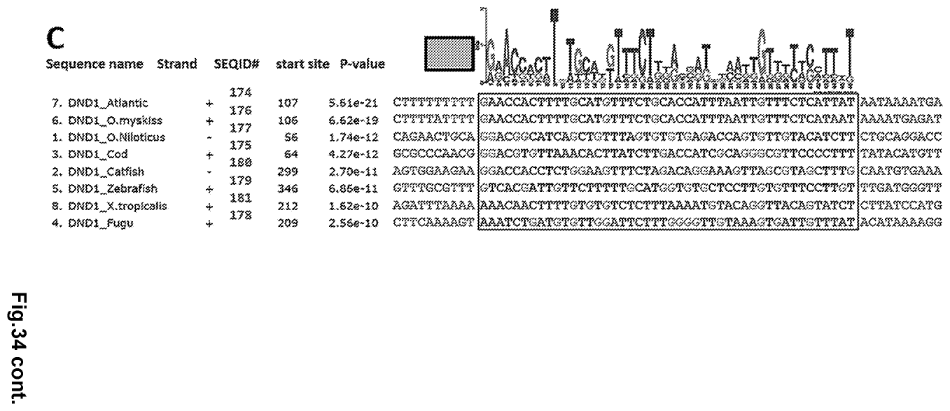

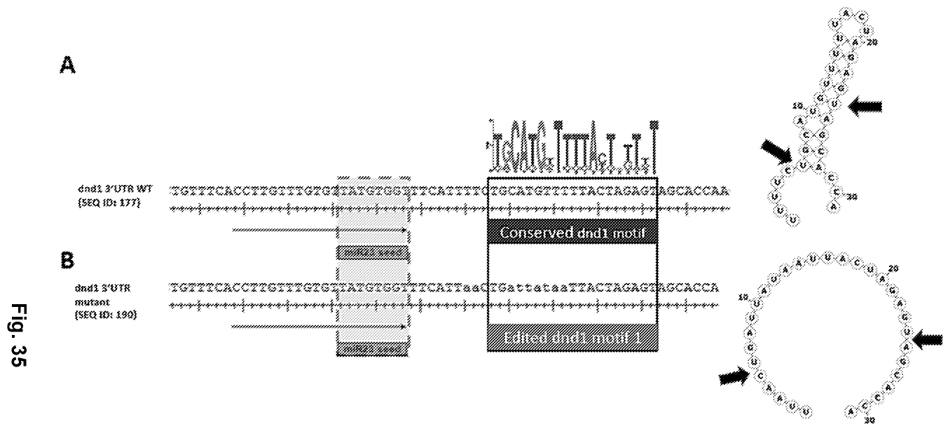

Details of the selected mutant alleles including the size of indel and predicted cDNA and protein changes are summarized in Table 3 and described in FIGS. 13-31, 33, and 35. Mutations in the 3′UTR regions of nos3 (FIG. 33) and dnd1 (FIG. 35), selectively removed (deletions) or replaced (allelic substitution) putative regulatory motifs. Finally, mutations in miRNA-202 were selected to completely or partially remove the miR-202-5p seed sequence (FIG. 31).

Heterozygous tilapia carrying these mutations appear healthy and differentiated into fertile adults of both sexes. The absence of a reproductive phenotype in these sexually mature F1 generation is not unexpected given the presence of a wild type allele of each targeted gene in all cells of selected mutant.

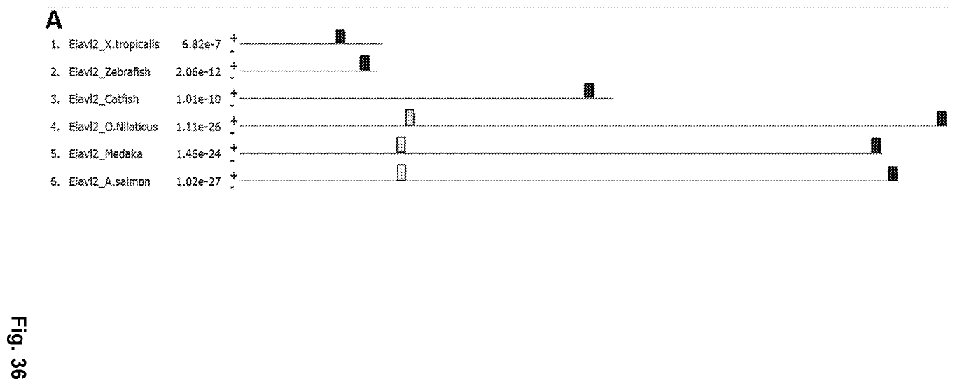

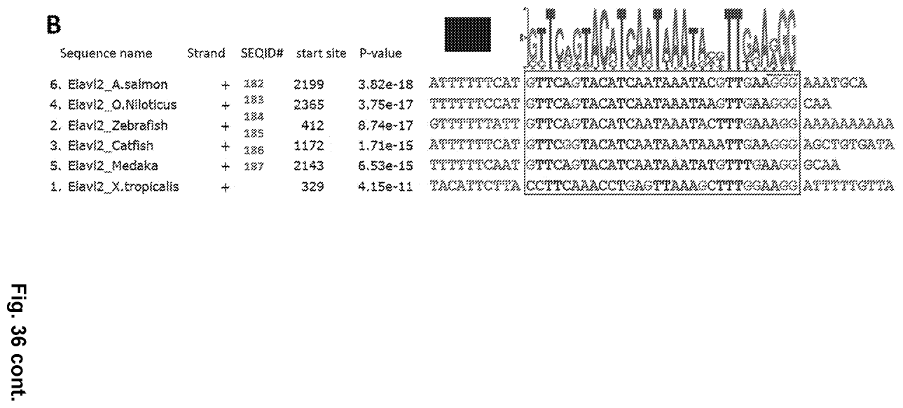

Given the apparent critical role of the genes targeted in PGC development, we further tested whether they represent a dosage-dependent mechanism. To this end, we investigated whether decreasing the maternal dose of functional mRNA/protein decreases the number of PGCs. Indeed, in oocytes of hemizygous mutant females, both alleles are expressed but only one code for a functional protein. Thus, if the targeted gene works in a dose dependent manner, we should expect the progeny from hemizygous females crossed to wild type males to show reduction in the number of PGC. We found that hemizygous mutant for KHSRP (KSHRP^{Δ17/+}) and ElavL1 (ElavL1^{Δ3k/+}) produced embryos progeny with a normal PGC count (FIG. 36). In contrast, we measured a significant PGCs reduction in the progeny from TIAR^{i11/+}, TIA1^{Δ10/+}, DHX9^{Δ7/+}, Igf2pb3^{Δ2/+}, Elavl2^{Δ8/+}, Ptpb1a^{Δ1.5k/+}, Hnrnpab^{Δ8/+}, Rbm24^{Δ7/+}, TDRD6^{Δ10/+} and miR202-5p^{Δ7/+}miR202-5p^{Δ8/+} as well as nos3 3′UTR motif1^{Δ32/+} and nos3 3′UTR motif1^{Δ8/+} (a reduction of 40-50%), revealing strong gene-dosage sensitivity (FIG. 37).

To further investigate the possibility of a zygotic effect of the mutation in early developmental processes, we scored the viability of embryos progeny from hemizygous mutant female crossed with hemizygous mutant male. We anticipated that approximately 25% of the embryo progeny are homozygous for the mutant allele.

Under white light stereomicroscope, we measured that ˜25% of the larvae from the KIFSB family developed severe craniofacial deformities, curved body with bent tails (FIG. 10). These deformed larvae were genotyped and found to carry the KIF5B^{Δ1/Δ1} allele. Mortality in F2 homozygous KIF5B^{Δ1/Δ1} mutant reached 95% at 7 days post fertilization and all homozygous mutant KIF5B died at 30 days of age. We did not observe apparent morphological somatic defects for all other gene targeted and approximately 25% of homozygous mutant were identified amongst surviving and anatomically indistinguishable sibling progeny.

To learn more about possible function of the genes targeted at later developmental stage, we raised each clutches of embryos to adulthood and analyzed the sex ratios, fertility and gonadal morphology of homozygous, hemizygous and WT sibling progeny.

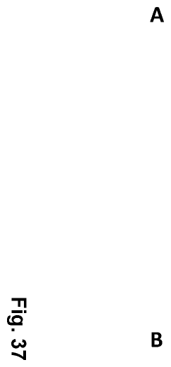

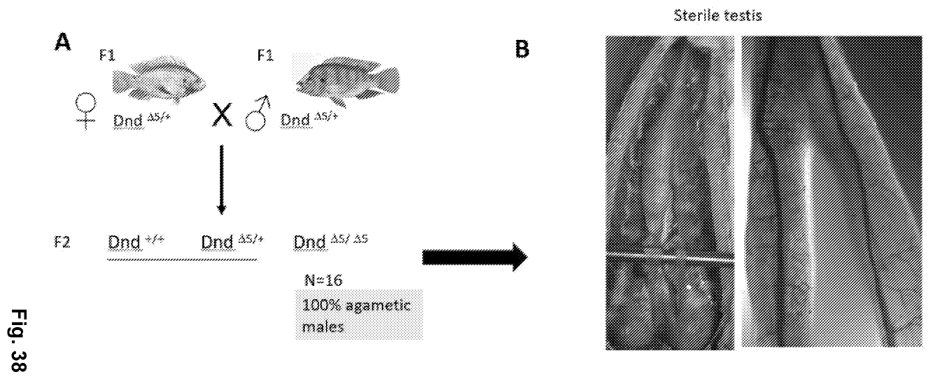

Consistent with the phenotype observed in F0 generations, the lack of zygotic nos3 and dnd1 mRNA resulted in sterility phenotypes. We found that nos3-knockout (nos3^{Δ5/Δ5}) developed into fish of both sexes. We found nos3^{Δ5/Δ5} female to be agametic with a string like ovary (FIG. 39). Furthermore, nos3 deficient male showed partially translucid testes compared to the pink colored opaque testes in WT and hemizygous mutant. At 6 months of age, sperm from nos3^{Δ5/Δ5} male concentration was dramatically reduced; however, we found no defect in sperm morphology, motility or functionality. Thus, nos3^{Δ5/Δ5} males show delayed maturation but remained fertile.

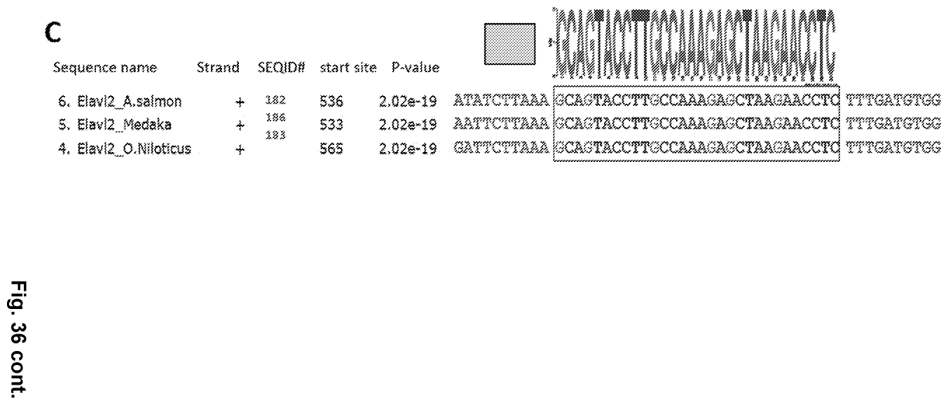

We only raised dnd1 KO tilapia (dnd1) and all developed into males (n=17). These males showed translucid testes and were agametic, as confirmed by cellular and molecular analysis of their testes (FIG. 38).

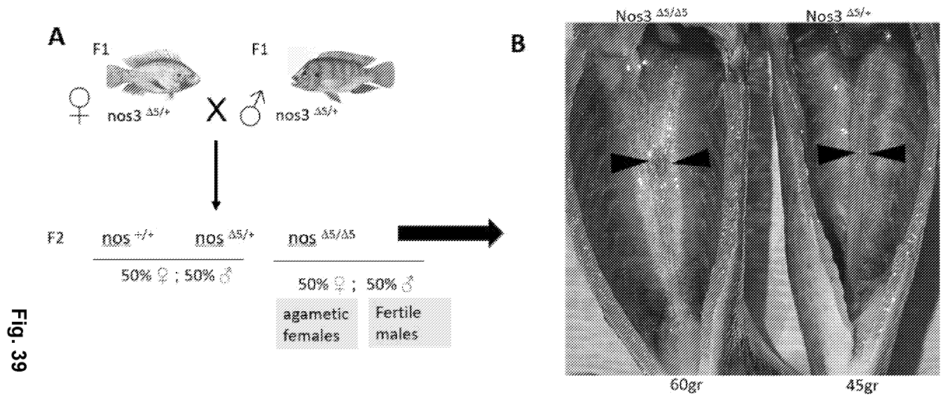

We further show that the RNA binding protein Elavl2 is fundamental for gametogenesis both in males and females because loss-of-function mutation results in complete abrogation of gametes in both sexes as evidence by morphological and molecular analysis of their gonads (FIG. 40).

### Example 13—Single Homozygous KO Genes with Maternal Effect Sterility Phenotypes

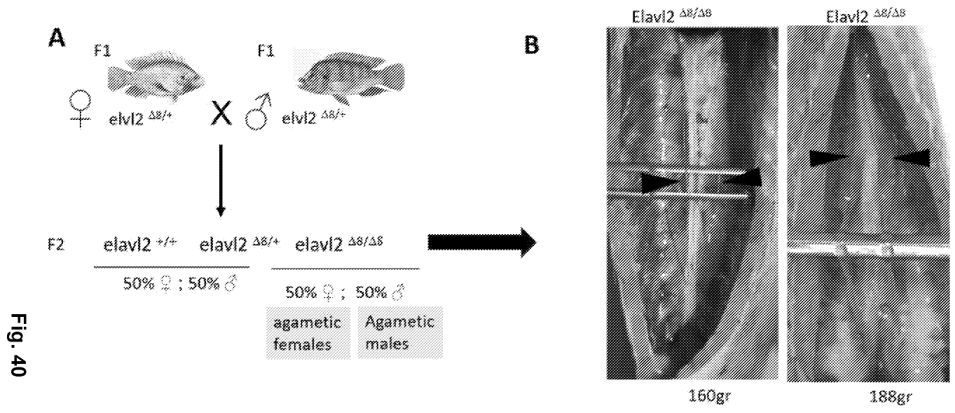

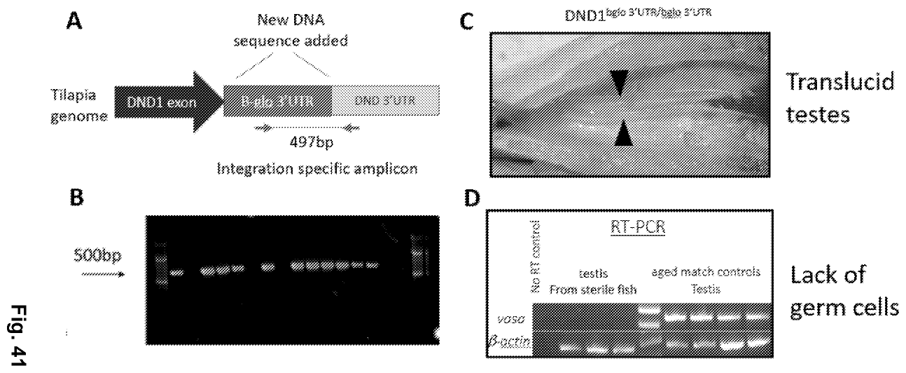

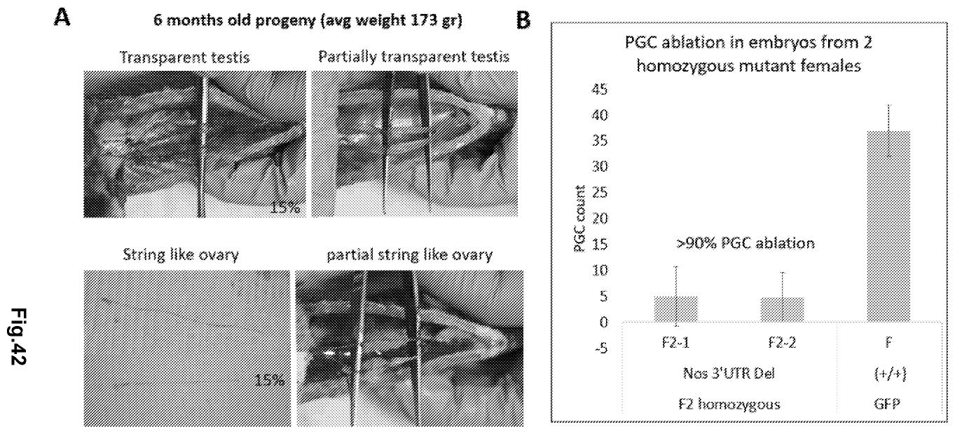

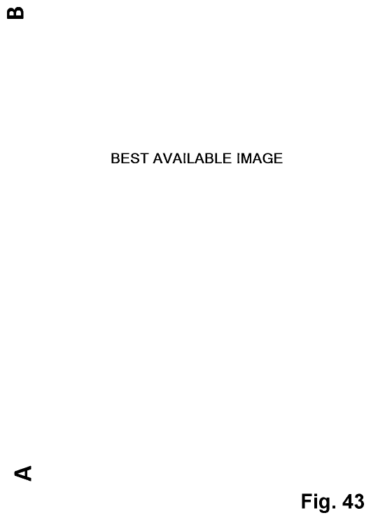

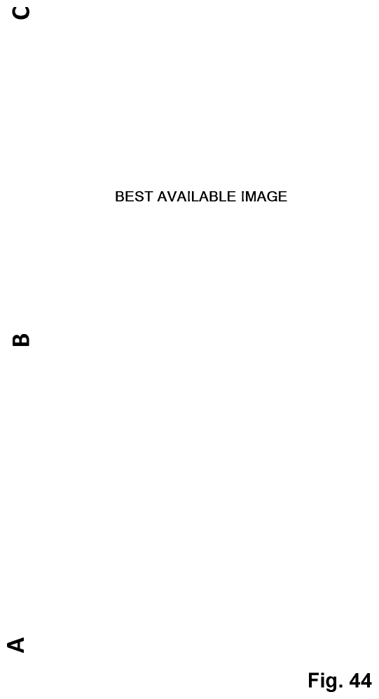

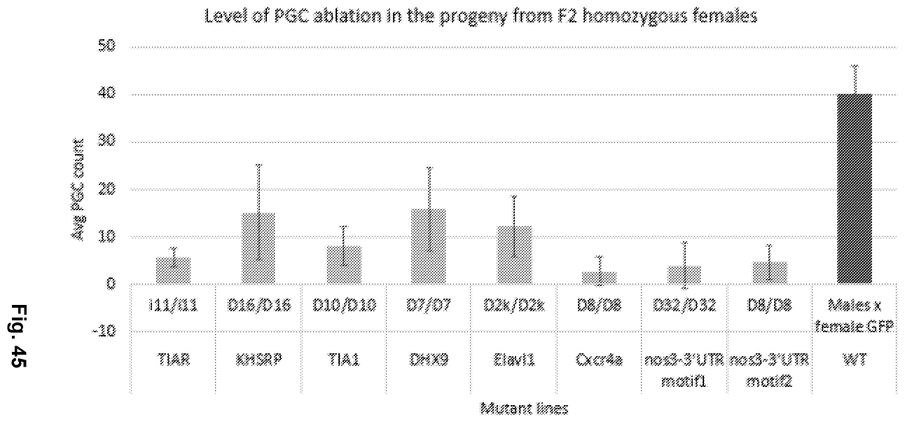

For all other genes targeted, we recovered all anticipated genotypes at the expected Mendelian frequencies with no obvious phenotypes through adulthood. To measure the full strength of the maternal effect sterility phenotype, we crossed homozygous mutant females with WT males and analyzed the embryos progeny. We observed strong PGC reduction in the progeny of females homozygous for the following alleles TIAR, KHSRP, TIA1, DHX9, Elavl1, Cxcr4 (FIG. 45). PGC depleted progeny from mutant females were raised to adulthood and their gonads were analyzed for size and alterations. We found atrophic ovaries with string like structures as well as translucid germ cell depleted testis consistent with the severe PGC loss in embryos. In contrast, progeny from F2 mutant males developed normal sized gonads. For example, we measured that TIAR1 homozygous mutant female produced progeny with a mean gonadosomatic index 10-20 folds lower than controls (progeny from homozygous mutant male) (FIG. 43). Compared to null mutation in nos3 coding sequence, deletion of the motif1 sequence located in nos3 3′UTR did not result in female sterility, suggesting that this motif is not required for the maintenance of oogonial stem cells. Importantly however, those females produce embryos with severe PGCs ablation (FIGS. 41 and 44). The Maternal effect phenotype for the remaining genes are still under investigation. The incomplete PGC ablation phenotype resulting from single gene inactivation suggest that these genes participate in complex pathway with significant genetic redundancy.

### Example 14—Dissecting the Genetic Architecture of PGC Formation

To better understand the genetic architecture of PGCs development and determine a functional order of action of genes involves in these processes, we established double mutant lines and compared the PGCs count in the progeny from single gene or double gene loss-of function phenotypes. Furthermore, to determine if existing mutations govern the post-transcriptional regulation of nos3, we study the effect single mutations in a transgenic line of tilapia expressing the proapoptotic gene bax fused to nos3 3′UTR under the control of an oocyte specific promoter (MSC transgenic line). We previously established that MSC female produce embryos lacking PGCs from ectopic maternal expression of BAX in these cells (Lauth and BUCHANAN 2016).

We found merely additive PGC effect (no epistasis) in tilapia lines carrying MSC-khrsp^{Δ16/Δ16}, MSC-DHX9^{Δ7/Δ7}, MSC-TIAR^{ι11/ι11} suggesting that these genes do not interact with nos3′UTR (FIG. 44). Indeed, the MSC was designed to exploit the oocyte's own cellular machinery to drive expression of bax:nos33′UTR to PGCs of embryos progeny. If the genes targeted were involved in the post-transcriptional regulation of nos3 3′UTR the proapoptotic protein Bax expression would not be restricted to PGCs, limiting the MSC-PGCs ablation capacity. We conclude that DHX9, TIAL and KHSRP are neither directly nor indirectly involved in the localization of nos3.

### Example 15—Tilapia Germ Plasm Genes with Pleiotropic Phenotypes not Restricted to PGCs Development

Our mutagenesis screen uncovered new germ plasm genes whose inactivation in tilapia prevent the development of fertile female. We found that inactivation of Hnrnph1 and Rbms resulted in embryonic lethality. Our results further agree with earlier finding that embryos deficient for Kif5Ba exhibit a mix of moderately to severely ventralized phenotypes (Campbell, Heim et al. 2015).

Our results show that the zygotic function of nos3 in tilapia is required for the maintenance of oogonial stem cell, with nos3^{Δ5/Δ5} mutant females developing string like agametic ovaries at maturity, while mutant males remain fertile (FIG. 39). Interestingly, our nos3 mutant tilapia female did not sex-reverted to a male phenotype. In this regard, our results disagree with those of Li et al. (Li, Yang et al. 2014) and indicate a germ cell independent sex determination mechanism in tilapia.

Our results confirm the findings of a previous study in Atlantic salmon showing that zygotic dnd1 expression is required for the continued maintenance of germ cells and that maternally contributed dnd1 mRNA and/or protein cannot rescue the zygotic function of this gene (Wargelius, Leininger et al. 2016).

We generated loss of function mutation in ElavL2 which encodes a protein that shows significant similarity to the product of the Drosophila elav gene (embryonic lethal, abnormal visual system), the absence of which causes multiple structural defects and embryonic lethality. Elavl2 was found to be abundantly expressed in zebrafish brain as well as in PGCs during early embryonic development (Thisse and Thisse 2004, Mickoleit, Banisch et al. 2011). We were therefore surprised to see that tilapia ElavL2^{Δ8/Δ8} homozygous mutants are perfectly viable, developing into sterile male and female (FIG. 40). Thus, like nos3, dnd1, vasa and piwi-like genes, Elavl2 show essential zygotic function that ensure the maintenance of adult germ cell.

### Example 16—Novel RNA Binding Proteins Involved in PGC Formation

Somewhat surprisingly, we successfully identified genes whose loss-of-function mutations produced severe defect in PGCs development with no other obvious phenotype to adulthood, indicating that they are not required for viability or fertility. Here, we describe for the first time the defects caused by TIA1, TIAR, KHSRP, Rbm24, Rbm42, DHX9, Igf2pb3, Hnrnph1 and EIavL1 loss of function mutations in any animal species where germ cells are specified by maternal inheritance (e.g all fish, many insects and frog species). Embryos derived from mutant mothers for these genes had, on average, between 60% and up to 88% of PGC number reduction. In some, but not all maternal mutant genotypes this reduction correlated with an increase in variance of this quantitative trait. An increase variance could indicate a role of buffering agent to stabilize gene regulatory network controlling germ cell number. Mice lacking TIAL1 exhibit partial embryonic lethality and defective germ cell maturation (Beck et al., 1998), implicating TIA1 proteins in regulation of essential aspects of vertebrate development. We also describe the first defect caused by the inactivation of Hook2, Tdrd6, dnd1 and KIFSB in tilapia.

### Example 17—Identification and Functional Analysis of 3′UTR Regulatory Motifs

To solve the problem associated with pleiotropic function of essential protein required for germ cell maintenance, we investigated the possibility to deactivate selectively the maternal gene function without affecting its zygotic activity. We specially investigated the 3′UTR function of tilapia nos3 and dnd1 which are respectively required in embryos and adults for the formation and continued maintenance of the germ line. Given the possible involvement of Elavl2 in PGC formation (Mickoleit, Banisch et al. 2011), and its requirement for germ line maintenance in adult (our study), we further included Elavl23′UTR to our analysis.

To interrogate the contribution of tilapia dnd1 3′UTR in maintaining adult germ cells, we performed a 3′UTR swapping experiment with the 3′UTR of the tilapia β-globin gene. The expression of cytoplasmic β-globin gene is generally believed to be constitutive and ubiquitous in all cell type and expected to lack cis-acting motifs necessary for PGCs expression (Herpin, Nakamura et al. 2009). We found that β-globin 3′UTR cannot be used as an alternative 3′UTR to maintain the zygotic function of dnd1, suggesting that specific post-transcriptional regulations are necessary for DND1 activity in the zygote.

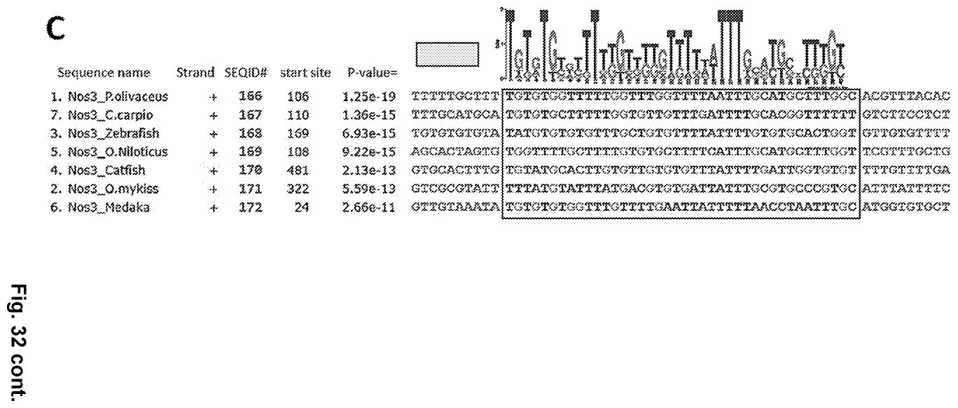

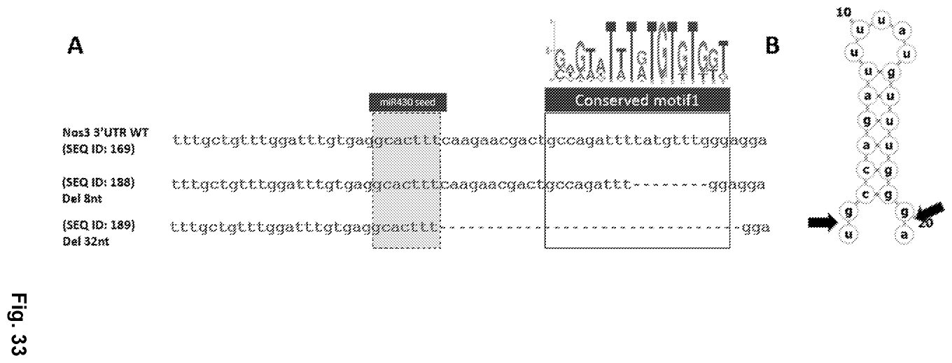

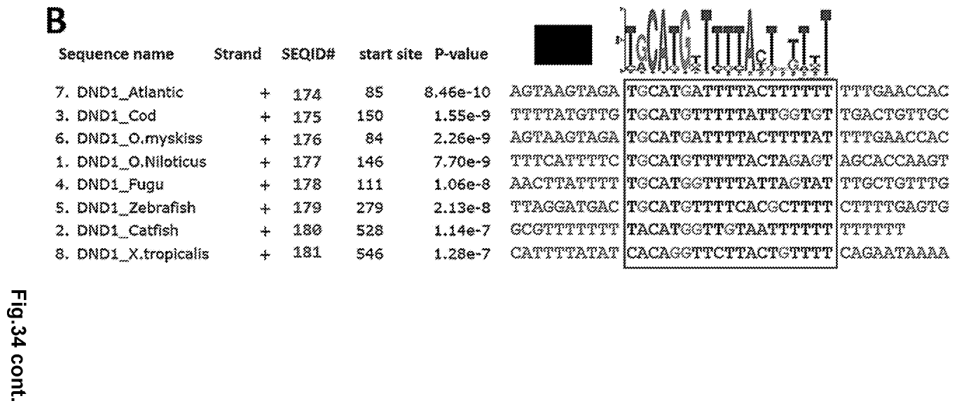

RNA Localization to germ plasm is mediated by 3′UTR specific cis-regulatory elements whose requirement for the zygotic function remain untested. To first map candidate regulatory elements, we imputed the 3′UTR sequences of varied nos3, dnd1 and Elavl2 transcripts across different species into a web-based software motif discovery algorithm. Despite the low sequence similarities in multiple sequence alignments, and 3-9 folds variation in their length, we successfully identified varied conserved motifs in the 3′UTR for these orthologous genes. The result of nos33′UTR sequences analysis reveal two conserved motifs, one of which was present in all nos33′UTR sequences analyzed (FIG. 32). The result of dnd1 3′UTR analysis is a set of two predicted binding motifs found at varied location across all teleost species examined as well as in Xenopus tropicalis (FIG. 34). Finally, analysis of Elavl2 3′UTR identified 2 motifs, one of which was present at the same location in the 3′UTR of all species examined (FIGS. 35A and B). The second Elavl2 motif (Elavl2 motif2) is perfectly conserved in Atlantic Salmon, Medaka and Nile tilapia (FIG. 36 panel C). Because RNA regulatory elements typically entail a combination of a loosely defined primary sequence within the context of a secondary structure (Keene and Tenenbaum 2002) we performed computational studies of these regions using an RNA folding algorithm (Kerpedjiev, Hammer et al. 2015). Amongst the different motifs, nos3-motif1 and dnd1-motif1 were jointly recognized by several programs analyzing similarities in RNA sequence and folding predict. The sequence alignments, motif logos (graphic representation of the relative frequency of nucleotides at each position) and predicted secondary structures for these motifs are shown in FIGS. 32 and 33.

To further evaluate the plausibility of these regions we performed a scan for consequential pairing of seed target for miR-430, miR-23 and miR-101. miR430 is the most abundant miR in early zebrafish embryo and is known to inhibit nos3 and tdrd7 mRNAs in somatic cells (Mishima, Giraldez et al. 2006). These conserved miRNA families have been detected in unfertilized eggs and early embryos in many teleost species (Ramachandra, Salem et al. 2008) suggesting an important conserved role, possibly regulating germ plasm RNA. We found two putative oni-miR-23 sites in tilapia dnd1 3′UTR, one miR-430 and one miR-101 site in tilapia nos3 3′UTR located in closed proximity to the conserved predicted binding motifs1 and 2 of tilapia nos3 and dnd1 3′UTR. Without wishing to be bound by a theory, our analysis suggests a mechanism in which conserved cis-acting motifs and trans-acting RNA binding factors form mRNA-protein complexes (mRNPs). These interactions may protect against miRNA degradation in a germ plasm specific manner. Taken together, of the 6 binding motifs, dnd1-1 and nos3-1 were prioritized for further investigation in this study.

As initially hypothesized and in contrast to nos3 loss of function mutation, we found that the disruption of nos3-3′UTR motif-1 does not impair the zygotic function of the gene. We observed that motif-1 deficient females develop a functional ovary. Importantly, we confirmed that this motif is required for the maternal function of the gene. We observed that motif-1 deficient females produce PGC depleted embryos that grew into sexually delayed and/or agametic adults (FIGS. 41B and A). The occurrence of this 18-mer and other motifs in the 3′-UTR of orthologs genes over a wide range of organisms can explain the functional interchangeability of 3′UTR across lower vertebrates ranging from fish to frogs.

We propose that a similar approach can be used for the prediction of binding motifs target of RNA-binding proteins and anticipate that such systematic identification will identify valuable target for modification to achieve deregulation of additional maternal genes governing the formation of PGCs.

We speculate that inactivation of other conserved 3′UTR regulatory sequences will not result in pleiotropic phenotypes detrimental to the survival, sex determination or fertility of the homozygous mutant female. The conserved nature of cis-acting elements renders these sequences specifically attractive as target to achieve the same maternal effect phenotypes in different aquaculture species of fish.

### Example 18—Analysis of miR-202-5p Targeted Modification

We further describe for the first time the effect caused by miR-202-5p inactivation on PGCs development. This miR202 is evolutionary conserved and has two mature transcripts, miR-202-5p and miR-202-3p with miR-202-5p representing the dominant arm in ovaries during late vitellogenesis of zebrafish (Vaz, Wee et al. 2015) marine medaka (Presslauer, Bizuayehu et al. 2017), rainbow trout (Juanchich, Le Cam et al. 2013), tilapia (Xiao, Zhong et al. 2014), Atlantic halibut (Bizuayehu, Babiak et al. 2012) and Xenopus tropicalis (Armisen, Gilchrist et al. 2009). It was recently reported that the inactivation of miR-202 (combined loss of miR202-3p and miR202-5p) in medaka result in sterile female lacking eggs or subfertile female laying reduced number of abnormal and non-viable eggs. The reproductive phenotype reflect an impaired folliculogenesis (Gay, Bugeon et al. 2018). Interestingly, our F0 and F1 miR-202 mutant females produced viable PGC depleted progeny.

### REFERENCES

(1) Carlson, D. F., S. C. Fahrenkrug, and X. Lauth, Control of sexual maturation in animals. 2013, Google Patents.
(2) Koga, A., et al., Insertion of a novel transposable element in the tyrosinase gene is responsible for an albino mutation in the medaka fish, Oryzias latipes. Molecular and General Genetics MGG, 1995. 249(4): p. 400-405.
(3) Krauss, J., et al., transparent, a gene affecting stripe formation in Zebrafish, encodes the mitochondrial protein Mpv17 that is required for iridophore survival. Biology open, 2013. 2(7): p. 703-710.
(4) Slanchev, K., et al., Control of Dead end localization and activity— implications for the function of the protein in antagonizing miRNA function. Mechanisms of development, 2009. 126(3): p. 270-277.
(5) Kedde, M., et al., RNA-Binding Protein Dnd1 Inhibits MicroRNA Access to Target mRNA. Cell, 2007. 131(7): p. 1273-1286.
(6) Li, M., et al., Conserved elements in the nanos3 3′ UTR of olive flounder are responsible for the selective retention of RNA in germ cells. Comparative Biochemistry and Physiology Part B: Biochemistry and Molecular Biology, 2016. 198: p. 66-72.
(7) Skugor, A., et al., Conserved mechanisms for germ cell-specific localization of nanos3 transcripts in teleost species with aquaculture significance. Marine biotechnology, 2014. 16(3): p. 256-264.
(8) Oka, K., et al., Genotyping of 38 insertion/deletion polymorphisms for human identification using universal fluorescent PCR. Molecular and cellular probes, 2014. 28(1): p. 13-18.
(9) Raz, E., The function and regulation of vasa-like genes in germ-cell development. Genome Biol, 2000. 1(3): p. 1017.1-1017.6.
(10) Armisen, J., et al. (2009). “Abundant and dynamically expressed miRNAs, piRNAs, and other small RNAs in the vertebrate Xenopus tropicalis.” Genome research 19(10): 1766-1775.
(11) Bizuayehu, T. T., et al. (2012). “Sex-biased miRNA expression in Atlantic halibut (Hippoglossus hippoglossus) brain and gonads.” Sexual Development 6(5): 257-266.
(12) Campbell, P. D., et al. (2015). “Kinesin-1 interacts with Bucky ball to form germ cells and is required to pattern the zebrafish body axis.” Development 142(17): 2996-3008.
(13) Eshel, O., et al. (2014). “Identification of male-specific amh duplication, sexually differentially expressed genes and microRNAs at early embryonic development of Nile tilapia (Oreochromis niloticus).” BMC genomics 15(1): 774.
(14) Gay, S., et al. (2018). “MiR-202 controls female fecundity by regulating medaka oogenesis.” PLoS genetics 14(9): e1007593.
(15) Giraldez, A. J., et al. (2006). “Zebrafish MiR-430 Promotes Deadenylation and Clearance of Maternal mRNAs.” Science 312(5770): 75-79.
(16) Herpin, A., et al. (2009). “A highly conserved cis-regulatory motif directs differential gonadal synexpression of Dmrt1 transcripts during gonad development.” Nucleic acids research 37(5): 1510-1520.
(17) Juanchich, A., et al. (2013). “Identification of differentially expressed miRNAs and their potential targets during fish ovarian development.” Biology of reproduction 88(5): 128, 121-111.
(18) Keene, J. D. and S. A. Tenenbaum (2002). “Eukaryotic mRNPs may represent posttranscriptional operons.” Molecular cell 9(6): 1161-1167.
(19) Kerpedjiev, P., et al. (2015). “Forna (force-directed RNA): simple and effective online RNA secondary structure diagrams.” Bioinformatics 31(20): 3377-3379.
(20) Koga, A., et al. (1995). “Insertion of a novel transposable element in the tyrosinase gene is responsible for an albino mutation in the medaka fish, Oryzias latipes.” Molecular and General Genetics MGG 249(4): 400-405.
(21) Krauss, J., et al. (2013). “transparent, a gene affecting stripe formation in Zebrafish, encodes the mitochondrial protein Mpv17 that is required for iridophore survival.” Biology open 2(7): 703-710.
(22) Lauth, X. and J. T. B. T. BUCHANAN (2016). Maternally induced sterility in animals, Google Patents.
(23) Li, M., et al. (2014). “Efficient and heritable gene targeting in tilapia by CRISPR/Cas9.” Genetics 197 (2): 591-599.
(24) Mickoleit, M., et al. (2011). “Regulation of hub mRNA stability and translation by miR430 and the dead end protein promotes preferential expression in zebrafish primordial germ cells.” Developmental dynamics 240(3): 695-703.
(25) Mishima, Y., et al. (2006). “Differential Regulation of Germline mRNAs in Soma and Germ Cells by Zebrafish miR-430.” Current Biology 16(21): 2135-2142.
(26) Presslauer, C., et al. (2017). “Dynamics of miRNA transcriptome during gonadal development of zebrafish.” Scientific reports 7: 43850.
(27) Ramachandra, R. K., et al. (2008). “Cloning and characterization of microRNAs from rainbow trout (Oncorhynchus mykiss): their expression during early embryonic development.” BMC developmental biology 8(1): 41.
(28) Thisse, B. and C. Thisse (2004). “Fast release clones: a high throughput expression analysis.” ZFIN direct data submission 2004.
(29) Vaz, C., et al. (2015). “Deep sequencing of small RNA facilitates tissue and sex associated microRNA discovery in zebrafish.” BMC genomics 16(1): 950.
(30) Wargelius, A., et al. (2016). “Dnd knockout ablates germ cells and demonstrates germ cell independent sex differentiation in Atlantic salmon.” Scientific reports 6: 21284.
(31) Weidinger, G., et al. (2003). “dead end, a Novel Vertebrate Germ Plasm Component, Is Required for Zebrafish Primordial Germ Cell Migration and Survival.” Current Biology 13(16): 1429-1434.
(32) Xiao, J., et al. (2014). “Identification and characterization of microRNAs in ovary and testis of Nile tilapia (Oreochromis niloticus) by using solexa sequencing technology.” PloS one 9(1): e86821.
(33) Zhang, J., et al. (2017). “MiR-202-5p is a novel germ plasm-specific microRNA in zebrafish.” Scientific reports 7(1): 7055.

```
SEQUENCE LISTING
LENGTH: 40
TYPE: DNA
ORGANISM: Artificial Sequence
OTHER INFORMATION: Description of Artificial Sequence:
Forward tailed Primer (NED)
SEQUENCE: 1
SEQ ID NO 1
TAGGAGTGCAGCAAGCATGTGAATTTCCATTCGTGAACCG
LENGTH: 21
TYPE: DNA
ORGANISM: Artificial Sequence
OTHER INFORMATION: Description of Artificial Sequence: Primer
SEQUENCES
SEQ ID NO 2
gaagacaTAGCGCGTTATATG
LENGTH: 38
TYPE: DNA
ORGANISM: Artificial Sequence
OTHER INFORMATION: Description of Artificial Sequence:
Forward tailed Primer (FAM)
SEQUENCES
SEQ ID NO 3
TGTAAAACGACGGCCAGTTTTGCATATGGGCAGACATC
LENGTH: 22
TYPE: DNA
ORGANISM: Artificial Sequence
OTHER INFORMATION: Description of Artificial Sequence: Primer
SEQUENCES
SEQ ID NO 4
agtctcagatcttaaccatata
LENGTH: 42
TYPE: DNA
ORGANISM: Artificial Sequence
OTHER INFORMATION: Description of Artificial Sequence:
Forward tailed Primer (NED)
SEQUENCE: 5
SEQ ID NO 5
TAGGAGTGCAGCAAGCATtataattcattgttgtgggttgta
LENGTH: 22
TYPE: DNA
ORGANISM: Artificial Sequence
OTHER INFORMATION: Description of Artificial Sequence: Primer
SEQUENCE: 6
SEQ ID NO 6
Tgacacattggctgagactttc
LENGTH: 39
TYPE: DNA
ORGANISM: Artificial Sequence
OTHER INFORMATION: Description of Artificial Sequence:
Forward tailed primer (FAM)
SEQUENCE: 7
SEQ ID NO 7
TGTAAAACGACGGCCAGTCCATTCTGAAGTTATCCTTTT
LENGTH: 20
TYPE: DNA
ORGANISM: Artificial Sequence
OTHER INFORMATION: Description of Artificial Sequence: Primer
SEQUENCE: 8
SEQ ID NO 8
TCATAGCTCCCTCCTGTGGC
LENGTH: 37
TYPE: DNA
ORGANISM: Artificial Sequence
OTHER INFORMATION: Description of Artificial Sequence:
Forward tailed Primer (NED)
SEQUENCE: 9
SEQ ID NO 9
TAGGAGTGCAGCAAGCATtctttcacagGGTCCACCG
LENGTH: 23
TYPE: DNA
ORGANISM: Artificial Sequence
OTHER INFORMATION: Description of Artificial Sequence: Primer
SEQUENCE: 10
SEQ ID NO 10
TGACGAGATATCTCCACAAATGC
LENGTH: 40
TYPE: DNA
ORGANISM: Artificial Sequence
OTHER INFORMATION: Description of Artificial Sequence:
Forward tailed primer (FAM)
SEQUENCE: 11
SEQ ID NO 11
TGTAAAACGACGGCCAGTTGATTTGAATCCAGAGATTACT
LENGTH: 22
TYPE: DNA
ORGANISM: Artificial Sequence
OTHER INFORMATION: Description of Artificial Sequence: Primer
SEQUENCE: 12
SEQ ID NO 12
tggttggactgaaacatattgt
LENGTH: 39
TYPE: DNA
ORGANISM: Artificial Sequence
OTHER INFORMATION: Description of Artificial Sequence:
Forward tailed Primer (NED)
SEQ ID NO 13
SEQUENCE: 13
TAGGAGTGCAGCAAGCATtgtccttcagGTTGATTACAG
LENGTH: 21
TYPE: DNA
ORGANISM: Artificial Sequence
OTHER INFORMATION: Description of Artificial Sequence: Primer
SEQUENCE: 14
SEQ ID NO 14
gtcaaactcacTCTACTCCAA
LENGTH: 41
TYPE: DNA
ORGANISM: Artificial Sequence
OTHER INFORMATION: Description of Artificial Sequence:
Forward tailed Primer (NED)
SEQUENCE: 15
SEQ ID NO 15
TAGGAGTGCAGCAAGCATGGCTTCAACTACATTGGGATGGG
LENGTH: 22
TYPE: DNA
ORGANISM: Artificial Sequence
OTHER INFORMATION:
Description of Artificial Sequence: Primer
SEQUENCE: 16
SEQ ID NO 16
GGGAGGTTTCCAAAGCCAGCAT
LENGTH: 37
TYPE: DNA
ORGANISM: Artificial Sequence
OTHER INFORMATION: Description of Artificial Sequence:
Forward tailed primer (FAM)
SEQUENCE: 17
SEQ ID NO 17
TGTAAAACGACGGCCAGTttctcagGGGACGGCAGCG
LENGTH: 22
TYPE: DNA
ORGANISM: Artificial Sequence
OTHER INFORMATION: Description of Artificial Sequence: Primer
SEQUENCE: 18
SEQ ID NO 18
GTCTCTCTTGGCGTAATACTCC
LENGTH: 41
TYPE: DNA
ORGANISM: Artificial Sequence
OTHER INFORMATION: Description of Artificial Sequence:
Forward tailed Primer (NED)
SEQUENCE: 19
SEQ ID NO 19
TAGGAGTGCAGCAAGCATGGCAATGGAGAAGCTGAATGGAT
LENGTH: 22
TYPE: DNA
ORGANISM: Artificial Sequence
OTHER INFORMATION: Description of Artificial Sequence: Primer
SEQUENCE: 20
SEQ ID NO 20
GAACCAGCATGCGTAGCGGGAT
LENGTH: 40
TYPE: DNA
ORGANISM: Artificial Sequence
OTHER INFORMATION: Description of Artificial Sequence:
Forward tailed primer (FAM)
SEQUENCE: 21
SEQ ID NO 21
TGTAAAACGACGGCCAGTagaagttctaatgcacctccaa
LENGTH: 23
TYPE: DNA
ORGANISM: Artificial Sequence
OTHER INFORMATION: Description of Artificial Sequence: Primer
SEQUENCE: 22
SEQ ID NO 22
GTTCATAGCAGCCATGTCACTCT
LENGTH: 41
TYPE: DNA
ORGANISM: Artificial Sequence
OTHER INFORMATION: Description of Artificial Sequence:
Forward tailed Primer (NED)
SEQUENCE: 23
SEQ ID NO 23
TAGGAGTGCAGCAAGCATCGCTAAAGGAGCTGCTGGAAATg
LENGTH: 21
TYPE: DNA
ORGANISM: Artificial Sequence
OTHER INFORMATION: Description of Artificial Sequence: Primer
SEQUENCE: 24
SEQ ID NO 24
ACTGCTGAAGAGGCTGCGTAG
LENGTH: 41
TYPE: DNA
ORGANISM: Artificial Sequence
OTHER INFORMATION: Description of Artificial Sequence:
Forward tailed primer (FAM)
SEQUENCE: 25
SEQ ID NO 25
TGTAAAACGACGGCCAGTGGGAGCTCATCCTCTGGTTGGTG
LENGTH: 21
TYPE: DNA
ORGANISM: Artificial Sequence
OTHER INFORMATION: Description of Artificial Sequence: Primer
SEQUENCE: 26
SEQ ID NO 26
TGCCCCTTGCTGGTCTTGAAT
LENGTH: 41
TYPE: DNA
ORGANISM: Artificial Sequence
OTHER INFORMATION: Description of Artificial Sequence:
Forward tailed primer (FAM)
SEQUENCE: 27
SEQ ID NO 27
TGTAAAACGACGGCCAGTttttctttgtctctttagCAGGT
LENGTH: 19
TYPE: DNA
ORGANISM: Artificial Sequence
OTHER INFORMATION: Description of Artificial Sequence: Primer
SEQUENCE: 28
SEQ ID NO 28
GTTTTACTGTCCTCTACGC
LENGTH: 41
TYPE: DNA
ORGANISM: Artificial Sequence
OTHER INFORMATION: Description of Artificial Sequence:
Forward tailed Primer (NED)
SEQUENCE: 29
SEQ ID NO 29
TAGGAGTGCAGCAAGCATttgtgttttaacagCAGGTTCTC
LENGTH: 22
TYPE: DNA
ORGANISM: Artificial Sequence
OTHER INFORMATION: Description of Artificial Sequence: Primer
SEQUENCE: 30
SEQ ID NO 30
CACTTGTTTGTGTTAAAGTCGC
LENGTH: 39
TYPE: DNA
ORGANISM: Artificial Sequence
OTHER INFORMATION: Description of Artificial Sequence:
Forward tailed primer (FAM)
SEQUENCE: 31
SEQ ID NO 31
TGTAAAACGACGGCCAGTTGGGATAGTTGGTAATGGATT
LENGTH: 22
TYPE: DNA
ORGANISM: Artificial Sequence
OTHER INFORMATION: Description of Artificial Sequence: Primer
SEQUENCE: 32
SEQ ID NO 32
TTGATGGTGTAGATCATGTGCA
LENGTH: 41
TYPE: DNA ORGANISM: Artificial Sequence
OTHER INFORMATION: Description of Artificial Sequence:
Forward tailed Primer (NED)
SEQUENCE: 33
SEQ ID NO 33
TAGGAGTGCAGCAAGCATGTCTGTGCACATGATCTACACCA
LENGTH: 23
TYPE: DNA
ORGANISM: Artificial Sequence
OTHER INFORMATION: Description of Artificial Sequence: Primer
SEQUENCE: 34
SEQ ID NO 34
ACTGTCCATATGACGTTACTTTC
LENGTH: 41
TYPE: DNA
ORGANISM: Artificial Sequence
OTHER INFORMATION: Description of Artificial Sequence:
Forward tailed Primer (NED)
SEQ ID NO 35
SEQUENCE: 35
TAGGAGTGCAGCAAGCATCCAATGGCAACGACAGCAAAAAG
LENGTH: 23
TYPE: DNA
ORGANISM: Artificial Sequence
OTHER INFORMATION: Description of Artificial Sequence: Primer
SEQUENCE: 36
SEQ ID NO 36
tgtgtacagtgtgtgtacCTGGT
LENGTH: 41
TYPE: DNA
ORGANISM: Artificial Sequence
OTHER INFORMATION: Description of Artificial Sequence:
Forward tailed primer (FAM)
SEQUENCE: 37
SEQ ID NO 37
TGTAAAACGACGGCCAGTTCTCTGGACGGTCAGAACATCTA
LENGTH: 23
TYPE: DNA
ORGANISM: Artificial Sequence
OTHER INFORMATION: Description of Artificial Sequence: Primer
SEQUENCE: 38
SEQ ID NO 38
ctgcatcacagtttttgagcaca
LENGTH: 36
TYPE: DNA
ORGANISM: Artificial Sequence
OTHER INFORMATION: Description of Artificial Sequence:
Forward tailed Primer (NED)
SEQUENCE: 39
SEQ ID NO 39
TAGGAGTGCAGCAAGCATATGAACGGAATGGTTTGG
LENGTH: 20
TYPE: DNA
ORGANISM: Artificial Sequence
OTHER INFORMATION: Description of Artificial Sequence: Primer
SEQUENCE: 40
SEQ ID NO 40
TAGCTCGGCTCATGTCACAC
LENGTH: 40
TYPE: DNA
ORGANISM: Artificial Sequence
OTHER INFORMATION: Description of Artificial Sequence:
Forward tailed primer (FAM)
SEQUENCE: 41
SEQ ID NO 41
TGTAAAACGACGGCCAGTCCCGCGAATGTGCACTAACGAG
LENGTH: 23
TYPE: DNA
ORGANISM: Artificial Sequence
OTHER INFORMATION: Description of Artificial Sequence: Primer
SEQUENCE: 42
SEQ ID NO 42
TCTTGGTTCTTCAGCCAGTGGGA
LENGTH: 40
TYPE: DNA
ORGANISM: Artificial Sequence
OTHER INFORMATION: Description of Artificial Sequence:
Forward tailed Primer (NED)
SEQUENCE: 43
SEQ ID NO 43
TAGGAGTGCAGCAAGCATTTTCCCAATTCCTCCACCCAAG
LENGTH: 21
TYPE: DNA
ORGANISM: Artificial Sequence
OTHER INFORMATION: Description of Artificial Sequence: Primer
SEQUENCE: 44
SEQ ID NO 44
GTGAAACAGAACTGCAGGACG
LENGTH: 41
TYPE: DNA
ORGANISM: Artificial Sequence
OTHER INFORMATION: Description of Artificial Sequence:
Forward tailed Primer (NED)
SEQ ID NO 45
SEQUENCE: 45
TAGGAGTGCAGCAAGCATATGTCTGAGTCAGAGCAACAGTA
LENGTH: 20
TYPE: DNA
ORGANISM: Artificial Sequence
OTHER INFORMATION: Description of Artificial Sequence: Primer
SEQUENCE: 46
SEQ ID NO 46
TCCCTCCGTGCCGCCGTTTT
LENGTH: 41
TYPE: DNA
ORGANISM: Artificial Sequence
OTHER INFORMATION: Description of Artificial Sequence:
Forward tailed primer (FAM)
SEQUENCE: 47
SEQ ID NO 47
TGTAAAACGACGGCCAGTGAAGAAGGATCCAGTAAAGAAAA
LENGTH: 23
TYPE: DNA
ORGANISM: Artificial Sequence
OTHER INFORMATION: Description of Artificial Sequence: Primer
SEQUENCE: 48
SEQ ID NO 48
TGGAAGCTCTATGGTCTCAATct
LENGTH: 41
TYPE: DNA
ORGANISM: Artificial Sequence
OTHER INFORMATION: Description of Artificial Sequence:
Forward tailed Primer (NED)
SEQUENCE: 49
SEQ ID NO 49
TAGGAGTGCAGCAAGCATtttgttctgtctccttgtctccc
LENGTH: 23
TYPE: DNA
ORGANISM: Artificial Sequence
OTHER INFORMATION: Description of Artificial Sequence: Primer
SEQUENCE: 50
SEQ ID NO 50
acaacgagggcatgacacttacG
LENGTH: 39
TYPE: DNA
ORGANISM: Artificial Sequence
OTHER INFORMATION: Description of Artificial Sequence:
Forward tailed primer (FAM)
SEQUENCE: 51
SEQ ID NO 51
TGTAAAACGACGGCCAGTCCAAGATGGCCAAAAACAAGC
LENGTH: 23
TYPE: DNA
ORGANISM: Artificial Sequence
OTHER INFORMATION: Description of Artificial Sequence: Primer
SEQUENCE: 52
SEQ ID NO 52
ggaagtagcaatgcagacggaca
LENGTH: 36
TYPE: DNA
ORGANISM: Artificial Sequence
OTHER INFORMATION: Description of Artificial Sequence:
Forward tailed Primer (NED)
SEQUENCE: 53
SEQ ID NO 53
TAGGAGTGCAGCAAGCATCACACAACCGACTCAAGT
LENGTH: 21
TYPE: DNA
ORGANISM: Artificial Sequence
OTHER INFORMATION: Description of Artificial Sequence: Primer
SEQUENCE: 54
SEQ ID NO 54
Tttactcgtccagctgaccgg
LENGTH: 41
TYPE: DNA
ORGANISM: Artificial Sequence
OTHER INFORMATION: Description of Artificial Sequence:
Forward tailed primer (FAM)
SEQUENCE: 55
SEQ ID NO 55
TGTAAAACGACGGCCAGTTTCCCCATACCTTGACTATACTG
LENGTH: 17
TYPE: DNA
ORGANISM: Artificial Sequence
OTHER INFORMATION: Description of Artificial Sequence: Primer
SEQUENCE: 56
SEQ ID NO 56
GCGGTGGCGAGCGGCTG
LENGTH: 39
TYPE: DNA
ORGANISM: Artificial Sequence
OTHER INFORMATION: Description of Artificial Sequence:
Forward tailed Primer (NED)
SEQUENCE: 57
SEQ ID NO 57
TAGGAGTGCAGCAAGCATACCTCCACCCATGATGCTCCC
LENGTH: 23
TYPE: DNA
ORGANISM: Artificial Sequence
OTHER INFORMATION: Description of Artificial Sequence: Primer
SEQUENCE: 58
SEQ ID NO 58
CATTGAAACCATATCACCAACct
LENGTH: 41
TYPE: DNA
ORGANISM: Artificial Sequence
OTHER INFORMATION: Description of Artificial Sequence:
Forward tailed primer (FAM)
SEQUENCE: 59
SEQ ID NO 59
TGTAAAACGACGGCCAGTtgccaaaATGTCATCAATCTAG
LENGTH: 23
TYPE: DNA
ORGANISM: Artificial Sequence
OTHER INFORMATION: Description of Artificial Sequence: Primer
SEQUENCE: 60
SEQ ID NO 60
CCCAGGGGACTGAATGTCTTTAG
LENGTH: 41
TYPE: DNA
ORGANISM: Artificial Sequence
OTHER INFORMATION: Description of Artificial Sequence:
Forward tailed Primer (NED)
SEQUENCE: 61
SEQ ID NO 61
TAGGAGTGCAGCAAGCATAATTGTCTGCACTTATAGATGTC
LENGTH: 22
TYPE: DNA
ORGANISM: Artificial Sequence
OTHER INFORMATION: Description of Artificial Sequence: Primer
SEQUENCE: 62
SEQ ID NO 62
tccgttatgaaGCTCTTCCACC
LENGTH: 39
TYPE: DNA
ORGANISM: Artificial Sequence
OTHER INFORMATION: Description of Artificial Sequence:
Forward tailed primer (FAM)
SEQUENCE: 63
SEQ ID NO 63
TGTAAAACGACGGCCAGTCTCGGTCACCAGGTGTCTGAT
LENGTH: 21
TYPE: DNA
ORGANISM: Artificial Sequence
OTHER INFORMATION: Description of Artificial Sequence: Primer
SEQUENCE: 64
SEQ ID NO 64
TCTTCTCGCAGCTGACTGCAC
LENGTH: 41
TYPE: DNA
ORGANISM: Artificial Sequence
OTHER INFORMATION: Description of Artificial Sequence:
Forward tailed Primer (NED)
SEQUENCE: 65
SEQ ID NO 65
TAGGAGTGCAGCAAGCATAAGCTCAGCCTCAGCGAATCTCT
LENGTH: 23
TYPE: DNA
ORGANISM: Artificial Sequence
OTHER INFORMATION: Description of Artificial Sequence: Primer
SEQUENCE: 66
SEQ ID NO 66
ttttcctaagtacttatgtacca
LENGTH: 39
TYPE: DNA
ORGANISM: Artificial Sequence
OTHER INFORMATION: Description of Artificial Sequence:
Forward tailed primer (FAM)
SEQUENCE: 67
SEQ ID NO 67
TGTAAAACGACGGCCAGTgttccagtgtccagaatcggg
LENGTH: 18
TYPE: DNA
ORGANISM: Artificial Sequence
OTHER INFORMATION: Description of Artificial Sequence: Primer
SEQUENCE: 68
SEQ ID NO 68
ctGGTGGAATACCTCTGC
LENGTH: 40
TYPE: DNA
ORGANISM: Artificial Sequence
OTHER INFORMATION: Description of Artificial Sequence:
Forward tailed primer (FAM)
SEQUENCE: 69
SEQ ID NO 69
TGTAAAACGACGGCCAGTCTCCGTGTACGCCAAGTCCAGA
LENGTH: 23
TYPE: DNA
ORGANISM: Artificial Sequence
OTHER INFORMATION: Description of Artificial Sequence: Primer
SEQUENCE: 70
SEQ ID NO 70
Gacagtgttataatccttcaatg
LENGTH: 21
TYPE: DNA
ORGANISM: Artificial Sequence
OTHER INFORMATION: Description of Artificial Sequence: Primer
SEQUENCE: 71
SEQ ID NO 71
ctccttttgcagGTATGTGGG
LENGTH: 21
TYPE: DNA
ORGANISM: Artificial Sequence
OTHER INFORMATION: Description of Artificial Sequence: Primer
SEQUENCE: 72
SEQ ID NO 72
TGTGAAGACCTGCAGAATGAG
LENGTH: 20
TYPE: DNA
ORGANISM: Artificial Sequence
OTHER INFORMATION: Description of Artificial Sequence: Primer
SEQUENCE: 73
SEQ ID NO 73
GGTAGAGGCCAAGGGAACTG
LENGTH: 19
TYPE: DNA
ORGANISM: Artificial Sequence
OTHER INFORMATION: Description of Artificial Sequence: Primer
SEQUENCE: 74
SEQ ID NO 74
GCAGGGATGGAGAAAGTCA
LENGTH: 23
TYPE: DNA
ORGANISM: Artificial Sequence
OTHER INFORMATION: Description of Artificial Sequence: Primer
SEQUENCE: 75
SEQ ID NO 75
CGCGACTTTGTCAACTATCTGGT
LENGTH: 18
TYPE: DNA
ORGANISM: Artificial Sequence
OTHER INFORMATION: Description of Artificial Sequence: Primer
SEQUENCE: 76
SEQ ID NO 76
Caggaacagcttcctgac
LENGTH: 15
TYPE: DNA
ORGANISM: Artificial Sequence
OTHER INFORMATION: Description of Artificial Sequence: Primer
SEQUENCE: 77
SEQ ID NO 77
cccctgctggatACC
LENGTH: 19
TYPE: DNA
ORGANISM: Artificial Sequence
OTHER INFORMATION: Description of Artificial Sequence: Primer
SEQUENCE: 78
SEQ ID NO 78
ACGCGCGCACGAACCTGAT
LENGTH: 19
TYPE: DNA
ORGANISM: Artificial Sequence
OTHER INFORMATION: Description of Artificial Sequence: Primer
SEQUENCE: 80
SEQ ID NO 80
AAGCTTCCCAGCGACATCA
LENGTH: 23
TYPE: DNA
ORGANISM: Artificial Sequence
OTHER INFORMATION: Description of Artificial Sequence: Primer
SEQUENCE: 81
SEQ ID NO 81
tgtgtacagtgtgtgtacCTGGT
LENGTH: 19
TYPE: DNA
ORGANISM: Artificial Sequence
OTHER INFORMATION: Description of Artificial Sequence: Primer
SEQUENCE: 82
SEQ ID NO 82
AAGACGAGTCGTTTCAAAA
LENGTH: 18
TYPE: DNA
ORGANISM: Artificial Sequence
OTHER INFORMATION: Description of Artificial Sequence: Primer
SEQUENCE: 83
SEQ ID NO 83
CAGAACATGCGGTCAGGA
LENGTH: 19
TYPE: DNA
ORGANISM: Artificial Sequence
OTHER INFORMATION: Description of Artificial Sequence: Primer
SEQUENCE: 84
SEQ ID NO 84
GATCCGCGGGATCACTGCC
LENGTH: 20
TYPE: DNA
ORGANISM: Artificial Sequence
OTHER INFORMATION: Description of Artificial Sequence: Primer
SEQUENCE: 85
SEQ ID NO 85
CTGGGCTACAGCCTTCTGAG
LENGTH: 22
TYPE: DNA
ORGANISM: Artificial Sequence
OTHER INFORMATION: Description of Artificial Sequence: Primer
SEQUENCE: 86
SEQ ID NO 86
tgccagcctaaaatacctcagc
LENGTH: 27
TYPE: DNA
ORGANISM: Artificial Sequence
OTHER INFORMATION: Description of Artificial Sequence: Primer
SEQUENCE: 87
SEQ ID NO 87
Gaaacatacaataattttttgaaacat
(wild-type KIF5B)
LENGTH: 6289 bp and 962 aa
TYPE: cDNA (SEQ ID NO: 88) and Protein (SEQ ID NO: 90)
ORGANISM: Nile tilapia
SEQ ID NOs 88 and 90
      1 TACTTTGGGCAGCTGCTGTGAGTTTTTGTGTGCATTTGCTGATTAATGGAGATTTCATTA 60
          ............................................................
    61 CAGAAAAACAGTAAGGAGGGGAGCGCCTGCCGGGGTGACGTCATTGTGATCCACATCCAC 120
          ............................................................
  121 GGTAGCAGGAGGGGTGAGGTGGCCAGCCGCTGATCCACCGGTATCATGGCTTCCTAACAG 180
          ............................................................
  181 GACGGGGGGAAGTGACTGAGTGGAAAAACAAGGATTTTTGTTGAATGTCCAACTGATCAT 240
          ............................................................
  241 CGCCCTTTCTAGAACTTGAGATTGGGACAGAGGGGCTCGGCCTCCCTTTTCGCCTCTCAC 300
          ............................................................
  301 GGCCGCCCCTGAGATCCAGTATATACTTTAATTCTTCTCGTGAATTTCCATTCGTGAACC 360
          ............................................................
  361 GTGGAAGATGGCCGACCCGGCGGAGTGCACCATCAAAGTGATGTGCCGTTTTAGGCCCCT 420
          .......-M--A--D--P--A--E--C--T--I--K--V--M--C--R--F--R--P--L 18
  421 GAACAGCTCCGAAGTGACCAGGGGCGACAAGTACATTCCCAAGTTTCAAGGGGAAGATAC 480
    18 --N--S--S--E--V--T--R--G--D--K--Y--I--P--K--F--Q--G--E--D--T 38
  481 CTGCATTATCGGGGGTAAACCTTACATGTTTGACAGAGTGTTTCAGTCAAATACAACACA 540
    38 --C--I--I--G--G--K--P--Y--M--F--D--R--V--F--Q--S--N--T--T--Q 58
  541 AGAACAAGTGTACAACGCCTGTGCCCAAAAGATTGTAAAAGATGTTCTCGAGGGTTATAA 600
    58 --E--Q--V--Y--N--A--C--A--Q--K--I--V--K--D--V--L--E--G--Y--N 78
  601 TGGGACAATTTTTGCATATGGGCAGACATCATCTGGTAAAACACACACCATGGAGGGGAA 660
    78 --G--T--I--F--A--Y--G--Q--T--S--S--G--K--T--H--T--M--E--G--N 98
  661 TCTCCATGACACAGATTCAATGGGAATCATCCCCAGGATAGTGCAAGACATCTTCAACTA 720
    98 --L--H--D--T--D--S--M--G--I--I--P--R--I--V--Q--D--I--F--N--Y 118
  721 CATCTATTCCATGGACGAAAACCTGGAGTTTCATATCAAAGTTTCATATTTTGAAATCTA 780
  118 --I--Y--S--M--D--E--N--L--E--F--H--I--K--V--S--Y--F--E--I--Y 138
  781 CTTAGACAAGATCCGGGACCTTTTGGACGTGTCAAAGACCAATTTGTCAGTGCATGAAGA 840
  138 --L--D--K--I--R--D--L--L--D--V--S--K--T--N--L--S--V--H--E--D 158
  841 CAAAAACAGAGTACCTTATGTCAAGGGCTGCACTGAGAGATTTGTCTGCAGCCCAGATGA 900
  158 --K--N--R--V--P--Y--V--K--G--C--T--E--R--F--V--C--S--P--D--E 178
  901 GGTCATGGATACAATTGATGAAGGCAAAGCTAACAGACATGTAGCAGTTACAAACATGAA 960
  178 --V--M--D--T--I--D--E--G--K--A--N--R--H--V--A--V--T--N--M--N 198
  961 CGAGCACAGCTCCAGGAGTCACAGTATCTTCCTGATCAACGTTAAACAGGAGAATACTCA 1020
  198 --E--H--S--S--R--S--H--S--I--F--L--I--N--V--K--Q--E--N--T--Q 218
1021 AACAGAGCAGAAGCTCAGTGGAAAACTCTACCTGGTAGATCTGGCTGGTAGTGAAAAGGT 1080
  218 --T--E--Q--K--L--S--G--K--L--Y--L--V--D--L--A--G--S--E--K--V 238
1081 CAGTAAAACAGGTGCCGAGGGAGCAGTGCTGGATGAAGCCAAGAACATAAACAAGTCCCT 1140
  238 --S--K--T--G--A--E--G--A--V--L--D--E--A--K--N--I--N--K--S--L 258
1141 GTCATCCCTGGGAAATGTCATCTCTGCGTTGGCTGAAGGAACGGCCTACATCCCTTACCG 1200
  258 --S--S--L--G--N--V--I--S--A--L--A--E--G--T--A--Y--I--P--Y--R 278
1201 AGACAGCAAGATGACCCGTATCCTGCAGGACTCGCTGGGCGGTAACTGTCGAACCACCAT 1260
  278 --D--S--K--M--T--R--I--L--Q--D--S--L--G--G--N--C--R--T--T--I 298
1261 TGTCATCTGCTGCTCACCTTCCTCCTTTAATGAGGCTGAAACCAAATCCACCCTAATGTT 1320
  298 --V--I--C--C--S--P--S--S--F--N--E--A--E--T--K--S--T--L--M--F 318
1321 CGGGCAGAGAGCAAAGACCATCAAGAACACAGTGACAGTGAACATTGAGCTGACAGCAGA 1380
  318 --G--Q--R--A--K--T--I--K--N--T--V--T--V--N--I--E--L--T--A--E 338
1381 GCAGTGGAAGCAGAAGTATGAGCGAGAGAAGGAGAAGAACAAGACCCTGAGGAATACCAT 1440
  338 --Q--W--K--Q--K--Y--E--R--E--K--E--K--N--K--T--L--R--N--T--I 358
1441 CACGTGGTTGGAGAATGAGCTGAACCGCTGGAGAAATGGTGAGAGCGTGCCAGTGGAGGA 1500
  358 --T--W--L--E--N--E--L--N--R--W--R--N--G--E--S--V--P--V--E--E 378
1501 GCAGTTTGATAAGGAGAAAGCCAACGCCGAGGTGCTGGCCCTGGATAATATTATAAACGA 1560
  378 --Q--F--D--K--E--K--A--N--A--E--V--L--A--L--D--N--I--I--N--D 398
1561 CAAGGCGGCCTCGACACCCAACGTGCCCGGCGTTCGCCTCACTGACGTGGAGAAGGACAA 1620
  398 --K--A--A--S--I--P--N--V--P--G--V--R--L--T--D--V--E--K--D--K 418
1621 GTGTGAAGCAGAGCTGGCCAAACTCTATAAACAGCTGGATGATAAGGATGAGGAAATCAA 1680
  418 --C--E--A--E--L--A--K--L--Y--K--Q--L--D--D--K--D--E--E--I--N 438
1681 CCAGCAGAGCCAGCTGGCTGAGAAGCTGAAACAGCAGATGCTGGACCAGGAGGAGCTTCT 1740
  438 --Q--Q--S--Q--L--A--E--K--L--K--Q--Q--M--L--D--Q--E--E--L--L 458
1741 AGCCTCTTCCCGCCGTGATCACGAGAACCTCCAGGCAGAGCTGAACCGCCTCCAGGCTGA 1800
  458 --A--S--S--R--R--D--H--E--N--L--Q--A--E--L--N--R--L--Q--A--E 478
1801 AAACGAAGCCTCAAAGGAGGAGGTGAAGGAGGTGCTGCAGGCCCTGGAAGAGCTGGCTGT 1860
  478 --N--E--A--S--K--E--E--V--K--E--V--L--Q--A--L--E--E--L--A--V 498
1861 CAATTATGACCAGAAGAGCCAAGAGGTGGAGGATAAAACCAAGGAGTTTGAGGCCATCAG 1920
  498 --N--Y--D--Q--K--S--Q--E--V--E--D--K--T--K--E--F--E--A--I--S 518
1921 TGAGGAGCTCAGCCAGAAATCGTCCATCCTGTCATCTCTGGACTCGGAGCTTCAGAAGCT 1980
  518 --E--E--L--S--Q--K--S--S--I--L--S--S--L--D--S--E--L--Q--K--L 538
1981 GAAGGAGATGTCCAACCACCAGAAGAAGAGGGTGACTGAAATGATGTCATCACTGCTTAA 2040
  538 --K--E--M--S--N--H--Q--K--K--R--V--T--E--M--M--S--S--L--L--K 558
2041 AGACCTAGCTGAGATTGGCATCGCTGTAGGCAGCAATGACATTAAGCAACACGACGGTGG 2100
  558 --D--L--A--E--I--G--I--A--V--G--S--N--D--I--K--Q--H--D--G--G 578
2101 CAGCGGTCTGATTGACGAGGAGTTTACAGTGGCCCGTCTGTACATCAGCAAGATGAAGTC 2160
  578 --S--G--L--I--D--E--E--F--T--V--A--R--L--Y--I--S--K--M--K--S 598
2161 AGAAGTGAAGACGATGGTGAAACGCTGCAAGCAGCTAGAGGGAACCCAGGCAGAAAGCAA 2220
  598 --E--V--K--T--M--V--K--R--C--K--Q--L--E--G--T--Q--A--E--S--N 618
2221 CAAGAAGATGGATGAGAACGAGAAGGAACTGGCCGCCTGCCAGCTACGCATCTCCCAGCA 2280
  618 --K--K--M--D--E--N--E--K--E--L--A--A--C--Q--L--R--I--S--Q--H 638
2281 CGAGGCTAAAATCAAGTCCTTGACTGAGTACCTGCAAAATGTAGAGCAGAAGAAGAGGCA 2340
  638 --E--A--K--I--K--S--L--T--E--Y--L--Q--N--V--E--Q--K--K--R--Q 658
2341 GTTGGAGGAAAATGTGGACGCTCTCAATGAGGAACTTGTCAAGATCAGTGCTCAAGAGAA 2400
  658 --L--E--E--N--V--D--A--L--N--E--E--L--V--K--I--S--A--Q--E--K 678
2401 AGTCCATGCTATGGAGAAAGAGAACGAGATCCAGACTGCCAATGAAGTCAAGGAAGCAGT 2460
  678 --V--H--A--M--E--K--E--N--E--I--Q--T--A--N--E--V--K--E--A--V 698
2461 GGAGAAGCAGATCCACTCCCATCGTGAAGCTCATCAGAAACAGATCAGCAGCCTGAGAGA 2520
  698 --E--K--Q--I--H--S--H--R--E--A--H--Q--K--Q--I--S--S--L--R--D 718
2521 TGAGCTGGACAACAAGGAGAAGCTCATCACCGAGCTGCAGGATCTGAATCAGAAGATCAT 2580
  718 --E--L--D--N--K--E--K--L--I--T--E--L--Q--D--L--N--Q--K--I--M 738
2581 GCTGGAGCAGGAGAGGCTCAGAGTGGAGCATGAGAAGCTTAAATCCACCGATCAGGAGAA 2640
  738 --L--E--Q--E--R--L--R--V--E--H--E--K--L--K--S--T--D--Q--E--K 758
2641 GAGCCGCAAGCTGCACGAGCTCACGGTGATGCAGGACAGGAGGGAGCAGGCCAGACAGGA 2700
  758 --S--R--K--L--H--E--L--I--V--M--Q--D--R--R--E--Q--A--R--Q--D 778
2701 CCTGAAGGGTCTGGAAGAGACAGTGGCTAAAGAGCTGCAGACTCTGCACAACCTGAGGAA 2760
  778 --L--K--G--L--E--E--T--V--A--K--E--L--Q--I--L--H--N--L--R--K 798
2761 ACTCTTTGTCCAGGACCTGGCCACCCGAGTGAAAAAGAGCGCTGAGATGGACTCGGATGA 2820
  798 --L--F--V--Q--D--L--A--T--R--V--K--K--S--A--E--M--D--S--D--D 818
2821 CACAGGTGGGAGTGCAGCTCAGAAACAGAAAATTTCCTTTCTTGAGAACAATCTTGAACA 2880
  818 --T--G--G--S--A--A--Q--K--Q--K--I--S--F--L--E--N--N--L--E--Q 838
2881 GCTCACCAAGGTTCACAAACAGCTGGTGCGTGATAATGCAGACCTGCGCTGTGAGCTTCC 2940
  838 --L--T--K--V--H--K--Q--L--V--R--D--N--A--D--L--R--C--E--L--P 858
2941 TAAACTGGAAAAGCGTCTTCGAGCTACGGCTGAGCGGGTCAAGGCCTTGGAGTCTGCTTT 3000
  858 --K--L--E--K--R--L--R--A--T--A--E--R--V--K--A--L--E--S--A--L 878
3001 GAAGGAAGCCAAGGAGAACGCCGCCCGCGATCGCAAGCGCTACCAGCAGGAAGTGGACCG 3060
  878 --K--E--A--K--E--N--A--A--R--D--R--K--R--Y--Q--Q--E--V--D--R 898
3061 CATCAAAGAGGCCGTCAGAGCCAAGAACATGGCCAGGAGGGGACATTCAGCCCAGATTGC 3120
  898 --I--K--E--A--V--R--A--K--N--M--A--R--R--G--H--S--A--Q--I--A 918
3121 CAAACCCATCAGGCCTGGGCAGCAGCCAGTAGCATCCCCCACCCACCCCAACATTAACCG 3180
  918 --K--P--I--R--P--G--Q--Q--P--V--A--S--P--T--H--P--N--I--N--R 938
3181 CAGTGGAGGAGGCTTCTACCAGAACAGCCAGACGGTGTCCATCAGAGGGGGCAGCAGCAA 3240
  938 --S--G--G--G--F--Y--Q--N--S--Q--T--V--S--I--R--G--G--S--S--K 958
3241 GCCTGACAAGAACTGAAGAGCAGCAGAACAGAAGGACGACACCACAGAAGAAGCCAATAT 3300
  958 --P--D--K--N--*-............................................ 962
3301 CACCCCCCGCCCACCCCGACAACCTGTCATTCCATTACAGCGAACAGACTCCTCGTCGCT 3360
          ............................................................
3361 GCTTTGGAACCACGAAGGAGTTTCTGAAATATAAATATATATATATAAATATTCCCAGCT 3420
          ............................................................
3421 TGTACAGCTCCAGCCCCCCCACCACCACACCTCCACCTACCCACCTCCCTCTCCCCCGAA 3480
          ............................................................
3481 GTTCTAATCATGACTCATCTCTTTTTCTCTTACTGGATATAATAAAAGAAAGAAGACAAC 3540
          ............................................................
3541 CGTTTTAATTTACAAAAAGCCAAGATAATATTCTTATTCAGGCAACCAAACGCAGTCTTG 3600
          ............................................................
3601 GGCGCAGCCTCGGCGAGCGAAACCGCAACGCGACTCGAATGTGTAGCTTCGGGTTTGTTG 3660
          ............................................................
3661 ATTTTGTTTAGTTTTTTTTCTTTTTCGTTTTGCACAGTCTGTCGTCATCTGTCGTGCGAG 3720
          ............................................................
3721 TAGTTCTGCACTGTGCCAAGCTGAATGTAACGGTCGAAAGATCCAAAAAATTCATAGAAA 3780
          ............................................................
3781 TAAACACCTAATATTAAAAAAGACCAAAAAAAACGAAGAGGAGACCCTACAGTGAGAAAC 3840
          ............................................................
3841 AGCTTGACCCTATAAAGCTAACCTCTGTACAGTTCATTGTTATTATTATTATAATTATTA 3900
          ............................................................
3901 TTATTATTATTATCATTGGCTGTTAACCACACTTTTCTCTGGGTAGATTTTACATGCTTC 3960
          ............................................................
3961 TTTAAGGGAAATACAAAAAAAGTACAAAAAAATGTTTTGAATTGACTAGATGGCGTCGAG 4020
          ............................................................
4021 CACTACTGTTCTGTCTGATCTTGTTGTACAGTTGTAAAATTGGCACTACTGCACACGTTT 4080
          ............................................................
4081 CCCCGGAGAGACGAGGCTAAACACAAGGATTAAAATAAAGCCAAGAAGACGTGCGACAGT 4140
          ............................................................
4141 GTACCGTAGGTGTATTTACCTAAATACCTGTGGAGGCCAACTGTTTTTAATATTAAGTTA 4200
          ............................................................
4201 AAAAAAACTATACTCGTTAATGGTGGCTTCATGAGAAGGATGCAAAGAATGTAGAATGAA 4260
          ............................................................
4261 GGGAAAAGAGGAGGAGGATCCGGTTAAGACAACAGACTTCCACCTTTAAGCATAGCCTAT 4320
          ............................................................
4321 GCTACGTAGCTAAATGTACTGTTTTTACTTCTCTCGGTGGTTAATAATGACGTGTTAATG 4380
          ............................................................
4381 GAAGCTGTTTAATATCTCTCTGCACATTGGAGCACATAGATTGCAAGTGTATAGATGGAA 4440
          ............................................................
4441 ATAGCACACGATGCTCGTCCTGTCCTCGCTGGGTCCCTGTCGGTAACTCGTCTCCTTTCA 4500
          ............................................................
4501 CTCGCGTATTCAGGACCCGTTCTTTTTTTGGTTCTTGTTTCTAGTTTGACTGTTGAATCC 4560
          ............................................................
4561 CTGCAGTGTCATGTTTTCTTTTTCAAGGGTGCCTCGCTTCTTCAGTCTTCCCTCCCCACA 4620
          ............................................................
4621 CCTTATAGATTAAAAGACCTGTAACTGTATGCGCCCCCTTTCAGTTGTAAATTTGCAGAG 4680
          ............................................................
4681 TGTCATGCTGGGTTTTATGTACTGTATCTCTTTCTTTGTTCCATAGATGGTGTAGATTTC 4740
          ............................................................
4741 TATTTCAAAGTGGGGGGTAACACCGGGCGGGTGGAGTGGGATCGTTCAGTTGATGAGTTA 4800
          ............................................................
4801 GACCTTCGCCACTGATCAGCCAGTCAGGGAGAGCGGGGTCTATAATTGCACAATGATCTT 4860
          ............................................................
4861 CTGTCATCATCTGGAAGAAGGGTTTATTTAACTAAATCTAACCAGGGGTGTAGATTTTTT 4920
          ............................................................
4921 GTGTTTTTGTTCTTTGTTGTTTTTTTAGTGGGTTTTTTTAAATTGTTATTAATTTCCCAT 4980
          ............................................................
4981 CACATCTTTATTTTAACCCTGTGAAGCCCCACTGCATTTGGCAAAGAGCTTGTTGTGATT 5040
          ............................................................
5041 GTAACCCCAGCATGAGAACACTAACATCTTGTGCAAGTGCAATATACTGTAAAATACACT 5100
          ............................................................
5101 GTATATCAGTCGGCCGGCAGGTGTGATCAGGGTGTGGTTGTACCTGCCCACTCCTCCTTT 5160
          ............................................................
5161 TTGTGTTGCATTTTGTTTCACTGGTGTCAAGTCCTCGGTGTGTGTTTTCTTTCTTTGATC 5220
          ............................................................
5221 ACTCTTTCTGAAAAGCTGAGACATGTTGCAGATCTTTTTGTTTAGTTTAGTTTTATATAA 5280
          ............................................................
5281 ACGTCATATTCTATATCAAATCTACTGCAGCTGCTGTAATGGAAAGTTAACAAAAGTGCA 5340
          ............................................................
5341 CAGATTGTATAAAAAATCACATATGACCCAAAGTTTTGAGTTTGAAGCTTTTTAGGAAGA 5400
          ............................................................
5401 CTGGTCAAAGAAACTTCTACTGAAAAAGGATACGTTTTGAGACTGGTGGGACGAATGTAG 5460
          ............................................................
5461 CTGGAAAACAAAAGGAGGGGAAATGATTTTGGCTAAAACCTGTTATCTCCATACAGGAAA 5520
          ............................................................
5521 GCTGGGTGTAAAATAGCACTTCTTTAGCTGCACTCAGATAAAAACACTCCACATGTGGCT 5580
          ............................................................
5581 GTTTTTGAGTGGAGGAGGGGAAGAAAAGTTTTTGACAACCGCTTGTTGTCGCTGAAGTGT 5640
          ............................................................
5641 ATTCAGTTGTAATAATTACACTCTGCAAGATGCAGGGAGGAGTAGCTCCCTCGCATCTAT 5700
          ............................................................
5701 GACAGGACAGTGTTTGGTGTCTTATCGAGACGGTTTATACCCTCTGTGTAACCTTCTAGA 5760
          ............................................................
5761 TTTAGCTGAGACATTGCAGCGTGGACCTCAAAATGTTCATCCTTTGACCTCCCACCAAAA 5820
          ............................................................
5821 CTGGATACGAAATGGGGAATAAATACAGCAAAAAGATAAATACTTGTTTACCTAAATTAA 5880
          ............................................................
5881 ATTTTGCATTAATTCTAAATTAAAAGTGTAGCCACTTTTTTTTATTACTGTGTAATAGTT 5940
          ............................................................
5941 GGTCAGTTTTTAAAAGGACAGTTTTGGGGCTCCATCAGTGGACAAGGTACTGGATCATTC 6000
          ............................................................
6001 CTGGAGAACTGGGGCACAAATGGCTGGGCTCTGATATGGACGGAGACGGGACGTTAATGA 6060
          ............................................................
6061 GATCACAGTTTTGGATTGACTGCATGATGTAAATGTATGTGTGATTAAATAATTATGAGG 6120
          ............................................................
6121 AAAAAAAACTGTCCCCTCTGTGTTCTGTCATTTGACTCTTGTGAATGTGGAGATGGGTTT 6180
          ............................................................
6181 CACAGGGCTGTTTCTGTTTTACGTACATACACTGGTCGACAGTTTTTCTTTTTTCGGTTT 6240
          ............................................................
6241 GGGGCTTCACTCTGAGAACTCATTTGGAATTGGAGAAGGGGTCTTCTTT 6289
          .................................................
(KIF5B mutant allele-1 nt deletion)
LENGTH: 6289 bp(−1 bp) and 110 aa
TYPE: cDNA (SEQ ID NO: 89) and Protein (SEQ ID NO: 91)
ORGANISM: Nile tilapia
SEQ ID NOs 89 and 91
      1 TACTTTGGGCAGCTGCTGTGAGTTTTTGTGTGCATTTGCTGATTAATGGAGATTTCATTA 60
          ............................................................
    61 CAGAAAAACAGTAAGGAGGGGAGCGCCTGCCGGGGTGACGTCATTGTGATCCACATCCAC 120
          ............................................................
  121 GGTAGCAGGAGGGGTGAGGTGGCCAGCCGCTGATCCACCGGTATCATGGCTTCCTAACAG 180
          ............................................................
  181 GACGGGGGGAAGTGACTGAGTGGAAAAACAAGGATTTTTGTTGAATGTCCAACTGATCAT 240
          ............................................................
  241 CGCCCTTTCTAGAACTTGAGATTGGGACAGAGGGGCTCGGCCTCCCTTTTCGCCTCTCAC 300
          ............................................................
  301 GGCCGCCCCTGAGATCCAGTATATACTTTAATTCTTCTCGTGAATTTCCATTCGTGAACC 360
          ............................................................
  361 GTGGAAGATGGCCGACCCGGCGGAGTGCACCATCAAAGTGATGTGCCGTTTTAGGCCCCT 420
          .........-M--A--D--P--A--E--C--T--I--K--V--M--C--R--F--R P--L 18
  421 GAACAGCTCCGAAGTGACCAGGGGCGACAAGTACATTCCCAAGTTTCAAGGGGAAGATAC 480
    18 --N--S--S--E--V--T--R--G--D--K--Y--I--P--K--F--Q--G--E--D--T 38
  481 CTGCATTATCGGGGGTAAACCTTACATGTTTGACAGAGTGTTTCAGTCAAATACAACACA 540
    38 --C--I--I--G--G--K--P--Y--M--F--D--R--V--F--Q--S--N--T--T--Q 58
  541 AGAACAAGTGTACAACGCCTGTGCCCAAAAGATTGTAAAAGATGTTCTCGAGGGTTATAA 600
    58 --E--Q--V--Y--N--A--C--A--Q--K--I--V--K--D--V--L--E--G--Y--N 78
  601 TGGGACAATTTTTGCATATGGGCAGACATCATCTGGTAAAACACACACCATGGAGGGGAA 660
    78 --G--I--I--F--A--Y--G--Q--T--S--S--G--K--T--H--T--M--E--G--N 98
  661 TCTCCATGACACAGATTCAATGGGAATCATCCCCAGATAGTGCAAGACATCTTCAACTAG 720
    98 --L--H--D--T--D--S--M--G--I--I--P--R--* 110
(wild-type TIAR)
LENGTH: 2520 bp and 382 aa
TYPE: cDNA (SEQ ID NO: 92) and Protein (SEQ ID NO: 94)
ORGANISM: Nile tilapia
SEQ ID NOs 92 and 94
      1 GGAAATTTCTTCACAGTGACATCTGAGCTCAGATTCGAGAAAGGTCCTGGTGGTTCGCGC 60
          ............................................................
    61 CACTGCCTTGAGCCGTCAAATCTCGGCATTGAAAACAAGCGTACCTTTGCATTGCATTTC 120
          ............................................................
  121 AAAATAAGAGTTTCGTATGCAGCTTCCTTTTCCAAAATTAATAAAATAAGTACACATTAG 180
          ............................................................
  181 GTTTGCTCTTTCGGCTTTTTACAGTTAATTTTTTAAAAATGGTGTCATTCAGAAGTAACG 240
          ............................................................
  241 GTCTTTAAGAAATTTTCAATTTTTTACTATTAAGAACGCAAAAAGCCTTTTATTACTTCA 300
          ............................................................
  301 ACCTTATGTGACGGGTCTCTTCCTGCACACGCACGTACGTACTCTGGACTCTCACAGTGT 360
          ............................................................
  361 GACGTATGCTCTGGCGCCCGGTTAGCTAGCTTCTAAGCTAGTTAGCTAGTTGTGGTTTCT 420
          ............................................................
  421 AATTGCCAGTTAATACCAGCTATAACTAGCTAGTAAGTGGCGTTTTCTTCCCTGGTTACT 480
          ............................................................
  481 GTCAGCATCGACTATGGACGACGAAACCCACCCCAGAACCCTGTATGTGGGAAACCTCTC 540
          ............................................................
  481 GTCAGCATCGACTATGGACGACGAAACCCACCCCAGAACCCTGTATGTGGGAAACCTCTC 540
          .............-M--D--D--E--T--H--P--R--T--L--Y--V--G--N--L--S 16
  541 CAGGGATGTAACAGAAATTCTGATCCTGCAGCTCTTCACCCAGATAGGACCATGCAAAAG 600
    16 --R--D--V--T--E--I--L--I--L--Q--L--F--T--Q--I--G--P--C--K--S 36
  601 CTGTAAAATGATCACAGAGCACACGAGCAATGATCCCTATTGCTTTGTGGAGTTCTTTGA 660
    36 --C--K--M--I--T--E--H--T--S--N--D--P--Y--C--F--V--E--F--F--E 56
  661 ACACAGAGATGCTGCTGCAGCCCTTGCAGCCATGAATGGGAGGAAGATATTAGGAAAGGA 720
    56 --H--R--D--A--A--A--A--L--A--A--M--N--G--R--K--I--L--G--K--E 76
  721 GGTTAAAGTAAATTGGGCCACCACTCCAAGTAGCCAGAAGAAAGACACATCCAATCACTT 780
    76 --V--K--V--N--W--A--T--T--P--S--S--Q--K--K--D--T--S--N--H--F 96
  781 CCATGTTTTTGTGGGTGATTTGAATCCAGAGATTACTACTGAGGATGTCAGGGTTGCGTT 840
    96 --H--V--F--V--G--D--L--N--P--E--I--T--T--E--D--V--R--V--A--F 116
  841 TGCACCATTTGGGAAAATATCGGATGCCCGAGTTGTGAAGGACATGACGACAGGCAAATC 900
  116 --A--P--F--G--K--I--S--D--A--R--V--V--K--D--M--T--T--G--K--S 136
  901 AAAGGGGTATGGATTTGTGTCCTTCTACAACAAACTGGATGCAGAGAATGCCATTATTAA 960
  136 --K--G--Y--G--F--V--S--F--Y--N--K--L--D--A--E--N--A--I--I--N 156
  961 CATGTCGGGACAGTGGCTCGGAGGGCGCCAAATCAGGACTAACTGGGCTACGCGCAAACC 1020
  156 --M--S--G--Q--W--L--G--G--R--Q--I--R--T--N--W--A--T--R--K--P 176
1021 TCCAGCTCCTAAGAGCACTCAGGACAATGGTTCAAAGCAGCTGAGGTTCGATGACGTAGT 1080
  176 --P--A--P--K--S--T--Q--D--N--G--S--K--Q--L--R--F--D--D--V--V 196
1081 GAATCAATCCAGTCCACAGAACTGCACTGTGTACTGTGGAGGGATCCAATCAGGGCTATC 1140
  196 --N--Q--S--S--P--Q--N--C--T--V--Y--C--G--G--I--Q--S--G--L--S 216
1141 AGAACATCTAATGCGACAGACCTTCTCACCATTCGGTCAGATAATGGAAGTCAGGGTTTT 1200
  216 --E--H--L--M--R--Q--T--F--S--P--F--G--Q--I--M--E--V--R--V F 236
1201 CCCAGAGAAAGGATATTCTTTCATCAGGTTTTCCTCCCATGACAGTGCTGCCCATGCCAT 1260
  236 --P--E--K--G--Y--S--F--I--R--F--S--S--H--D--S--A--A--H--A--I 256
1261 TGTTTCAGTAAACGGCACAGTCATTGAAGGACACGTAGTGAAGTGCTTCTGGGGCAAAGA 1320
  256 --V--S--V--N--G--T--V--I--E--G--H--V--V--K--C--F--W--G--K--E 276
1321 ATCACCCGACATGGCAAAAAGCCCACAGCAGGTTGATTACAGTCAGTGGGGACAGTGGAA 1380
  276 --S--P--D--M--A--K--S--P--Q--Q--V--D--Y--S--Q--W--G--Q--W--N 296
1381 CCAGGTCTATGGGAATCCGCAGCAGCAGCAGCAGCAGCAGCAGCAGCAGCAGTATGGGCA 1440
  296 --Q--V--Y--G--N--P--Q--Q--Q--Q--Q--Q--Q--Q--Q--Q--Q--Y--G--Q 316
1441 GTATGTGACCAATGGGTGGCAAATGCCCTCTTACAACATGTATGGCCAGACATGGAACCA 1500
  316 --Y--V--T--N--G--W--Q--M--P--S--Y--N--M--Y--G--Q--T--W--N--Q 336
1501 GCAAGGATTTGGAGTAGAGCAGTCCCAGTCAACAGCCTGGATGGGAGGCTTTGGATCTCC 1560
  336 --Q--G--F--G--V--E--Q--S--Q--S--T--A--W--M--G--G--F--G--S--P 356
1561 ATCAGCCCAGGCTGCAGCCCCGCCTGGAACAGTCATGTCCAGCCTAGCCAACTTCGGCAT 1620
  356 --S--A--Q--A--A--A--P--P--G--T--V--M--S--S--L--A--N--F--G--M 376
1621 GGCTGGCTACCAAACGCAGTGAGAAGGCCCTAACCTCAATGTAATACCAAGGCGACACAG 1680
  376 --A--G--Y--Q--T--Q--*-...................................... 382
1681 CACTCTTACATTTGGACAGCTGCTGTGGTAAAAGAAGGGTGGGGCCATATTCACAAAGCC 1740
          ............................................................
1741 TTTCTAGCTAATAGTTGCTCCTACACAATTACAGTATACAACAGAAGGGAGCACACCCCT 1800
          ............................................................
1801 GTGCAATATTACATAAATGTCAGGTGATCAGAGCTGTACTGTCCAACAGTAATTGTGTTA 1860
          ............................................................
1861 CTTATGAGTACAGCAGTATGGTATTTGCTCTCATGCAAAGTTAAAAATGAATGGTATATA 1920
          ............................................................
1921 TTACCTTTATAATAAATATGTTTATATAATATTTGCTTTTCTTCATCAGAGGAAATACCC 1980
          ............................................................
1981 TTTCAATTCACGCAATAATGCTTTTACTGATACAGTTTTGACTCTTTTGAAATCTAGGTA 2040
          ............................................................
2041 ATATTGTACAGGTGTGTATTCGCTTTTGGTGTAGAGTGTATGATTTTAATCATAGTCAAT 2100
          ............................................................
2101 TAAACTCAGAACATTTAAAAAAAAAAGTTTGTTTCATTATTATTCCTAATTCTGTTTAAA 2160
          ............................................................
2161 AGAGAAAAAAAAGTGCTCTTGGGCTTCTCAAGTAAAGCCAAACCAACTGTTTTTGTGTGA 2220
          ............................................................
2221 GTACGTGTTTAAGGACACGCAGTCTTTATGAGTGCTAGACCATGTGAAACATTGAGGATT 2280
          ............................................................
2281 CATGCCTACAGGGTAGAGATCTTGGCAGAGACAGGACTTACGATGCCTTTCCTTTCATCA 2340
          ............................................................
2341 CACATTTAACAGGTTGACCAGTGGGCCAACCCATTAAAACATCAATTTAGTTCTTAAATC 2400
          ............................................................
2401 TAATTTGTTACTCAATACAAATTCTTTCATATTTTACAAAACAAAAGGGCCTTAGAGAAA 2460
          ............................................................
2461 ATGTACGTGTAACATAGCGCACACTTTTTAAATGCCACTATTATTATTTTATTTTTTTTT 2520
          ............................................................
(TIAR mutant allele-11 nt insertion)
LENGTH: 2520 bp(−11 bp) and 119 aa
TYPE: cDNA (SEQ ID NO: 93) and Protein (SEQ ID NO: 95)
ORGANISM: Nile tilapia
SEQ ID NOs 93 and 95
      1 GGAAATTTCTTCACAGTGACATCTGAGCTCAGATTCGAGAAAGGTCCTGGTGGTTCGCGC 60
          ............................................................
    61 CACTGCCTTGAGCCGTCAAATCTCGGCATTGAAAACAAGCGTACCTTTGCATTGCATTTC 120
          ............................................................
  121 AAAATAAGAGTTTCGTATGCAGCTTCCTTTTCCAAAATTAATAAAATAAGTACACATTAG 180
          ............................................................
  181 GTTTGCTCTTTCGGCTTTTTACAGTTAATTTTTTAAAAATGGTGTCATTCAGAAGTAACG 240
          ............................................................
  241 GTCTTTAAGAAATTTTCAATTTTTTACTATTAAGAACGCAAAAAGCCTTTTATTACTTCA 300
          ............................................................
  301 ACCTTATGTGACGGGTCTCTTCCTGCACACGCACGTACGTACTCTGGACTCTCACAGTGT 360
          ............................................................
  361 GACGTATGCTCTGGCGCCCGGTTAGCTAGCTTCTAAGCTAGTTAGCTAGTTGTGGTTTCT 420
          ............................................................
  421 AATTGCCAGTTAATACCAGCTATAACTAGCTAGTAAGTGGCGTTTTCTTCCCTGGTTACT 480
          ............................................................
  481 GTCAGCATCGACTATGGACGACGAAACCCACCCCAGAACCCTGTATGTGGGAAACCTCTC 540
          .............-M--D--D--E--T--H--P--R--T--L--Y--V--G--N--L--S 16
  541 CAGGGATGTAACAGAAATTCTGATCCTGCAGCTCTTCACCCAGATAGGACCATGCAAAAG 600
    16 --R--D--V--T--E--I--L--I--L--Q--L--F--T--Q--I--G--P--C--K--S 36
  601 CTGTAAAATGATCACAGAGCACACGAGCAATGATCCCTATTGCTTTGTGGAGTTCTTTGA 660
    36 --C--K--M--I--T--E--H--T--S--N--D--P--Y--C--F--V--E--F--F--E 56
  661 ACACAGAGATGCTGCTGCAGCCCTTGCAGCCATGAATGGGAGGAAGATATTAGGAAAGGA 720
    56 --H--R--D--A--A--A--A--L--A--A--M--N--G--R--K--I--L--G--K--E 76
  721 GGTTAAAGTAAATTGGGCCACCACTCCAAGTAGCCAGAAGAAAGACACATCCAATCACTT 780
    76 --V--K--V--N--W--A--T--T--P--S--S--Q--K--K--D--T--S--N--H--F 96
  781 CCATGTTTTTGTGGGTGATTTGAATCCAGAGATTACTACTGAGGATGTCAGGGTTGCGTT 840
    96 --H--V--F--V--G--D--L--N--P--E--I--T--T--E--D--V--R--V--A--F 116
  841 TGCACCAATATAATTATGTTTGGGAAAATATCGGATGCCCGAGTTGTGAAGGACATGACG 900
  116 --A--P--I--* 119
(wild-type KHSRP)
LENGTH: 2085 bp and 695 aa
TYPE: cDNA (SEQ ID NO: 96) and Protein (SEQ ID NO: 98)
ORGANISM: Nile tilapia
SEQ ID NOs 96 and 98
      1 ATGTCTGATTACAGCTCTCTGCCATCAAATGGAGTCGGAGCAGGAATGAAAAACGACGCT 60
      1 -M--S--D--Y--S--S--L--P--S--N--G--V--G--A--G--M--K--N--D--A- 20
    61 TTCGCAGATGCCGTTCAGCGAGCCAGACAGATTGCAGCTAAAATTGGTGGTGACGGTGTG 120
    21 -F--A--D--A--V--Q--R--A--R--Q--I--A--A--K--I--G--G--D--G--V- 40
  121 CCCCTGACAACAAACAACGGAGGAGCTGAGAGCTATCCGTTCACATCACAGAAACGATCC 180
    41 -P--L--T--T--N--N--G--G--A--E--S--Y--P--F--T--S--Q--K--R--S- 60
  181 CTGGAAGAAGGAGATGAACCCGATGCCAAGAAGGTAGCATCACAGAGTGAAACTATTGGA 240
    61 -L--E--E--G--D--E--P--D--A--K--K--V--A--S--Q--S--E--T--I--G- 80
  241 GCTCAGCTAGCTGCTCTGTCCCAGCAAAGTGTAAGGCCCTCCACAATGACAGAAGAGTGC 300
    81 -A--Q--L--A--A--L--S--Q--Q--S--V--R--P--S--T--M--T--E--E--C- 100
  301 AGGGTGCCTGATAGCATGGTTGGGCTCATCATTGGGCGAGGAGGCGAACAGATTAACAAA 360
  101 -R--V--P--D--S--M--V--G--L--I--I--G--R--G--G--E--Q--I--N--K- 120
  361 ATTCAGCAAGAATCTGGCTGCAAAGTCCAAATTGCTCATGACAGCGTGGGTCTGCCAGAA 420
  121 -I--Q--Q--E--S--G--C--K--V--Q--I--A--H--D--S--V--G--L--P--E- 140
  421 AGAAGTATTTCCCTCACAGGATCACCCGATGCCATACAGAGAGCCAGGGCACTTCTAGAT 480
  141 -R--S--I--S--L--T--G--S--P--D--A--I--Q--R--A--R--A--L--L--D- 160
  481 GATATTGTGTCCAGAGGTCACGAGTCAACCAACGGTCAGTCAAGTTCCATGCAAGAGATG 540
  161 -D--I--V--S--R--G--H--E--S--T--N--G--Q--S--S--S--M--Q--E--M- 180
  541 ATAATCCCTGCTGGAAAGGCTGGCCTTATTATCGGCAAAGGAGGAGAGACTATCAAACAA 600
  181 -I--I--P--A--G--K--A--G--L--I--I--G--K--G--G--E--T--I--K--Q- 200
  601 CTGCAGGAGCGAGCTGGAGTCAAAATGATTCTTATCCAAGATGCGTCGCAGCCACCCAAC 660
  201 -L--Q--E--R--A--G--V--K--M--I--L--I--Q--D--A--S--Q--P--P--N- 220
  661 ATAGATAAACCTCTTCGTATCATTGGAGACCCATACAAAGTCCAGCAAGCTAAGGAGATG 720
  221 -I--D--K--P--L--R--I--I--G--D--P--Y--K--V--Q--Q--A--K--E--M- 240
  721 GTTAATGAGATCCTACAGGAGAGGGATCATCAGGGTTTTGGAGAGAGGAACGAATATGGA 780
  241 -V--N--E--I--L--Q--E--R--D--H--Q--G--F--G--E--R--N--E--Y--G- 260
  781 TCAAGGATGGGAGGAGGGGGCATAGAAATAGCTGTCCCGCGGCACTCTGTGGGAGTTGTG 840
  261 -S--R--M--G--G--G--G--I--E--I--A--V--P--R--H--S--V--G--V--V- 280
  841 ATTGGTCGCAGTGGAGAGATGATCAAGAAGATCCAGAGTGATGCTGGCGTGAAAATACAG 900
  281 -I--G--R--S--G--E--M--I--K--K--I--Q--S--D--A--G--V--K--I--Q- 300
  901 TTTAAACCAGATGATGGTACAGGTCCTGATAAGATTGCTCATATTATGGGTCCACCAGAC 960
  301 -F--K--P--D--D--G--T--G--P--D--K--I--A--H--I--M--G--P--P--D- 320
  961 CAGTGTCAGCACGCTGCCTCGATCATCACTGACCTGCTACAGAGCATCCGTGCCAGAGAG 1020
  321 -Q--C--Q--H--A--A--S--I--I--T--D--L--L--Q--S--I--R--A--R--E- 340
1021 GAGGGTGGGCAAGGGGGTCCACCGGGTCCCGGTGCTGGTATGCCACCTGGTGGCCGAGGG 1080
  341 -E--G--G--Q--G--G--P--P--G--P--G--A--G--M--P--P--G--G--R--G- 360
1081 CAGGGTAGAGGCCAAGGGAACTGGGGTCCACCAGGAGGTGAGATGACTTTCTCCATCCCT 1140
  361 -Q--G--R--G--Q--G--N--W--G--P--P--G--G--E--M--T--F--S--I--P- 380
1141 GCTCACAAATGTGGGCTTGTTATTGGCAGAGGAGGAGAGAATGTCAAGTCCATCAACCAG 1200
  381 -A--H--K--C--G--L--V--I--G--R--G--G--E--N--V--K--S--I--N--Q- 400
1201 CAAACTGGTGCATTTGTGGAGATATCTCGTCAGCCACCTCCAAACGGTGACCCGAATTTC 1260
  401 -Q--T--G--A--F--V--E--I--S--R--Q--P--P--P--N--G--D--P--N--F- 420
1261 AAACTGTTCACCATCAGAGGGTCTCCACAACAGATAGATCATGCAAAGCAGCTTATAGAA 1320
  421 -K--L--F--T--I--R--G--S--P--Q--Q--I--D--H--A--K--Q--L--I--E- 440
1321 GAGAAGATTGAGGCTCCATTGTGTCCTGTGGGTGGTGGTCCTGGTCCAGGAGGGCCACCT 1380
  441 -E--K--I--E--A--P--L--C--P--V--G--G--G--P--G--P--G--G--P--P- 460
1381 GGTCCAATGGGTCCCTATAATCCGAACCCTTATAATGCAGGGCCTCCTGGTGGAGCTCCT 1440
  461 -G--P--M--G--P--Y--N--P--N--P--Y--N--A--G--P--P--G--G--A--P- 480
1441 CATGGAGCTGCACCAGGTGGTCCCCAGTATTCTCAGGGTTGGGGAAATGCCTATCAGCAG 1500
  481 -H--G--A--A--P--G--G--P--Q--Y--S--Q--G--W--G--N--A--Y--Q--Q- 500
1501 TGGCAAGCCCCAAATCCATATGACCCCAATAAGGCCGCAGCAGACCCAAATGCAGCATGG 1560
  501 -W--Q--A--P--N--P--Y--D--P--N--K--A--A--A--D--P--N--A--A--W- 520
1561 GCAGCCTACTATGCACAATACTATGGGCAGCAGCCCGGGGGCACAATGCCAGCTCAGAAT 1620
  521 -A--A--Y--Y--A--Q--Y--Y--G--Q--Q--P--G--G--T--M--P--A--Q--N- 540
1621 CCAGGAGCTCCTGCAGCAGGAGCATCACCAGGAGACCAGAGCCAGGCAGCCCAGACTGCT 1680
  541 -P--G--A--P--A--A--G--A--S--P--G--D--Q--S--Q--A--A--Q--T--A- 560
1681 GGGGGTCAGCCAGACTACACTAAGGCTTGGGAAGAGTATTATAAGAAGATGGGCATGAGC 1740
  561 -G--G--Q--P--D--Y--T--K--A--W--E--E--Y--Y--K--K--M--G--M--S- 580
1741 ACAGCAGCAGCCCCCACAGCAGCTGCAGCAGGAGGAGCTGCACCTGGTGGCCAGCAGGAC 1800
  581 -T--A--A--A--P--T--A--A--A--A--G--G--A--A--P--G--G--Q--Q--D- 600
1801 TACAGTGCAGCCTGGGCTGAGTACTACAGACAGCAGGCTGCCTACTATGAACAGACAGGC 1860
  601 -Y--S--A--A--W--A--E--Y--Y--R--Q--Q--A--A--Y--Y--E--Q--T--G- 620
1861 CAGGCTCCTGGACAGGCAGCTGCTCCACAGCAGGGACAACAGAGTACGTTGGAACTGTTT 1920
  621 -Q--A--P--G--Q--A--A--A--P--Q--Q--G--Q--Q--S--T--L--E--L--F 640
1921 TTTTGTTTTGTTTTTGTTTTTTTTAATATAAACCAGTTTTTTTGTCTTTTTTATTCCTCC 1980
  641 -F--C--F--V--F--V--F--F--N--I--N--Q--F--F--C--L--F--Y--S--S- 660
1981 CATTTGTTTTGTTTTTTACAGCTTGTTTTTAAATACTTAAGTAAAATGCCTAAAATGAAA 2040
  661 -H--L--F--C--F--L--Q--L--V--F--K--Y--L--S--K--M--P--K--M--K- 680
2041 GTTATCCTTTCAAGTTTAATGTTTTTATTCATATTTGAAAGTTTT 2085
  681 -V--I--L--S--S--L--M--F--L--F--I--F--E--S--F- 695
(KHSRP mutant allele- 17 nt deletion)
LENGTH: 2085 bp(−17 bp) and 410 aa
TYPE: cDNA (SEQ ID NO: 97) and Protein (SEQ ID NO: 99)
ORGANISM: Nile tilapia
SEQ ID NOs 97 and 99
        1 ATGTCTGATTACAGCTCTCTGCCATCAAATGGAGTCGGAGCAGGAATGAAAAACGACGCT 60
        1 -M--S--D--Y--S--S--L--P--S--N--G--V--G--A--G--M--K--N--D--A- 20
      61 TTCGCAGATGCCGTTCAGCGAGCCAGACAGATTGCAGCTAAAATTGGTGGTGACGGTGTG 120
      21 -F--A--D--A--V--Q--R--A--R--Q--I--A--A--K--I--G--G--D--G--V- 40
    121 CCCCTGACAACAAACAACGGAGGAGCTGAGAGCTATCCGTTCACATCACAGAAACGATCC 180
      41 -P--L--T--T--N--N--G--G--A--E--S--Y--P--F--T--S--Q--K--R--S- 60
    181 CTGGAAGAAGGAGATGAACCCGATGCCAAGAAGGTAGCATCACAGAGTGAAACTATTGGA 240
      61 -L--E--E--G--D--E--P--D--A--K--K--V--A--S--Q--S--E--T--I--G- 80
    241 GCTCAGCTAGCTGCTCTGTCCCAGCAAAGTGTAAGGCCCTCCACAATGACAGAAGAGTGC 300
      81 -A--Q--L--A--A--L--S--Q--Q--S--V--R--P--S--T--M--T--E--E--C- 100
    301 AGGGTGCCTGATAGCATGGTTGGGCTCATCATTGGGCGAGGAGGCGAACAGATTAACAAA 360
    101 -R--V--P--D--S--M--V--G--L--I--I--G--R--G--G--E--Q--I--N--K- 120
    361 ATTCAGCAAGAATCTGGCTGCAAAGTCCAAATTGCTCATGACAGCGTGGGTCTGCCAGAA 420
    121 -I--Q--Q--E--S--G--C--K--V--Q--I--A--H--D--S--V--G--L--P--E- 140
    421 AGAAGTATTTCCCTCACAGGATCACCCGATGCCATACAGAGAGCCAGGGCACTTCTAGAT 480
    141 -R--S--I--S--L--T--G--S--P--D--A--I--Q--R--A--R--A--L--L--D- 160
    481 GATATTGTGTCCAGAGGTCACGAGTCAACCAACGGTCAGTCAAGTTCCATGCAAGAGATG 540
    161 -D--I--V--S--R--G--H--E--S--T--N--G--Q--S--S--S--M--Q--E--M- 180
    541 ATAATCCCTGCTGGAAAGGCTGGCCTTATTATCGGCAAAGGAGGAGAGACTATCAAACAA 600
    181 -I--I--P--A--G--K--A--G--L--I--I--G--K--G--G--E--T--I--K--Q- 200
    601 CTGCAGGAGCGAGCTGGAGTCAAAATGATTCTTATCCAAGATGCGTCGCAGCCACCCAAC 660
    201 -L--Q--E--R--A--G--V--K--M--I--L--I--Q--D--A--S--Q--P--P--N- 220
    661 ATAGATAAACCTCTTCGTATCATTGGAGACCCATACAAAGTCCAGCAAGCTAAGGAGATG 720
    221 -I--D--K--P--L--R--I--I--G--D--P--Y--K--V--Q--Q--A--K--E--M- 240
    721 GTTAATGAGATCCTACAGGAGAGGGATCATCAGGGTTTTGGAGAGAGGAACGAATATGGA 780
    241 -V--N--E--I--L--Q--E--R--D--H--Q--G--F--G--E--R--N--E--Y--G- 260
    781 TCAAGGATGGGAGGAGGGGGCATAGAAATAGCTGTCCCGCGGCACTCTGTGGGAGTTGTG 840
    261 -S--R--M--G--G--G--G--I--E--I--A--V--P--R--H--S--V--G--V--V- 280
    841 ATTGGTCGCAGTGGAGAGATGATCAAGAAGATCCAGAGTGATGCTGGCGTGAAAATACAG 900
    281 -I--G--R--S--G--E--M--I--K--K--I--Q--S--D--A--G--V--K--I--Q- 300
    901 TTTAAACCAGATGATGGTACAGGTCCTGATAAGATTGCTCATATTATGGGTCCACCAGAC 960
    301 -F--K--P--D--D--G--T--G--P--D--K--I--A--H--I--M--G--P--P--D- 320
    961 CAGTGTCAGCACGCTGCCTCGATCATCACTGACCTGCTACAGAGCATCCGTGCCAGAGAG 1020
    321 -Q--C--Q--H--A--A--S--I--I--T--D--L--L--Q--S--I--R--A--R--E- 340
1021 GAGGGTGGGCAAGGGGGTCCACCGGGTCCCGGTGCTGGTATGCCACCTGGTGGCCGAGGG 1080
  341 -E--G--G--Q--G--G--P--P--G--P--G--A--G--M--P--P--G--G--R--G- 360
1081 CAGGGTAGAGGCCAAGGGAACTGGGGTGGTGAGATGACTTTCTCCATCCCTGCTCACAAAT 1140
  361 -Q--G--R--G--Q--G--N--W--G--G--E--M--T--F--S--I--P--A--H--K-- 380
1141 GTGGGCTTGTTATTGGCAGAGAATGTCAAGTCCATCAACCAGCAAACTGGTGCATTTGTGG 1200
  381 -C--G--L--V--I--G--R--E--C--Q--V--H--Q--P--A--N--W--C--I--C-- 400
1201 AGATATCTCGTCAGCCACCTCCAAACGGTGACCCGAATTTCAAACTGTTCACCATCAGAGG 1260
  401 G--D--I--S--S--A--T--S--K--R--* 410
(wild-type DHX9)
LENGTH: 4280 bp and 1286 aa
TYPE: cDNA (SEQ ID NO: 100) and Protein (SEQ ID NO: 102)
ORGANISM: Nile tilapia
SEQ ID NOs 100 and 102
      1 GACGATTCTCCCTCCGGCCTGAGGGGGCGCTGATGCACCGGGAGTTTATTTATTTTTTAA 60
          ............................................................
    61 CCGAAAGTGAAGTGCAGCCGGAGGAAGCCAAGGCTGCTAGGCTACCGGTGCTTAGCTGCT 120
          ............................................................
  121 GAAGTCTGGAGCAGCTTTTGCATTTTTCTGACCTGACTATTAACGGGTTCACGGAATAGG 180
          ............................................................
  181 AGGACCCTCCTGTCAGTCCACCATGGCGGACATCAAGAACTTCCTGTACGCCTGGTGTGG 240
          ......................-M--A--D--I--K--N--F--L--Y--A--W--C--G 13
  241 GAAAAAGAAGCTGACTCCAAACTACGACATCCGAGCAGCGGGCAACAAAAACAGGCAGAA 300
    13 --K--K--K--L--T--P--N--Y--D--I--R--A--A--G--N--K--N--R--Q--K 33
  301 GTTTATGTGTGAGGTCCGAGTCGATGGCTTCAACTACATTGGGATGGGAAACTCCACCAA 360
    33 --F--M--C--E--V--R--V--D--G--F--N--Y--T--G--M--G--N--S--T--N 53
  361 TAAGAAGGACGCGCAGACCAACGCCGCCCGCGACTTTGTCAACTATCTGGTCCGAATAGG 420
    53 --K--K--D--A--Q--T--N--A--A--R--D--F--V--N--Y--L--V--R--I--G 73
  421 AGAGATGAACGCAGCAGAGGTCCCGGCCATCGGGGTGAGCACGCCCATCGCAGATCAACC 480
    73 --E--M--N--A--A--E--V--P--A--I--G--V--S--T--P--I--A--D--Q--P 93
  481 TGATGCAGCTGGAGATGCTGGCTTTGGAAACCTCCCTTCTAGCGGTCCTCTACCACCTCA 540
    93 --D--A--A--G--D--A--G--F--G--N--L--P--S--S--G--P--L--P--P--H 113
  541 CCTGGTAGTGAAAGCTGAGCAAGGGGACGGCAGCGTCAGTGGGCCGGTTCCAGGAGTGAC 600
  113 --L--V--V--K--A--E--Q--G--D--G--S--V--S--G--P--V--P--G--V--T 133
  601 CGGACTGGGTTATGCAGGAGGAGGAAACTCCGGTTGGGGCAGAGGAGGAAGTGACGGAGG 660
  133 --G--L--G--Y--A--G--G--G--N--S--G--W--G--R--G--G--S--D--G--G 153
  661 AGCTCAGTGGGACCGAGGAGCCAACCTGAAGGAGTATTACGCCAAGAGAGACGAACAGGA 720
  153 --A--Q--W--D--R--G--A--N--L--K--E--Y--Y--A--K--R--D--E--Q--E 173
  721 AGCACAGGCGACTCTGGAGTCGGAGGAAGTGGATCTGAACGCTAACCTTCACGGAAACTG 780
  173 --A--Q--A--T--L--E--S--E--E--V--D--L--N--A--N--L--H--G--N--W 193
  781 GACTCTGGAGAACGCCAAGGCCCGTCTGAACCAGTTCTTCCAGAAGGAGAAAACCAGTGC 840
  193 --T--L--E--N--A--K--A--R--L--N--Q--F--F--Q--K--E--K--T--S--A 213
  841 TGAGTATAAATACAGCCAAGTGGGACCGGACCACAACAGGAGCTTCATAGCAGAGATGCA 900
  213 --E--Y--K--Y--S--Q--V--G--P--D--H--N--R--S--F--I--A--E--M--Q 233
  901 GCTTTTTGTGAAGCAGCTTGGCAGAAGGATCACGGCTCGAGAGCACGGCTCCAACAAGAA 960
  233 --L--F--V--K--Q--L--G--R--R--I--T--A--R--E--H--G--S--N--K--K 253
  961 GCTGGCGGCTCAGTCGTGCGCTCTGTCTCTGGTCCGACAGCTGTATCACCTGGGAGTCAT 1020
  253 --L--A--A--Q--S--C--A--L--S--L--V--R--Q--L--Y--H--L--G--V--I 273
1021 CGAGGCGTACTCTGGGGTCACCAAGAAGAAGGAGGGAGAAACTTTGGAGGCGTTTGAGGT 1080
  273 --E--A--Y--S--G--V--T--K--K--K--E--G--E--I--L--E--A--F--E--V 293
1081 CAACGTGTCTCCAGACCTGCAGCAGCAGCTGGCCTCTGTGGTCCAGGAGCTCGGAGTCAG 1140
  293 --N--V--S--P--D--L--Q--Q--Q--L--A--S--V--V--Q--E--L--G--V--S 313
1141 CGTCCCCCCACCGCCTGCAGACCCCAGCAGCCCGGTGTCTCTGGCTCAGGGGAAGCTGGC 1200
  313 --V--P--P--P--P--A--D--P--S--S--P--V--S--L--A--Q--G--K--L--A 333
1201 GTACTTCGAGCCGTCACAGAGGCAGACCGGAGCCGGAGTCGTCCCCTGGTCGCCTCCTCA 1260
  333 --Y--F--E--P--S--Q--R--Q--T--G--A--G--V--V--P--W--S--P--P--Q 353
1261 GGTCAACTGGAACCCCTGGACCAGCAGCAACATCGACGAGGGGCCGCTGGCCTACTGCAC 1320
  353 --V--N--W--N--P--W--T--S--S--N--I--D--E--G--P--L--A--Y--C--I 373
1321 TCCAGAGCAGATCAGCGGCGACCTGCACGACGAGCTGAAGTACCAGCTGGAGCATGATGA 1380
  373 --P--E--Q--I--S--G--D--L--H--D--E--L--K--Y--Q--L--E--H--D--E 393
1381 AAACCTGCAGAAGATCCTGATGGAACGCGAGCAGCTGCCCGTCAAACAGTTTGAGGAGGA 1440
  393 --N--L--Q--K--I--L--M--E--R--E--Q--L--P--V--K--Q--F--E--E--E 413
1441 GATCATGGCGGCCATCGACAAAAGCCCTGTGGTGATCATCAGAGGAGCGACGGGCTGCGG 1500
  413 --I--M--A--A--I--D--K--S--P--V--V--I--I--R--G--A--T--G--C--G 433
1501 TAAAACCACTCAGGTTCCTCAGTACATCCTGGACCGCTTCATCAAGGGGGGCCGAGCATC 1560
  433 --K--T--T--Q--V--P--Q--Y--I--L--D--R--F--I--K--G--G--R--A--S 453
1561 GGACTGCAACATCGTGGTCACCCAGCCCAGACGGATCAGCGCCGTGTCCGTGGCTGAGAG 1620
  453 --D--C--N--I--V--V--T--Q--P--R--R--I--S--A--V--S--V--A--E--R 473
1621 GGTCGCCTTTGAGAGAGCAGAGGATCTTGGGAAAAGCTGTGGCTACAGCGTCCGATTTGA 1680
  473 --V--A--F--E--R--A--E--D--L--G--K--S--C--G--Y--S--V--R--F--E 493
1681 GTCCGTCCTCCCTCGACCCCACGCCAGTGTCCTCTTCTGCACCGTCGGTGTTCTTCTGCG 1740
  493 --S--V--L--P--R--P--H--A--S--V--L--F--C--T--V--G--V--L--L--R 513
1741 GAAGCTGGAAGCAGGAATCAGAGGCATCAGTCACGTCATCGTTGATGAGATCCACGAGAG 1800
  513 --K--L--E--A--G--I--R--G--I--S--H--V--I--V--D--E--I--H--E--R 533
1801 AGACATCAACACGGACTTCCTCATGGTGGTCCTCAGAGACGTGGTCCAGGCCTACCCGGA 1860
  533 --D--I--N--T--D--F--L--M--V--V--L--R--D--V--V--Q--A--Y--P--D 553
1861 CGTGCGCATCATCCTCATGTCGGCCACCATCGACACCACCATGTTCAGAGAGTACTTCTT 1920
  553 --V--R--I--I--L--M--S--A--T--I--D--T--T--M--F--R--E--Y--F--F 573
1921 CAGCTGCCCCGTCATTGAGGTGTTTGGTCGCACCTTCCCCGTCCAAGAGTATTTCCTGGA 1980
  573 --S--C--P--V--I--E--V--F--G--R--T--F--P--V--Q--E--Y--F--L--E 593
1981 GGACTGCATCCAGATGACAAAGTTTGTGCCTCCACCGATGGACCGAAAGAAGAAAGACAA 2040
  593 --D--C--I--Q--M--T--K--F--V--P--P--P--M--D--R--K--K--K--D--K 613
2041 AGACGAGGAGGGAGGAGACGACGACACTAACTGTAATGTGATCTGCGGGCCGGAGTATAC 2100
  613 --D--E--E--G--G--D--D--D--I--N--C--N--V--I--C--G--P--E--Y--T 633
2101 GCCGGAGACGAAGCATTCGATGGCTCAGATCAATGAGAAGGAAACGTCCTTCGAGCTGGT 2160
  633 --P--E--T--K--H--S--M--A--Q--I--N--E--K--E--T--S--F--E--L--V 653
2161 GGAGGCGCTACTGAAGTACATCGAGACGCTGCAGGTGGCCGGCGCCGTGCTCGTCTTCCT 2220
  653 --E--A--L--L--K--Y--T--E--T--L--Q--V--A--G--A--V--L--V--F--L 673
2221 CCCCGGCTGGAACCTCATCTACTCCATGCAGAGACACCTGGAGAGCAACCCACACTTCGG 2280
  673 --P--G--W--N--L--I--Y--S--M--Q--R--H--L--E--S--N--P--H--F--G 693
2281 AAGCAACCGGTACCGAATCCTGCCGCTGCACTCTCAGATACCTCGAGAGGAGCAGAGGAG 2340
  693 --S--N--R--Y--R--I--L--P--L--H--S--Q--I--P--R--E--E--Q--R--R 713
2341 GGTGTTTGAACCAGTTCCTGATGACATCAGAAAGGTGATCCTGTCCACCAACATCGCCGA 2400
  713 --V--F--E--P--V--P--D--D--I--R--K--V--I--L--S--T--N--I--A--E 733
2401 GACGAGCATCACCATCAACGATGTCGTCTACGTCGTCGACTCCTGCAAGCAGAAAGTGAA 2460
  733 --T--S--I--T--I--N--D--V--V--Y--V--V--D--S--C--K--Q--K--V--K 753
2461 GCTGTTCACCTCCCACAACAATATGACCAACTACGCCACCGTCTGGGCCTCCAAGACCAA 2520
  753 --L--F--T--S--H--N--N--M--T--N--Y--A--T--V--W--A--S--K--T--N 773
2521 CCTGGAGCAGAGGAAAGGTCGAGCCGGCAGAGTCCGACCGGGGTTCTGCTTCCACCTCTG 2580
  773 --L--E--Q--R--K--G--R--A--G--R--V--R--P--G--F--C--F--H--L--C 793
2581 CAGCCGCGCTCGATTCGACAAGTTGGAGACTCACATGACTCCAGAGATCTTCAGAACTCC 2640
  793 --S--R--A--R--F--D--K--L--E--T--H--M--T--P--E--I--F--R--T--P 813
2641 GCTGCATGAAATTGCCCTGAGCATCAAACTGCTGAGACTCGGAGGCATCGGCCACTTCCT 2700
  813 --L--H--E--I--A--L--S--I--K--L--L--R--L--G--G--I--G--H--F--L 833
2701 GTCTAAGGCCATCGAGCCACCGCCGCTGGACGCCGTCATCGAGGCCGAACACACCTTGAA 2760
  833 --S--K--A--I--E--P--P--P--L--D--A--V--I--E--A--E--H--T--L--K 853
2761 AGAGCTGGACGCCCTGGACTCCAACGACGAGCTGACCCCTCTGGGGCGGATTCTGGCTCG 2820
  853 --E--L--D--A--L--D--S--N--D--E--L--T--P--L--G--R--I--L--A--R 873
2821 GCTGCCCATCGAACCTCGGCTGGGGAAGATGATGATCATGGGCTGCATCTTCCACGTCGG 2880
  873 --L--P--I--E--P--R--L--G--K--M--M--I--M--G--C--I--F--H--V--G 893
2881 CGATGCAATGTGCACCATCTCGGCCGCCACCTGTTTCCCAGAGCCTTTCATCAGCGAGGG 2940
  893 --D--A--M--C--T--I--S--A--A--T--C--F--P--E--P--F--I--S--E--G 913
2941 GAAGCGTCTCGGCTTCGTGCACAGAAACTTTGCTGGCAGTCGTTTCTCGGATCACGTGGC 3000
  913 --K--R--L--G--F--V--H--R--N--F--A--G--S--R--F--S--D--H--V--A 933
3001 GCTGCTGTCCGTGTTCCAGGCCTGGGACGACGTCAGGATTAACGGAGAGGAGGCGGAGAG 3060
  933 --L--L--S--V--F--Q--A--W--D--D--V--R--I--N--G--E--E--A--E--S 953
3061 TCGCTTCTGTGACCACAAACGTCTCAACATGTCGACTCTGAGGATGACCTGGGAGGCCAA 3120
  953 --R--F--C--D--H--K--R--L--N--M--S--T--L--R--M--T--W--E--A--K 973
3121 AGTCCAGCTGAAGGAGATCCTGGTGAACTCTGGATTTCCTGAAGAGTGTCTCATGACGCA 3180
  973 --V--Q--L--K--E--I--L--V--N--S--G--F--P--E--E--C--L--M--T--Q 993
3181 GATGTTCAACACGGTGGGGCCGGACAACAACCTGGACGTGGTGGTCTCTCTGCTCACCTT 3240
  993 --M--F--N--T--V--G--P--D--N--N--L--D--V--V--V--S--L--L--T--F 1013
3241 CGGCTCGTACCCCAACGTCTGCTACCACAAAGAGAAGAGGAAGATCCTGACCACCGAGGG 3300
1013 --G--S--Y--P--N--V--C--Y--H--K--E--K--R--K--I--L--T--T--E--G 1033
3301 GCGCAACGCCCTCATCCACAAATCCTCCGTCAACTGTCCCTTCAGCAGCCACGACATGAT 3360
1033 --R--N--A--L--I--H--K--S--S--V--N--C--P--F--S--S--H--D--M--I 1053
3361 CTACCCGTCGCCATTCTTCGTCTTCGGCGAGAAGATCCGAACCAGAGCGATCTCGGCCAA 3420
1053 --Y--P--S--P--F--F--V--F--G--E--K--I--R--T--R--A--I--S--A--K 1073
3421 AGGGATGACTCTGGTCAGTCCTCTGCAGCTGCTGCTGTTCGCCTGCAAGAAGGTGACCTC 3480
1073 --G--M--T--L--V--S--P--L--Q--L--L--L--F--A--C--K--K--V--T--S 1093
3481 TAACGGAGAGATCGTGGAGCTCGACGACTGGATCAAACTGAAGATTGCTCACGAGGTGGC 3540
1093 --N--G--E--I--V--E--L--D--D--W--I--K--L--K--I--A--H--E--V--A 1113
3541 GGGGAGCATCCTGGCTCTGCGGGCCGCCCTGGAGGCGGTGGTGGTGGAGGTGACCAAAGA 3600
1113 --G--S--I--L--A--L--R--A--A--L--E--A--V--V--V--E--V--T--K--D 1133
3601 CCCGGAGTACATCAGACAGATGGACCAAACCAACGAGCGGCTCCTGAACGTCATCAGACA 3660
1133 --P--E--Y--I--R--Q--M--D--Q--T--N--E--R--L--L--N--V--I--R--H 1153
3661 CGTCTCCAAACCGTCGGCGGCCGGGCTCAACATGATGGCCAACAACCAGAGGATGGGAGA 3720
1153 --V--S--K--P--S--A--A--G--L--N--M--M--A--N--N--Q--R--M--G--D 1173
3721 CGGTCCACGACCTCCGAAGATGCCGCGTTTTGATGGAGGAGGCGGCGGCAGAGGTTACCA 3780
1173 --G--P--R--P--P--K--M--P--R--F--D--G--G--G--G--G--R--G--Y--Q 1193
3781 AGGAGGAGGAGGCTACAGGGGAGGAGGAGGAGGAGGGGGATACAGAGGAGGTGGAGGATA 3840
1193 --G--G--G--G--Y--R--G--G--G--G--G--G--G--Y--R--G--G--G--G--Y 1213
3841 TGGAGGAGGAGGAGGAGGGGGATACAGAGGAGGTGGAGGATATGGAGGAGGAGGAGGAGG 3900
1213 --G--G--G--G--G--G--G--Y--R--G--G--G--G--Y--G--G--G--G--G--G 1233
3901 GGGATACAGAGGAGGTGGCGGAGGATACAGGGGTGGTGGAGGATATGGAGGATACAGAGG 3960
1233 --G--Y--R--G--G--G--G--G--Y--R--G--G--G--G--Y--G--G--Y--R--G 1253
3961 AGGTGGTGGTTATGGTGGTGGAGGAGGTGGTTATAGGGGAGGTGGTTATAGAGGAGGAGG 4020
1253 --G--G--G--Y--G--G--G--G--G--G--Y--R--G--G--G--Y--R--G--G--G 1273
4021 CAGCAGTTATGGAGGAGGTGGAGGATGCAGAGGAGGATACTAAGGTGAAAAATCAGTCAT 4080
1273 --S--S--Y--G--G--G--G--G--C--R--G--G--Y--*- 1286
4081 CTCGTGTCTTCTTCTTCTTCTTCTTTAGTTTATTGAGTAAAAGATTAATGTGAAATCGAC 4140
          ............................................................
4141 CGTTGCAGTTAAAACGATGTTTGACTGGAACCTGCTGATGTTTGTTTTTATGGTCTGTAA 4200
          ............................................................
4201 ATGAAAACGTCCCCAATAAATCTGTCATGTTCCCTCATCGCGTTGGCTCATTTTTCCTCT 4260
          ............................................................
4261 TCACACATTTAAAGTCTGAA 4280
          ....................
(DHX9 mutant allele-7 nt deletion)
LENGTH: 4280 bp(−7 bp) and 82 aa
TYPE: cDNA (SEQ ID NO: 101) and Protein (SEQ ID NO: 103)
ORGANISM: Nile tilapia
SEQ ID NOs 101 and 103
      1 GACGATTCTCCCTCCGGCCTGAGGGGGCGCTGATGCACCGGGAGTTTATTTATTTTTTAA 60
          ............................................................
    61 CCGAAAGTGAAGTGCAGCCGGAGGAAGCCAAGGCTGCTAGGCTACCGGTGCTTAGCTGCT 120
          ............................................................
  121 GAAGTCTGGAGCAGCTTTTGCATTTTTCTGACCTGACTATTAACGGGTTCACGGAATAGG 180
          ............................................................
  181 AGGACCCTCCTGTCAGTCCACCATGGCGGACATCAAGAACTTCCTGTACGCCTGGTGTGG 240
          ......................-M--A--D--I--K--N--F--L--Y--A--W--C--G 13
  241 GAAAAAGAAGCTGACTCCAAACTACGACATCCGAGCAGCGGGCAACAAAAACAGGCAGAA 300
    13 --K--K--K--L--I--P--N--Y--D--I--R--A--A--G--N--K--N--R--Q--K 33
  301 GTTTATGTGTGAGGTCCGAGTCGATGGCTTCAACTACATTGGGATGGGAAACTCCACCAA 360
    33 --F--M--C--E--V--R--V--D--G--F--N--Y--I--G--M--G--N--S--I--N 53
  361 TAAGAAGGACGCGCAGACCAACGCCGCCCGCGACTTTGTCAACTATCTGGTCCGAATAGG 420
    53 --K--K--D--A--Q--I--N--A--A--R--D--F--V--N--Y--L--V--R--I--G 73
  421 AGAGATGAACGCAGCAGAGGTCCCGGGGTGAGCACGCCCATCGCAGATCAACCTGATGCA 480
    73 --E--M--N--A--A--E--V--P--G--*- 82
LENGTH: 3664 bp and 387 aa
TYPE: cDNA (SEQ ID NO: 104) and Protein (SEQ ID NO: 106)
ORGANISM: Nile tilapia
(wild-type TIA1)
SEQ ID NOs 104 and 106
      1 CCTGTGTGACACGTAGAGAATAAAAATGTGGGGGCGCATCTTTGTGTGTGGGAGCAGGAG 60
          ............................................................
    61 CGCTTGATTTTGGCTTAATTTCAGCGCGCAGGTTGACGCTGCTGACGCCGCTCCTCCGCC 120
          ............................................................
  121 ATCTTCAACTTCCTATTGTTTGCATCAGACTGAGGCTGTCTGCGGTGTGTGCCAGAGAGA 180
          ............................................................
  181 GCAGAGTCGACCGCGGATATATTATTAAATAGTAGATTTAGTCTTTACGTTCGGGTCGCT 240
          ............................................................
  241 AAAGTTCAGCACAAACCATTTGTATGTCACTGGATTAAAAGCTTTCTCAGGACGAAACCA 300
          ............................................................
  301 CTAAACCTTGATGATGGAGGACGATCAACCCAGAACCTTGTATGTGGGGAATCTGTCCAG 360
          ..........-M--M--E--D--D--Q--P--R--T--L--Y--V--G--N--L--S--R 17
  361 GGATGTCACCGAGCCCCTCATTCTGCAGGTCTTCACACAGATAGGCCCCTGCAAGAGCTG 420
    17 --D--V--T--E--P--L--I--L--Q--V--F--T--Q--I--G--P--C--K--S--C 37
  421 TAAAATGATAGTCGATACAGCTGGCAATGATCCGTACTGCTTCGTGGAGTTCTATGACCA 480
    37 --K--M--I--V--D--T--A--G--N--D--P--Y--C--F--V--E--F--Y--D--H 57
  481 CAGGCATGCTGCTGCCTCATTGGCAGCTATGAATGGAAGGAAAATAATGGGTAAGGAAGT 540
    57 --R--H--A--A--A--S--L--A--A--M--N--G--R--K--I--M--G--K--E--V 77
  541 CAAAGTCAACTGGGCCACGACACCAACCAGCCAGAAAAAAGACACAAGTAATCATTTTCA 600
    77 --K--V--N--W--A--T--T--P--T--S--Q--K--K--D--T--S--N--H--F--H 97
  601 TGTTTTTGTTGGCGACCTCAGCCCAGAAATAACCACAGAAGACGTCAAAGCTGCCTTTGG 660
    97 --V--F--V--G--D--L--S--P--E--I--T--T--E--D--V--K--A--A--F--G 117
  661 TCCATTCGGCAGGATATCAGATGCTCGTGTTGTGAAAGACATGGCTACAGGGAAATCTAA 720
  117 --P--F--G--R--I--S--D--A--R--V--V--K--D--M--A--T--G--K--S--K 137
  721 AGGCTATGGCTTCGTGTCTTTCTTTAACAAATGGGATGCAGAGAATGCCATTCAGCACAT 780
  137 --G--Y--G--F--V--S--F--F--N--K--W--D--A--E--N--A--I--Q--H--M 157
  781 GGGGGGGCAGTGGTTAGGAGGCAGACAGATTCGAACTAACTGGGCCACAAGAAAGCCTCC 840
  157 --G--G--Q--W--L--G--G--R--Q--I--R--T--N--W--A--T--R--K--P--P 177
  841 CGCCCCAAAGACCACCCATGAAAAIAACTCCAAGCATCTCTCTTTTGATGAAGTAGTGAA 900
  177 --A--P--K--T--T--H--E--N--N--S--K--H--L--S--F--D--E--V--V--N 197
  901 TCAGTCCAGCCCCAGTAACTGCACTGTGTACTGTGGTGGAGTCAGCACAGGACTGACGGA 960
  197 --Q--S--S--P--S--N--C--T--V--Y--C--G--G--V--S--T--G--L--T--E 217
  961 GCAACTAATGAGACAGACCTTCTCCCCCTTTGGACAAATCATGGAAGTCAGAGTTTTTCC 1020
  217 --Q--L--M--R--Q--T--F--S--P--F--G--Q--I--M--E--V--R--V--F--P 237
1021 TGACAAAGGATATTCATTTGTCAGGTTCAACTCCCATGAGTCAGCAGCCCATGCCATTGT 1080
  237 --D--K--G--Y--S--F--V--R--F--N--S--H--E--S--A--A--H--A--I--V 257
1081 GTCCGTGAATGGCTCTTCTATAGAGGGGCACATAGTCAAATGCTACTGGGGTAAAGAGAC 1140
  257 --S--V--N--G--S--S--I--E--G--H--I--V--K--C--Y--W--G--K--E--T 277
1141 CCCAGACATGATGAACTCCATGCAGCAGATGCCTGTGCCACAACAAAACAAGATGGGCTT 1200
  277 --P--D--M--M--N--S--M--Q--Q--M--P--V--P--Q--Q--N--K--M--G--F 297
1201 TGCTGCAGCTCAGCCTTATGGCCAGTGGGGACAGTGGTACGGCAATGGGCCCCAGATTGG 1260
  297 --A--A--A--Q--P--Y--G--Q--W--G--Q--W--Y--G--N--G--P--Q--I--G 317
1261 CCAGTATGTCCCCAACGGGTGGCAGGTCCCCACCTACGGTGTCTACGGGCAGGCTTGGAA 1320
  317 --Q--Y--V--P--N--G--W--Q--V--P--T--Y--G--V--Y--G--Q--A--W--N 337
1321 TCAGCAGGGCTTCAATCACTTACCGGCCAGTGCTGGGTGGACTGGCATGAGCGCCATCAG 1380
  337 --Q--Q--G--F--N--H--L--P--A--S--A--G--W--T--G--M--S--A--I--S 357
1381 TAACGGTGGGGTTATGGAGCCTACACAGGGATTGAATGGGAGTATGCTAGCCAACCAGCC 1440
  357 --N--G--G--V--M--E--P--T--Q--G--L--N--G--S--M--L--A--N--Q--P 377
1441 CGGTATGGGAGCCGCAGGATACCCCACACACTGATAAGTGGGCAGGGTGGGAGATTTGTC 1500
  377 --G--M--G--A--A--G--Y--P--T--H--*-.......................... 387
1501 AACCATCAGCCTCTTGCTGGCTGTACGGTGCCCTGCGGGGCTGTGTAACACTGCCTCCAT 1560
          ............................................................
1561 TTTGTGGCAGGACTGAGACTTTACTGGGATGTGGAACCTAATGAGAAGGGTGACGTCTGT 1620
          ............................................................
1621 GGAGATGTAAATGGGATTTCTTGGGGTGGGCTGAGGTAACGGGAGCCAGGGAGCAGCAGT 1680
          ............................................................
1681 TTGACCCACACAGGTATTTACACCATTTGTGGTAGGAAAGACTGGCCATGAACCAGGGCT 1740
          ............................................................
1741 CTTACCATTTTTAAGTTAACTGTAAATGAATTATAAAACTGTAAAGGAGAATCTCTTTTT 1800
          ............................................................
1801 TTCCTGGGTTTTACAGATTGCCTCCATTTTCACTTCTTTCCTCTCGACCACTGAGAGGTT 1860
          ............................................................
1861 TCTTTTCTCTTTTTCTTTTTTTTGGAACTGAGTCATGCTAAGTTATGATCCTTAATTATC 1920
          ............................................................
1921 TGAGGAATGGAAATTTGTTCTAATTTTCTCTTGGATTAAAAACAATTGCAGGGATTGTTG 1980
          ............................................................
1981 CCACTGCTGTTTCTCTGTAAGGGCAGATTAATATTGCACAGTTCTTTCCTCTCTTGGATT 2040
          ............................................................
2041 TCCCAGAAAAATTTGACTACCAAGAGCATTTTTCTTTTTTTCTTTTCTTTTTTGCATTCC 2100
          ............................................................
2101 ATTTCTCCTTCATATCTTTCTGACAGCCTCAAAACTTTTTTCGCCACGTGTAAATAACCA 2160
          ............................................................
2161 TCCATTCATTTGAAACGATGTAAGTAAAATGCTACTGTTAACTGTGGGTGCTTGTTTTTC 2220
          ............................................................
2221 TTTTTTTTGTTTTTGTTTTGATAACTCGACAGTTAACTCGAACATTGTACGTAGCAGAGT 2280
          ............................................................
2281 GGCACCATCAAAGGTGACACTGGCACAGTGCAACACGCGACTCTTCCATGCAGGGATAAG 2340
          ............................................................
2341 ACAGCATTGCTATGCAGTGCATACTTTAAAATTTAACACGATTCAAACGTTAAAGTGTGA 2400
          ............................................................
2401 ACATGTTTGACACTTCTGATGTTTCTTTCTTTTTTTTTTTCTTTTTTTTTTAAATATCTA 2460
          ............................................................
2461 TTGAAACGCCAGTATTTTATATCAGACAAATCTGAGTGTATTCAGCTTTACACTTGCTCT 2520
          ............................................................
2521 TTTTGCCAGAGAGATGGAGAGGCCTACATTGTGTAACTGTTGCCTTATAGAGCTGGTTTC 2580
          ............................................................
2581 TTTTAGCTGACAAGATACTCTTTTTAATTAGGCAGTGCCTACAGACCTTTTCAGACCTTT 2640
          ............................................................
2641 TTGTTTCGAAAGGTGTTAGTCCTGAGAACCGATGACTCTGCTACTGTAATCAATGTTTTC 2700
          ............................................................
2701 TTGCTTTGTCCAATTAAAATGCTAATGCACATAAACCACACTTTGTGTTTGTTTGCCCCC 2760
          ............................................................
2761 TTTGTTTCTTTTTGGTCATATTAGAGCATCAAATGGAAAAACTGCATCTTGACAACTGTG 2820
          ............................................................
2821 TCCAAAACTCTAAGCACATCACACAAAACATCTGGAAAAGTCTTGCTGATCATAGCCTGC 2880
          ............................................................
2881 CAGTACTTGACCACACGACCACATTTGTTATGAAGAAAACCTGCTGATCTGTATCATGGA 2940
          ............................................................
2941 GCAGTTCAGCCAAATGTGTGGGGTTTTTTTAAGCCACCGGTCGCTTTAATCTTCTAACAT 3000
          ............................................................
3001 CTGCAGCCTTGTGTGTGTTTAAGACATTAGATTCTGTCCGGCTGAACCAGAGGAACTTTA 3060
          ............................................................
3061 TTTGGTGCCAAAGCGCACAGATAACAGATATCTCACCAAAATGTAGAAATGTGGGCAAAC 3120
          ............................................................
3121 ATAAATCAGGTCATGTGATCCCAAAACTCTTAATGGCTTCAAAGGTGAAAATGAAGCACA 3180
          ............................................................
3181 TAAGTGTTTTTTATAATCATATTACAGTAAGTCAGTCACACTGCAGCTAAAACTAGAGCT 3240
          ............................................................
3241 TAAAAAAAAGAACTTAAAGCCTTAGTTTTAGGGCACTACGTGCATAAAATTTTACAGTTC 3300
          ............................................................
3301 ATAAAGTAAATGAGCCACAGCTGAGATGGATTCAGCACAAAAAATGTTGAAGATACAATT 3360
          ............................................................
3361 TTAATTTTAATAAAAACAAAACTGTGCCTTCAGGTTGTCTGTTTGACTTTAACATTCGGT 3420
          ............................................................
3421 TCATTAAAGCACTGGATTGTATTCATTTATTTACATCTCATTTATTCCAGTTCATAAAAC 3480
          ............................................................
3481 AAAAAGGATTTCCCACAGTTCTACCACCACCTTCTGGCTGGAGCGTTTTATAGTTTGTCA 3540
          ............................................................
3541 GAGGACATTTGGAAAAAAAAAAAAAGAAAAAAAAAAAGCACATCCATATGTTTTCTCAGA 3600
          ............................................................
3601 AAGTGATGTTTGTTCCAAACCCTAAAAACACAATGCAAAGACTTGCTGGGGATTATGTTT 3660
          ............................................................
3661 CAAT 3664
          ....
(TIA1 mutant allele-10 nt deletion)
LENGTH: 3664 bp(−10 bp) and 27 aa
TYPE: cDNA (SEQ ID NO: 105) and Protein (SEQ ID NO: 107)
ORGANISM: Nile tilapia
SEQ ID NOs 105 and 107
      1 CCTGTGTGACACGTAGAGAATAAAAATGTGGGGGCGCATCTTTGTGTGTGGGAGCAGGAG 60
          ............................................................
    61 CGCTTGATTTTGGCTTAATTTCAGCGCGCAGGTTGACGCTGCTGACGCCGCTCCTCCGCC 120
          ............................................................
  121 ATCTTCAACTTCCTATTGTTTGCATCAGACTGAGGCTGTCTGCGGTGTGTGCCAGAGAGA 180
          ............................................................
  181 GCAGAGTCGACCGCGGATATATTATTAAATAGTAGATTTAGTCTTTACGTTCGGGTCGCT 240
          ............................................................
  241 AAAGTTCAGCACAAACCATTTGTATGTCACTGGATTAAAAGCTTTCTCAGGACGAAACCA 300
          ............................................................
  301 CTAAACCTTGATGATGGAGGACGATCAACCCAGAACCTTGTATGTGGGGAATCTGTCACC 360
          ........-M--M--E--D--D--Q--P--R--T--L--Y--V--G--N--L--P--S 17
  361 GAGCCCCTCATTCTGCAGGTCTTCACACAGATAGGCCCCTGCAAGAGCTGTAAAATGATA 420
    17 --S--P--S--F--C--R--S--S--H--R--* 27
(wild-type Iaf2bp3)
LENGTH: 2288 bp and 589 aa
TYPE: cDNA (SEQ ID NO: 108) and Protein (SEQ ID NO: 110)
ORGANISM: Nile tilapia
SEQ ID NOs 108 and 110
      1 ATGAATAAGCTATACATTGGCAACGTAAGCGCAGAGGCGAGCGAGGAGGACTTCGAAACT 60
      1 -M--N--K--L--Y--I--G--N--V--S--A--E--A--S--E--E--D--F--E--T- 20
    61 ATCTTTGAGCAGTGGAAGATTCCGCACAGTGGTCCATTTCTTGTCAAAACTGGCTATGCG 120
    21 -I--F--E--Q--W--K--I--P--H--S--G--P--F--L--V--K--T--G--Y--A- 40
  121 TTTGTGGATTGCCCGGACGAGAAGGCAGCAATGAAGGCCATCGATGTTCTTTCAGGTAAA 180
    41 -F--V--D--C--P--D--E--K--A--A--M--K--A--I--D--V--L--S--G--K- 60
  181 GTTGAACTTCACGGAAAAGTTCTTGAAGTGGAGCACTCGGTCCCTAAACGTCAAAGGAGC 240
    61 -V--E--L--H--G--K--V--L--E--V--E--H--S--V--P--K--R--Q--R--S- 80
  241 TGTAAGCTGCAGATCAGGAACATCCCGCCTCACATGCAGTGGGAGGTTTTGGATGGTATG 300
    81 -C--K--L--Q--I--R--N--I--P--P--H--M--Q--W--E--V--L--D--G--M- 100
  301 CTTGCTCAGTATGGTGCAGTACAGAGCTGTGAACAAGTAAACACTGATACAGAGACTGCA 360
  101 -L--A--Q--Y--G--A--V--Q--S--C--E--Q--V--N--T--D--T--E--T--A- 120
  361 GTTGTCAATGTTCGGTATGCTACCAAGGACCAGGCTAGGCTGGCAATGGAGAAGCTGAAT 420
  121 -V--V--N--V--R--Y--A--T--K--D--Q--A--R--L--A--M--E--K--L--N- 140
  421 GGATCTATGATGGAGAACTCTACCTTGAAAGTGTCCTATATCCCAGATGAGACAGCGACA 480
  141 -G--S--M--M--E--N--S--T--L--K--V--S--Y--I--P--D--E--T--A--T- 160
  481 CCAGAGGGTCCTCCAGCAGGGGGCCGGAGAGGCTTTAATGCCCGCGGACCCCCTCGGTCT 540
  161 -P--E--G--P--P--A--G--G--R--R--G--F--N--A--R--G--P--P--R--S- 180
  541 GGCTCTCCGGGTTTGGGCGCCCGGCCTAAAGTGCAGTCAGACATCCCGCTACGCATGCTG 600
  181 -G--S--P--G--L--G--A--R--P--K--V--Q--S--D--I--P--L--R--M--L- 200
  601 GTTCCCACGCAGTTTGTAGGGGCAATCATTGGCAAGGAGGGTGCCACTATCCGCAACATC 660
  201 -V--P--T--Q--F--V--G--A--I--I--G--K--E--G--A--T--I--R--N--I- 220
  661 ACCAAACAGACCCACTCAAAGATTGACATCCACAGAAAAGAGAACGCAGGTGCTGCAGAG 720
  221 -T--K--Q--T--H--S--K--I--D--I--H--R--K--E--N--A--G--A--A--E- 240
  721 AAACCCATCACTATTCACTCAACCCCTGATGGCTGTTCGAACGCTTGCAAAACCATCATG 780
  241 -K--P--I--T--I--H--S--T--P--D--G--C--S--N--A--C--K--T--I--M- 260
  781 GACATCATGCAGAAGGAAGCCCTTGACACAAAGTTTACTGAGGAGATCCCACTAAAGATC 840
  261 -D--I--M--Q--K--E--A--L--D--I--K--F--T--E--E--I--P--L--K--I- 280
  841 CTTGCACACAACAGCTTTGTGGGAAGATTAATAGGTAAAGAAGGACGCAACCTGAAGAAA 900
  281 -L--A--H--N--S--F--V--G--R--L--I--G--K--E--G--R--N--L--K--K- 300
  901 ATTGAGCAGGAAACGGGGACCAAGATCACAATCTCACCTCTTCAGGACCTAACCCTGTAC 960
  301 -I--E--Q--E--T--G--T--K--I--T--I--S--P--L--Q--D--L--I--L--Y- 320
  961 AACCCAGAACGGACCATCACAGTAAAGGGCTCCATTGAGGCATGTGCAAAAGCTGAGGAG 1020
  321 -N--P--E--R--T--I--T--V--K--G--S--I--E--A--C--A--K--A--E--E- 340
1021 GAAGTGATGAAGAAGATCAGGGAATCCTATGAGAGTGACATGGCTGCTATGAACCTCCAA 1080
  341 -E--V--M--K--K--I--R--E--S--Y--E--S--D--M--A--A--M--N--L--Q- 360
1081 TCCAACTTGATTCCAGGCTTGAATCTGAATGCTTTAGGTTTGTTCCCCACTACAGCACCA 1140
  361 -S--N--L--I--P--G--L--N--L--N--A--L--G--L--F--P--T--T--A--P- 380
1141 GGCATGGGTCCCTCCATGTCCAGTATCACACCTCCTGGAGCCCATGGTGGATCCTCATCA 1200
  381 -G--M--G--P--S--M--S--S--I--T--P--P--G--A--H--G--G--S--S--S- 400
1201 TTTGGACAGGGACACCCAGAATCGGAGACTGTTCACCTGTTCATTCCTGCACTTGCAGTG 1260
  401 -F--G--Q--G--H--P--E--S--E--T--V--H--L--F--I--P--A--L--A--V- 420
1261 GGCGCCATCATTGGAAAACAGGGTCAACACATCAAACAGCTGTCACACTTTGCCGGAGCC 1320
  421 -G--A--I--I--G--K--Q--G--Q--H--I--K--Q--L--S--H--F--A--G--A- 440
1321 TCAATCAAGATCGCCCCTGCAGAAGGAATGGATGCCAAGCAGAGGATGGTTATCATTGTC 1380
  441 -S--I--K--I--A--P--A--E--G--M--D--A--K--Q--R--M--V--I--I--V- 460
1381 GGACCACCAGAGGCTCAGTTTAAGGCTCAGTGTCGAATCTTTGGCAAGTTAAAAGAAGAG 1440
  461 -G--P--P--E--A--Q--F--K--A--Q--C--R--I--F--G--K--L--K--E--E- 480
1441 AATTTCTTTGGACCTAAGGAAGAGGTGAAGCTGGAGGCGCATATCAAGGTTCCCGCCTTT 1500
  481 -N--F--F--G--P--K--E--E--V--K--L--E--A--H--I--K--V--P--A--F- 500
1501 GCTGCTGGACGAGTTATTGGGAAGGGCGGGAAAACGGTAAACGAACTGCAGAACTTGACC 1560
  501 -A--A--G--R--V--I--G--K--G--G--K--T--V--N--E--L--Q--N--L--T- 520
1561 TGTGCAGAAGTGGTGGTGCCCCGAGACCAGACGCCTGACGAGAACGACCAGGTTATAGTA 1620
  521 -C--A--E--V--V--V--P--R--D--Q--I--P--D--E--N--D--Q--V--I--V- 540
1621 AAGATCAGCGGACACTTCTTTGCATGCCAGCTGGCCCAGAGGAAGATTCAGGAGATCCTA 1680
  541 -K--I--S--G--H--F--F--A--C--Q--L--A--Q--R--K--I--Q--E--I--L- 560
1681 GCCCAGGTGAGGAGGCAGCAGCAGCAACAACAGCAGCAGCAGCTTAAGCCTACATCTGGA 1740
  561 -A--Q--V--R--R--Q--Q--Q--Q--Q--Q--Q--Q--Q--L--K--P--T--S--G- 580
1741 CCCCAAGCTCCAATGCCACGCAGGAAATAA.............................. 1770
  581 -P--Q--A--P--M--P--R--R--K--*-.............................. 589
1801 GAATCTGCCAGAAGACTCGTCAGAAGGACAGATGCAGCAGAGTCCAGGAGGGGGAGAAGA 1860
          ............................................................
1861 CGATGACGGCAGTGGGTCCTAATGCTCATCTCAGGGGTTAAAGGTTGTTGGAGCCCAACC 1920
          ............................................................
1921 AAACATCCTCCCCTCCTTGTCTTACTTGGGACTGCGCGGCTGATTTAAAAAAACAAAAAA 1980
          ............................................................
1981 AAGGAAGGAAAAAACAAAAAAAGAGAGACCCTGCGCCTCTAAAAGCTCCACCCACTCCGC 2040
          ............................................................
2041 CTCTCTGCATCTCTGCGAGAATGTACTCCTGAGGGCTCCCACCGTCGTCACCTGCCCTCA 2100
          ............................................................
2101 CAAGTGCACAACCCTCAACCGCTCTACTCCTCCCCCAAAGGATGTGTTTAAACTTGTATT 2160
          ............................................................
2161 TTTTTTCTTTTTACACTAGAAACACAAAGAAGAAATAAGGACCCCCGCCCCCTTCCTATC 2220
          ............................................................
2221 ACCGCCTTGGTGTTGTACTTTAAACATGACAAGATGTTTTGGTTGACTTCAGATTTAGTG 2280
          ............................................................
2281 AACACCTG 2288
          ........
(Igf2 bp3 mutant allele-2 nt insertion)
LENGTH: 2288 bp(−2 bp) and 206 aa
TYPE: cDNA (SEQ ID NO: 109) and Protein (SEQ ID NO: 111)
ORGANISM: Nile tilapia
SEQ ID Nos 109 and 111
      1 ATGAATAAGCTATACATTGGCAACGTAAGCGCAGAGGCGAGCGAGGAGGACTTCGAAACT 60
      1 -M--N--K--L--Y--I--G--N--V--S--A--E--A--S--E--E--D--F--E--T- 20
    61 ATCTTTGAGCAGTGGAAGATTCCGCACAGTGGTCCATTTCTTGTCAAAACTGGCTATGCG 120
    21 -I--F--E--G--W--K--I--P--H--S--G--P--F--L--V--K--T--G--Y--A- 40
  121 TTTGTGGATTGCCCGGACGAGAAGGCAGCAATGAAGGCCATCGATGTTCTTTCAGGTAAA 180
    41 -F--V--D--C--P--D--E--K--A--A--M--K--A--I--D--V--L--S--G--K- 60
  181 GTTGAACTTCACGGAAAAGTTCTTGAAGTGGAGCACTCGGTCCCTAAACGTCAAAGGAGC 240
    61 -V--E--L--H--G--K--V--L--E--V--E--H--S--V--P--K--R--G--R--S- 80
  241 TGTAAGCTGCAGATCAGGAACATCCCGCCTCACATGCAGTGGGAGGTTTTGGATGGTATG 300
    81 -C--K--L--G--I--R--N--I--P--P--H--M--G--W--E--V--L--D--G--M- 100
  301 CTTGCTCAGTATGGTGCAGTACAGAGCTGTGAACAAGTAAACACTGATACAGAGACTGCA 360
  101 -L--A--G--Y--G--A--V--G--S--C--E--G--V--N--T--D--T--E--T--A- 120
  361 GTTGTCAATGTTCGGTATGCTACCAAGGACCAGGCTAGGCTGGCAATGGAGAAGCTGAAT 420
  121 -V--V--N--V--R--Y--A--T--K--D--G--A--R--L--A--M--E--K--L--N- 140
  421 GGATCTATGATGGAGAACTCTACCTTGAAAGTGTCCTATATCCCAGATGAGACAGCGACA 80
  141 -G--S--M--M--E--N--S--T--L--K--V--S--Y--I--P--D--E--T--A--T- 160
  481 CCAGAGGGTCCTCCAGCAGGGGGCCGGAGAGGCTTTAATGGAGAGCCGGACCCCCTCGGT 540
  161 -P--E--G--P--P--A--G--G--R--R--G--F--N--G--E--P--D--P--L--G- 180
    41 CTGGCTCTCCGGGTTTGGGCGCCCGGCCTAAAGTGCAGTCAGACATCCCGCTACGCATGC 600
  181 -L--A--L--R--V--W--A--P--G--L--K--C--S--G--T--S--R--Y--A--C- 200
  601 TGGTTCCCACGCAGTTTGTAGGGGCAATCATTGGCAAGGAGGGTGCCACTATCCGCAACA 660
  201 -W--F--P--R--S--L--*- 206
(wild-type Elavl1)
LENGTH: 1894 bp and 359 aa
TYPE: cDNA (SEQ ID NO: 112) and Protein (SEQ ID NO: 114)
ORGANISM: Nile tilapia
SEQ ID NOs 112 and 114
      1 CTATTTTACAGAACTAGAAGAAGAAGGAGAGGAGGAGATCTCGCGATACTTCACTGGGCG 60
          ............................................................
    61 GCAGTTGGTTCTTTGTGTGCAGCAGGGAACGTGCGTGTTAGGATCGACAGATCATCTCAT 120
          ............................................................
  121 CTTCCAACTGCGGATCGATATCTGACCCAATCACACCAGATCACCTGTCCGGACTCCCAC 180
          ............................................................
  181 AGACGCAGTTAACTTCCTGAATCATTTCCATGGCGCAAAGACGAGGACACATCAGGTACC 240
          ...............................-M--A--Q--R--R--G--H--I--R--Y 10
  241 TGAAGGTGTGTGAGGTTCAGAACTCTCAGGGTGATGTCAGAGACGCTTCGCTCGCCGCTA 300
    11 L--K--V--C--E--V--Q--N--S--Q--G--D--V--R--D--A--S--L--A A-- 30
  301 AAGGAGCTGCTGGAAATGAGCTGTACGATAACGGGTACGGCGAGCAGATGATGGAGGACG 360
    31 K--G--A--A--G--N--E--L--Y--D--N--G--Y--G--E--Q--M--M--E--D-- 50
  361 AAGACGCGCGCACGAACCTGATTGTGAACTACCTGCCGCAGAGCATGAGCCAGGACGAGC 420
      1 E--D--A--R--T--N--L--I--V--N--Y--L--P--Q--S--M--S--Q--D--E-- 70
  421 TACGCAGCCTCTTCAGCAGTGTTGGCGAGGTCGAGTCTGCCAAGCTCATCCGCGACAAAG 480
    71 L--R--S--L--F--S--S--V--G--E--V--E--S--A--K--L--I--R--D--K-- 90
  481 TGGCAGGCCACAGTTTAGGTTACGGCTTTGTTAACTTTGTTAACCCTAGTGATGCAGAGA 540
    91 V--A--G--H--S--L--G--Y--G--F--V--N--F--V--N--P--S--D--A--E-- 110
    41 GGGCTATCAGTACCCTCAATGGCCTGAGGCTACAGTCTAAAACTATCAAGGTTTCATTTG 600
  111 R--A--I--S--T--L--N--G--L--R--L--Q--S--K--T--I--K--V--S--F-- 130
  601 CACGGCCGAGTTCGGACGCCATCAAAGATGCGAACCTGTATATCAGTGGTTTGCCACGGA 660
  131 A--R--P--S--S--D--A--I--K--D--A--N--L--Y--I--S--G--L--P--R-- 150
  661 CTCTCAGTCAGCAGGACGTGGAGGACATGTTCTCGCACTACGGTCGCATCATCAATTCTA 720
  151 T--L--S--Q--Q--D--V--E--D--M--F--S--H--Y--G--R--I--I--N--S-- 170
  721 GAGTGTTAGTGGACCAGGCTTCAGGTCTGTCACGTGGCGTGGCCTTCATCCGCTTTGATA 780
  171 R--V--L--V--D--Q--A--S--G--L--S--R--G--V--A--F--I--R--F--D-- 190
  781 AGAGGGCTGAGGCCGATGACGCTGTCAAACACCTGAACGGACACACGCCTCCCGGCAGCG 840
  191 K--R--A--E--A--D--D--A--V--K--H--L--N--G--H--T--P--P--G--S-- 210
  841 CTGAGCCAATCACGGTCAAGTTTGCTGCCAATCCCAACCAGGCCAGGAACTCCCAGATGA 900
  211 A--E--P--I--T--V--K--F--A--A--N--P--N--Q--A--R--N--S--Q--M-- 230
  901 TGTCACAGATGTATCATGGCCAATCACGACGTTTTGGGGGGCCCGTCCATCACCAGGCAC 960
  231 M--S--Q--M--Y--H--G--Q--S--R--R--F--G--G--P--V--H--H--Q--A-- 250
  961 AAAGGTTCCGGTTTTCTCCAATGAGCACCGACCACATGAGCGGAGGGGGTGGGGCCTCGG 1020
  251 Q--R--F--R--F--S--P--M--S--T--D--H--M--S--G--G--G--G--A--S-- 270
1021 GGAGCTCATCCTCTGGTTGGTGCATCTTCATCTACAACCTGGGCCAGGAAGCTGACGAGG 1080
  271 G--S--S--S--S--G--W--C--I--F--I--Y--N--L--G--Q--E--A--D--E-- 290
1081 CCATGCTGTGGCAGATGTTTGGCCCGTTCGGCGCAGTCTTGAATGTGAAAGTGATCCGAG 1140
  291 A--M--L--W--Q--M--F--G--p F--G--A--V--L--N--V--K--V--I--R-- 310
1141 ATTTTAACACCAATAAGTGCAAAGGCTTTGGCTTTGTTACAATGGCAAACTATGAGGAAG 1200
  311 D--F--N--T--N--K--C--K--G--F--G--F--V--T--M--A--N--Y--E--E-- 330
1201 CTGCCATGGCGATCCACAGCCTGAACGGGTACCGCCTGGGGGACAAAGTCCTGCAGGTCT 1260
  331 A--A--M--A--I--H--S--L--N--G--Y--R--L--G--D--K--V--L--Q--V-- 350
1261 CATTCAAGACCAGCAAGGGGCACAAATAGAGGAGGGGGCGAGGCTAAAACTAATAACAGG 1320
  351 S--F--K--T--S--K--G--H--K--*- ............................... 359
1321 TGTTTTTGTTTTTGTTTTTTGTCTGTTTTGTCAGTTTTTCCCAGCATGCCCTGTTTCTTT 1380
          ............................................................
1381 ATGTCAGTAAGTAATTTTTCTGACTGTGTGGGCGTTCATCCACAATAAAGGACTGAAACC 1440
          ............................................................
1441 TGCAGTATGACTGACAGCTGACTGTCACCATGGTTATGAACATAACTGGAGTTGTATCAA 1500
          ............................................................
1501 TTTCTGCAGGTTTACATTTGGGGTCAAAGGATACGGAAACTAAATCTGCTCTTTTCTGAT 1560
          ............................................................
1561 TTGAGTAAAACGTTCAGTTGGTTTTATGTACAGTTTATGTAAATGATGTCATGGTAACCA 1620
          ............................................................
1621 CTGACAACCGATTAAAGGATTAAAAGTTTGGACAGGTAACCTGACGTTATCATGTCAGGT 1680
          ............................................................
1681 GATCAGGTCAGTGTTAGACGATTAGTTTCATGTTGTACAGGTGAGGTAGAGGAATGCACC 1740
          ............................................................
1741 TGATGAACAGGTAACTGATGTGAAGTCAATTTTCATTTGTTTTATTTTTGTATTGCAGCT 1800
          ............................................................
1801 TCATTGTGACATTTATTCAGCAATAAATCTGTTATTGTGAAAACATAACCTGTGTCTGAA 1860
          ............................................................
1861 TGTTTGTCTCCCCTTTGTCTGAATTTCTTTAAAC 1894
(Elavl1 mutant allele-3K nt deletion)
LENGTH: 1894 bp(−3 kb) and 105 aa
TYPE: cDNA (SEQ ID NO: 113) and Protein (SEQ ID NO: 115)
ORGANISM: Nile tilapia
SEQ ID NOs 113 and 115
      1 CTATTTTACAGAACTAGAAGAAGAAGGAGAGGAGGAGATCTCGCGATACTTCACTGGGCG 60
          ............................................................
    61 GCAGTTGGTTCTTTGTGTGCAGCAGGGTACGTGCGTGTTAGGATCGACAGATCATCTCAT 120
          ............................................................
  121 CTTCCAACTGCGGATCGATATCTGACCCAATCACACCAGATCACCTGTCCGGACTCCCAC 180
          ............................................................
  181 AGACGCAGTTAACTTCCTGAATCATTTCCATGGCGCAAAGACGAGGACACATCAGGTACC 240
          ...............................-M--A--Q--R--R--G--H--I--R--Y 10
  241 TGAAGGTGTGTGAGGTTCAGAACTCTCAGGGTGATGTCAGAGACGCTTCGCTCGCCGCTA 300
    11 L--K--V--C--E--V--Q--N--S--Q--G--D--V--R--D--A--S--L--A--A-- 30
  301 AAGGAGCTGCTGGAAATGAGCTGTACGATAACGGGTACGGCGAGTTCGGCGCAGTCTTGA 360
    31 K--G--A--A--G--N--E--L--Y--D--N--G--Y--G--E--F--G--A--V--L-- 50
  361 ATGTGAAAGTGATCCGAGATTTTAACACCAATAAGTGCAAAGGCTTTGGCTTTGTTACAA 420
      1 N--V--K--V--I--R--D--F--N--T--N--K--C--K--G--F--G--F--V--T-- 70
  421 TGGCAAACTATGAGGAAGCTGCCATGGCGATCCACAGCCTGAACGGGTACCGCCTGGGGG 480
    71 M--A--N--Y--E--E--A--A--M--A--I--H--S--L--N--G--Y--R--L--G-- 90
  481 ACAAAGTCCTGCAGGTCTCATTCAAGACCAGCAAGGGGCACAAAATAGAGGAGGGGGCGA 540
    91 D--K--V--L--Q--V--S--F--K--T--S--K--G H--K--* 105
(wild-type Elavl2)
LENGTH: 1119 bp and 372 aa
TYPE: cDNA (SEQ ID NO: 116) and Protein (SEQ ID NO: 118)
ORGANISM: Nile tilapia
SEQ ID Nos 116 and 118
      1 CAGGTAATTGCTGCCATGGAAACACAGCTATCCAATGGGCCCACTTGCAACAACACAAGC 60
      1 -Q--V--I--A--A--M--E--T--Q--L--S--N--G--P--T--C--N--N--T--S- 20
    61 AACGGTCCTTCAACTATCACAAACAACTGCTCCTCACCTGTAGAGTCAGGGAGCGTAGAG 120
    21 -N--G--P--S--T--I--T--N--N--C--S--S--P--V--E--S--G--S--V--E- 40
  121 GACAGTAAAACTAACTTGATAGTCAACTATCTGCCTCAGAACATGACCCAGGAGGAACTG 180
    41 -D--S--K--T--N--L--I--V--N--Y--L--P--Q--N--M--T--Q--E--E--L- 60
  181 AAGAGTTTGTTTGGGAGCATCGGAGAAATTGAGTCCTGTAAACTAGTTCGAGACAAAATC 240
    61 -K--S--L--F--G--S--I--G--E--I--E--S--C--K--L--V--R--D--K--I- 80
  241 ACAGGGCAGAGCCTAGGCTATGGATTTGTGAATTATGTGGACCCAAAGGATGCAGAAAAG 300
    81 -T--G--Q--S--L--G--Y--G--F--V--N--Y--V--D--P--K--D--A--E--K- 100
  301 GCCATCAATACCTTAAATGGCTTGAGACTTCAGACCAAAACCATCAAGGTTTCCTATGCG 360
  101 -A--I--N--T--L--N--G--L--R--L--Q--T--K--T--I--K--V--S--Y--A- 120
  361 CGTCCAAGCTCCGCCTCCATCAGAGATGCAAATTTATACGTCAGTGGCCTGCCAAAAACT 420
  121 -R--P--S--S--A--S--I--R--D--A--N--L--Y--V--S--G--L--P--K--T- 140
  421 ATGACTCAGAAGGAACTGGAGCAGCTCTTCTCTCAGTACGGACGCATTATTACCTCACGC 480
  141 -M--T--Q--K--E--L--E--Q--L--F--S--Q--Y--G--R--I--I--T--S--R- 160
  481 ATTCTGGTGGACCAGGTGACTGGTGTTTCCAGAGGAGTTGGCTTCATTCGTTTTGACCGG 540
  161 -I--L--V--D--Q--V--T--G--V--S--R--G--V--G--F--I--R--F--D--R- 180
    41 CGAGTTGAGGCTGAGGAGGCCATCAAGGGTCTGAACTGTCAGAAGCCGCCTGGTGCCACC 600
  181 -R--V--E--A--E--E--A--I--K--G--L--N--C--Q--K--P--P--G--A--T- 200
  601 GAACCCATTACAGTCAAGTTTGCAAACAACCCGAGCCAAAAGACCAGCCAGGCACTGCTG 660
  201 -E--P--I--T--V--K--F--A--N--N--P--S--Q--K--T--S--Q--A--L--L- 220
  661 TCCCAGCTCTATCAGTCACCCAATCGAAGGTACCCAGGACCCCTCGCACAGCAGGCACAA 720
  221 -S--Q--L--Y--Q--S--P--N--R--R--Y--P--G--P--L--A--Q--Q--A--G- 240
  721 CGCTTCAGGTTGGACAATCTGCTGAACATGGCCTACGGAGTCAAAAGCTCTATGGCAGTA 780
  241 -R--F--R--L--D--N--L--L--N--M--A--Y--G--V--K--S--S--M--A--V- 260
  781 TTGTGTAGCAGGTTCTCCCCGATGGCCATTGACGGGGTGACCAGCTTGGCTGGCATCAAC 840
  261 -L--C--S--R--F--S--P--M--A--I--D--G--V--T--S--L--A--G--I--N- 280
  841 ATCCCGGGGCACGCGGGCACTGGCTGGTGCATCTTCGTCTACAACCTGGCTCCGGACGCA 900
  281 -I--P--G--H--A--G--T--G--W--C--I--F--V--Y--N--L--A--P--D--A- 300
  901 GATGAAAGCATCCTTTGGCAGATGTTCGGGCCGTTTGGTGCTGTCACAAACGTCAAGGTT 960
  301 -D--E--S--I--L--W--Q--M--F--G--P--F--G--A--V--T--N--V--K--V- 320
  961 ATCCGCGACTTTAACACAAACAAGTGCAAAGGATTTGGTTTTGTCACCATGACTAATTAC 1020
  321 -I--R--D--F--N--T--N--K--C--K--G--F--G--F--V--T--M--T--N--Y- 340
1021 GACGAGGCAGCTGTGGCCATCGCCAGCTTGAATGGATACCGCCTTGGGGACAGAGTTCTG 1080
  341 -D--E--A--A--V--A--I--A--S--L--N--G--Y--R--L--G--D--R--V--L- 360
1081 CAAGTGTCATTCAAAACCAACAAAACACACAAAGCCTGA 1119
  361 -Q--V--S--F--K--T--N--K--T--H--K--A--*- 372
(Elavl2 mutant allele-8 nt deletion)
LENGTH: 1119 bp(−8 bp) and 40 aa
TYPE: cDNA (SEQ ID NO: 117) and Protein (SEQ ID NO: 119)
ORGANISM: Nile tilapia
SEQ ID Nos 117 and 119
      1 CAGGTAATTGCTGCCATGGAAACACAGCTATCCAACTTGCAACAACACAAGCAACGGTCC 60
      1 -Q--V--I--A--A--M--E--T--Q--L--S--N--L--Q--Q--H--K--Q--R--S- 20
    61 TTCAACTATCACAAACAACTGCTCCTCACCTGTAGAGTCAGGGAGCGTAGAGGACAGTAA 120
    21 -F--N--Y--H--K--G--L--L--L--T--C--R--V--R--E--R--R--G--G--*- 40
(wild-type Cxcr4a)
LENGTH: 1996 bp and 382 aa
TYPE: cDNA (SEQ ID NO: 120) and Protein (SEQ ID NO: 122)
ORGANISM: Nile tilapia
SEQ ID NOs 120 and 122
      1 TTACAACAAGTACAGAAGTTTTATAGCGACCCTATTTGGGAGCATCCTACTTCTGCTCCT 60
          ............................................................
    61 CCCTCCCTTTCGGGGGAGGAGTTAATGGCACGGAATACTATTTATTGGCAGGCGCTGAAA 120
          ............................................................
  121 CAAACAACTTATCGTCGTGCGTCTCATGCCAACGTTTACTCACTAGTCCCACAGTTGGAT 180
          ............................................................
  181 TGTTATCACCATGGATGAAACCGTGGAGTTCAAATATGACATTGATTTTACCAGCAACAC 240
          ..........-M--D--E--T--V--E--F--K--Y--D--I--D--F--T--S--N--T 17
  241 TTCTGACAACATATCAGAAGGGTCTGGATTTGATTTTGGAGACCTGAATTTACCGGAGAT 300
    17 --S--D--N--I--S--E--G--S--G--F--D--F--G--D--L--N--L--P--E--I 37
  301 CTGTGGCCAGACATTCAGCAATGACTTCAACAAAATCTTCCTACCCACAGTGTACGGAAT 360
    37 --C--G--Q--T--F--S--N--D--F--N--K--I--F--L--P--T--V--Y--G--I 57
  361 AATATCCATTCTTGGGATAGTTGGTAATGGATTAGTTGTACTAGTCATGGGTTACCAGAA 420
    57 --I--S--I--L--G--I--V--G--N--G--L--V--V--L--V--M--G--Y--Q--K 77
  421 AAAGGTCAAAACAATGACGGACAAGTACCGGCTCCATCTGTCTGTTGCTGACCTCCTGTT 480
    77 --K--V--K--T--M--T--D--K--Y--R--L--H--L--S--V--A--D--L--L--F 97
  481 TGTCCTCACTCTGCCCTTCTGGGCTGTGGATGCAGCCAAAAACTGGTACTTTGGAGGTTT 540
    97 --V--L--T--L--P--F--W--A--V--D--A--A--K--N--W--Y--F--G--G--F 117
  541 CCTCTGCGTGTCTGTGCACATGATCTACACCATCAACCTGTACAGTAGCGTGCTGATTCT 600
  117 --L--C--V--S--V--H--M--I--Y--I--I--N--L--Y--S--S--V--L--I--L 137
  601 GGCCTTCATCAGTCTGGACAGATACTTGGCAGTTGTACGGGCTACCAACAGCCAAGCCAC 660
  137 --A--F--I--S--L--D--R--Y--L--A--V--V--R--A--T--N--S--Q--A--T 157
  661 GAGGAAGCTTCTTGCAAACAGAGTGATCTACGTGGGTGTGTGGCTGCCGGCAACCATTCT 720
  157 --R--K--L--L--A--N--R--V--I--Y--V--G--V--W--L--P--A--T--I--L 177
  721 GACCATACCTGACATGGTGTTTGCAAGAGTGCAGAGCATGAGCTCTTCAAATATCTACTT 780
  177 --T--I--P--D--M--V--F--A--R--V--Q--S--M--S--S--S--N--I--Y--F 197
  781 CAAAGAAGAAAGCGAGGACACGGCAGACTCCAGGACTATCTGCCAGCGCATGTATCCAGT 840
  197 --K--E--E--S--E--D--T--A--D--S--R--T--I--C--Q--R--M--Y--P--V 217
  841 GGAAAGTAACGTCATATGGACAGTTGTTTTCCGTTTCCAGCACATCCTGGTGGGCTTCGT 900
  217 --E--S--N--V--I--W--T--V--V--F--R--F--Q--H--I--L--V--G--F--V 237
  901 TCTGCCCGGCTTGGTTATCCTCATCTGCTACTGCATTATCATAACAAAGCTGGCACAAGG 960
  237 --L--P--G--L--V--I--L--I--C--Y--C--I--I--I--T--K--L--A--Q--G 257
  961 CGCAAAGGGCCAGACACTGAAGAAAAAGGCGCTGAAGACCACAATCATTCTAATCTTTTG 1020
  257 --A--K--G--Q--T--L--K--K--K--A--L--K--X--T--I--I--L--I--F--C 277
1021 TTTTTTTTGTTGCTGGCTCCCCTACTGTGTTGGCATCTTTTTGGACAACCTCGTGATGCT 1080
  277 --F--F--C--C--W--L--P--Y--C--V--G--I--F--L--D--N--L--V--M--L 297
1081 GAATGTGCTCTCCCCCTCATGTGAACTGCAGCAAGCGCTGGACAAGTGGATTTCTGTCAC 1140
  297 --N--V--L--S--P--S--C--E--L--Q--Q--A--L--D--K--W--I--S--V--T 317
1141 TGAGGCGCTAGCCTATTTTCACTGCTGCCTAAACCCCATCCTCTATGCTTTCTTGGGAGT 1200
  317 --E--A--L--A--Y--F--H--C--C--L--N--P--I--L--Y--A--F--L--G--V 337
1201 TAAGTTTAAGAAATCAGCTAAGAGTGCACTGACAGCGAGCAGCAGATCAAGTCAGAAAGT 1260
  337 --K--F--K--K--S--A--K--S--A--L--T--A--S--S--R--S--S--Q--K--V 357
1261 GACTCTCATGACAAAAAAGCGAGGGCCAATTTCATCTGTGTCAACCGAGTCGGAGTCTTC 1320
  357 --T--L--M--T--K--K--R--G--P--I--S--S--V--S--T--E--S--E--S--S 377
1321 AAGTGTTTTGTCAAGTTAACTGTCAGCCTCGGAGTCTGTGACTTGATACTCTCAGGAGTG 1380
  377 --S--V--L--S--S--*-......................................... 382
1381 AAAAAGCTAAGCTGTAATTTCAAAGAACTACAATCTGTACAAATGTAAATGAAAGAGTTT 1440
          ............................................................
1441 TTATACGTGAAGATTTTTTTTGTGTGTGTGTGCCTTTGTACTTCAATCGTGGTTCAATCT 1500
          ............................................................
1501 TTTGTGGTTCTTATTTTCTGTATTTTATTTTCTGCTCCTCAAAGCAGATCGTGTCCTCAG 1560
          ............................................................
1561 GCAGTGCCTCATTTCCATTCATTCAGTTTTACAATCAATACCCATTGTCCTAGTTTTTAT 1620
          ............................................................
1621 CCCATAGTCTTTGATGCTGTATCAAAGCTCAGACACACAATGTCCTTTCTGGGGGGTTTT 1680
          ............................................................
1681 AGGACTGGTAGCTGCTTCTGGAAACATGTACATAGTTTGTAGCATATGTGTGTTTGCACC 1740
          ............................................................
1741 TAAGCTGTGCAATTATCTGAAAGCTATAATTTATTGCTGTCATACACACTGGAGTTTTGT 1800
          ............................................................
1801 AAAAGTCCTTCAAAATGATTTTTTTGTGCTGTTTTTTATGTGTTTGTATTGAAAATAAAA 1860
          ............................................................
1861 GAAACTCAAACATATTTTGTGGTGCGCCTTTTCACTGGCTTCTACTCCCGTGTGTGCATG 1920
          ............................................................
1921 TGTATTATCAAGGGGGTTGGGGGAGTGTCACACAAATGTCACCACCTGAACTGGCTGAAA 1980
          ............................................................
1981 AGCAGGAGGAATGAAC 1996
          ................
(Cxcr4a mutant allele-8 nt deletion)
LENGTH: 1996 bp(−8 bp) and 177 aa
TYPE: cDNA (SEQ ID NO: 121) and Protein (SEQ ID NO: 123)
ORGANISM: Nile tilapia
SEQ ID Nos 121 and 123
      1 TTACAACAAGTACAGAAGTTTTATAGCGACCCTATTTGGGAGCATCCTACTTCTGCTCCT 60
          ............................................................
    61 CCCTCCCTTTCGGGGGAGGAGTTAATGGCACGGAATACTATTTATTGGCAGGCGCTGAAA 120
          ............................................................
  121 CAAACAACTTATCGTCGTGCGTCTCATGCCAACGTTTACTCACTAGTCCCACAGTTGGAT 180
          ............................................................
  181 TGTTATCACCATGGATGAAACCGTGGAGTTCAAATATGACATTGATTTTACCAGCAACAC 240
          ..........-M--D--E--T--V--E--F--K--Y--D--I--D--F--T--S--N--T 17
  241 TTCTGACAACATATCAGAAGGGTCTGGATTTGATTTTGGAGACCTGAATTTACCGGAGAT 300
    17 --S--D--N--I--S--E--G--S--G--F--D--F--G--D--L--N--L--P--E--I 37
  301 CTGTGGCCAGACATTCAGCAATGACTTCAACAAAATCTTCCTACCCACAGTGTACGGAAT 360
    37 --C--G--Q--T--F--S--N--D--F--N--K--I--F--L--P--T--V--Y--G--I 57
  361 AATATCCATTCTTGGGATAGTTGGTAATGGATTAGTTGTACTAGTCATGGGTTACCAGAA 420
    71 --I--S--I--L--G--I--V--G--N--G--L--V--V--L--V--M--G--Y--Q--K 77
  421 AAAGGTCAAAACAATGACGGACAAGTACCGGCTCCATCTGTCTGTTGCTGACCTCCTGTT 480
    77 --K--V--K--T--M--T--D--K--Y--R--L--H--L--S--V--A--D--L--L--F 97
  481 TGTCCTCACTCTGCCCTTCTGGGCTGTGGATGCAGCCAAAAACTGGTACTTTGGAGGTTT 540
    97 --V--L--T--L--P--F--W--A--V--D--A--A--K--N--W--Y--F--G--G--F 117
  541 CCTCTGCGTGTCTGTGCACATGATCTACACCATCAACCTGTACAGTAGCGTGCTGATTCT 600
  117 --L--C--V--S--V--H--M--I--Y--T--I--N--L--Y--S--S--V--L--I--L 137
  601 GGCCTTCATCAGTCTGGACAGATACTTGGCAGTTGTACGGGCTACCAACAGCCAAGCCAC 660
  137 --A--F--I--S--L--D--R--Y--L--A--V--V--R--A--T--N--S--Q--A--T 157
  661 GAGGAAGCTTCTTGCAAACAGAGTGATCTACGTGGGTGCCGGCAACCATTCTGACCATAC 720
  157 --R--K--L--L--A--N--R--V--I--Y--V--G--A--G--N--H--S--D--H--T 177
  721 CTGACATGGTGTTTGCAAGAGTGCAGAGCATGAGCTCTTCAAATATCTACTTCAAAGAAG 780
  177 --*- 177
(wild-type Ptbp1a)
LENGTH: 6015 bp and 538 aa
TYPE: cDNA (SEQ ID NO: 124) and Protein (SEQ ID NO: 127)
ORGANISM: Nile tilapia
SEQ ID NOs 124 and 127
      1 ATGGACGGCAGTGTCCACCACGATATAACAGTTGGCACCAAGAGAGGATCTGACGAACTT 60
      1 -M--D--G--S--V--H--H--D--I--T--V--G--T--K--R--G--S--D--E--L- 20
    61 TTCTCCAGCGTCTCCAGCAACCCTTATATCATGAGCACCACAGCCAATGGCAACGACAGC 120
    21 -F--S--S--V--S--S--N--P--Y--I--M--S--T--T--A--N--G--N--D--S- 40
  121 AAAAAGTTCAAAGGTGACATAAGAGGCCCCAGCGTGCCATCCAGGGTCATCCACATCCGC 180
    41 -K--K--F--K--G--D--I--R--G--P--S--V--P--S--R--V--I--H--I--R- 60
  181 AAGCTTCCCAGCGACATCACAGAGGCGGAGGTGATCAGCCTCGGCGTGCCTTTTGGAGAC 240
    61 -K--L--P--S--D--I--T--E--A--E--V--I--S--L--G--V--P--F--G--D- 80
  241 GTCACCAACCTGCTGATGCTCAAAGCCAAGAACCAGGCCTTTTTAGAGATGAACTCAGAG 300
    81 -V--T--N--L--L--M--L--K--A--K--N--Q--A--F--L--E--M--N--S--E- 100
  301 GAAGCAGCTCAGAACCTGGTGGGTTATTACTCCACCATGGTGCCGATCATCAGGCACCAT 360
  101 -E--A--A--Q--N--L--V--G--Y--Y--S--T--M--V--P--I--I--R--H--H- 120
  361 CCAGTCTATGTACAGTTTTCCAACCACAAGGAGCTCAAGACTGACAACTCCCCAAACCAG 420
  121 -P--V--Y--V--Q--F--S--N--H--K--E--L--K--T--D--N--S--P--N--Q- 140
  421 GAGAGGGCTCAGGCAGCTCTTCGGGCTCTGAGTTCATCTCACGTGGACACGGCGGCGGTG 480
  141 -E--R--A--Q--A--A--L--R--A--L--S--S--S--H--V--D--T--A--A--V- 160
  481 GCTCCGAGCACAGTACTGAGGGTGGTGGTGGAGAACCTCATCTATCCCGTTACCCTGGAC 540
  161 -A--P--S--T--V--L--R--V--V--V--E--N--L--I--Y--P--V--T--L--D- 180
  541 GCCCTGTGCCAGATCTTCTCAAAGTTTGGCACCGTGCTAAGGATCATCATCTTCACAAAG 600
  181 -A--L--C--Q--I--F--S--K--F--G--T--V--L--R--I--I--I--F--T--K- 200
  601 AACAATCAGTTCCAGGCTCTGCTGCAGTATTCGGACGGCGCCTCAGCCCAGGCGGCCAAA 660
  201 -N--N--Q--F--Q--A--L--L--Q--Y--S--D--G--A--S--A--Q--A--A--K- 220
  661 CTGTCTCTGGACGGTCAGAACATCTATAATGGCTGCTGTACTCTGAGGATCAGCTTCTCC 720
  221 -L--S--L--D--G--Q--N--I--Y--N--G--C--C--T--L--R--I--S--F--S- 240
  721 AAACTCACCAGTCTCAACGTCAAATACAACAACGAGAAGAGCCGAGACTTCACCAGACCA 780
  241 -K--L--T--S--L--N--V--K--Y--N--N--E--K--S--R--D--F--T--R--P- 260
  781 GACCTTCCCACTGGAGACGGCCAGCCCACCATGGAACATACGGCCATGGCTACAGCCTTT 840
  261 -D--L--P--T--G--D--G--Q--P--T--M--E--H--T--A--M--A--T--A--F- 280
  841 ACTCCAGGCATCATCTCTGCTGCTCCATACGCTGGAGCCACCCACGCTTTCCCACCAGCC 900
  281 -T--P--G--I--I--S--A--A--P--Y--A--G--A--T--H--A--F--P--P--A- 300
  901 TTCACCCTGCAGCCTGCTGTGTCCTCCCCCTATCCAGGCCTTGCGGTCCCCGCTCTGCCC 960
  301 -F--T--L--Q--P--A--V--S--S--P--Y--P--G--L--A--V--P--A--L--P- 320
  961 GGAGCCCTGGCCTCTCTGTCCCTCCCTGGGGCCACCAGATTGGGATTCCCTCCAATCCCT 1020
  321 -G--A--L--A--S--L--S--L--P--G--A--T--R--L--G--F--P--P--I--P- 340
1021 GCTGGGCACTCTGTCTTGCTGGTCAGCAATCTCAACCCTGAGAGAGTTACGCCCCACTGC 1080
  341 -A--G--H--S--V--L--L--V--S--N--L--N--P--E--R--V--T--P--H--C- 360
1081 CTCTTTATTCTCTTCGGTGTCTATGGAGATGTCATGAGAGTGAAGATTCTGTTCAACAAG 1140
  361 -L--F--I--L--F--G--V--Y--G--D--V--M--R--V--K--I--L--F--N--K- 380
1141 AAAGAAAACGCTCTGGTTCAGATGTCTGACAGCACACAGGCTCAGCTAGCCATGAGCCAC 1200
  381 -K--E--N--A--L--V--Q--M--S--D--S--T--Q--A--Q--L--A--M--S--H- 400
1201 CTGAATGGCCAGCGGCTGCACGGGAAGCCTGTGCGCATCACTCTGTCCAAACACACGAGC 1260
  401 -L--N--G--Q--R--L--H--G--K--P--V--R--I--T--L--S--K--H--T--S- 420
1261 GTTCAGCTTCCTCGCGAAGGGCACGAGGACCAGGGCCTGACCAAAGACTACAGCAACTCC 1320
  421 -V--Q--L--P--R--E--G--H--E--D--Q--G--L--T--K--D--Y--S--N--S- 440
1321 CCCTTGCACCGCTTCAAGAAGCCCGGCTCCAAGAATTATTCCAACATCTTCCCGCCTTCT 1380
  441 -P--L--H--R--F--K--K--P--G--S--K--N--Y--S--N--I--F--P--P--S- 460
1381 GCCACCTTACACCTTTCCAACATTCCCCCTTCTGTGGTGGAAGATGATCTGAAGATGCTG 1440
  461 -A--T--L--H--L--S--N--I--P--P--S--V--V--E--D--D--L--K--M--L- 480
1441 TTTGCCAGCTCAGGAGCCGTGGTCAAAGCCTTCAAATTCTTCCAGAAGGACCATAAAATG 1500
  481 -F--A--S--S--G--A--V--V--K--A--F--K--F--F--Q--K--D--H--K--M- 500
1501 GCTCTAATCCAGGTGGGCTCTGTGGAGGAGGCCATCGAGTCCCTCATAGAATTCCACAAC 1560
  501 -A--L--I--Q--V--G--S--V--E--E--A--I--E--S--L--I--E--F--H--N- 520
1561 CATGATTTGGGAGAGAACCACCACCTGCGAGTCTCCTTCTCCAAATCCTCAATCTGA... 1617
  521 -H--D--L--G--E--N--H--H--L--R--V--S--F--S--K--S--S--I--*-... 538
1621 ACCATCTGAATCCTCAGAGTCAGCACGACAGTATCTACCACACTCCAATCATTCCACCAC 1680
          ............................................................
1681 ATGTCTGTAGAAAACACAGTAAAGTCTGGATTAATGTTAAATTATTATAATTATTATTAT 1740
          ............................................................
1741 AATTATTATTATTATTATTATTATTATTATTATTATTATTATTATTATCATCATTATGCT 1800
          ............................................................
1801 TTTTTTTTAAATAAGTATTTCGGTTCTTTGCCTTCTGACAGAATTAAGGTCTCTCAAGGA 1860
          ............................................................
1861 GAAATCTGACTTTATTCTCTCAAATTTTATAAACTAGAGAATAAAGTTATAGTTCTGACC 1920
          ............................................................
1921 TGTTCAGGCTTTCTGGGATAAAGCCGGAGTTCTTCGGTTAGTCAGAATTCAGACAGCAAA 1980
          ............................................................
1981 GTTGTTACCTTTTACCTTGCTTTTGGGCTTTGTTCCCACACTGAGGGATTAAAGTCAGCC 2040
          ............................................................
2041 TTCTTATCCAAAGATGTTACGTTTTTCAGATTCAGACTTTTACTTTGATCTTAAATTAGC 2100
          ............................................................
2101 CCAAGTTTTACAGGTGGCCCTGTTCCTTTTTTAGCTCTCCCTTTAAAAGTTCCAGCTCTG 2160
          ............................................................
2161 CTTGTAAACGATCAGAGGTCAGATGTCCGCTCAGGCCTGCAGGTCCAAGTCTGGCCCCGT 2220
          ............................................................
2221 AAAGAGAAGCCTGGCTAGACCTTCACATGATCCCTGTGCCTTATTCCTGGAGTGTGAATG 2280
          ............................................................
2281 ACCGTGACTATGTCATGTTTAAGAGAAAGAGGAGGTTTACAGTTGAAAAGGTTTACTGTT 2340
          ............................................................
2341 AATGAGCTGTATTACAGATATATCTGCGTTTTCTGTCTCTAGTGTTTTGTACCACTGTGT 2400
          ............................................................
2401 TACTGTAGTGTGAAACATGAACTGATTGTCTTTTAGCAGGTTTGTTGGGTCTTTAGTCCT 2460
          ............................................................
2461 AAATGCATTGTTTTTCTTTTTGACTTCTTTATTTCTGTGTTTGCAATCATGTGTTAATCA 2520
          ............................................................
2521 AATGTTGTAGCAATATTTTAATCATTCCTGATATAACTGTTTTTGTTTTAGTTTTTTTGG 2580
          ............................................................
2581 GTGCCTCTGTGAGCTCGGCCTTTCACGGCGTGCAGACAAATGTTTTGTCTAGTTGGTGAA 2640
          ............................................................
2641 TCTGGTCAGGTTGTCTTGTGTGTCGCCTCTCTGGATGGTTTTATTTATAAGTTTGTGATC 2700
          ............................................................
2701 CACTACAGCTGAAACCAAAAATGGCCTTCAGGATGCAAACAAATATGTCTGCCTCAGGTT 2760
          ............................................................
2761 TCTGGTTTTATGGACTACAGAAAGACTGCAAGCTGGTTTCAGCTTTCTTATTTTCCTGTG 2820
          ............................................................
2821 AGAATGCGGAATGTTTTTATTCATTTCTTAATTGCAAAACCAGATGTTTGGAGTGCCTTG 2880
          ............................................................
2881 GACGCAGCACTGAGTTGTAATCAGGCATTAATTTCTCTGTGTACTGATGTGACAGTTTGG 2940
          ............................................................
2941 TAGGGAGGAAGCCCACAGTCCTCCGAAACCACAAAATGCTGGTTTGATCTGTTTGTCTTA 3000
          ............................................................
3001 ATATGAATATTGTTATTTTCTCATTCCAGCTGCTCAGATGTTCAACTGAACTTCAAAAAG 3060
          ............................................................
3061 ACAAAGATTCTTCACTGACCATGGTTCATTTAAACAGGTTCATTGTGGTGCCTTCAGTAG 3120
          ............................................................
3121 AGCTTGGAGGGTTTGTGTGTTCACTTTGTCACTAGGTGAGGAGAAAATGGTGATTGTGTC 3180
          ............................................................
3181 CCAGTTTACTCCCTCCCTACATACCCAGACCAAAGGTGCGGGTGGGCGGGTCATTTCAGA 3240
          ............................................................
3241 GTCAAACAAATAAACTGTAGCCATGTTGCACCTGAATTTGGACATGACAAAAACCCCTTC 3300
          ............................................................
3301 TCCATTTGTACCTACCTACCGACTCGCCACAACCCGACTCGGATCGACTGGTTGTCCATT 3360
          ............................................................
3361 ACAATCCAGTACCACCTAACATGCGTCATTGTTGTTATAGCAACCCCTCTCAAGGCCCTG 3420
          ............................................................
3421 TGACGTAATTAGTATGCGACACGAACGCTGCAACAAAGGTGGCAGTCGAGGCAACGATAT 3480
          ............................................................
3481 ACCTGCTGCTTAGTCTGTGGCCTTTTGTCAGACTCGGAACCACGACATTGGTTTTTTTGT 3540
          ............................................................
3541 AGCTATTTCACTCGTTGCTGGGTTTCAAAAGTGGCCGTTTAAATTTTTGTGAACGAGACG 3600
          ............................................................
3601 GTCTCATGACTCATCAAATGAAATGACGGCACTGACCCACCAGTCAGTGGCATGCAGTCT 3660
          ............................................................
3661 GACATAACATTTAGTACATGCTCGGATCCCTTGGAATCCCTGCCAAGTAGGTACTATTTT 3720
          ............................................................
3721 AGTACCTGGTATTAGGAACTATCACCTAATAGAAAACCCTGGCAAGTCGATCTAATGGAA 3780
          ............................................................
3781 AAGGGGCTAAAGGTAAATCTTACGAGGTCCCTGCACATTGTGATTTCAGAGTTTGTATGG 3840
          ............................................................
3841 CTGTGCGAGGAATTTGAGACAGTTTCTAAAATTCACAGCTATATGTAACAGATAGGCACA 3900
          ............................................................
3901 TGACTCTGAGACATGTCAGCGATAACAACCAAACCCTCATGTACTTAAAAAAAAAAAACT 3960
          ............................................................
3961 TTTTACCATCTTCTTTCATTTAACTTTTCAAATGGCTTTAATTCTTACTCATTAAAATAC 4020
          ............................................................
4021 TCATGTGCGTATTATTAGCAAGAGAAGTCTGAGCTCCTAAAAAACCAACTCAAATAATGA 4080
          ............................................................
4081 AGACACATGTTCACTGAACAGAGGGTGTTTGTATTGGTGATTGATCAACTTAAACTGTCC 4140
          ............................................................
4141 GTTTTCTTCCACTATCAGACTGTGATGTGCAGTTCAGGGTTGTGATAAACCAGCTCATTG 4200
          ............................................................
4201 CAGTTCACACAAACCTTTCTGATTATGTGAGGTGCAAGTCTGACCTGAACCTGGCTGCTG 4260
          ............................................................
4261 TTACCACAGTGTCAGGGACCTTCATCTGTCAAACTGATAGACGTGCTGCATGCTGTTCAT 4320
          ............................................................
4321 CACTACATGGAGGCTGGGAGACACATTAGGACACAGTTCCCTTTAATCTGAAACCGCAGC 4380
          ............................................................
4381 CTTTCCGTTGGGACAGATCCCACAGGTGACTGGAATGCATCACAAAACCTGGTCAGGTGA 4440
          ............................................................
4441 GGTGGGAATGGGACCAGTCAGTCAGTAACAGGAGGGAGGGGCATTCAGCTGTACATACAC 4500
          ............................................................
4501 GTTTTATACCACAGTTCAGTGTCTAAAGGGCGATTGTCAGTTTTCTATCTGATGAAATCT 4560
          ............................................................
4561 GTATATTTTGATTAAAGTGTGAAATCGGTTCTGAGCCTTACATTGTTTGTGTCTAAAAGA 4620
          ............................................................
4621 AAGATTAAATCACTTTTTAATCTAAACCTCTAGGCCTTTGTTATCTGTCATCAGTGCGGT 4680
          ............................................................
4681 TTATATCAGTGTCTTTCACAAGTCTTGTGTGCGTAGAGTTGTTTGTTCATGTTAAACACT 4740
          ............................................................
4741 TTGTTTGATGACATTGTTGTTAGCCATGGCTGATCAGAGCTTTTTAATGAGTGTTTATTG 4800
          ............................................................
4801 ATGATATGACTTCTGATTGCACTGCCAACCAGAATTTAGTCTGATCCAAGGTAACTTGGT 4860
          ............................................................
4861 GTTTCTAATTTTTTTTTTTTTTACTTAAACAGGCAGGAGGTTACTGGGTTACACTGGATC 4920
          ............................................................
4921 AATGCAGTAAAATATAAGAAAATAAGTTGTATTTTATTTTATATTCTATGAGACCGTCTC 4980
          ............................................................
4981 ATTTGGGGAAGTTACTGCAACGTCACCATTAATAATACACTTAAATTCAAATACAAAGAT 5040
          ............................................................
5041 TCAGCCAGTTCACTTCAGTAAAAACAACCATTATGCCAAGAGCACAACATTAGTGGCTCA 5100
          ............................................................
5101 AAAACAGTTAGAGCAGGCTTCACGCATTTCTCTCTGAAAACCGCGTGGCAATTCAAATAA 5160
          ............................................................
5161 ATGAGAGGAGGCTGAAGGAAAAATAAATATACTTTTGATATAAGAAATGGCTGATAGGAA 5220
          ............................................................
5221 TTGTAGAGCAGTGTGCACATTATTCTGATTAAGACTAAAGGAAGATTTATGCAAAGGAAA 5280
          ............................................................
5281 ACTGCATTACAATAGTTCAAACTATTCCACTATACAAACCTTAAACAGCTGACCTTTATT 5340
          ............................................................
5341 TTTACTGCTTTCTACATAGTAGATACATAGTGAAGATGGATGTGAAGCAAGATGCATGGA 5400
          ............................................................
5401 ATTATGCAGCAAAGAAAAAACTCTAAATACAAATACATATCAGAAAAAGTTGGAACAGTA 5460
          ............................................................
5461 TGGTAAACACAAATTAAAAAAAAAAGTTTTGCCTGGTCAACTTCATTTCATTTGTAACTG 5520
          ............................................................
5521 TACATCCTTTCCTGTCATTCAGACCTGCAACACATTCCAAAAAAAGGTTGGGATAGGAGC 5580
          ............................................................
5581 AATTTAGCTCTAGTAATCAGGTAAATTGGTTAAATAATGATGTGATTTGTAACAGGTGAT 5640
          ............................................................
5641 TGTAACTATGATTTGGTACAAAAGCAGCATTCAAGAAACATCTAGTCCTTTAGGAGCAAA 5700
          ............................................................
5701 GATGGGCCGAGGATCGCCAGTTTGCCAACAAATGCGTGAGGAAAATTATTGAAATGTGTA 5760
          ............................................................
5761 AAAGCAATATTCAGGAAGAGATTTGGATATTTCACCTTAAACAGTGCATAACGTAATTAA 5820
          ............................................................
5821 AAGATTCAAGGAATCTGGAGGAATTTCAGTGTGTAAAGAACAAGTTCAGCTTAGGCTTGC 5880
          ............................................................
5881 GCCCAACTGTTATCTCCGATCCCTCAGGCAGCACTGCGTCAAGAATTGTCATTCATCTAT 5940
          ............................................................
5941 AAGTGATATCACCACATGGGCTCAAGTCTACTTTAGCAAACTTTTCTCATGTGCGTAGTT 6000
          ............................................................
6001 GCATCCATAAATGCC 6015
          ...............
(Pt bpla mutant allele-13 nt deletion)
LENGTH: 6015 bp (−13 bp) and 80 aa
TYPE: cDNA (SEQ ID NO: 125) and Protein (SEQ ID NO: 128)
ORGANISM: Nile tilapia
SEQ ID NOs 125 and 128
      1 ATGGACGGCAGTGTCCACCACGATATAACAGTTGGCACCAAGAGAGGATCTGACGAACTT 60
      1 -M--D--G--S--V--H--H--D--I--T--V--G--T--K--R--G--S--D--E--L- 20
    61 TTCTCCAGCGTCTCCAGCAACCCTTATATCATGAGCACCACAGCCAATGGCAACGACAGC 120
    21 -F--S--S--V--S--S--N--P--Y--I--M--S--T--T--A--N--G--N--D--S- 40
  121 AAAAAGTTCAAAGGTGACATAAGAGGCCCCAGCGTGCCATCCAGGGTCATCCACATCCGC 180
    41 -K--K--F--K--G--D--I--R--G--P--S--V--P--S--R--V--I--H--I--R- 60
  181 AAGCTTCCCAGCGACATCACAGAGGCGGAGGTGTGCCTTTTGGAGACGTCACCAACCTGC 240
    61 -K--L--P--S--D--I--T--E--A--E--V--C--L--L--E--T--S--P--T--C- 80
  241 TGATGCTCAAAGCCAAGAACCAGGCCTTTTTAGAGATGAACTCAGAGGAAGCAGCTCAGA 300
    81 -*- 80
(Pt bp1a mutant allele-1.5 knt deletion)
LENGTH: 6015 bp(−1.5 kb) and 346 aa
TYPE: cDNA (SEQ ID NO: 126) and Protein (SEQ ID NO: 129)
ORGANISM: Nile tilapia
SEQ ID NOs 126 and 129
      1 ATGGACGGCAGTGTCCACCACGATATAACAGTTGGCACCAAGAGAGGATCTGACGAACTT 60
      1 -M--D--G--S--V--H--H--D--I--T--V--G--T--K--R--G--S--D--E--L- 20
    61 TTCTCCAGCGTCTCCAGCAACCCTTATATCATGAGCACCACAGCCAATGGCAACGACAGC 120
    21 -F--S--S--V--S--S--N--P--Y--I--M--S--T--T--A--N--G--N--D--S- 40
  121 AAAAAGTTCAAAGGTGACATAAGAGGCCCCAGCGTGCCATCCAGGGTCATCCACATCCGC 180
    41 -K--K--F--K--G--D--I--R--G--P--S--V--P--S--R--V--I--H--I--R- 60
  181 AAGCTTCCCAGCGACATCACAGAGGCGGAGGTGATCAGAGACGGCCAGCCCACCATGGAA 240
    61 -K--L--P--S--D--I--T--E--A--E--V--I--R--D--G--Q--P--T--M--E- 80
  241 CATACGGCCATGGCTACAGCCTTTACTCCAGGCATCATCTCTGCTGCTCCATACGCTGGA 300
    81 -H--T--A--M--A--T--A--F--T--P--G--I--I--S--A--A--P--Y--A--G- 100
  301 GCCACCCACGCTTTCCCACCAGCCTTCACCCTGCAGCCTGCTGTGTCCTCCCCCTATCCA 360
  101 -A--T--H--A--F--P--P--A--F--T--L--Q--P--A--V--S--S--P--Y--P- 120
  361 GGCCTTGCGGTCCCCGCTCTGCCCGGAGCCCTGGCCTCTCTGTCCCTCCCTGGGGCCACC 420
  121 -G--L--A--V--P--A--L--P--G--A--L--A--S--L--S--L--P--G--A--T- 140
  421 AGATTGGGATTCCCTCCAATCCCTGCTGGGCACTCTGTCTTGCTGGTCAGCAATCTCAAC 480
  141 -R--L--G--F--P--P--I--P--A--G--H--S--V--L--L--V--S--N--L--N- 160
  481 CCTGAGAGAGTTACGCCCCACTGCCTCTTTATTCTCTTCGGTGTCTATGGAGATGTCATG 540
  161 -P--E--R--V--T--P--H--C--L--F--I--L--F--G--V--Y--G--D--V--M- 180
  541 AGAGTGAAGATTCTGTTCAACAAGAAAGAAAACGCTCTGGTTCAGATGTCTGACAGCACA 600
  181 -R--V--K--I--L--F--N--K--K--E--N--A--L--V--Q--M--S--D--S--T- 200
  601 CAGGCTCAGCTAGCCATGAGCCACCTGAATGGCCAGCGGCTGCACGGGAAGCCTGTGCGC 660
  201 -Q--A--Q--L--A--M--S--H--L--N--G--Q--R--L--H--G--K--P--V--R- 220
  661 ATCACTCTGTCCAAACACACGAGCGTTCAGCTTCCTCGCGAAGGGCACGAGGACCAGGGC 720
  221 -I--T--L--S--K--H--T--S--V--Q--L--P--R--E--G--H--E--D--Q--G- 240
  721 CTGACCAAAGACTACAGCAACTCCCCCTTGCACCGCTTCAAGAAGCCCGGCTCCAAGAAT 780
  241 -L--T--K--D--Y--S--N--S--P--L--H--R--F--K--K--P--G--S--K--N- 260
  781 TATTCCAACATCTTCCCGCCTTCTGCCACCTTACACCTTTCCAACATTCCCCCTTCTGTG 840
  261 -Y--S--N--I--F--P--P--S--A--T--L--H--L--S--N--I--P--P--S--V- 280
  841 GTGGAAGATGATCTGAAGATGCTGTTTGCCAGCTCAGGAGCCGTGGTCAAAGCCTTCAAA 900
  281 -V--E--D--D--L--K--M--L--F--A--S--S--G--A--V--V--K--A--F--K- 300
  901 TTCTTCCAGAAGGACCATAAAATGGCTCTAATCCAGGTGGGCTCTGTGGAGGAGGCCATC 960
  301 -F--F--Q--K--D--H--K--M--A--L--I--Q--V--G--S--V--E--E--A--I- 320
  961 GAGTCCCTCATAGAATTCCACAACCATGATTTGGGAGAGAACCACCACCTGCGAGTCTCC 1020
  321 -E--S--L--I--E--F--H--N--H--D--L--G--E--N--H--H--L--R--V--S- 340
1021 TTCTCCAAATCCTCAATCTGA 1041
  341 -F--S--K--S--S--I--*- 346
(wild-type Nos3)
LENGTH: 660 bp and 219 aa
TYPE: cDNA (SEQ ID NO: 130) and Protein (SEQ ID NO: 132)
ORGANISM: Nile tilapia
SEQ ID NOs 130 and 132
      1 ATGAACGGAATGGTTTGGGGATTCCTTCATCACCTGCCACGCGTTATGGAGTCCGACGGC 60
      1 -M--N--G--M--V--W--G--F--L--H--H--L--P--R--V--M--E--S--D--G- 20
    61 AAAAGTTTCCAGCCCTGGCGAGACTACATGGGACTGTGTGATACAATCAGAGATATCTTG 120
    21 -K--S--F--Q--P--W--R--D--Y--M--G--L--C--D--T--I--R--D--I--L- 40
  121 GGTCGCAGCACCGTCTCCGAGTCCTCTCAGCCTGTGTCCAAAGCTCATCACACGGAGTGT 180
    41 -G--R--S--T--V--S--E--S--S--Q--P--V--S--K--A--H--H--T--E--C- 60
  181 GACATGAGCCGAGCTATGGTATCTTTGCGCATTAACGCAGCTCGCCAAAGTGGCCTCGGA 240
    61 -D--M--S--R--A--M--V--S--L--R--I--N--A--A--R--Q--S--G--L--G- 80
  241 GCAGAGAGTGCGCCGGATCCCTGCTCCCGCGAATGTGCACTAACGAGTTCCCCTGCTCGC 300
    81 -A--E--S--A--P--D--P--C--S--R--E--C--A--L--T--S--S--P--A--R- 100
  301 ATGGATCCAGTGGATGGTGTGGCGTATGCACCAAACGCAATCGATCTGAAATTGATGCAA 360
  101 -M--D--P--V--D--G--V--A--Y--A--P--N--A--I--D--L--K--L--M--Q- 120
  361 AACCCGCCGGGCTCCCGGGGGCCAAAAGATCGAAAGAAGACGAGTCGTTTCAAAACACCC 420
  121 -N--P--P--G--S--R--G--P--K--D--R--K--K--T--S--R--F--K--I--P- 140
  421 GAGGCAGTCTTACCCACTCCTGACCGCATGTTCTGCAGCTTTTGTAAACACAACGGAGAG 480
  141 -E--A--V--L--P--T--P--D--R--M--F--C--S--F--C--K--H--N--G--E- 160
  481 TCTGAGCTGGTCTACGGATCCCACTGGCTGAAGAACCAAGAAGGAGATGTTTTGTGTCCC 540
  161 -S--E--L--V--Y--G--S--H--W--L--K--N--Q--E--G--D--V--L--C--P- 180
  541 TTTCTGCGGCAGTATGTGTGTCCTCTGTGTGGCGCCACAGGGGCCAAAGCTCACACCAAG 600
  181 -F--L--R--Q--Y--V--C--P--L--C--G--A--T--G--A--K--A--H--T--K- 200
  601 CGTTTCTGCCCCAAAGTGGACAGCGCATACAGCTCCGTGTACGCCAAGTCCAGACGCTGA 660
  201 -R--F--C--P--K--V--D--S--A--Y--S--S--V--Y--A--K--S--R--R--*- 219
(Nos3 mutant allele-5 nt deletion)
LENGTH: 660 bp(−5 pb) and 145 aa
TYPE: cDNA (SEQ ID NO: 131) and Protein (SEQ ID NO: 133)
ORGANISM: Nile tilapia
SEQ ID NOs 131 and 133
      1 ATGAACGGAATGGTTTGGGGATTCCTTCATCACCTGCCACGCGTTATGGAGTCCGACGGC 60
      1 -M--N--G--M--V--W--G--F--L--H--H--L--P--R--V--M--E--S--D--G- 20
    61 AAAAGTTTCCAGCCCTGGCGAGACTACATGGGACTGTGTGATACAATCAGAGATATCTTG 120
    21 -K--S--F--Q--P--W--R--D--Y--M--G--L--C--D--T--I--R--D--I--L- 40
  121 GGTCGCAGCACCGTCTCCGAGTCCTCTCAGCCTGTGTCCAAAGCTCATCACACGGAGTGT 180
    41 -G--R--S--T--V--S--E--S--S--Q--P--V--S--K--A--H--H--T--E--C- 60
  181 GACATGAGCCGAGCTATGGTATCTTTGCGCATTAACGCAGCTCGCCAAAGTGGCCTCGGA 240
    61 -D--M--S--R--A--M--V--S--L--R--I--N--A--A--R--Q--S--G--L--G- 80
  241 GCAGAGAGTGCGCCGGATCCCTGCTCCCGCGAATGTGCACTAACGAGTTCCCCTGCTCGC 300
    81 -A--E--S--A--P--D--P--C--S--R--E--C--A--L--T--S--S--P--A--R- 100
  301 ATGGATCCAGTGGATGGTGTGGCGTATGCACCAAACGCAATCGATCTGAAATTGATGCAA 360
  101 -M--D--P--V--D--G--V--A--Y--A--P--N--A--I--D--L--K--L--M--Q- 120
  361 AACCCGCCGGGCTCCCGGGGGCCAAAAGATCGAAAGAAGACGAGTCGTTTCAAAACACCC 420
  121 -N--P--P--G--S--R--G--P--K--D--R--K--K--T--S--R--F--K--T--P- 140
  421 AGTCTTACCCACTCCTGACCGCATGTTCTGCAGCTTTTGTAAACACAACGGAGAGTCTGA 480
  141 -S--L--T--H--S--*- 145
(wild-type dndP
LENGTH: 1653 bp and 320 aa
TYPE: cDNA (SEQ ID NO: 134) and Protein (SEQ ID NO: 136)
ORGANISM: Nile tilapia
SEQ ID NOs 134 and 136
      1 AGACAATGCACAATAGGTTACAAAAAAGTTTAAAAGCAGTCCTCCATACACAGCCGTTTG 60
          ............................................................
    61 GTATTTGTGACAAAATTTCATTCCATACCTTAGCGACGGGCTATGCTAGGCCCCGCCCAC 120
          ............................................................
  121 GGCTCAGTGGGCACTAAAGACATAGCATCGAGTGTACGCTGGACTACTGCAGTTGGAAAC 180
          ............................................................
  181 GGGCTACAAAGTGGCGTCGCTGTGCGCACAAACACGCTGAGACGATGGAAAACACGCAAA 240
          ............................................-M--E--N--T--Q-- 5
  241 GCCAGGTGCTGAACCTTGAACGGGTGCAGGCCCTGGAAATCTGGTTGAAAGCAACCAACA 300
      6 S--Q--V--L--N--L--E--R--V--Q--A--L--E--I--W--L--K--A--T--N-- 25
  301 CAAAGCTGACTCAAGTTAATGGCCAGAGGAAATATGGAGGACCACCTGAGGTGTGGGAAG 360
    26 T--K--L--T--Q--V--N--G--Q--R--K--Y--G--G--P--P--E--V--W--E-- 45
  361 GTCCCACACCGGGACCGCGCTGTGAAGTCTTCATCAGCCAGATCCCACGGGACACGTATG 420
    46 G--P--T--P--G--P--R--C--E--V--F--I--S--Q--I--P--R--D--T--Y-- 65
  421 AGGACATCCTTATTCCCCTGTTCAGCTCCATTGGGCCACTCTGGGAGTTCCGGCTGATGA 480
    66 E--D--I--L--I--P--L--F--S--S--I--G--P--L--W--E--F--R--L--M-- 85
  481 TGAACTTCAGTGGGCAGAACCGCGGCTTTGCGTATGCCAAATATGGCTCAGCTGCTATAG 540
    86 M--N--F--S--G--Q--N--R--G--F--A--Y--A--K--Y--G--S--A--A--I-- 105
  541 CTGTTGAAGCCATACGACAGCTGCACGGTCACATGGTGGAGCCTGGCTACCGCATCAGTG 600
  106 A--V--E--A--I--R--Q--L--H--G--H--M--V--E--P--G--Y--R--I--S-- 125
  601 TACGGCGGAGCACAGAGAAGCGACACCTTTGTATTGGAGGTCTGCCTGCTTCCACTAGAC 660
  126 V--R--R--S--T--E--K--R--H--L--C--I--G--G--L--P--A--S--T--R-- 145
  661 AAGAAGGCATACTGCAGGTGCTGCGTATGCTGGTAGAGGGGGTGGAGAGAGTTTCCCTGA 720
  146 Q--E--G--I--l--Q--V--L--R--M--L--V--E--G--V--E--R--V--S--L-- 165
  721 AGGCCGGACCTGGTATAGAGGGGGTATCTGCTACTGTTGCTTTCTCATCTCACCATGCAG 780
  166 K--A--G--P--G--I--E--G--V--S--A--T--V--A--F--S--S--H--H--A-- 185
  781 CTTCTATGGCTAAGAAAGTGCTGGTGGAAGCATTTAAGAAGCAGTTTGCAATGTGTGTGT 840
  186 A--S--M--A--K--K--V--L--V--E--A--F--K--K--Q--F--A--M--C--V-- 205
  841 CAGTCAAGTGGCAGCCAACAGAGAAGCCAAACCCTGACGAGCCACGATGCCCTCAGAAAC 900
  206 S--V--K--W--Q--P--T--E--K--P--N--P--D--E--P--R--C--P--Q--K-- 225
  901 GTGCAAAGAGCCTGTTGCCGTCACACCTAGGGCCCCTGCACCACAGTTCTCCACAACCCT 960
  226 R--A--K--S--L--L--P--S--H--L--G--P--L--H--H--S--S--P--Q--P-- 245
  961 CAGGCCCGCCTTCATTCCTGACCCTCCCTGCATCCATACCCGCAGGTTTCTGCAGAGCAG 1020
  246 S--G--P--P--S--F--L--T--L--P--A--S--I--P--A--G--F--C--R--A-- 265
1021 TGGGAGGGCCCACTGCTCCTCAGCTCGCTCACCCTACATGCTCTTTTCCCAATTCCTCCA 1080
  266 V--G--G--P--T--A--P--Q--L--A--H--P--T--C--S--F--P--N--S--S-- 285
1081 CCCAAGGCCATCTTGTATTTGCAGCATCCCCAGTGATGCTTCTCAGTGCAGATCCGCGGG 1140
  286 T--Q--G--H--L--V--F--A--A--S--P--V--M--L--L--S--A--D--P--R-- 305
1141 ATCACTGCCGCTTTCAAGGGGTTGGTCATGATCTTACCGGGTCCTAATGCCAGCACCATG 1200
  306 D--H--C--R--F--Q--G--V--G--H--D--L--T--G--S--*-............. 320
1201 CTAGAGGAGGCTCAGAAGGCTGTAGCCCAGCAGGTCCTGCAGAAGATGTACAACACTGGT 1260
          ............................................................
1261 CTCACACACTAAACAGCTGATGCCGTCCTGCAGTTCTGTTTCACCTTGTTTGTGTTATGT 1320
          ............................................................
1321 GGTTTCATTTTCTGCATGTTTTTACTAGAGTAGCACCAAGTTTGTTTCTCTGACTATAAC 1380
          ............................................................
1381 TTGTGGTTTGTTTTATGCATGATTTTTACTGrACATTAGTGTTCTGTGTTACTGGATTGG 1440
          ............................................................
1441 TTCTCATTTTAATTAAATGAGCTTTGAAAAGAAAGTGTCGGCGTTTCTTTCAAATTAATG 1500
          ............................................................
1501 AAAGATTTAAATTAACTTAGGAAAATGGTAAAGCAGTTATTATTGTCTCACTTCATGCTG 1560
          ............................................................
1561 TTATGAACCCTAGTGATTCTCATCCAGACCTTTACGTATCTTTGAAGGTTGTGGATTGAG 1620
          ............................................................
1621 ACTAACCCCCCTCAGTGGTTTGGCATTTTAAAC 1653
          .................................
(dnd mutant allele-5 nt deletion)
LENGTH: 1653 bp (−5 pb) and 324 aa
TYPE: cDNA (SEQ ID NO: 135) and Protein (SEQ ID NO: 137)
ORGANISM: Nile tilapia
SEQ ID NOs 135 and 137
      1 AGACAATGCACAATAGGTTACAAAAAAGTTTAAAAGCAGTCCTCCATACACAGCCGTTTG 60
          ............................................................
    61 GTATTTGTGACAAAATTTCATTCCATACCTTAGCGACGGGCTATGCTAGGCCCCGCCCAC 120
          ............................................................
  121 GGCTCAGTGGGCACTAAAGACATAGCATCGAGTGTACGCTGGACTACTGCAGTTGGAAAC 180
          ............................................................
  181 GGGCTACAAAGTGGCGTCGCTGTGCGCACAAACACGCTGAGACGATGGAAAACACGCAAA 240
          ............................................-M--E--N--T--Q-- 5
  241 GCCAGGTGCTGAACCTTGAACGGGTGCAGGCCCTGGAAATCTGGTTGAAAGCAACCAACA 300
      6 S--Q--V--L--N--L--E--R--V--Q--A--L--E--I--W--L--K--A--T--N-- 25
  301 CAAAGCTGACTCAAGTTAATGGCCAGAGGAAATATGGAGGACCACCTGAGGTGTGGGAAG 360
    26 T--K--L--T--Q--V--N--G--Q--R--K--Y--G--G--P--P--E--V--W--E-- 45
  361 GTCCCACACCGGGACCGCGCTGTGAAGTCTTCATCAGCCAGATCCCACGGGACACGTATG 420
    46 G--P--T--P--G--P--R--C--E--V--F--I--S--Q--I--P--R--D--T--Y-- 65
  421 AGGACATCCTTATTCCCCTGTTCAGCTCCATTGGGCCACTCTGGGAGTTCCGGCTGATGA 480
    66 E--D--I--L--I--P--L--F--S--S--I--G--P--L--W--E--F--R--L--M-- 85
  481 TGAACTTCAGTGGGCAGAACCGCGGCTTTGCGTATGCCAAATATGGCTCAGCTGCTATAG 540
    86 M--N--F--S--G--Q--N--R--G--F--A--Y--A--K--Y--G--S--A--A--I-- 105
  541 CTGTTGAAGCCATACGACAGCTGCACGGTCACATGGTGGAGCCTGGCTACCGCATCAGTG 600
  106 A--V--E--A--I--R--Q--L--H--G--H--M--V--E--P--G--Y--R--I--S-- 125
  601 TACGGCGGAGCACAGAGAAGCGACACCTTTGTATTGGAGGTCTGCCTGCTTCCACTAGAC 660
  126 V--R--R--S--T--E--K--R--H--L--C--I--G--G--L--P--A--S--T--R-- 145
  661 AAGAAGGCATACTGCAGGTGCTGCGTATGCTGGTAGAGGGGGTGGAGAGAGTTTCCCTGA 720
  146 Q--E--G--I--L--Q--V--L--R--M--L--V--E--G--V--E--R--V--S--L-- 165
  721 AGGCCGGACCTGGTATAGAGGGGGTATCTGCTACTGTTGCTTTCTCATCTCACCATGCAG 780
  166 K--A--G--P--G--I--E--G--V--S--A--T--V--A--F--S--S--H--H--A-- 185
  781 CTTCTATGGCTAAGAAAGTGCTGGTGGAAGCATTTAAGAAGCAGTTTGCAATGTGTGTGT 840
  186 A--S--M--A--K--K--V--L--V--E--A--F--K--K--Q--F--A--M--C--V-- 205
  841 CAGTCAAGTGGCAGCCAACAGAGAAGCCAAACCCTGACGAGCCACGATGCCCTCAGAAAC 900
  206 S--V--K--W--Q--P--I--E--K--P--N--P--D--E--P--R--C--P--Q--K-- 225
  901 GTGCAAAGAGCCTGTTGCCGTCACACCTAGGGCCCCTGCACCACAGTTCTCCACAACCCT 960
  226 R--A--K--S--L--L--P--S--H--L--G--P--L--H--H--S--S--P--Q--P-- 245
  961 CAGGCCCGCCTTCATTCCTGACCCTCCCTGCATCCATACCCGCAGGTTTCTGCAGAGCAG 1020
  246 S--G--P--P--S--F--L--T--L--P--A--S--I--P--A--G--F--C--R--A-- 265
1021 TGGGAGGGCCCACTGCTCCTCAGCTCGCTCACCCTACATGCTCTTTTCCCAATTCCTCCA 1080
  266 V--G--G--P--T--A--P--Q--L--A--H--P--T--C--S--F--P--N--S--S-- 285
1081 CCCAAGGCCATCTTGTATTTGCAGCATCCCCAGTGATGCTTCTCAGTGCAGATCCGCGGG 1140
  286 T--Q--G--H--L--V--F--A--A--S--P--V--M--L--L--S--A--D--P--R-- 305
1141 ATCACTGCCGCTTTCAAGGGGTTGGTCATGATCGGGTCCTAATGCCAGCACCATGCTAGA 1200
  306 D--H--C--R--F--Q--G--V--G--H--D--R--V--L--M--P--A--P--C--*-- 324
(wild-type Hnrnpab)
LENGTH: 999 bp and 332 aa
TYPE: cDNA (SEQ ID NO: 138) and Protein (SEQ ID NO: 140)
ORGANISM: Nile tilapia
SEQ ID NOs 138 and 140
      1 ATGTCTGAGTCAGAGCAACAGTACATGGAAACATCGGAAAACGGCCACGAAGTCGACGAT 60
      1 -M--S--E--S--E--Q--Q--Y--M--E--T--S--E--N--G--H--E--V--D--D- 20
    61 GATTTTAACGGAGCCGGCCTCACTGAGGAGGGGAATGACGACGACGGCGCCACCGCGAAT 120
    21 -D--F--N--G--A--G--L--T--E--E--G--N--D--D--D--G--A--T--A--N- 40
  121 GACTGCGGAGAGGACGCAGGGCCCGAGGAAGACGACAATTCGCAAAACGGCGGCACGGAG 180
    41 -D--C--G--E--D--A--G--P--E--E--D--D--N--S--Q--N--G--G--T--E- 60
  181 GGAGGCCAGATCGACGCCAGCAAGGGCGAGGAGGATGCCGGGAAAATGTTCGTTGGAGGT 240
    61 -G--G--Q--I--D--A--S--K--G--E--E--D--A--G--K--M--F--V--G--G- 80
  241 CTCAGCTGGGACACAAGCAAGAAGGATCTTAAAGACTACTTCTCTAAATTTGGCGAGGTG 300
    81 -L--S--W--D--T--S--K--K--D--L--K--D--Y--F--S--K--F--G--E--V- 100
  301 ACAGACTGCACCATCAAGATGGACCAGCAGACAGGCCGGTCAAGAGGCTTTGGTTTCATT 360
  101 -T--D--C--T--I--K--M--D--Q--Q--T--G--R--S--R--G--F--G--F--I- 120
  361 CTGTTTAAAGATGCAGCCAGCGTAGAAAAGGTTCTTGAACAGAAGGAGCACAGGCTAGAT 420
  121 -L--F--K--D--A--A--S--V--E--K--V--L--E--Q--K--E--H--R--L--D- 140
  421 GGGAGACAGATTGACCCCAAGAAAGCCATGGCCATGAAGAAGGATCCAGTAAAGAAAATC 480
  141 -G--R--Q--I--D--P--K--K--A--M--A--M--K--K--D--P--V--K--K--I- 160
  481 TTTGTGGGCGGACTCAACCCTGATACTTCAAAGGAAGTCATTGAGGAGTACTTTGGGACC 540
  161 -F--V--G--G--L--N--P--D--T--S--K--E--V--I--E--E--Y--F--G--T- 180
  541 TTTGGAGAGATTGAGACCATAGAGCTTCCACAGGACCCAAAGACAGAGAAGAGGAGGGGA 600
  181 -F--G--E--I--E--T--I--E--L--P--Q--D--P--K--T--E--K--R--R--G- 200
  601 TTCGTATTCATCACGTACAAGGAAGAGGCTCCCGTGAAGAAAGTCATGGAGAAGAAGTAC 660
  201 -F--V--F--I--T--Y--K--E--E--A--P--V--K--K--V--M--E--K--K--Y- 220
  661 CACAATGTTGGTGGTAGCAAGTGTGAAATTAAAATCGCGCAGCCCAAAGAGGTCTACCTG 720
  221 -H--N--V--G--G--S--K--C--E--I--K--I--A--Q--P--K--E--V--Y--L- 240
  721 CAGCAGCAGTATGGTGCCCGTGGATATGGCGGACGTGGGCGAGGACGTGGAGGCCAGGGC 780
  241 -Q--Q--Q--Y--G--A--R--G--Y--G--G--R--G--R--G--R--G--G--Q--G- 260
  781 CAGAACTGGAATCAAGGCTACAACAACTACTGGAACCAGGGATACAACCAGGGCTATGGT 840
  261 -Q--N--W--N--Q--G--Y--N--N--Y--W--N--Q--G--Y--N--Q--G--Y--G- 280
  841 TATGGACAGCAAGGCTACGGATATGGTGGCTATGGTGGCTATGACTACTCTGCTGGTTAT 900
  281 -Y--G--Q--Q--G--Y--G--Y--G--G--Y--G--G--Y--D--Y--S--A--G--Y- 300
  901 TACGGCTATGGGGGTGGCTACGATTACAACCAGGGCAATACAAGCTATGGGAAAACTCCA 960
  301 -Y--G--Y--G--G--G--Y--D--Y--N--Q--G--N--T--S--Y--G--K--T--P- 320
  961 AGACGTGGAGGCCACCAGAGTAGCTACAAGCCATACTGA 999
  321 -R--R--G--G--H--Q--S--S--Y--K--P--Y--*- 332
(Hnrnpab mutant allele-8 nt deletion)
LENGTH: 999 bp (−8 bp) and 29 aa
TYPE: cDNA (SEQ ID NO: 139) and Protein (SEQ ID NO: 141)
ORGANISM: Nile tilapia
SEQ ID NOs 139 and 141
      1 ATGTCTGAGTCAGAGCAACAGTACATGGAAACATCGGAAAACGGCCACGAAGTCGACGAT 60
      1 -M--S--E--S--E--Q--Q--Y--M--E--T--S--E--N--G--H--E--V--D--D- 20
    61 GATTTTAACGGAGCCGGCCTCGGGGAATGACGACGACGGCGCCACCGCGAATGACTGCGG 120
    21 -D--F--N--G--A--G--L--G--E--*- 29
(wild-type Hermes)
LENGTH: 525 bp and 174 aa
TYPE: cDNA (SEQ ID NO: 142) and Protein (SEQ ID NO: 144)
ORGANISM: Nile tilapia
SEQ ID NOs 142 and 144
      1 CAGGTCCGAACACTATTTGTCAGTGGGCTACCACTGGATATTAAACCGCGGGAGCTCTAC 60
      1 -Q--V--R--T--L--F--V--S--G--L--P--L--D--I--K--P--R--E--L--Y- 20
    61 CTCCTCTTCAGACCATTTAAGGGCTATGAAGGCTCCTTGATAAAGCTCACTTCTAAACAG 120
    21 -L--L--F--R--P--F--K--G--Y--E--G--S--L--I--K--L--T--S--K--Q- 40
  121 CCAGTGGGGTTTGTCAGTTTTGACAGTCGATCAGAGGCGGAGGCTGCTAAGAATGCCTTG 180
    41 -P--V--G--F--V--S--F--D--S--R--S--E--A--E--A--A--K--N--A--L- 60
  181 AACGGGGTACGATTTGACCCAGAGATTCCCCAGACTCTGCGGCTGGAGTTCGCCAAGGCC 240
    61 -N--G--V--R--F--D--P--E--I--P--Q--T--L--R--L--E--F--A--K--A- 80
  241 AACACCAAGATGGCCAAAAACAAGCTGGTTGGCACTCCCAACCCCCCACCTTCTCAGCAG 300
    81 -N--T--K--M--A--K--N--K--L--V--G--T--P--N--P--P--P--S--Q--Q- 100
  301 AGCCCCGGGCCACAGTTCATAAGCAGAGACCCATATGAGCTCACAGTGCCTGCTCTCTAT 360
  101 -S--P--G--P--Q--F--I--S--R--D--P--Y--E--L--T--V--P--A--L--Y- 120
  361 CCCAGCAGCCCAGACGTGTGGGCCTCATACCCGCTGTACCCGGCGGAGCTGTCGCCGGCC 420
  121 -P--S--S--P--D--V--W--A--S--Y--P--L--Y--P--A--E--L--S--P--A- 140
  421 CTTCCACCCGCTTTCACCTACCCCTCCTCGCTCCACGCTCAGATTCGTTGGCTCCCGCCT 480
  141 -L--P--P--A--F--T--Y--P--S--S--L--H--A--Q--I--R--W--L--P--P- 160
  481 GCAGATGGAACTCCTCAGGGATGGAAGTCCAGGCAGTTCTGCTGA 525
  161 -A--D--G--T--P--Q--G--W--K--S--R--Q--F--C--*- 174
(Hermes mutant allele-16 nt insertion)
LENGTH: 525 bp(+16 bp) and 61 aa
TYPE: cDNA (SEQ ID NO: 143) and Protein (SEQ ID NO: 145)
ORGANISM: Nile tilapia
SEQ ID NOs 143 and 145
      1 CAGGTCCGAACACTATTTGTCAGTGGGCTACCACTGGATATTAAACCGCGGGAGCTCTAC 60
      1 -Q--V--R--T--L--F--V--S--G--L--P--L--D--I--K--P--R--E--L--Y- 20
    61 CTCCTCTTCAGACCATTTAAGGGCTATGAAGGCTCCTTGATAAAGCTCACTTCTAAACAG 120
    21 -L--L--F--R--P--F--K--G--Y--E--G--S--L--I--K--L--T--S--K--Q- 40
  121 CCAGTGGGGTTTGTCAGTTTTGACAGTCGATCAGAGTCGATCACACCTACGATCGGAGGC 180
    41 -P--V--G--F--V--S--F--D--S--R--S--E--S--I--T--P--T--I--G--G- 60
  181 TGCTAAGAATGCCTTGAACGGGGTACGATTTGACCCAGAGATTCCCCAGACTCTGCGGC 240
    61 -C--*- 61
(wild-type RBM24)
LENGTH: 708 bp and 235 aa
TYPE: cDNA (SEQ ID NO: 146) and Protein (SEQ ID NO: 148)
ORGANISM: Nile tilapia
SEQ ID NOs 146 and 148
      1 ATGCACGCGGCACAGAAAGACACCACCTTCACCAAGATCTTTGTGGGAGGTCTTCCTTAT 60
      1 -M--H--A--A--Q--K--D--I--T--F--T--K--I--F--V--G--G--L--P--Y- 20
    61 CACACAACCGACTCAAGTCTGAGAAAATACTTCGAGGTGTTTGGCGACATCGAAGAGGCC 120
    21 -H--T--T--D--S--S--L--R--K--Y--F--E--V--F--G--D--I--E--E--A- 40
  121 GTCGTTATCACTGACCGGCAGACGGGCAAATCCAGAGGTTATGGATTCGTGACCATGGCA 180
    41 -V--V--I--T--D--R--Q--T--G--K--S--R--G--Y--G--F--V--T--M--A- 60
  181 GACCGGGCCTCTGCCGACCGAGCCTGCAAGGACCCCAACCCCATAATAGATGGAAGGAAA 240
    61 -D--R--A--S--A--D--R--A--C--K--D--P--N--P--I--I--D--G--R--K- 80
  241 GCCAATGTGAACCTGGCGTATCTTGGGGCCAAACCCAGAGTCATTCAGCCAGGCTTTGCA 300
    81 -A--N--V--N--L--A--Y--L--G--A--K--P--R--V--I--Q--P--G--F--A- 100
  301 TTTGGTGTGCCTCAGATCCATCCAGCATTCATCCAGAGACCTTACGGGATCCCAGCTCAT 360
  101 -F--G--V--P--Q--I--H--P--A--F--I--Q--R--P--Y--G--I--P--A--H- 120
  361 TATGTCTTCCCTCAAGCCTTTGTCCAACCCAGCGTGGTGATCCCTCATGTACAACCGTCT 420
  121 -Y--V--F--P--Q--A--F--V--Q--P--S--V--V--I--P--H--V--Q--P--S- 140
  421 GCGGCTACAGCAACAGCTGCTGCTGCCACTTCCCCATACCTTGACTATACTGGAGCAGCT 480
  141 -A--A--T--A--T--A--A--A--A--T--S--P--Y--L--D--Y--T--G--A--A- 160
  481 TATGCCCAGTACTCCGCAGCTGCTGCGACTGCTGCTGCTGCAGCTGCTGCCTATGAGCAG 540
  161 -Y--A--Q--Y--S--A--A--A--A--T--A--A--A--A--A--A--A--Y--E--Q- 180
  541 TATCCGTACGCAGCTTCACCAGCACCGACGAGCTACATGACTACAGCGGGGTATGGGTAC 600
  181 -Y--P--Y--A--A--S--P--A--P--T--S--Y--M--T--T--A--G--Y--G--Y- 200
  601 ACTGTCCAGCAGCCGCTCGCCACCGCTGCCACCCCAGGAGCAGCTGCTGCTGCTGCGGCC 660
  201 -T--V--Q--Q--P--L--A--I--A--A--T--P--G--A--A--A--A--A--A--A- 220
  661 TTCAGTCAGTACCAGCCTCAGCAGCTCCAGACAGATCGCATGCAGTAA 708
  221 -F--S--Q--Y--Q--P--Q--Q--L--Q--T--D--R--M--Q--*- 235
(RBM24 mutant allele-7 nt deletion)
LENGTH: 708 bp (−7 bp) and 54 aa
TYPE: cDNA (SEQ ID NO: 147) and Protein (SEQ ID NO: 149)
ORGANISM: Nile tilapia
SEQ ID NOs 147 and 149
      1 ATGCACGCGGCACAGAAAGACACCACCTTCACCAAGATCTTTGTGGGAGGTCTTCCTTAT 60
      1 -M--H--A--A--Q--K--D--T--T--F--T--K--I--F--V--G--G--L--P--Y- 20
    61 CACACAACCGACTCAAGTCTGAGAAAATACTTCGAGGTGTTTGGCGACATCGAAGAGGCC 120
    21 -H--T--T--D--S--S--L--R--K--Y--F--E--V--F--G--D--I--E--E--A- 40
  121 GTCGTTACCGGCAGACGGGCAAATCCAGAGGTTATGGATTCGTGACCATGGCAGACCGGG 180
    41 -V--V--T--G--R--R--A--N--P--E--V--M--D--S--*- 54
(wild-type RBM42)
LENGTH: 1227 bp and 408 aa
TYPE: cDNA (SEQ ID NO: 150) and Protein (SEQ ID NO: 152)
ORGANISM: Nile tilapia
SEQ ID NOs 150 and 152
      1 ATGGCGCTCAAGTCCGGCGAGGAGCGTCTGAAGGAGATGGAGGCTGAGATGGCGCTCTTT 60
      1 -M--A--L--K--S--G--E--E--R--L--K--E--M--E--A--E--M--A--L--F- 20
    61 GAGCAGGAGGTTCTCGGTGGTCCAGTACCAGTATCAGGAAGTCCACCTGTCATGGAGGCA 120
    21 -E--Q--E--V--L--G--G--P--V--P--V--S--G--S--P--P--V--M--E--A- 40
  121 GTACCTGTAGCTCTGGCTGTCCCAACAGTTCCAGTGGTGCGACCCATTATAGGAACCAAC 180
    41 -V--P--V--A--L--A--V--P--T--V--P--V--V--R--P--I--I--G--T--N- 60
  181 ACCTACAGACAGGTCCAGCAGACATTAGAAGCCAGAGCTGCAACTTTTGTTGGACCTCCA 240
    61 -T--Y--R--Q--V--Q--Q--T--L--E--A--R--A--A--T--F--V--G--P--P- 80
  241 CCACAAGCCTTTGTGGGACCAGTTCCTCCAGTACGTCCTCCTCCTCCCATGATGAGACCG 300
    81 -P--Q--A--F--V--G--P--V--P--P--V--R--P--P--P--P--M--M--R--P- 100
  301 GCTTTTGTTCCACATATTCTGCAAAGACCAGTGTTGTCAGGTGGTCAGAGGTTACAGATG 360
  101 -A--F--V--P--H--I--L--Q--R--P--V--L--S--G--G--Q--R--L--Q--M- 120
  361 ATGCGTGGTCCTCCAGTAGCACCTCCTTTGCCTCGACCTCCTCCACCTCCACCCATGATG 420
  121 -M--R--G--P--P--V--A--P--P--L--P--R--P--P--P--P--P--P--M--M- 140
  421 CTCCCTCCTTCCCTGCAGGGCCCAATGCCTCAGGGACCCTCTCAGCCCATCCAACCCATG 480
  141 -L--P--P--S--L--Q--G--P--M--P--Q--G--P--S--Q--P--I--Q--P--M- 160
  481 GCTGCTCCACCTCAGGTTGGTGATATGGTTTCAATGGTGTCAGGCCCACCTACACGACAA 540
  161 -A--A--P--P--Q--V--G--D--M--V--S--M--V--S--G--P--P--T--R--Q- 180
  541 GTAGCCTCACTTCCTGTCAAACCAACACCATCAATCATCCAGGCAGCACCAACTGTGTAC 600
  181 -V--A--S--L--P--V--K--P--T--P--S--I--I--Q--A--A--P--T--V--Y- 200
  601 GTTGCTCCTCCTGCCCATGTTGGACTAAAAAGAAATGAAGTTCACGCTCAGAGACAGGCC 660
  201 -V--A--P--P--A--H--V--G--L--K--R--N--E--V--H--A--Q--R--Q--A- 220
  661 CGAATGGAAGAACTGGCAGCGCGGGTGGCCGAGCAGCAGGCTGCAGTGATGGCTGCAGGT 720
  221 -R--M--E--E--L--A--A--R--V--A--E--Q--Q--A--A--V--M--A--A--G- 240
  721 CTGCTCAGCAAGAAGGAGAGCGAGGACAGCAGCACGGTGATTGGACCAAGTATGCCGGAG 780
  241 -L--L--S--K--K--E--S--E--D--S--S--T--V--I--G--P--S--M--P--E- 260
  781 CCTGAACCCCCCCAAACTGAGAAAATGGAAACTACTACTGAAGACAAAAAAAAGGCAAAA 840
  261 -P--E--P--P--Q--T--E--K--M--E--T--T--T--E--D--K--K--K--A--K- 280
  841 ACAGAGAAGGTGAAGAAGTGTATCCGCACAGCAGCAGGGACGACCTGGGAGGACCAGAGT 900
  281 -T--E--K--V--K--K--C--I--R--T--A--A--G--T--T--W--E--D--Q--S- 300
  901 CTGCTGGAATGGGAATCAGACGACTTCCGTATTTTCTGTGGTGATCTTGGTAACGAGGTT 960
  301 -L--L--E--W--E--S--D--D--F--R--I--F--C--G--D--L--G--N--E--V- 320
  961 AATGATGACATACTGGCCAGAGCCTTCAGCAGATACCCATCTTTCCTCAAAGCTAAGGTG 1020
  321 -N--D--D--I--L--A--R--A--F--S--R--Y--P--S--F--L--K--A--K--V- 340
1021 GTCAGAGACAAACGGACTGGAAAAACCAAAGGCTACGGTTTTGTGAGCTTCAAAGATCCA 1080
  341 -V--R--D--K--R--T--G--K--T--K--G--Y--G--F--V--S--F--K--D--P- 360
1081 AATGATTACGTGAGAGCCATGAGAGAGATGAACGGGAAGTACGTTGGTAGCCGTCCCATC 1140
  361 -N--D--Y--V--R--A--M--R--E--M--N--G--K--Y--V--G--S--R--P--I- 380
1141 AAACTGAGGAAGAGCATGTGGAAGGACCGCAACATTGAAGTGGTTCGCAAGAAACAAAAA 1200
  381 -K--L--R--K--S--M--W--K--D--R--N--I--E--V--V--R--K--K--Q--K- 400
1201 GAGAAGAAGAAACTGGGCCTCAGATAG 1227
  401 -E--K--K--K--L--G--L--R--*- 408
(RBM42 mutant allele-7 nt deletion)
LENGTH: 1227 bp (−7 pb) and 178 aa
TYPE: cDNA (SEQ ID NO: 151) and Protein (SEQ ID NO: 153)
ORGANISM: Nile tilapia
SEQ ID NOs 151 and 153
      1 ATGGCGCTCAAGTCCGGCGAGGAGCGTCTGAAGGAGATGGAGGCTGAGATGGCGCTCTTT 60
      1 -M--A--L--K--S--G--E--E--R--L--K--E--M--E--A--E--M--A--L--F- 20
    61 GAGCAGGAGGTTCTCGGTGGTCCAGTACCAGTATCAGGAAGTCCACCTGTCATGGAGGCA 120
    21 -E--Q--E--V--L--G--G--P--V--P--V--S--G--S--P--P--V--M--E--A- 40
  121 GTACCTGTAGCTCTGGCTGTCCCAACAGTTCCAGTGGTGCGACCCATTATAGGAACCAAC 180
    41 -V--P--V--A--L--A--V--P--T--V--P--V--V--R--P--I--I--G--T--N- 60
  181 ACCTACAGACAGGTCCAGCAGACATTAGAAGCCAGAGCTGCAACTTTTGTTGGACCTCCA 240
    61 -T--Y--R--Q--V--Q--Q--I--L--E--A--R--A--A--T--F--V--G--P--P- 80
  241 CCACAAGCCTTTGTGGGACCAGTTCCTCCAGTACGTCCTCCTCCTCCCATGATGAGACCG 300
    81 -P--Q--A--F--V--G--P--V--P--P--V--R--P--P--P--P--M--M--R--P- 100
  301 GCTTTTGTTCCACATATTCTGCAAAGACCAGTGTTGTCAGGTGGTCAGAGGTTACAGATG 360
  101 -A--F--V--P--H--I--L--Q--R--P--V--L--S--G--G--Q--R--L--Q--M- 120
  361 ATGCGTGGTCCTCCAGTAGCACCTCCTTTGCCTCGACCTCCTCCACCTCCACCCATGATG 420
  121 -M--R--G--P--P--V--A--P--P--L--P--R--P--P--P--P--P--P--M--M- 140
  421 CTCCCTCCTTCCCTGCAGGGCCCAATGCCTCAGGGACCCTCTCAGCCCATCCAGCTGCTC 480
  141 -L--P--P--S--L--Q--G--P--M--P--Q--G--P--S--Q--P--I--Q--L--L- 160
  481 CACCTCAGGTTGGTGATATGGTTTCAATGGTGTCAGGCCCACCTACACGACAAGTAGCCT 540
  161 -H--L--R--L--V--I--W--F--Q--W--C--Q--A--H--L--H--D--K--*- 178
(wild-type TDRD6)
LENGTH: 4890 bp and 1630 aa
TYPE: cDNA (SEQ ID NO: 154) and Protein (SEQ ID NO: 156)
ORGANISM: Nile tilapia
SEQ ID NOs 154 and 156
      1 ATGTCATCAATCTTAGGACTCCCTACACGAGGATCAGATGTAACTGTTCTCATATCCAGG 60
      1 -M--S--S--I--L--G--L--P--T--R--G--S--D--V--T--V--L--I--S--R- 20
    61 GTCCACGTGCATCCCTTTTGTGTACTTGTGGAATTCTGGGGAAAATTTAGCCTGGAGAGG 120
    21 -V--H--V--H--P--F--C--V--L--V--E--F--W--G--K--F--S--L--E--R- 40
  121 ACTGCAGAGTATGAACGTCTAGCTAAAGACATTCAGTCCCCTGGGGACACTTTTCAAGAA 180
    41 -T--A--E--Y--E--R--L--A--K--D--I--Q--S--P--G--D--I--F--Q--E- 60
  181 CTGGAAGGAAAACCTGGTGACCAGTGCTTGGTTCAAATTGAGAGTATTTGGTATAGGGCT 240
    61 -L--E--G--K--P--G--D--Q--C--L--V--Q--I--E--S--I--W--Y--R--A- 80
  241 CGCATAGTCTCAAGTAATGGCTCGAAATACACAGTGTTTCTCATTGACAAAGGAACAACA 300
    81 -R--I--V--S--S--N--G--S--K--Y--T--V--F--L--I--D--K--G--T--T- 100
  301 TGCCGTGCCATCACAAGTAGGCTTGCATGGGGTAAAAAGGAGCATTTCCAACTGCCTCCT 360
  101 -C--R--A--I--T--S--R--L--A--W--G--K--K--E--H--F--Q--L--P--P- 120
  361 GAAGTGGAGTTTTGTGTGCTAGCTAACGTGCTACCACTGTCACTTGAGAACAAATGGTCC 420
  121 -E--V--E--F--C--V--L--A--N--V--L--P--L--S--L--E--N--K--W--S- 140
  421 CCAGTGGCTCTTGAATTTCTGAAATCTCTTCCTGGGAAGTGTGTGTCAGCACATGTGCAG 480
  141 -P--V--A--L--E--F--L--K--S--L--P--G--K--C--V--S--A--H--V--Q- 160
  481 GAAGTACTAGTCCTGAACAGAACATTCCTCCTGCACATACCTTGCATATCCAAACAAATG 540
  161 -E--V--L--V--L--N--R--I--F--L--L--H--I--P--C--I--S--K--Q--M- 180
  541 TATGAGATGGGATTTGCCAAGAAACTATCCCCAAACATCTTCCAGGACTTTGTCCTAAAG 600
  181 -Y--E--M--G--F--A--K--K--L--S--P--N--I--F--Q--D--F--V--L--K- 200
  601 TCAGTGCAGTCCCATAGTGGAGCTGAGGTTTCTCCAGAGATCAAACGGCTGTCCGTGGGA 660
  201 -S--V--Q--S--H--S--G--A--E--V--S--P--E--I--K--R--L--S--V--G- 220
  661 CCTGTTGAACAACTGCACAAGCAAGGGGTGTTCATGTACCCAGAGCTACAGGGAGGAACT 720
  221 -P--V--E--Q--L--H--K--Q--G--V--F--M--Y--P--E--L--Q--G--G--I- 240
  721 GTAGAGACTGTCGTTGTAACAGAAGTGACAAATCCACAGAGGATTTTTTGCCAGTTAAAG 780
  241 -V--E--T--V--V--V--T--E--V--T--N--P--Q--R--I--F--C--Q--L--K- 260
  781 GTCTTCTCTCAAGAGCTGAAGAAACTGTCTGATCAACTTACACAGAGTTGCGAAGGGAGA 840
  261 -V--F--S--Q--E--L--K--K--L--S--D--Q--L--T--Q--S--C--E--G--R- 280
  841 ATGCCCAATTGCATTATAGGCCCAGAAATGATTGGGTTTCCATGTTCTGCAAGGGGAAGT 900
  281 -M--P--N--C--I--I--G--P--E--M--I--G--F--P--C--S--A--R--G--S- 300
  901 GATGGCAAATGGTACCGCTCTGTTCTACAGCAGGTATTTCCAACCAGTAACATGGTGGAA 960
  301 -D--G--K--W--Y--R--S--V--L--Q--Q--V--F--P--T--S--N--M--V--E- 320
  961 GTATTGAATGTTGACAGTGGAACCAAAGAGTTTGTTAAAGTGGACAATGTAAGGTCACTG 1020
  321 -V--L--N--V--D--S--G--T--K--E--F--V--K--V--D--N--V--R--S--L- 340
1021 GCTGCAGAGTTCTTTAGGATGCCAGTTGTCACTTACATCTGCTCTCTCCATGGAGTTATT 1080
  341 -A--A--E--F--F--R--M--P--V--V--T--Y--I--C--S--L--H--G--V--I- 360
1081 GACAAAGGGGTAGGATGGACAACCACAAAAATTGACTACCTCAAGTCTCTCCTGCTGTAC 1140
  361 -D--K--G--V--G--W--T--T--T--K--I--D--Y--L--K--S--L--L--L--Y- 380
1141 AAGACGATGATTGCCAAATTTGAGTACCAAAGCATCTCAGAGGGTGTTCACTATGTCACT 1200
  381 -K--T--M--I--A--K--F--E--Y--Q--S--I--S--E--G--V--H--Y--V--T- 400
1201 CTTTATGGGGATGACAATACAAACATGAACATCTTGTTTGGTTCCAAACAGGGCTGTTTG 1260
  401 -L--Y--G--D--D--N--T--N--M--N--I--L--F--G--S--K--Q--G--C--L- 420
1261 CTGGACTGTGAAAAAACACTGGGAGATTATGCTATCCTCAACACAGCACACAGGCAACCG 1320
  421 -L--D--C--E--K--T--L--G--D--Y--A--I--L--N--T--A--H--R--Q--P- 440
1321 CATCCAGCCCAGCAAGAAAGAAAAATGCTAACTCCTGGAGAAGTTATGGAAGAAAAAGAA 1380
  441 -H--P--A--Q--Q--E--R--K--M--L--T--P--G--E--V--M--E--E--K--E- 460
1381 GGGAAAGCAGTTGCAGAGAGGGTGCCTGCTGAAGTTCTTCTGCTAAACTCTTCACATGTG 1440
  461 -G--K--A--V--A--E--R--V--P--A--E--V--L--L--L--N--S--S--H--V- 480
1441 GCAGTTGTTCAGCATGTAACAAACCCATCAGAGTTTTACATCCAAACGCAAAACTATACA 1500
  481 -A--V--V--Q--H--V--T--N--P--S--E--F--Y--I--Q--T--Q--N--Y--T- 500
1501 AAGCAGTTGAATGAATTAATGGATACTGTCTGCCAACTGTACAAAGATTCTGTGAATAAA 1560
  501 -K--Q--L--N--E--L--M--D--T--V--C--Q--L--Y--K--D--S--V--N--K- 520
1561 GGATCTGTTAGAATTCCAACTGTTGGACTCTACTGTGCAGCCAAAGCAGCAGATGGTGAT 1620
  521 -G--S--V--R--I--P--T--V--G--L--Y--C--A--A--K--A--A--D--G--D- 540
1621 TTCTACAGAGCAACTGTGACTAAAGTTGGTGAGACACAAGTCGAGGTATTCTTTGTTGAT 1680
  541 -F--Y--R--A--T--V--T--K--V--G--E--T--Q--V--E--V--F--F--V--D- 560
1681 TATGGAAATACAGAAGTGGTCGATAGGAGAAACCTCAGGATACTTCCTGCTGAGTTCAAA 1740
  561 -Y--G--N--T--E--V--V--D--R--R--N--L--R--I--L--P--A--E--F--K- 580
1741 AAGCTGCCACGGTTGGCACTAAAATGTACTCTGGCTGGTGTCAGACCTAAAGATGGGAGA 1800
  581 -K--L--P--R--L--A--L--K--C--T--L--A--G--V--R--P--K--D--G--R- 600
1801 TGGAGTCAGAGTGCCTCTGTCTTTTTCAGAAAAGCAGTAACCGATAAAGAACTAAAAGTC 1860
  601 -W--S--Q--S--A--S--V--F--F--R--K--A--V--T--D--K--E--L--K--V- 620
1861 CATGTACTGGCCAAATATGATAGTGGCTATGTTGTCCATCTGACAGATCCTAAAGCAGAG 1920
  621 -H--V--L--A--K--Y--D--S--G--Y--V--V--H--L--T--D--P--K--A--E- 640
1921 GGAGAACAACAAGTCAGTACACTGTTGTGTAATTCTGGTCTTGCTGAAAAGGCTGACAAA 1980
  641 -G--E--Q--Q--V--S--T--L--L--C--N--S--G--L--A--E--K--A--D--K- 660
1981 CCAGGGCAGTGCAAAAACACAATGCATCCTGCTATTACGCCTCCCACACAATATCCAGAT 2040
  661 -P--G--Q--C--K--N--T--M--H--P--A--I--T--P--P--T--Q--Y--P--D- 680
2041 GCCAGCCCACCATGTGGGAATAGGGACACTGGATTGGCTCTCCAGGTCCAAAACATAATT 2100
  681 -A--S--P--P--C--G--N--R--D--T--G--L--A--L--Q--V--Q--N--I--I- 700
2101 GGCCTTAGCCAGAAAGAAGGAAGAATGGCTACCTTTAAGGAACACATGTTTCCCATCGGA 2160
  701 -G--L--S--Q--K--E--G--R--M--A--T--F--K--E--H--M--F--P--I--G- 720
2161 AGTGTCCTTGATGTCAATGTGTCCTTCATTGAAAGCCCAAATGACTTTTGGTGCCAGCTA 2220
  721 -S--V--L--D--V--N--V--S--F--I--E--S--P--N--D--F--W--C--Q--L- 740
2221 GTTTATAATGCAGGACTCTTGAAATTGCTCATGGATGACATACAGGCACACTATGCAGGC 2280
  741 -V--Y--N--A--G--L--L--K--L--L--M--D--D--I--Q--A--H--Y--A--G- 760
2281 AGTGAGTTTCAGCCAAATGTCGAAATGGCTTGTGTTGCTCGTCACCCTGGTAACGGATTG 2340
  761 -S--E--F--Q--P--N--V--E--M--A--C--V--A--R--H--P--G--N--G--L- 780
2341 TGGTACAGGGCCCTTGTGATTCATAAACATGAAACTCATGTGGATGTGTTGTTTGTTGAC 2400
  781 -W--Y--R--A--L--V--I--H--K--H--E--T--H--V--D--V--L--F--V--D- 800
2401 TATGGCCAGACAGAGACAGTCTCCTTCCAAGACCTGAGGAGAATCAGCCCAGAATTTCTT 2460
  801 -Y--G--Q--T--E--T--V--S--F--Q--D--L--R--R--I--S--P--E--F--L- 820
2461 ACTCTGCATGGTCAGGCTTTTCGATGCAGTCTGTTAAATCCCATTGACCCTACATCTGCT 2520
  821 -T--L--H--G--Q--A--F--R--C--S--L--L--N--P--I--D--P--T--S--A- 840
2521 GTAACTGAGTGGAGCGAAGAGGCAGTAGAAAGGTTTAAAAACTTTGTGGACTCGGCTGCT 2580
  841 -V--T--E--W--S--E--E--A--V--E--R--F--K--N--F--V--D--S--A--A- 860
2581 TCCAACTTTGTGATTCTGAAATGCACCATATATGCTGTCATGTGCAGTGAGCAGAAGATT 2640
  861 -S--N--F--V--I--L--K--C--T--I--Y--A--V--M--C--S--E--Q--K--I- 880
2641 GTTTTCAACATTGTGGATCTAGAAACTCCATTTGAGAGTATTTGCACTAGTGTGGTAAAT 2700
  881 -V--F--N--I--V--D--L--E--T--P--F--E--S--I--C--T--S--V--V--N- 900
2701 GTCATGAAAAGTACACCTCCCAAAAAAGCTACAGGAGCATCTTTTCGTCTGGATACATAC 2760
  901 -V--M--K--S--T--P--P--K--K--A--T--G--A--S--F--R--L--D--T--Y- 920
2761 TATTACTCAACACACAATGTCAAAACTGGGATGGAGGAAGAGGTCACAGTGACATGTGTG 2820
  921 -Y--Y--S--T--H--N--V--K--T--G--M--E--E--E--V--T--V--T--C--V- 940
2821 AACAATGTCAGTCAGTTCTACTGCCAGCTTGAAAAGAATGCTGATGTGATAAATGACCTC 2880
  941 -N--N--V--S--Q--F--Y--C--Q--L--E--K--N--A--D--V--I--N--D--L- 960
2881 AAGATGAAAGTGAGCAGTTTTTGTCGTCAGCTTGAGAATGTAAAGCTTCCAGCAGTCTTT 2940
  961 -K--M--K--V--S--S--F--C--R--Q--L--E--N--V--K--L--P--A--V--F- 980
2941 GGAACTCTGTGCTTTGCAAGATATACTGATGGGCAGTGGTACAGAGGGCAGATCAAGGCC 3000
  981 -G--T--L--C--F--A--R--Y--T--D--G--Q--W--Y--R--G--Q--I--K--A- 1000
3001 ACAAAGCCAGCACTCCTGGTTCACTTTGTGGATTACGGGGACACTATTGAAGTAGATAAA 3060
1001 -T--K--P--A--L--L--V--H--F--V--D--Y--G--D--T--I--E--V--D--K- 1020
3061 TCTGACTTGCTCCCAGTTCCCAGAGAGGCAAATGACATCATGTCTGTGCCTGTGCAAGCA 3120
1021 -S--D--L--L--P--V--P--R--E--A--N--D--I--M--S--V--P--V--Q--A- 1040
3121 GTAGTGTGTGGTCTTTCTGATGTTCCTGCTAATGTTTCCAGTGAGGTGAACAGCTGGTTT 3180
1041 -V--V--C--G--L--S--D--V--P--A--N--V--S--S--E--V--N--S--W--F- 1060
3181 GAGACAACTGCAACAGAATGCAAATTCCGGGCGCTAGTAGTAGCCAGAGAACCTGATGGG 3240
1061 -E--T--T--A--T--E--C--K--F--R--A--L--V--V--A--R--E--P--D--G- 1080
3241 AAAGTCCTAGTTGAGCTCTATCTTGGAAACACTCAGATCAATTCAAAGCTCAAGAAAAAG 3300
1081 -K--V--L--V--E--L--Y--L--G--N--T--Q--I--N--S--K--L--K--K--K- 1100
3301 TTTCATATTGAGATGCACACAGAAAGCCAGGTTGTCTGCCATGGTTGGAGAGCTTTTGAG 3360
1101 -F--H--I--E--M--H--T--E--S--Q--V--V--C--H--G--W--R--A--F--E- 1120
3361 GCTTCACCGAGTTATTCGCAAAAGACAAAATGCACCACAAAAATGGAAGGGGATGATGGG 3420
1121 -A--S--P--S--Y--S--Q--K--T--K--C--T--T--K--M--E--G--D--D--G- 1140
3421 AAATCTAACGAAATGAACCTTTGGAACAAAACAACAAAGTCAGTTCATGAAAATGGTCAA 3480
1141 -K--S--N--E--M--N--L--W--N--K--T--T--K--S--V--H--E--N--G--Q- 1160
3481 AGGATCAAGAGTCCGCGACTAGAGTTGTACACACCTCCACAGCAAAGGGAGTCATCTGCT 3540
1161 -R--I--K--S--P--R--L--E--L--Y--T--P--P--Q--Q--R--E--S--S--A- 1180
3541 GGTGGCAATGTCAGATCTTCAGATCTTCCAACAGATGCCAAGAAACTCAAGTCAACAGTA 3600
1181 -G--G--N--V--R--S--S--D--L--P--T--D--A--K--K--L--K--S--T--V- 1200
3601 AATGGCACAGAATCCCAAAAGGAAAGTAATGCTGAAAAGCTTCCTAAACTTTCAGACTTG 3660
1201 -N--G--T--E--S--Q--K--E--S--N--A--E--K--L--P--K--L--S--D--L- 1220
3661 CCCTCAAATTTTATCACACCTGGTATGGTAGCAGATGTCTACGTGTCACATTGCAACAGC 3720
1221 -P--S--N--F--I--T--P--G--M--V--A--D--V--Y--V--S--H--C--N--S- 1240
3721 CCAGTAAGTTTCCACGTGCAGTGTGTAAGCGATGAGGATCATATATATTCCCTGGTAGAA 3780
1241 -P--V--S--F--H--V--Q--C--V--S--D--E--D--H--I--Y--S--L--V--E- 1260
3781 AAGCTCAATGACCCCAGTTCAACTGCAGAAACCAACGGGCTCAAAGATGTGCGTCCAGAT 3840
1261 -K--L--N--D--P--S--S--T--A--E--T--N--G--L--K--D--V--R--P--D- 1280
3841 GACCTTGTTCAAGCACAGTTCACAGATGATTCCTCATGGTACCGAGCAGTTGTAAGAGAA 3900
1281 -D--L--V--Q--A--Q--F--T--D--D--S--S--W--Y--R--A--V--V--R--E- 1300
3901 CTTCACGGTGATGCAATGGCTCTCATTGAGTTTGTTGATTTTGGCAATACAGCCATGACT 3960
1301 -L--H--G--D--A--M--A--L--I--E--F--V--D--F--G--N--T--A--M--T- 1320
3961 CCACTTTCCAAGATGGGCAGACTCCACAAGAATTTCTTGCAGCTGCCGATGTACAGCACA 4020
1321 -P--L--S--K--M--G--R--L--H--K--N--F--L--Q--L--P--M--Y--S--T- 1340
4021 CACTGTATGCTGAGTGATGCTGCTGGTCTTGGGGAAGAGGTTGTAGTTGATCCAGATGTG 4080
1341 -H--C--M--L--S--D--A--A--G--L--G--E--E--V--V--V--D--P--D--V- 1360
4081 GTGTCAACTTTCAAAGAAAAGATTTCTAGTAGTGGAGAAAAAGTGTTCAAGTGCCAGTTT 4140
1361 -V--S--T--F--K--E--K--I--S--S--S--G--E--K--V--F--K--C--Q--F- 1380
4141 GTCAGGAAGATTGGGTCTGTGTGGGAAGTTAACCTTGAAGACAATGGTGTGAAGGTTACG 4200
1381 -V--R--K--I--G--S--V--W--E--V--N--L--E--D--N--G--V--K--V--T- 1400
4201 TATAAAGTGCCTACTGCAGATCCTGAAATCACTTCAGAGAAACTTGAGCAAGTAAAGGAA 4260
1401 -Y--K--V--P--T--A--D--P--E--I--T--S--E--K--L--E--Q--V--K--E- 1420
4261 GAGCCATCCCAGGTGTCTGATATCAGAGAAGTGCCAGAGAGATCAGTGCTGAGCCACTGC 4320
1421 -E--P--S--Q--V--S--D--I--R--E--V--P--E--R--S--V--L--S--H--C- 1440
4321 TCCCCACATAACTTTCTAGAAGACCTTTTTGAGGGGCATAAATTGGAAGCCTATGTCACA 4380
1441 -S--P--H--N--F--L--E--D--L--F--E--G--H--K--L--E--A--Y--V--T- 1460
4381 GTTATAAATGATGATCAGACTTTCTGGTGTCAGTCTGCTAGTTCAGAAGAACATGATGAG 4440
1461 -V--I--N--D--D--Q--T--F--W--C--Q--S--A--S--S--E--E--H--D--E- 1480
4441 ATCTTATTAGGTCTCTCAGAAGTTGAGAATTCAACAGGTCAGAACTATATTGATCCAGAT 4500
1481 -I--L--L--G--L--S--E--V--E--N--S--T--G--Q--N--Y--I--D--P--D- 1500
4501 GCTCTCGTTCCTGGAAGTCTATGTGTTGCTCGCTTTTTAGATGATGAGTTTTGGTATCGT 4560
1501 -A--L--V--P--G--S--L--C--V--A--R--F--L--D--D--E--F--W--Y--R- 1520
4561 GCAGAGGTCATTGACAAAAATGAGGGTGAGCTCTCTGTTTTCTTTTTGGACTATGGAAAC 4620
1521 -A--E--V--I--D--K--N--E--G--E--L--S--V--F--F--L--D--Y--G--N- 1540
4621 AAGGCTAGAGTCAGCATAACAGATGTGAGAGAAATGCCACCTTGCTTGTTGAAGATTCCA 4680
1541 -K--A--R--V--S--I--T--D--V--R--E--M--P--P--C--L--L--K--I--P- 1560
4681 CCACAGGCATTTTTGTGTGAGCTTGAAGGCTTTGATGCTTTATGTGGATATTGGGAAAGT 4740
1561 -P--Q--A--F--L--C--E--L--E--G--F--D--A--L--C--G--Y--W--E--S- 1580
4741 GGAGCAAAGGTTGAATTGTCTGCACTTATAGATGTCAAACTGTTGCAGTTGACTGTCACA 4800
1581 -G--A--K--V--E--L--S--A--L--I--D--V--K--L--L--Q--L--T--V--T- 1600
4801 AAACTAGCAAGAGCTACAGGAACAATCTTTGTGCAGGTGGAATGCGAAGGTCAGGTGATC 4860
1601 -K--L--A--R--A--T--G--T--I--F--V--Q--V--E--C--E--G--Q--V--I- 1620
4861 AACGAGTTGATGAAAACCTGGTGGAAGAGC 4890
1621 -N--E--L--M--K--T--W--W--K--S- 1630
(TDRD6 mutant allele-10 nt deletion)
LENGTH: 4890 bp (−10 bp) and 43 aa
TYPE: cDNA (SEQ ID NO: 154) and Protein (SEQ ID NO: 156)
ORGANISM: Nile tilapia
SEQ ID NOs 155 and 157
      1 ATGTCATCAATCTTAGGACTCCCTACACGAGGATCAGATGTAACTGTTCTCATATCCAGG 60
      1 -M--S--S--I--L--G--L--P--T--R--G--S--D--V--T--V--L--I--S--R- 20
    61 GTCCACGTGCATCCCTTTTGTGTACTTGTGGAAAATTTAGCCTGGAGAGGACTGCAGAGT 120
    21 -V--H--V--H--P--F--C--V--L--V--E--N--L--A--W--R--G--L--Q--S- 40
  121 ATGAACGTCTAGCTAAAGACATTCAGTCCCCTGGGGACACTTTTCAAGAACTGGAAGGAA 180
    41 -M--N--V--*- 43
(wild-type Hook2)
LENGTH: 4002 bp and 708 aa
TYPE: cDNA (SEQ ID NO: 158) and Protein (SEQ ID NO: 160)
ORGANISM: Nile tilapia
SEQ ID NOs 158 and 160
      1 GCATAATCCATCGCCTTGGAAACGCTCTAATACGGAAGCTCGCGAGGCCCATAGGAGCCG 60
          ............................................................
    61 AAACGCGAAGGTTGTCAGGAGCAGCAGGAGGAGGCCACGGCTGGACAGTGTCTGACGTGG 120
          ............................................................
  121 AAAGTGTCAGCACTGAGTAAGAAACTTCGGGCCAAAACAAGCCTCGAGAACAAAATCCCC 180
          ............................................................
  181 ACAGTTCTCTGTAAGCTCCTGCGAGTTTCACAGAGGACAGCACAATGAGTCTGGATAAGG 240
          ............................................-M--S--L--D--K-- 5
  241 CGAAGCTGTGTGATTCACTGTTAACCTGGTTACAGACATTTCAGGTGCCATCGTGCAACA 300
      6 A--K--L--C--D--S--L--L--T--W--L--Q--T--F--Q--V--P--S--C--N-- 25
  301 GCAAGCATGACCTGACAAGCGGAGTGGCCATTGCACACGTACTGCACAGAATAGACCCTT 360
    26 S--K--H--D--L--T--S--G--V--A--I--A--H--V--L--H--R--I--D--P-- 45
  361 CTTGGTTTAATGAGACATGGCTAGGCAGGATCAAGGAGGAGAGCGGGGCCAACTGGCGCC 420
    46 S--W--F--N--E--T--W--L--G--R--I--K--E--E--S--G--A--N--W--R-- 65
  421 TCAAGGTCAGCAACTTGAAAAAGATTCTGAAAAGCATGATGGAATATTATCACGATGTGC 480
    66 L--K--V--S--N--L--K--K--I--L--K--S--M--M--E--Y--Y--H--D--V-- 85
  481 TCGGTCACCAGGTGTCTGATGAGCATATGCCAGACGTGAACCTGATAGGAGAGATGGGAG 540
    86 L--G--H--Q--V--S--D--E--H--M--P--D--V--N--L--I--G--E--M--G-- 105
  541 ATGTCACAGAACTGGGAAAGCTGGTACAGCTCGTGTTGGGTTGTGCAGTCAGCTGCGAGA 600
  106 D--V--T--E--L--G--K--L--V--Q--L--V--L--G--C--A--V--S--C--E-- 125
  601 AGAAACAAGAGCAAATCCAGCAGATAATGACACTCGAGGAATCTGTCCAGCATGTTGTGA 660
  126 K--K--Q--E--Q--I--Q--Q--I--M--T--L--E--E--S--V--Q--H--V--V-- 145
  661 TGACTGCCATTCAGGAACTCTTATCAAAGGAGCCGTCATCTGAACCGGGAAGCCCAGAGA 720
  146 M--T--A--I--Q--E--L--L--S--K--E--P--S--S--E--P--G--S--P--E-- 165
  721 CCTACGGGGATTTTGACTATCAGTCCAGGAAGTATTATTTTCTGAGTGAGGAGGCAGACG 780
  166 T--Y--G--D--F--D--Y--Q--S--R--K--Y--Y--F--L--S--E--E--A--D-- 185
  781 AGAAGGACGACCTGAGCCAGCGCTGTCGAGACCTTGAACATCAGCTGTCAGTGGCTCTGG 840
  186 E--K--D--D--L--S--Q--R--C--R--D--L--E--H--Q--L--S--V--A--L-- 205
  841 AGGAGAAGATGTCCCTGCAGGCAGAGACACGCTCCCTGAAAGAGAAGCTCAGCCTCAGCG 900
  206 E--E--K--M--S--L--Q--A--E--T--R--S--L--K--E--K--L--S--L--S-- 225
  901 AATCTCTGGATGCCTCCACCACTGCCATCACTGGCAAGAAGCTGCTGCTGCTGCAGAGTC 960
  226 E--S--L--D--A--S--T--T--A--I--T--G--K--K--L--L--L--L--Q--S-- 245
  961 AGATGGAGCAGCTTCAGGAGGAAAACTACAGACTGGAGAACGGCAGAGACGACATGCGTG 1020
  246 Q--M--E--Q--L--Q--E--E--N--Y--R--L--E--N--G--R--D--D--M--R-- 265
1021 TGCGGGCAGAGATACTGGAGCGCGAGGTGGCCGAGCTGCAACTACGGAACGAAGAGCTGA 1080
  266 V--R--A--E--I--L--E--R--E--V--A--E--L--Q--L--R--N--E--E--L-- 285
1081 CCAGCCTGGCGCAGGAGGCCCAGGCCCTCAAAGATGAGATGGACATCCTCAGGCACTCGT 1140
  286 T--S--L--A--Q--E--A--Q--A--L--K--D--E--M--D--I--L--R--H--S-- 305
1141 CTGACCGGGTGAACCAGCTCGAGGCGATGGTGGAGACGTACAAGAGGAAGCTGGAGGACC 1200
  306 S--D--R--V--N--Q--L--E--A--M--V--E--T--Y--K--R--K--L--E--D-- 325
1201 TGGGAGACCTGCGCAGGCAGGTGCGCCTCCTGGAAGAGCGCAACACCGTGTACATGCAGC 1260
  326 L--G--D--L--R--R--Q--V--R--L--L--E--E--R--N--T--V--Y--M--Q-- 345
1261 GCACGTGCGAGCTGGAGGAGGAGCTTCGCAGGGCCAACGCTGTCCGCAGTCAGCTGGACA 1320
  346 R--T--C--E--L--E--E--E--L--R--R--A--N--A--V--R--S--Q--L--D-- 365
1321 CCTACAAGAGACAGGCTCATGAGCTTCACACCAAGCACTCAGCAGAGGCCATGAAAGCTG 1380
  366 T--Y--K--R--Q--A--H--E--L--H--T--K--H--S--A--E--A--M--K--A-- 385
1381 AGAAGTGGCAGTTTGAGTACAAGAACCTTCACGACAAGTACGACGCACTGCTGAAGGAGA 1440
  386 E--K--W--Q--F--E--Y--K--N--L--H--D--K--Y--D--A--L--L--K--E-- 405
1441 AAGAACGTCTGATCGCAGAAAGAGACACACTGCGGGAGACAAACGATGAGCTCAGGTGTG 1500
  406 K--E--R--L--I--A--E--R--D--T--L--R--E--T--N--D--E--L--R--C-- 425
1501 CACAAGTCCAGCAGAGGTATCTCAGTGGAGCAGGAGGCTTGTGTGACAGCGGTGACACGG 1560
  426 A--Q--V--Q--Q--R--Y--L--S--G--A--G--G--L--C--D--S--G--D--T-- 445
1561 TTGAAAACCTGGCTGCAGAGATCATGCCAACTGAGATCAAGGAGACAGTTGTTCGCCTCC 1620
  446 V--E--N--L--A--A--E--I--M--P--T--E--I--K--E--T--V--V--R--L-- 465
1621 AAAGTGAAAACAAGATGCTGTGCGTCCAGGAGGAGACCTACCGACAGAAACTTGTGGAAG 1680
  466 Q--S--E--N--K--M--L--C--V--Q--E--E--T--Y--R--Q--K--L--V--E-- 485
1681 TTCAGGCTGAGCTGGAGGAGGCTCAACGCAGCAAGAATGGGCTAGAAACTCAGAACAGGC 1740
  486 V--Q--A--E--L--E--E--A--Q--R--S--K--N--G--L--E--T--Q--N--R-- 505
1741 TGAACCAGCAGCAGATCTCAGAGCTGCGTTCTCAGGTCGAGGAGCTCCAGAAAGCACTCC 1800
  506 L--N--Q--Q--Q--I--S--E--L--R--S--Q--V--E--E--L--Q--K--A--L-- 525
1801 AGGAGCAGGACAGCAAGAACGAGGACTCGTCCTTATTGAAGAAAAAGCTTGAGGAGCACC 1860
  526 Q--E--Q--D--S--K--N--E--D--S--S--L--L--K--K--K--L--E--E--H-- 545
1861 TGGAGAAGCTCCACGAGGCCCAGTCAGACCTGCAAAAGAAAAAAGAGGTCATTGACGACC 1920
  546 L--E--K--L--H--E--A--Q--S--D--L--Q--K--K--K--E--V--I--D--D-- 565
1921 TAGAGCCCAAAGTGGACAGCAACATGGCCAAGAAGATTGATGAACTCCAGGAGATCCTGC 1980
  566 L--E--P--K--V--D--S--N--M--A--K--K--I--D--E--L--Q--E--I--L-- 585
1981 GGAAGAAGGACGAGGACATGAAGCAGATGGAGCAGCGATACAAACGCTACGTGGAGAAGG 2040
  586 R--K--K--D--E--D--M--K--Q--M--E--Q--R--Y--K--R--Y--V--E--K-- 605
2041 CGAGAACGGTGATCAAAACCCTGGATCCTAAGCAGCAGCCAGTGACTCCTGACGTTCAGG 2100
  606 A--R--T--V--I--K--T--L--D--P--K--Q--Q--P--V--T--P--D--V--Q-- 625
2101 CCCTGAAAAACCAGCTGACAGAGAAGGAGAGAAGAATCCAGCATCTGGAGCATGATTATG 2160
  626 A--L--K--N--Q--L--T--E--K--E--R--R--I--Q--H--L--E--H--D--Y-- 645
2161 AGAAGAGCAGGGCCAGACACGACCAGGAGGAGAAACTCATCATCGGTGCCTGGTACAAGA 2220
  646 E--K--S--R--A--R--H--D--Q--E--E--K--L--I--I--G--A--W--Y--K-- 665
2221 TGGGAATGGCACTGCATCAGAAAGTGTCTGGTGAGCGGCTGGGTTCCTCCAACCAGGCCA 2280
  666 M--G--M--A--L--H--Q--K--V--S--G--E--R--L--G--S--S--N--Q--A-- 685
2281 TGTCCTTCCTCGCCCAGCAGAGACAACTAATCAACGCAAGGAGGGGCCTGACACGACACC 2340
  686 M--S--F--L--A--Q--Q--R--Q--L--I--N--A--R--R--G--L--T--R--H-- 705
2341 ACCCGAGATGAGACACTGAGGCGTGACAGTTACCCTCAAATGAAAAGCAAAGTGCACACA 2400
  706 H--P--R--*-................................................. 708
2401 AGGTGATCCATGTGAACTCTGAGTGTCTTTTGCCTTTTTATGCCTTCACTGGGATCACTG 2460
          ............................................................
2461 CGCCTCAGTGTTTGTCACGCTGCTGCTGCCCCCTGCTGGCTCTTACTGATATGAGAAGAT 2520
          ............................................................
2521 TTCTTTTCTCCGTTGGGCTCCAGAGCAGAAGCTCTCTGCTCTGTTAAAAAGTAGGAGTTA 2580
          ............................................................
2581 TAGGCCTTAAGAAGAGGCAACCTCACCTTTTAAGGTGACTTTTATTTCCCCTGTAGCCTC 2640
          ............................................................
2641 TTGGACTCACTAGTTTTTTTTTTTGTTTTTTTTTCTTGAACATTTATTTAAAATCCTTTT 2700
          ............................................................
2701 TTTTAATTTTTTTATGTTACACAGTGAAACAGAACTGGAACAAGTTTTGTCAGGTGCCAG 2760
          ............................................................
2761 TTTAAATGTGATAGATGATGGAGAAGTTTCACTACTCCGAGTGCTACAGAACAAAAGCTG 2820
          ............................................................
2821 CACAAGCTGTCCTCATACCTCCACTACAGATCGTCACGTTAACTACATCTTGGGTTTTAT 2880
          ............................................................
2881 GTGTTTGCTGATGATTTTTCTTCTTGTAGCGTTTTATTTTTAGTTTAAGTTTGAAGTACC 2940
          ............................................................
2941 TTTGGAAAAAAATGTAAAAAATCAAGCGGGTGTGTAACAGCTTTAGTCTAGATTTCTTCT 3000
          ............................................................
3001 GTATTCACTTTGAATGCTTCCCTTTTTTTTTTCCTTCTCAGCCTTAAATCTGAAACATGT 3060
          ............................................................
3061 CTTCTGTAAATATTTTCACAATGTACACCAAAGCACTTTCTCTCTAGAAGGGTGGGTTTG 3120
          ............................................................
3121 TTCAGTGCCTCGCAAAAGTCTCCCATGCTAGCCCTTTATTAGATGAGACTGAACACTGAC 3180
          ............................................................
3181 ATGTTTGCAGCGCCAACACTGTTTCTGTCACACTACAGGTACGTGCCCGTGTCTGGTGAT 3240
          ............................................................
3241 ATGACTTTTGTGTAATTTTTTTCTCTCTGTTGCCTCTAAAAAAAATTTTTATTTTTTTTA 3300
          ............................................................
3301 ATTCCTATCCATAAGACCTCCCCCATCAGGGGGTCTGATTGTGGGTCGGACCTAACTGCA 3360
          ............................................................
3361 CTCTCCACTTTAAACACAAAAACTGGAAAACACTATGCGAGAGTCTCTAATCATAAAAAC 3420
          ............................................................
3421 ACTAAAAAATATATAAAACTGAGTCAGCTGATGTCCTGTTTGCTGCTGCTAGGTGTTGTT 3480
          ............................................................
3481 CACGGCTGAGCTGGAAGGAAACGTGTTCTTCAATGCGCTGGAATTTTTCCTGTGACAGGA 3540
          ............................................................
3541 AATCGACAGCAGATTAAAAAGCCTGAGGCACATTTATCAGACACTACGTCTGCCTTTCTT 3600
          ............................................................
3601 TACAACCGCTGATCAAGTTGTTTTTGTGCGTCCCATATCAGAGCCGCTGTCCTGTGACTT 3660
          ............................................................
3661 GTACTTGCCTCTAACAGTTTGTGCTATGATCTACGAAGACCAGAGTCCTGCGGTTGTGTA 3720
          ............................................................
3721 AACACTTTTTATTTTTTTGTTCTACTGATGTTTTTTTTTTTCTTTTAAGTTGGTTTTTAT 3780
          ............................................................
3781 GGCGTAAAATTATTGCTCCACATATGCATGGTATGAAAGGTTGCATCATGAAATGGTCTA 3840
          ............................................................
3841 CTAGATTATACCATAATGTACTTGACACAGGGTTATATTATTTGTAGTCCTCTGTTCTAC 3900
          ............................................................
3901 TTTTTGCACTACAAATAAATGGGATCTTAAGTTAAAGATGGCATTTTGTGTTCTTCTTTT 3960
          ............................................................
3961 CAGTGCATTCAAAGGCACACTTTCACAGTCCCTTCTGATTTA 4002
          ..........................................
(Hook2 mutant allele-2 nt deletion)
LENGTH: 4002 bp (−2 pb) and 158 aa
TYPE: cDNA (SEQ ID NO: 159) and Protein (SEQ ID NO: 161)
ORGANISM: Nile tilapia
SEQ ID NOs 159 and 161
      1 GCATAATCCATCGCCTTGGAAACGCTCTAATACGGAAGCTCGCGAGGCCCATAGGAGCCG 60
          ............................................................
    61 AAACGCGAAGGTTGTCAGGAGCAGCAGGAGGAGGCCACGGCTGGACAGTGTCTGACGTGG 120
          ............................................................
  121 AAAGTGTCAGCACTGAGTAAGAAACTTCGGGCCAAAACAAGCCTCGAGAACAAAATCCCC 180
          ............................................................
  181 ACAGTTCTCTGTAAGCTCCTGCGAGTTTCACAGAGGACAGCACAATGAGTCTGGATAAGG 240
          ............................................-M--S--L--D--K-- 5
  241 CGAAGCTGTGTGATTCACTGTTAACCTGGTTACAGACATTTCAGGTGCCATCGTGCAACA 300
      6 A--K--L--C--D--S--L--L--T--W--L--Q--T--F--Q--V--P--S--C--N-- 25
  301 GCAAGCATGACCTGACAAGCGGAGTGGCCATTGCACACGTACTGCACAGAATAGACCCTT 360
    26 S--K--H--D--L--T--S--G--V--A--I--A--H--V--L--H--R--I--D--P-- 45
  361 CTTGGTTTAATGAGACATGGCTAGGCAGGATCAAGGAGGAGAGCGGGGCCAACTGGCGCC 420
    46 S--W--F--N--E--T--W--L--G--R--I--K--E--E--S--G--A--N--W--R-- 65
  421 TCAAGGTCAGCAACTTGAAAAAGATTCTGAAAAGCATGATGGAATATTATCACGATGTGC 480
    66 L--K--V--S--N--L--K--K--I--L--K--S--M--M--E--Y--Y--H--D--V-- 85
  481 TCGGTCACCAGGTGTCTGATGAGCATATGCCAGACGTGAACCTGATAGGAGATGGGAGAT 540
    86 L--G--H--Q--V--S--D--E--H--M--P--D--V--N--L--I--G--D--G--R-- 105
  541 GTCACAGAACTGGGAAAGCTGGTACAGCTCGTGTTGGGTTGTGCAGTCAGCTGCGAGAAG 600
  106 C--H--R--T--G--K--A--G--T--A--R--V--G--L--C--S--Q--L--R--E-- 125
  601 AAACAAGAGCAAATCCAGCAGATAATGACACTCGAGGAATCTGTCCAGCATGTTGTGATG 660
  126 E--T--R--A--N--P--A--D--N--D--T--R--G--I--C--P--A--C--C--D-- 145
  661 ACTGCCATTCAGGAACTCTTATCAAAGGAGCCGTCATCTGAACCGGGAAGCCCAGAGACC 720
  146 D--C--H--S--G--T--L--I--K--G--A--V--I--* 158
(nanos3 3′UTR)
LENGTH: 703 bp
TYPE: cDNA non-coding
ORGANISM: Japanese flounder (Paralichthys olivaceus)
SEQ ID NO 166
      1 AGCCAACAGGTGTCAGGTATATCGACAACAAGCCACTGCACAGAGGCCGCAGTTCTTTTT 60
          ............................................................
    61 ATGTGTGATTTTTATTTTAATAGCACTAGTGTTGTTTTTTGCTTTTGTGTGGTTTTTGGT 120
          ............................................................
  121 TTGGTTTTAATTTGCATGCTTTGGCACGTTTACACTGAGGCCTTCTGTGAAGCTGGCTGA 180
          ............................................................
  181 TCTTTCTGTGGGCCCTCTACTTCAAAAAGCGTCTGTTGGTGGATTTCGTGAGGTACTCTC 240
          ............................................................
  241 TTTCGACAACGACTGCCAGATATGTTTGGGAGGAGAAAAGGAAAAAATGTTTCTCAGGAA 300
          ............................................................
  301 ATTGTATGTTTGTTTTATTTATATTTTAAACGTGGCCATCTGATGTCCAGCCTCACTTTT 360
          ............................................................
  361 CCTGTCCATGCATTGAAGGATTTCAACACAAATACCAAAGCTTTATCAGACCTACATTCA 420
          ............................................................
  421 TCATTGGTAATAATTTTACTACAGCATTTAAACATCATGTGACCATGTCAGTATTTTAAA 480
          ............................................................
  481 TTTTTAAAATATCAGTGACTTGTTCTAGTTCTAAGGTGTGTGAGTGAATTCCTGTTCCTG 540
          ............................................................
  541 AGACATCCTGTTTTATTTTGAATATTCTATGTGTGGCTTTTCTAAAGGTAAAAAAAAAAA 600
          ............................................................
  601 AGCCGCTGTAACTCATCTGGCTTTGTGGGGGGGGGGAATCTTTGTGTGAATATTCTTGTA 660
          ............................................................
  661 GTTACACATGTCTAAAGTGAGTAAATCTGTGTTTGTATGCTTT 703
(nanos3 3′UTR)
LENGTH: 567 bp
TYPE: cDNA non-coding
ORGANISM: Common Carp (Cyprinus Carpio)
SEQ ID NO 167
      l ACCGGACGTTTCTGGCCACGGTCATACAAGAAGGACGTTTTTACGAGTAGTTTTAATATT 60
          ............................................................
    61 CCAGTTTTAATTGTTCAATCCATAATGGCTTGTGTGTAAGTTTGCATGCATGTGTGCTTT 120
          ............................................................
  121 TTGGTGTTGTTTGATTTTGCACGGTTTTTTGTCTTCCTCTTGTGTGCAGTGGTGTTTTTC 180
          ............................................................
  181 ACTCTAACAAACTTGTACACAAGCCAGTTGGCTTGCTACAGGTGCAACCACGTGTGAACT 240
          ............................................................
  241 AGCGCTTTCTTGTTAATTTTACTAAAAAAAAAAGTATCTTGTGATTAATCTGTGGTGAAA 300
          ............................................................
  301 TATATATAAACGCTTTTAGTGTTATTTACATGTGTTTCTCTTTAAAGCTGCCTATATTTT 360
          ............................................................
  361 GCATTAACACTTAAAAAAATCTCAGTCTTCTGTTTTATTTCTTTTTACAACATTTTGAAA 420
          ............................................................
  421 ACATTATCAGGTTTTGTTCACGTGACATCAGGAAGTTCATGTATATTTGTTTAAAAATGA 480
          ............................................................
  481 TTTACCTTGGGACAAAAACAAGAAAATGAACAGAACTTTTGGAACCCTGTGTTCATATCA 540
          ............................................................
  541 CAGCACTTAAGCTGAAATTGGTTCAGT 567
          ...........................
(nanos3 3′UTR)
LENGTH: 618 bp
TYPE: cDNA non-coding
ORGANISM: Zebrafish (Danio Rerio)
SEQ ID NO 168
      1 AGCGGACATTGATGCTCCGGTAGATTTGAAGAAACACTTTTTACCGCAGGTTTTAATGTT 60
          ............................................................
    61 TAAGTTTTAACTCTTTAATTGTTTGTTTGGTTGATACGCGGCGGATTGCGAGTTTGCATG 120
          ............................................................
  121 CATGTGTGCGTTCACTGTTTGATTTTGCACTTTTTTTGTGTGTGTGTATATGTGTGTGTT 180
          ............................................................
  181 TGCTGTGTTTTATTTTGTGTGCACTGGTGTTGTGTTTTCACTTGGTAACAAACTTGTACA 240
          ............................................................
  241 CAAGCCAGCAGGCTCGCTACAGGCGCAACCGCACTCAAAAACAAACCCTTTCATGCTTAT 300
          ............................................................
  301 TTGGTAAATACAATGTGTGTTTAGTCCTCCTTTTAAATGTCAGATTTTATGGTGTTGTAT 360
          ............................................................
  361 TTAAACAAAAAATTCAATGTTAATATTTAGATTTTAGTGATTTTATTATTGAAAACGGCT 420
          ............................................................
  421 TGTTTTGTATAAGTAACCTTTAAAAAAAGTTTTCTCCATTGCATTTAAATTCAGTTTGAA 480
          ............................................................
  481 AAACATAATCGCCATATTTTCATGTCGCTTGCTAAAATTCATGTACTACTTTCATCATTT 540
          ............................................................
  541 TATGTCAGTGTGTGATTTTTGACTTGTGATGGAGTGAAAAATGTGAGGAAAATATAAACA 600
          ............................................................
  601 TTTTCTCTAGACTTTAAA 618
          ..................
(nanos3 3′UTR)
LENGTH: 801 bp
TYPE: cDNA non-coding
ORGANISM: Nile tilapia (Oreochromis Niloticus)
SEQ ID NO 169
      1 ACCAGCAGGTGGCAAGGAGCAATAAGACACTACACAGAAGGCAGGACCCTCGTTTCGTTT 60
          ............................................................
    61 AGTGTGACTTTATTTTTTCTATTTGTGTATTTATTTTAGCACTAGTGTGGTTTTGCTTTT 120
          ............................................................
  121 GTGTGCTTTTCATTTGCATGCTTTGGTTCGTTTGCTGTGTAGCTGATTAGAGTTTCTTTG 180
          ............................................................
  181 CAGCTGGTCCTGCCAGCCTAAAATACCTCAGCTGTTTGCTGTTTGGATTTGTGAGGCACT 240
          ............................................................
  241 TTCAAGAACGACTGCCAGATTTTATGTTTGGGAGGAGGTTTGAAAAAAAAAAAAGAAGAC 300
          ............................................................
  301 ATGTTTCAAAAAATTATTGTATGTTTCTTTTACATACTTTTAAAACGTGGCCAGCTGATG 360
          ............................................................
  361 TCCAGTTTCATATTTCCTGTCCATGCATTGAAGGATTATAACACTGTCAAACATTATAAG 420
          ............................................................
  421 AGATGCAGTCATAATTAATAACTCTACTAAAGCAGGTAAAGCATCATGTGACCATGTCAG 480
          ............................................................
  481 CATTTTAAATTTTTAAAAATGAGTGACTAGTTCTTGTTCCTCTGATGTGTGCAAGTAGAC 540
          ............................................................
  541 CTCTGTTCTTGAGGATAGATTATTTTATTTTGAAAACTGTAATTGTGGCTTTTCTAAAAA 600
          ............................................................
  601 TGTTAACGCCGTTGTAGCTCTTTGTCGAAAAAGTCTGAAAATTTCTCTGTGGCTATTCTT 660
          ............................................................
  661 GTGTGCTAAAAAGTTATAAATAACTAAATTGGCTAAGTTTA 801
          .........................................
(nanos3 3′UTR)
LENGTH: 903 bp
TYPE: cDNA non-coding
ORGANISM: channel catfish (Ictalurus punctatus)
SEQ ID NO 170
      1 ACCGAAAATCTGAACCCCACTCTCACACTCGCTACCAAACTGTAGGTTATTCTTTTTTTT 60
          ............................................................
    61 TTTTTTTTACTTGGGAAGGTGAACAAGAAGCTTTAGACAAAAGCTGCACAGGTACGTCAG 120
          ............................................................
  121 CGGTCCTTAAAGTCCGGTACTGTACCTGGAATGCTTTTATATGTAGGCTTCAACTATATT 180
          ............................................................
  181 TTCAAAAGGTACTAAAGATGTACCAGTTATGATTCAATTTCAGAGAAAAGCTCAAGTACA 240
          ............................................................
  241 GTTGGTGCTTTTTATCTGAGAGTGGCTGTAGAAAGTTGTAAGTCCTTTTAAAAAAAAAAA 300
          ............................................................
  301 AAAAAAAAAAATCAGCATTATATTTTTAATGTCTGCATTACTGTGCTTATTATTATGGCT 360
          ............................................................
  361 TAGAGCTGTCGGGTTTAGTTGTTTGAAACTCCGGAATGACCTGCCCTGGGTTTACAGCTG 420
          ............................................................
  421 TAACACCTGGAACGCTGTGGGTGTCAAGAGTTTTGCTTTACTAACTTTGTGTGCACTTTG 480
          ............................................................
  481 TGTATGCACTTGTGTTGTGTGTTTATTTTGATTGGTGTGTTTTGTTTTGAAGCTGATTTC 540
          ............................................................
  541 TCTAACGAGCTTGTGCTCAGGCCTCTCTGGCTCATCACAGGTGCAGCATGTTACAGGTGC 600
          ............................................................
  601 GGGTCTATGCAGGGCTTCATGATGGGACCGTGGCTCTCCGACCTGCTATTTTTCTGCTCC 660
          ............................................................
  661 ATTTTATTGTCCATTCGAAGAACTTCTGACGTGTTGTGACTTTTTAAAGTGTTTTAGACC 720
          ............................................................
  721 ATTTGGGATTTGAGTTAATATACTTTATATGCATGTAACAAGCCTCAGTGCTGCATTTGT 780
          ............................................................
  781 TTTTATATATTATATAAGACGTAAGTGTTGGACTGTTTTGGTACGAATGACCTCGTCGAT 840
          ............................................................
  841 GCCTCTGAATCTTCTGCAATTCTGTAAGTTTCAATTTCTAATATATTTAAAGTGTGAGCT 900
          ............................................................
  901 CCA 903
          ...
(nanos3 3′UTR)
LENGTH: 1000 bp
TYPE: cDNA non-coding
ORGANISM: Rainbow trout (Oncorhynchus mykiss)
SEQ ID NO 171
      1 AAGCGCTAGCTCTGGTCCAGGCCGTTACTGCGCTACCCTCTAGACCTACTAACATAGTCA 60
          ............................................................
    61 ACTTGTTGTTGCGAGATGGGTGGAACCAGAGCTAGAGAAGCGCTGGAGAGACTTCAGGCT 120
          ............................................................
  121 GTTGTTTTGCAGACTTTCTTGAGCGTCTCTAGCGCACTGTATGCGGACAATTGTAAGAGG 180
          ............................................................
  181 ATTTACGAGTGGATATTTAGCTTAGACGCAGTTTGGAACAGGCTGACAAGTTTGTAGACT 240
          ............................................................
  241 GTAATTTCCTGTTAGTCGTGTGATTTTTATTATTTATTTCCTTGACTTTTTTTCGTGTGT 300
          ............................................................
  301 TTTCAACCATTGTCGCGTATTTTTATGTATTTATGACGTGTGATTATTTGCGTGCCCGTG 360
          ............................................................
  361 CATTTATTTTCAACACATTTTGGGTTTGAGTTTTTTTTTTTTTTTTTCTTTAAAGTGGAA 420
          ............................................................
  421 ATGTTTCTGTCTGTCTGTGACCAAACTAACTGTGTCTTTACATGTTGGTGTGAGATTTTG 480
          ............................................................
  481 TAAAACCTCAACCTCTTAATGTGTCGCCTACTGTGTCGCCCTTACCAACAAAACTCTCAC 540
          ............................................................
  541 TGCAGAATTAAACGTTTATCCCACGTTTTTCCCTGCTACATTGGGGAGGAAAAAACGGAA 600
          ............................................................
  601 GTGTTGTCTTGTTTTGAGACTTGTTTTCCTGGCTTTAAAACGACATGCTTTAATCTAACT 660
          ............................................................
  661 GTACATATGCAAGTTTACGGGCCTAAAATAACTATTAAAAGAACATTCCCATTGACACCA 720
          ............................................................
  721 AGACAACTTTTGTTTTATGACGTGGTGTGCTGAGCTACTTTGTATTTTTCATTTCCCATG 780
          ............................................................
  781 CTAGCTACTATTAAGAACCGTTATACTTTAATATATCTAGAAGTGTTTTTTTATTTTAAA 840
          ............................................................
  841 TTTGACTTTTCAACCTCGATGCATCTTACATTGGCTGTACAGGACTTTAATGTTAAGTTT 900
          ............................................................
  901 AATCTCACTTTAAAGAACTGCGCTACCCCATGTTGAATAGTATGTTTGTTTATTATGATA 960
          ............................................................
  961 ACATGTTTTCTATAATAATAATAATAATAACAATATCTGC 1000
(nanos3 3′UTR)
LENGTH: 124 bp
TYPE: cDNA non-coding
ORGANISM: Japanese medaka (Oryzias latipes)
SEQ ID NO 172
      1 AAAGACTCAATCCGTTGTAAATATGTGTGTGGTTTGTTTTGAATTATTTTTAACCTAATT 60
          ............................................................
    61 TGCATGGTGTGCTGTTGTAAAATTAATATTTTCAAAACATTAAAACCAGGTTGTCTTTGG 120
          ............................................................
  121 TTGC 124
          ....
(nanos3 3′UTR)
LENGTH: 400 bp
TYPE: cDNA non-coding
SEQ ID NO 173
ORGANISM: Tetraodon nigroviridis
      1 ACCACCGGCCGGAAAACAACTTCTTTATTAGTGATTGGTGCTTTATTTGCACGGGTGTTT 60
          ............................................................
    61 GTGTGTTTTTTTTTAATGATTGTGTGGTTTGATTTGTTACTTGCATGCTCTGCACGTTTG 120
          ............................................................
  121 CCGTGTAACCTCAGTCACGCCACGTCTTTGAGAGGACAGAGACGTGGCCTTCGGCTCTCT 180
          ............................................................
  181 TGCGTTTTTAATCCCTTTGCCCGGTCACTGACCTCAGAAAAGTCATTTTATTACACCAGC 240
          ............................................................
  241 ATTTTTTAAACGTGTGGCTAGTTCTAGTCCTACTTTTGTGTTTTATTTTGCGCAATATAA 300
          ............................................................
  301 AAAGGGCTTTTCTGGAAATGTCTCAAGGAAAAAAGTGTAAATAATTCCGTATTAATATTC 360
          ............................................................
  361 TTGTGATAATTGTGTGTATTTATGTTTTAAATTTACCTCG 400
          ........................................
(dnd1 3′UTR)
LENGTH: 173 bp
TYPE: cDNA non-coding
ORGANISM: Atlantic salmon (Salmo salat)
SEQ ID NO 174
      1 TGGTGTTGAAGCACAGATCCCCTACTTTGTTTTAATTATGAAAATACTTAAATGTTTTGC 60
          ............................................................
    61 ACTCTTTTATATTTAGTAAGTAGATGCATGATTTTACTTTTTTTTTGAACCACTTTTGCA 120
          ............................................................
  121 TGTTTCTGCACCATTTAArrGTTTCTCATTATAATAAAATGAGATTTGTCAAA 173
          .....................................................
(dnd1 3′UTR)
LENGTH: 500 bp
TYPE: cDNA non-coding
ORGANISM: Atlantic cod (Gadus morhua)
SEQ ID NO 175
      1 CTTGCAGCCCTCTGGCCGGGCACGGAGGGCATGCCGAAACAGGCTTGGTGAACGCGCCCA 60
          ............................................................
    61 ACGGGACGTGTTAAACACTTATCTTGACCATCGCAGGGCGTTCCCCTTTTATACATGTTC 120
          ............................................................
  121 GAAGAAAAAAATGCTTTGGTTTTATGTTGTGCATGTTTTTATTGGTGTTGACTGTTGCAT 180
          ............................................................
  181 GCTTTATATTTGTACCTAATTTAAATCTAAATAAGCTGCTGCTTGTCATTGTAGAAGAGT 240
          ............................................................
  241 ATGCAGAGTGGAGTTTTACAGAGATCTATTGGGAGGTTTGATATGAAAGACGTCGGTTCT 300
          ............................................................
  301 GCACCTTGGTGTGGACATGTTGGTTTGATCTTGCATGATTAAATGTCTTACCTACCATCC 360
          ............................................................
  361 TTGGTGTTGCACTGCTAGTCACTTTGTATTTTATTTACATAGGACATCAAAACATACGAT 420
          ............................................................
  421 AAAAGGGAAACGAACGCAACCACGGACTGAGTGCCGGACTTGGGGTGATCGGGCCTTCTC 480
          ............................................................
  481 AGTTTCTGTCCCCCTACCCT 500
          ....................
(dnd1 3′UTR)
LENGTH: 190 bp
TYPE: cDNA non-coding
ORGANISM: Rainbow trout (Onchrorincus myskiss)
SEQ ID NO 176
      1 TAGTGTTGAAGCACAGATCCCATACTTTGTTTTAATTATGAAAATACTTCATGTTTTGCA 60
          ............................................................
    61 CTCTTTTATACTTAGTAAGTAGATGCATGATTTTACTTTTATTTTGAACCACTTTTGCAT 120
          ............................................................
  121 GTTTCTGCACCATTTAATTGTTTCTCATAATAAAATGAGATTTGTCAAATGTCAAAAAAA 180
          ............................................................
  181 AAAAAAAAAA 190
          ..........
(dnd1 3′UTR)
LENGTH: 465 bp
TYPE: cDNA non-coding
ORGANISM: Nile tilapia (Oreochromis Niloticus)
SEQ ID NO 177
      1 TGCCAGCACCATGCTAGAGGAGGCTCAGAAGGCTGTAGCCCAGCAGGTCCTGCAGAAGAT 60
          ............................................................
    61 GTACAACACTGGTCTCACACACTAAACAGCTGATGCCGTCCTGCAGTTCTGTTTCACCTT 120
          ............................................................
  121 GTTTGTGTTATGTGGTTTCATTTTCTGCATGTTTTTACTAGAGTAGCACCAAGTTTGTTT 180
          ............................................................
  181 CTCTGACTATAACTTGTGGTTTGTTTTATGCATGATTTTTACTGTACATTAGTGTTCTGT 240
          ............................................................
  241 GTTACTGGATTGGTTCTCATTTTAATTAAATGAGCTTTGAAAAGAAAGTGTCGGCGTTTC 300
          ............................................................
  301 TTTCAAATTAATGAAAGATTTAAATTAACTTAGGAAAATGGTAAAGCAGTTATTATTGTC 360
          ............................................................
  361 TCACTTCATGCTGTTATGAACCCTAGTGATTCTCATCCAGACCTTTACGTATCTTTGAAG 420
          ............................................................
  421 GTTGTGGATTGAGACTAACCCCCCTCAGTGGTTTGGCATTTTAAAC 465
(dnd1 3′UTR)
LENGTH: 273 bp
TYPE: cDNA non-coding
ORGANISM: Fugu (Takifugu rubripes)
SEQ ID NO 178
      1 GCAGGCCTGCACAGTTCAGCAACTTCTACTGCACCGCTCAATACTGTTTTATTTCATAGA 60
          ............................................................
    61 GTTGTTCAAAAAACATTGATATGTATATTTTATTGCAGTTAACTTATTTTTGCATGGTTT 120
          ............................................................
  121 TATTAGTATTTGCTGTTTGTTCTGTTCTATGCATGCTTGTTGTTGTTGTGCTCAGTTAAT 180
          ............................................................
  181 CATTTTAAGTAAATGTGACTTCAAAAGTAAATCTGATGTGTTGGATTCTTTGGGGTTGTA 240
          ............................................................
  241 AAGTGATTGTTTATACATAAAAGGATCTCAGAA 273
(dnd1 3′UTR)
LENGTH: 527 bp
TYPE: cDNA non-coding
ORGANISM: Zebrafish (Danio rerio)
SEQ ID NO 179
  241 TTGAGTTGTTTTAGTCAGCCTCATCATATTAGGATGACTGCATGTTTTCACGCTTTTCTT 300
          ............................................................
  301 TTGAGTGTTTTTCACTGTATTTCGACTTCACTTTGGTTTGCGTTTGTCACGATTGTTCTT 360
          ............................................................
  361 TTTGCATGGTGTGCTCCTTGTGTTTCCTTGTTTGATGGGTTGTACTGACTATAAATGACT 420
          ............................................................
  421 TTTGTACAATAAATAAGTTGTTCGAGAAATGTTATTCCTGCAGGTTATTGTTCACTACAG 480
          ............................................................
  481 TCTAGTACTGTATCTGATGCACTGTTAATAAACACCTGTTGAAAATA 527
          ...............................................
(dnd1 3′UTR)
LENGTH: 552 bp
TYPE: cDNA non-coding
ORGANISM: Chanel catfish (Ictalurus punctatus)
SEQ ID NO 180
      1 GGTTTGACGTCAGATGCCGTTTTGTGTGAGAATGTGATTTCCTAAAGTAGAACATGGTCC 60
          ............................................................
    61 TGGATCAGCTTTCCTGTGTTTAATTTGTTGTCAGTACAAATCTCAAAGATGTCACAGTCA 120
          ............................................................
  121 CAGATGGAGCTTGTAGATGTATTGGAGCAGTTTCACCTCCAAGCTTGTTTCGTTAGAGAG 180
          ............................................................
  181 AACTAGATGACAGGAACCTGGATTGGCAATGTCTAAACTAATTTTTTAAAAAAACTAAGT 240
          ............................................................
  241 AAGATCCAAACATAAATAAGTAACTAACAATGAAATAAATAAAATCTCTTTCACATTGCA 300
          ............................................................
  301 AAGCTACGCTAACTTTCCTGTCTAGAAACTTCCAGAGGTGGTCCTTCTTCCACTGATGAC 360
          ............................................................
  361 ATCCCAGAGCAACGATTGGCTATGTAATTACAGAAACAACGAAATGATGAAAGATTTTGA 420
          ............................................................
  421 TCAAGTAACATGTTCAACTTAAAAAAAAAAAAAGAAAAACGTTCTCCTCCAGTTTCACGT 480
          ............................................................
  481 TCAGTATGAAGATCAGAAAATATGTAGAGTTGTTTCTGCGTTTTTTTTACATGGTTGTAA 540
          ............................................................
  541 TTTTTTTTTTTT
(dnd1 3′UTR)
LENGTH: 585 bp
TYPE: cDNA non-coding
ORGANISM: Xenope (Xenopus tropicalis)
SEQ ID NO 181
      1 CTGTTTTTCTTTCTGTGATGAGGACACCACATCAGAAGAGCTTACTTTTTTATAGTTTTG 60
          ............................................................
    61 TTGTCTTCAAATGATTTTATAGTTACCACCACCCGTTTAAAGGGAAATATTTTTACATCA 120
          ............................................................
  121 GTGTATAGCTAGTTTTCTTTCCCCTTTTTTTCACTATGATGTTTGCACTTTTTITATTTC 180
          ............................................................
  181 TGTGTGTGTACATTGCGCTTAAGATTTAAAAAAACAACTTTTGTGTGTCTCTTTAAAATG 240
          ............................................................
  241 TACAGGTTACAGTATCTCTTATCCATGTACAGCACTGAACACCGTGGAGTTTCCTATGTT 300
          ............................................................
  301 TATTTTAAAATGTATGCCAAACTATAGAAGGCAAAACTTGTGGTGTAAGGGTTrCAACAT 360
          ............................................................
  361 TTTTTCATTGCACAAAAATCAGGTTTATGAATGTATCTCTAGAAATCTTAGTAGTAGTAT 420
          ............................................................
  421 TAGCCACTGGTTGGCTAATTGATCTTTTATAAATCCTTCTTTTTTTTCTTGTGTGTGGTT 480
          ............................................................
  481 TTTAAGAAAACATGTTTAAATTATGTTTTGTATTTCTAGGATCAGTGTTTGCTAGCATTT 540
          ............................................................
  541 TATATCACAGGTTCTTACTGTTTTCAGAATAAAACAATACTACTC 485
          .............................................
(Elavl2 3′UTR)
LENGTH: 2235 bp
TYPE: cDNA non-coding
ORGANISM: Atlantic salmon (Salmo salar)
SEQ ID NO 182
TCTCATCTCGGATTGTTTATAGAAAACTCTACAGAAAATGGAAAAACCTAAAATGGA
AACCTACCGACCGTAAACTTTCTACATTAGCAAAAACATCTTTGTAAATCAGTGTTA
CGAAGGGAAACTATACTTAGCGTCCAACAGTGTTTTCCTTTCCTTCATTGCTAGGCT
TTGAAACTTCTTAAAATACTTTGCGTTAAACCAGAAAAAATACTGCAGGTTTTCACG
CCTGTCTTTAAAGCTGGACCTTTTAAAATGTTACTAAAGGTTTTTTTTTGTTTTGTT
GCACATCTGCTGTAAAACAGGAAGTTTTGTAAGGCTTTGTGTAGTGATTTTTCTTTT
GTATTCTTCTGTGTCCTGATTTGCCTTGGTGCTTTTTGCGTCTTAAGTGGTTAACGA
AGTATTTATTTTTGATTATTCAGTTTAAACAAATTGTTAGTATTATGTTTATTGTAA
TATGAGTTATTGGTTGGCTGCATAATATTGCTTATTGTAAAATTTAATGGAGGAAAA
ACACAAAAAAATATATCTTAAAGCAGTACCTTGCCAAAGAGCTAAGAACCTCTTTGA
TGTGGGTTTAAAAAGCATCTATTTTTATAAAAAGACAAATTTGGAGAAACTTTTTAC
TGGACCTGGAACAAATATTTTGACTTGGATACTTTGAGAAATATCTTCATATGACAC
CTTACCAGGAAGTTTGCAGTGGTTGACATTTCTGGAGAGATTTCTGATGTAAAAGAT
ACCTTTTGAGATCTTTGTATTATCTTTCGATTCTAGAATCAATGGCAGTGATGGTTT
CATTTGTATAATCACCTGTGGGTTGTCCTATCATCCTGGCTGTTATGACTAGCAACT
CCCATTCACTTTTGATTTGGAAATGCAGACAGAAAAAATACAAAGGTTTATTTGCAA
AAGTGCATGCAAAATTATAGTGGAAATATCTTCAAGGTAAAAGGGGGGGGGGATAAA
AATCAGTTCTTCCTAAAGAATTCCCTTCAAGACAGCGCTCATGGTGGTTTGTGGTGT
ACTTACTATATCATTTTGTCTTTATATTAAACAAATAAGGGTTTTACCTACTTTGTA
TGAAAGAGGGAGAGGACAAGCTGACCAAGGCAGCTGATTAAGTAAACGAGTTTGAAT
GAAAGATGGGGAAGATTACTCCTGGGCTTAGAGCTATGTAAATAAGTCCTTTTTTTT
TATGTTGAGTATTTAGCTAGCATATTTGTTATTTTTTTGGACTTTATGGCGAAGTGC
TTTTTTTATTTGAGTAACTAGTGTGATTTATTGATTTTTCTGGGGAGATATTGCCTT
ATTTTGATTTCAGTTCGACTTTGAAAGTTCACATTCAGTTAAAATACTTGATTTGTT
GTCCTATGGACTAGCATTACAGTGTCAGATTTGTTGGTGATTTTGGTCTTTAGATGG
TCTTGCTTCTGCTATTAAGAAAACTATAGACATTTAAGTTGGTTTGTTTTATATATA
AACATTATAGATATATATTTATGTGGTAAAGAATGGATATAAACCAGTTTTTAGCTT
TCTGATTACTTTTTTTTTTTCATTCATATAGACGTAATGCATAATAACCTGTCTTAA
AAATCGTAAAAGGTGTATTGCTTTATTCACTTGAAGCGGTTCCATGACCATATCAAA
AAGGGTTGCAAGAGATTGCCGAACAACATCTTGCTTCTCTCGAACAGAGGCTGGTCA
AGCCCTTAGATGCACTGAGTACTGCACCTGCATCCTGCTTTGTCTTGAGCTCATTAC
TAGTCATACGTCTCCTTATCGAGCAGTGTGCTGTGCATTATATATATATACATTTAT
ATATTTACCAACTGCTTTTCTTATACTTTTTCTCTTTTTTTTTTTGGTTAATTGTAC
AAGTTCAACTTTGATTATAGATTAGCTGTGACACTGCTGCTGTGGGGGAAGGGGCCC
CCATTTTCTCATGCCCGGCCTCTCACTGGTCTTGATTGAGGATAACTTGACGGACCC
GAGGGGGCTCTGACTAGCTAGGCATGGCAAAATGAGCCCCCCCACACCCACTTTCAA
TTCTAAATGTGAGAATTATTATTTATTTGAAGTTGTACAGTATTACTTGGTTCCACA
GCGGTTTTGGGATAGAATATATCTTGAGTATTTAAAAAAGGATGTACATGTTATTTT
CTTTGTGTTTGGAATACTTTGTATTTTTTCATGTTCAGTACATCAATAAATACGTTG
AAGGGAAATGCA
(Elavl2 3′UTR)
LENGTH: bp
TYPE: cDNA non-coding
ORGANISM: Nile tilapia (O.Niloticus)
SEQ ID NO 183
AACTCAGATTGTTTCTAGAAAACTCACCAGAAAATGGAAACAGAAAATGGAAGCGTA
TTGACCGTAACTTTCTACATTAGTAACAAGAGCTTTGTAAATCAGTGTTGCGAAGAA
AAACGATATTTAGCGTCCAACAATGTGATCTTTTTTCCTTTTTTTTTCCTTCTCTTT
TTTCCCATTGCTACACTTTGAATCTTCTCTATACTTTAAAACAGAAAATACCTGCAG
GTCTTGATGCCTGTCATGTTGACTTCTTGCTGTCTTTACAGATGGACCATCTAAAAT
GTTACTCTAGGTTTTGTCATTTTGTTGCACATCTGCTTTGAAACAGTAAGTCTTGTA
AGGTTATGTGTAGTGATTTTTCTTTGTACTTCTGTGTCCTGATTTGCCACAGGTGCG
TTTATGCCTTCGGTGGTTAGCAAGTACTTGCGTTGAACTATTTGCGGTTCTGTTAAT
TTTGTAAGTATTCTGTTTCCTGTAATATCAGTTGGTTATTGGTTGGCTGCATAATGT
TGCTTATTGTACAATTAACAGATAAAAAGACAAAAAAAAAAGATTCTTAAAGCAGTA
CCTTGCCAAAGAGCTAAGAACCTCTTTGATGTGGGTTTAAAAGCATCTATTTTTATA
AAAAGAAAAATTTGGAGAAACTTTTTACTGGACCTGGAACAAAATATTTTGACTTGG
ATACTTTGAGAAATATCTTCATATGACACCTTGTGAGCTTTTGAACTTTACAAGAAA
GTTTGCAGTGGTTGAAATTTCTGGAGAGATTTATGATGTAAAAGATACCTTTTGAGA
TCTTTGTATTACCTTTAGATTATAGAATCAGTGGCAGTGCTGGTTTCATTTGTAAAA
TCATCTGTGGGTACCCCCCTCCCCTCAGTCGTCTGGTCGTTACGGCTAGCGACTCGC
TTTCCGGTCTGATTTGGAAACGGACAAAACTTCAAAGGTTGATCTGCAAAAAGTGCA
TGAAAAATTAAAAACATGGAGATATAAAGGTAAATGGGGGGATTTAAAAAAAGGGAA
AAAAGAAAAATCAGTTCTTCCTCTAAGATTCCCTTCAGATGGAGCTCATGGTGTTTT
GTGGTGTATCTACAATATCATTAGACTGATTTTTGTCTTAATATCAACCGATGAGGG
TTTTTACATACTTTGTATGAAAGATTGAAGACAAGCCAGTGAAGGCAGCAGCATCAA
AAAAAACATCTAGTGTGACAAATAGAAGGGTTCCTCCTGAGCTTTGAGCTGTGTAAA
TAAGTCCTTTTTTATGTTGAGTACTTGGCAGACTTTGGTTTTCGTTTGGACTTCATT
GAAAAGTGTGATTATTATATACAACTTGATTTTTCTTTTCAGGACTGTGTAAGGTCT
TTTTTTGTTTTTGGATCTTTATTTATTTTCAGTTTCTCTTAAGTTAAAATACTTGAG
TTGTTGTCCTATGGACTAGCATTAGTGCATCGAATTTGTTGGTTGTTAGGTCTGTAG
ATAGTCTTGCTTCGTTAAAAAAAAAAAAAGGTATTAAGAAACTATAGACATTTGTTT
TTGTTTTGTTTTTTTTATATATAAACATTATAGATATATATTTATGTGGTAAAGAAT
GGATATAAACCACAGTTTTGAACTATTTGATTACTTTTTTCATACTCATATCTATAT
ATATATATAAATATATACGTAATGCATATAACCTGTCTTTAAAATCGTAAAAAGGTG
TATATTGCTTTATTCACTTTGGGGAAGGGCGGTGAAGCGGTTCCATTAACAATAGCA
AAGTTGGTTGCAGAAGTTTGTCAACATCTAGCTCATCTCGAACACACGAACGGAGGC
TGGTCGAGCCTTAGATGCACTGAATACTGCACCTGCATCCTGCTTGGTATTTCAACC
GATTATTAGTCATGCTTCCCCTTAAACGAGCAGTGTGCTATGCATTATATAATTATC
TTTGCAACTGCTTTTTCTTGTTTATCTATTCCTTCTTTGTGTTACTTGTACAAGTTA
AACTTTAAGTCTAGATGAGTTGTGATACTTGCTGCTGTAGGGAAGAGACAACATTTG
TCATGCCTGACCTGCCACTGCTGAGAATAAGTTTTGTTTTTCCTTTATGTAACCGCT
TGATGATTTTCTTTTTTTTTCTTTTTTTTTTGGGGTCTTGGGAAATTGTGCTGCAGG
TCTGGCATGAGGAAATGTTTCCTCCCATCCCTCTTTTCTCAATTCCTAATATGAGAG
TGATTATTTATTTGAAGTTGTATAGTCTGACTTGGTTCACAGCATTTTTGGAATAGA
ATCTTTTTGTTAAGTATTTAAAAGGATGTACATGTTCTTTACTTTGTGTTTGGATAC
TTTTGGATTTATTTTATTTTTTTCCATGTTCAGTACATCAATAAATAAGTTGAAGGG
CAA
(Elavl2 3′UTR)
LENGTH: 465 bp
TYPE: cDNA non-coding
ORGANISM: Zebrafish (Danio rerio)
SEQ ID NO 184
GGAGTGCCGACGTGCAGCGCTTTGCAAACGTGACTTTGCAATAATGACGGGACGCGT
ATATTATATTCTTTTCTTTTCTTAAAGTACTTTATCATTATTTTAAGCATTTGTTTA
ATGATTTACGTAGGATATAACAGCTGACTGTTTAAGTGTTTGTTTTTGGCGTGTGAT
CCTGAGGGCGTGATGCTGAGATGGAGAGCGCTGGTGTTCCCGTCTCTCCTCATGGGC
TTCTGCGGTGCA
GTCCACTGCATAATCTTTGTGCATGAATCTTTAGTTAAACCATTTCAGTTCGCTTTC
TGTGCTAAAGGCTCTTTGTGTTGAAAGATATATCTTTATGTTAAGCATTTAAATGAG
ACAAGATGTACGTGTTGTTTTGTGTTTGAAATTTGGGATTTGTTTTTGTTTTTTATT
GTTCAGTACATCAATAAATACTTTGAAAGGAAAAAAAAAAAAAAAAAAAAAAAA
LENGTH: 1264 bp
TYPE: cDNA non-coding
(Elavl2 3′UTR)
ORGANISM: Catfish (Ictalurus punctatus)
SEQ ID NO 185
GAATGAAGTGTTTGAAGGGAGAAGAGCTCCTGAGTTAATACCITACTGTAAATAAGT
ACTTTACGTTGAGTAATGTGTATCGTTTATTTTTTTCCCCAAGCAAGTGTTTTTTTG
TTTTGTTTTTTTTTTTGTTAAAGTACGTAGGGTAATTTTTGTTAGCTAATAATTTGG
TTTGCCTCTGAGAGTTGTTTAGTTGAGAGACTTTGTTTGATGCATTATTACTGTATC
AGATTTGTTGGTGGTTTTGTCCGTAGATAGTCTTGCTTCTGCGAAATGCTAGGCATT
TGAGTTTTTTTTTTTTTGTTGTTGTTTTTGTTTACTTGTTTTTTTTTTTCTTCCTTC
CTTCAGGTTTTTCTTTTCTTTCTTTAATCTATATATTATAAGTATTAAATATATTTG
TGGTTAAAAATTGAAGAGCCACAGTTTTGAACAAATACTTATTTGATTTAGTTTTTG
TATTCATGTTACACTCGATACATAATATAACCCATTTTAAAATAAAAAAAAAATAAA
ATAAAAAGTGTATATTGCTTAATCTTCATGAGGGGAGCCTGACTAGGCTTTTCCATG
ACCGTAGCAACACAATGGCGTGTTTTATTCTCCACTTACTGAAGGAAAGAGCCTTTA
TCAGTGCAGTTCAGGCTCCCCGGATGTGTGGCAGTGTGCTATGCATTACATTTATTT
TCCTTCTTTCTCTTTTTGGCTGTTTATTATTATTATTGTTTTTGTTTTTITGTTTGG
TTATATTTTCATTGATGAACTGTGGCTTTGTGCTGCTTGGGGACAGGGAGAGCTTTT
CATGTCATGTAGGGTTTAGTTTTCTGTTTAACTTTTTTTTGTTTTTTTTTTTTTCTT
CCCATAAACCACTGCAAGGCAGAGACTCTTCAGCTGCTAGTGTTTTAAACACGGCTA
AATTTGAGCTCGGATCCGTCTGTGGCGTGAAAAGCCTCCGTGTCCTGTCTGTAGTCT
CAGTTCTGTAGTGTGCATTATTTATTTCAAGTTGTACAGTATAACCTTATTCACAGC
TGAGTTTTGAATTTTGGGGTATATAATCTTTTTGTTCAGTATTTAAAAGAAATGGTA
TTACTAGATGTACATGTTCTTTTGCCTTCTTTTTTTTTTTTCTTTTTTTTTTTTCCT
CTTGTGTTTGGAATACTTTGTATTTTTTCATGTTCGGTACATCAATAAATAAATTGA
AGGGAGCTGTGATAGAATTGCTCTTCACTGTGTTTTATTGCACTTCCTTTCCCTTAG
TTTATTTTCC
(Elavl2 3′UTR)
LENGTH: 2176 bp
TYPE: cDNA non-coding
ORGANISM: Medaka (Oryzias latipes)
SEQ ID NO 186
AAGTAAGGGGGGGAAAAAAACCACTGAGATTGTTCTTAAAAAACAAAAAAACTCACC
AGAAAGAAGTGGAACATGGGAGCTTTTGACCGTAACTTTCTACATTAGTAAGAACAG
CTTTGTAATCGATATTTAGCCTCCAACATTGTGACTTTTTGTTTTCGTTGCTTTCAG
TCCTTAGTTTCAAGCAAAAAAGTAGTGCAGGTCTGAAGGCCTGTCGTGTTGCCGATG
GATCACCTGAAATGTTCTGGGTTTTGTCGTTTAGTTGCTCTTTGATTTGACCCAGTG
AGTCTTGTACGGCTGTGACTTTTTCTTTCTCTTCTGCTGTGTCCCCCAGTAGCTGCA
TCAGGCTTTTAGTGGTAAGCTAGTACTTCTGTTGGAGACTTTTTTTTTTTTTTTCCT
CTTTCCGTTCTGTTGGTTTCTCGTAATGCGTTGGTTATCGGTTGACTGCATCCAGTT
GCTTATTGTAAAACTTAGCCGATTAAAAAATAAAAAATACATACATAAAGGGAAAAA
GACAAAAAAAATTCTTAAAGCAGTACCTTGCCAAAGAGCTAAGAACCTCTTTGATGT
GGGTTTAAAAAGCATCTATTTTTATAAACAGAAAAATTTGGAGAAACTTTTTACTGG
ACCTGGAACAAAAAAAAAAATATTTTGACTTGGATACTTTGAGAAATATCTTCATAT
GACACCTTGTGAGCTTTTGAACTTTACAAGAAAGTTTGCAGTGGTCGACATTTCTGG
AGAGATGTTATGATGTAAAAGATACCTTTTGAGATCTTTGTATTACCTTTAGATTCT
AGAATCAGTGGCAGTGCTGGTTTCATTCGTCAAATCGTCTGTGGGTTCTCCTCCATC
CCGGTCGTCCGCTCGACCCCCAACGGTGCACTTTTCCCCCCTCCAGACAAAAGCGAA
CAGTGCATGCTAAACGACCGCTAGAGGAGATCTTCATGGGAATTAAATCAGTTCTTC
CTTTAAGATTCCCTTCAGACGGAGCCGCGGTGGTTTGTGGCGCACCCACGATGTATC
GTGAGACCAATCTTGGCTTGAAATGAATCATTTGTGGTTTTTAAATAGTTTGTACGA
CAGACTGACGGAGGCAGAGACAAAAAAAAACCCAACAAAAGCTCTGAAGAATCGGAT
GACTCCTAAGCGTTGAGCTGTGTAAATAAATCTTTTGTTTGTTTTGTTTATGTTGTG
TATTGGACACTTTTCTTTACAGTTGGATTCCTGGGTATGGAAAGTGATGATTTTTTT
TTTCTTTCTTTCTTTCTTGGAGTACGTGAGGTGTTTACTGTTTTTGAGTTGGCAAAA
CCTTAATTTATATTTTTGGTTTTCCTATGGACGAACACTGAAGTGCATCAAATTTGT
TGGTGGTTTGGTCTGTAGTTAGTCTTTGTTTGTTACAAAAAAAAGTATTCAACTATA
GACAGTTTTTTTTTAATATATAAACATTATAGATATATATTTATGTGGTGAAGAATG
GATATAAACCACATTTCTGGATTTTTTTTTTCATACTATGTAAAAACAATGCATATA
ACCTGTCTTTAAAAATCGTAAAAAAGGTGTCTATTGCTTTAGGAAGGACGGTGAAGC
AGAAACGAATAGCAGAAGTGATTGCAACAGTTGTTGGCGGCGGTGGGCGGAGCCTAG
CAGTCTCTGAATCCTGCATCCTTTCTGCTATTTCAACCCAGTCATGCTTCCCTTACT
GAGCAGTGTGCTATGCATTCAATGATCCTTTGCAACTGCTTTTTCTTTAGAGAACTT
CTTTGTGTTCCTTGTAAAAGTTCCTCTTTAAGTCTAGATGAGTTGTGATACTTGCTG
CTGTAGAGGAGGGTGGGGTGGGGGGGCATGCTTTTTACGCCTGACCCATCTGGGCTT
TTTTTGTTTTTTTAAAAATTCATCGATTTTTTTTTTTTTTTTTTTAATAGATTTGAT
CGTCCGGCGTGAAAACGTGTCCCCCTACGACCCCGGCCCCCCCATTCTTCTGATCTC
TATTCTTTAGTGAGAATGATTATTTATTTGAAGTTGTATAGTCCGACTCGGTTCACA
GCGTTTTTGGGAATAGAATATTTTTGTTGACTATTTAAAAGGATGTACATGTTCTTT
ACTTTGTGTTTGGATACTTTGACTTTTTTCAATGTTCAGTACATCAATAAATATGTT
TGAAGGGCAA
(Elavl2 3′UTR)
LENGTH: 485 bp
TYPE: cDNA non-coding
ORGANISM: Xenopus (X.tropicalis)
SEQ ID NO 187
CAAGTTAACTTCTCCCATTATATACACACATGCAACAAAGGCAAGTTGATAAACTTT
ATACTTTTTGAAATTGTCTTTGCAAGTAAGTGTTACACCAAAGTGTGTGGGTTTGAG
GGAGCCACGGCAAAATGAGATCATCATTTAGCATCTTTAGAATATGTGAGATTGTTA
TTGTTGGATTTTGGATTTTTATTTTATGTTTGTGTATGGACCTTGGGTAACAGGGTT
TTTACCGGTCATATTACATTATGCCTTCTATTGAGGGGGATTTTTTTTAGATATTTC
AGCAGTGGGAAGACGATTTATGTTCCGTTTTTTTACATTCTTACCTTCAAACCTGAG
TTAAAGCTTTGGAAGGATTTTTGTTAAAATGGTTAAGTATATGAAAGTTATTTCATT
TTTATTATAATTTATAAATGTGTAAAACCATATTTATTTTGCGGTTATTTAGGGAAT
TGGAGGACTCCACTATAAAAAAAAAAAAA
(nanos3 3′UTR-Del 8 nt)
LENGTH: 793 bp
TYPE: cDNA non-coding
ORGANISM: Nile tilapia (Oreochromis Niloticus)
SEQ ID NO 188
      1 ACCAGCAGGTGGCAAGGAGCAATAAGACACTACACAGAAGGCAGGACCCTCGTTTCGTTT 60
          ............................................................
    61 AGTGTGACTTTATTTTTTCTATTTGTGTATTTATTTTAGCACTAGTGTGGTTTTGCTTTT 120
          ............................................................
  121 GTGTGCTTTTCATTTGCATGCTTTGGTTCGTTTGCTGTGTAGCTGATTAGAGTTTCTTTG 180
          ............................................................
  181 CAGCTGGTCCTGCCAGCCTAAAATACCTCAGCTGTTTGCTGTTTGGATTTGTGAGGCACT 240
          ............................................................
  241 TTCAAGAACGACTGCCAGATTTGGAGGAGGTTTGAAAAAAAAAAAAGAAGACATGTTTCA 300
          ............................................................
  301 AAAAATTATTGTATGTTTCTTTTACATACTTTTAAAACGTGGCCAGCTGATGTCCAGTTT 360
          ............................................................
  361 CATATTTCCTGTCCATGCATTGAAGGATTATAACACTGTCAAACATTATAAGAGATGCAG 420
          ............................................................
  421 TCATAATTAATAACTCTACTAAAGCAGGTAAAGCATCATGTGACCATGTCAGAGATGCAG 480
          ............................................................
  481 ATTTTTAAAAATGAGTGACTAGTTCTTGTTCCTCTGATGTGTGCAAGTAGACCTCTGTTC 540
          ............................................................
  541 TTGAGGATAGATTATTTTATTTTGAAAACTGTAATTGTGGCTTTTCTAAAAATGTTAACG 600
          ............................................................
  601 CCGTTGTAGCTCTTTGTCGAAAAAGTCTGAAAATTTCTCTGTGGCTATTCTTGTGTGCTA 660
          ............................................................
  661 AAAAGTTATAAATAACTAAATTGGCTAAGTTTA 801
          .................................
(nanos3 3′UTR-Del 32 nt)
LENGTH: 769 bp
TYPE: cDNA non-coding
ORGANISM: Nile tilapia (Oreochromis Niloticus)
SEQ ID NO 189
      1 ACCAGCAGGTGGCAAGGAGCAATAAGACACTACACAGAAGGCAGGACCCTCGTTTCGTTT 60
          ............................................................
    61 AGTGTGACTTTATTTTTTCTATTTGTGTATTTATTTTAGCACTAGTGTGGTTTTGCTTTT 120
          ............................................................
  121 GTGTGCTTTTCATTTGCATGCTTTGGTTCGTTTGCTGTGTAGCTGATTAGAGTTTCTTTG 180
          ............................................................
  181 CAGCTGGTCCTGCCAGCCTAAAATACCTCAGCTGTTTGCTGTTTGGATTTGTGAGGCACT 240
          ............................................................
  241 TTGGAGGTTTGAAAAAAAAAAAAGAAGACATGTTTCAAAAAATTATTGTATGTTTCTTTT 300
          ............................................................
  301 ACATACTTTTAAAACGTGGCCAGCTGATGTCCAGTTTCATATTTCCTGTCCATGCATTGA 360
          ............................................................
  361 AGGATTATAACACTGTCAAACATTATAAGAGATGCAGTCATAATTAATAACTCTACTAAA 420
          ............................................................
  421 GCAGGTAAAGCATCATGTGACCATGTCAGCATTTTAAATTTTTAAAAATGAGTGACTAGT 480
          ............................................................
  481 TCTTGTTCCTCTGATGTGTGCAAGTAGACCTCTGTTCTTGAGGATAGATTATTTTATTTT 540
          ............................................................
  541 GAAAACTGTAATTGTGGCTTTTCTAAAAATGTTAACGCCGTTGTAGCTCTTTGTCGAAAA 600
          ............................................................
  601 AGTCTGAAAATTTCTCTGTGGCTATTCTTGTGTGCTAAAAAGTTATAAATAACTAAATTG 660
          ............................................................
  661 GCTAAGTTTA 769
          ..........
(dnd1 3′UTR-edited motif 1)
LENGTH: 465 bp
TYPE: cDNA non-coding
ORGANISM: Nile tilapia (Oreochromis Niloticus)
SEQ ID NO 190
      1 TGCCAGCACCATGCTAGAGGAGGCTCAGAAGGCTGTAGCCCAGCAGGTCCTGCAGAAGAT 60
          ............................................................
    61 GTACAACACTGGTCTCACACACTAAACAGCTGATGCCGTCCTGCAGTTCTGTTTCACCTT 120
          ............................................................
  121 GTTTGTGTTATGTGGTTTCATTAACTGATTATAATTACTAGAGTAGCACCAAGTTTGTTT 180
          ............................................................
  181 CTCTGACTATAACTTGTGGTTTGTTTTATGCATGATTTTTACTGTACATTAGTGTTCTGT 240
          ............................................................
  241 GTTACTGGATTGGTTCTCATTTTAATTAAATGAGCTTTGAAAAGAAAGTGTCGGCGTTTC 300
          ............................................................
  301 TTTCAAATTAATGAAAGATTTAAATTAACTTAGGAAAATGGTAAAGCAGTTATTATTGTC 360
          ............................................................
  361 TCACTTCATGCTGTTATGAACCCTAGTGATTCTCATCCAGACCTTTACGTATCTTTGAAG 420
          ............................................................
  421 GTTGTGGATTGAGACTAACCCCCCTCAGTGGTTTGGCATTTTAAAC 465
          ..............................................
```

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

What is claimed is:

1. A method of generating a sterile fish, crustacean, or mollusk, comprising the steps of:
breeding (i) a fertile hemizygous mutated female fish, crustacean, or mollusk with (ii) a fertile hemizygous mutated male fish, crustacean, or mollusk,
selecting a female progenitor that is homozygous by genotypic selection, and
breeding the homozygous female progenitor to produce the sterile fish, crustacean, or mollusk;
wherein the mutation disrupts the maternal-effect of a primordial germ cell (PGC) development gene, and
wherein the mutation that disrupts the maternal-effect of a PGC development gene does not impair the viability, sex determination, fertility, or a combination thereof, of a homozygous progenitor.

2. The method of claim 1, wherein the mutation comprises:
a mutation in a cis-acting 5′ or 3′ UTR regulatory sequence of the PGC development gene;
a mutation in a gene encoding an RNA binding protein involved in the post-transcriptional regulation of the PGC development gene;
a mutation in a gene involved in transport or formation of germ plasm;
a mutation in a gene involved in germ cell specification, maintenance, or migration; or
a combination thereof.

3. The method of claim 2, wherein the gene encoding an RNA binding protein involved in the post-transcriptional regulation of the PGC development gene is: Hnrnpab, Elavl1, Ptbp1a, Igf2bp3, Tia1, TIAR, Rbpms42, Rbpms24, KHSRP, or DHX9.

4. The method of claim 2, wherein the gene involved in transport or formation of germ plasm encodes a multi-tudor domain-containing protein, a kinesin-like protein, or an adaptor protein.

5. The method of claim 4, wherein the multi-tudor domain-containing protein is Tdrd6a.

6. The method of claim 4, wherein the adaptor protein is hook2.

7. The method of claim 2, wherein the gene involved in germ cell specification, maintenance, or migration is a gene expressing non-coding RNA.

8. The method of claim 7, wherein the non-coding RNA is miR202-5p.

9. The method of claim 2, wherein the mutation in a cis-acting 5′ or 3′ UTR regulatory sequence disrupts the maternal activity of the PGC development gene, and does not disrupt the function of the PGC development gene during later stages of development.

10. The method of claim 9, wherein the PGC development gene is nanos3, dnd1, Elavl2, or a piwi-like gene.

11. A fertile homozygous mutated female fish, crustacean, or mollusk for producing a sterile fish, crustacean, or mollusk, wherein the mutation disrupts the post-transcriptional regulation of a primordial germ cell (PGC) development gene to reduce the maternal-effect of the PGC development gene, and wherein the mutation that disrupts the post-transcriptional regulation of a PGC development gene does not impair somatic function of the gene.

12. The fertile homozygous mutated female fish, crustacean, or mollusk of claim 11, wherein the mutation comprises:
a mutation in a cis-acting 5′ or 3′ UTR regulatory sequence of the PGC development gene;
a mutation in a gene encoding an RNA binding protein involved in the post-transcriptional regulation of the PGC development gene;
a mutation in a gene involved in transport or formation of germ plasm;
a mutation in a gene involved in germ cell specification, maintenance, or migration; or
a combination thereof.

13. The fertile homozygous mutated female fish, crustacean, or mollusk of claim 12, wherein the gene encoding an RNA binding protein involved in the post-transcriptional regulation of the PGC development gene is: Hnrnpab, Elavl1, Ptbp1a, Igf2bp3, Tia1, TIAR, Rbpms42, Rbpms24, KHSRP, or DHX9.

14. The fertile homozygous mutated female fish, crustacean, or mollusk of claim 12, wherein the gene involved in transport or formation of germ plasm encodes a multi-tudor domain-containing protein, a kinesin-like protein, or an adaptor protein.

15. The fertile homozygous mutated female fish, crustacean, or mollusk of claim 14, wherein the multi-tudor domain-containing protein is Tdrd6a.

16. The fertile homozygous mutated female fish, crustacean, or mollusk of claim 14, wherein the adaptor protein is hook2.

17. The fertile homozygous mutated female fish, crustacean, or mollusk of claim 12, wherein the gene involved in germ cell specification, maintenance, or migration is a gene expressing non-coding RNA.

18. The fertile homozygous mutated female fish, crustacean, or mollusk of claim 17, wherein the non-coding RNA is miR202-5p.

19. The fertile homozygous mutated female fish, crustacean, or mollusk of claim 12, wherein the mutation in a cis-acting 5′ or 3′ UTR regulatory sequence disrupts the maternal activity of the PGC development gene, and does not disrupt the function of the PGC development gene during later stages of development.

20. The fertile homozygous mutated female fish, crustacean, or mollusk of claim 19, wherein the PGC development gene is nanos3, dnd1, Elavl2, or a piwi-like gene.

21. A method of breeding a fertile homozygous mutated female fish, crustacean, or mollusk to generate a sterile fish, crustacean, or mollusk, comprising the steps of:
breeding a fertile homozygous mutated female fish, crustacean, or mollusk with a wild-type male fish, crustacean, or mollusk, a hemizygous mutated male fish, crustacean, or mollusk, or a homozygous mutated male fish, crustacean, or mollusk to produce the sterile fish, crustacean, or mollusk,
wherein the mutation disrupts the maternal-effect of a primordial germ cell (PGC) development gene, and
wherein the mutation that disrupts the maternal-effect of a PGC development gene does not impair the viability, sex determination, fertility, or a combination thereof, of a homozygous progenitor.

22. The method of claim 21, wherein the mutation comprises:
a mutation in a cis-acting 5′ or 3′ UTR regulatory sequence of the PGC development gene;
a mutation in a gene encoding an RNA binding protein involved in the post-transcriptional regulation of the PGC development gene;
a mutation in a gene involved in transport or formation of germ plasm;
a mutation in a gene involved in germ cell specification, maintenance, or migration; or
a combination thereof.

23. The method of claim 22, wherein the gene encoding an RNA binding protein involved in the post-transcriptional regulation of the PGC development gene is: Hnrnpab, Elavl1, Ptbp1a, Igf2bp3, Tia1, TIAR, Rbpms42, Rbpms24, KHSRP, or DHX9.

24. The method of claim 22, wherein the gene involved in transport or formation of germ plasm encodes a multi-tudor domain-containing protein, a kinesin-like protein, or an adaptor protein.

25. The method of claim 24, wherein the multi-tudor domain-containing protein is Tdrd6a.

26. The method of claim 24, wherein the adaptor protein is hook2.

27. The method of claim 22, wherein the gene involved in germ cell specification, maintenance, or migration is a gene expressing non-coding RNA.

28. The method of claim 27, wherein the non-coding RNA is miR202-5p.

29. The method of claim 22, wherein the mutation in a cis-acting 5′ or 3′ UTR regulatory sequence disrupts the maternal activity of the PGC development gene, and does not disrupt the function of the PGC development gene during later stages of development.

30. The method of claim 29, wherein the PGC development gene is nanos3, dnd1, or a piwi-like gene.

31. A method of making a fertile homozygous mutated female fish, crustacean, or mollusk that generates a sterile fish, crustacean, or mollusk, comprising the steps of:
breeding (i) a fertile hemizygous mutated female fish, crustacean, or mollusk with (ii) a fertile hemizygous mutated male fish, crustacean, or mollusk or a homozygous mutated male fish male fish, crustacean, or mollusk, and
selecting a female progenitor that is homozygous by genotypic selection,
wherein the mutation disrupts the maternal-effect of a primordial germ cell (PGC) development gene, and
wherein the mutation that disrupts the maternal-effect of a PGC development gene does not impair the viability, sex determination, fertility, or a combination thereof, of a homozygous progenitor.

32. The method of claim 31, wherein the mutation comprises:
a mutation in a cis-acting 5′ or 3′ UTR regulatory sequence of the PGC development gene;
a mutation in a gene encoding an RNA binding protein involved in the post-transcriptional regulation of the PGC development gene;
a mutation in a gene involved in transport or formation of germ plasm;
a mutation in a gene involved in germ cell specification, maintenance, or migration; or
a combination thereof.

33. The method of claim 32, wherein the gene encoding an RNA binding protein involved in the post-transcriptional regulation of the PGC development gene is: Hnrnpab, Elavl1, Ptbp1a, Igf2bp3, Tia1, TIAR, Rbpms42, Rbpms24, KHSRP, or DHX9.

34. The method of claim 32, wherein the gene involved in transport or formation of germ plasm encodes a multi-tudor domain-containing protein, a kinesin-like protein, or an adaptor protein.

35. The method of claim 34, wherein the multi-tudor domain-containing protein is Tdrd6a.

36. The method of claim 34, wherein the adaptor protein is hook2.

37. The method of claim 32, wherein the gene involved in germ cell specification, maintenance, or migration is a gene expressing non-coding RNA.

38. The method of claim 37, wherein the non-coding RNA is miR202-5p.

39. The method of claim 32, wherein the mutation in a cis-acting 5′ or 3′ UTR regulatory sequence disrupts the maternal activity of the PGC development gene, and does not disrupt the function of the PGC development gene during later stages of development.

40. The method of claim 39, wherein the PGC development gene is nanos3, dnd1, Elm/12 or a piwi-like gene.
